(19) United States
(12) Patent Application Publication
(10) Pub. No.: US 2026/0167998 A1
(43) Pub. Date: Jun. 18, 2026

(54) MICROBIAL PRODUCTION OF MOGROL AND MOGROSIDES

(71) Applicant: Manus Bio Inc., Waltham, MA (US)

(72) Inventors: Michelle N. GOETTGE, Waltham, MA (US); Ryan N. Philippe, Waltham, MA (US); Ajikumar Parayil Kumaran, Waltham, MA (US); Christine Nicole S. Santos, Waltham, MA (US); Jason Eric Donald, Waltham, MA (US); Christopher Frei, Waltham, MA (US); Aaron Love, Waltham, MA (US); Kaitlin Chambers, Waltham, MA (US); Joyce Samson, Waltham, MA (US); Christopher Toomey, Waltham, MA (US)

(21) Appl. No.: 19/373,926

(22) Filed: Oct. 30, 2025

Related U.S. Application Data
(63) Continuation of application No. 17/785,488, filed on Jun. 15, 2022, now Pat. No. 12,480,146.

(51) Int. Cl. C12P 17/02; C12N 9/02; C12N 9/14; C12P 5/00; C12P 19/18; C12P 19/56

(52) U.S. Cl. CPC C12P 17/02; C12N 9/0077; C12N 9/14; C12P 5/007; C12P 19/18; C12P 19/56; C12Y 303/0201; C12Y 504/99008

(57) ABSTRACT

The present invention provides host cells and methods for making mogrol glycosides, including Mogroside V (Mog. V), Mogroside VI (Mog. VI), Iso-Mogroside V (Isomog. V), siamenoside, and glycosylation products that are minor products in Siraitia grosvenorii. The invention provides engineered enzymes and engineered host cells for producing mogrol glycosylation products, such as Mog. V, Mog. VI, and Isomog. V, at high purity and/or yield. The present technology further provides methods of making products containing mogrol glycosides, such as Mog. V, Mog. VI, and Isomog. V, including food products, beverages, oral care products, sweeteners, and flavoring products.

### CROSS-REFERENCE TO RELATED APPLICATIONS

This application claims priority to, and the benefit of, U.S. Provisional Application No. 63/085,557 filed Sep. 30, 2020, U.S. Provisional Application No. 63/075,631 filed Sep. 8, 2020, and U.S. Provisional Application No. 62/948,657 filed Dec. 16, 2019, the disclosures of which are hereby incorporated by reference in their entireties.

### DESCRIPTION OF THE TEXT FILE SUBMITTED ELECTRONICALLY

The instant application contains a sequence listing, which has been submitted in XML format via EFS-Web. The contents of the XML copy named “MAN-025C1 107590-5025_Sequence_Listing,” which was created on Jan. 13, 2026 and is 297,730 bytes in size, the contents of which are incorporated herein by reference in their entirety.

### BACKGROUND

Mogrosides are triterpene-derived specialized secondary metabolites found in the fruit of the Cucurbitaceae family plant Siraitia grosvenorii (a/k/a monkfruit or Luo Han Guo). Their biosynthesis in fruit involves a number of consecutive glycosylations of the aglycone mogrol. The food industry is increasing its use of mogroside fruit extract as a natural non-sugar food sweetener. For example, mogroside V (Mog. V) has a sweetening capacity that is ˜250 times that of sucrose (Kasai et al., Agric Biol Chem (1989)). Moreover, additional health benefits of mogrosides have been revealed in recent studies (Li et al., Chin J Nat Med (2014)).

A variety of factors are promoting a surge in interest in research and commercialization of the mogrosides and monkfruit in general, including, for example, the explosion in popularity of and demand for natural sweeteners; the difficulties in scalable sourcing of other promising natural sweeteners such as rebaudioside M (RebM) from the Stevia plant; the superior taste performance of Mog. V relative to other natural and artificial sweetener products on the market; and the medicinal potential of the plant and fruit.

Purified Mog. V has been approved as a high-intensity sweetening agent in Japan (Jakinovich et al., Journal of Natural Products (1990)) and the extract has gained GRAS status in the USA as a non-nutritive sweetener and flavor enhancer (GRAS 522). Extraction of mogrosides from the fruit can yield a product of varying degrees of purity, often accompanied by undesirable aftertaste. In addition, yields of mogroside from cultivated fruit are limited due to low plant yields and particular cultivation requirements of the plant. Mogrosides are present at about 1% in the fresh fruit and about 4% in the dried fruit (Li H B, et al., 2006). Mog. V is the main component, with a content of 0.5% to 1.4% in the dried fruit. Moreover, purification difficulties limit purity for Mog. V, with commercial products from plant extracts being standardized to about 50% Mog. V. It is highly likely that a pure Mog. V product will achieve greater commercial success than the blend, since it is less likely to have off flavors, will be easier to formulate into products, and has good solubility potential. It is therefore advantageous to be able to produce sweet mogroside compounds via biotechnological processes.

### SUMMARY

The present invention, in various aspects and embodiments, provides enzymes (including engineered enzymes), microbial strains, and methods for making mogrol and mogrol glycosides (“mogrosides”) using recombinant microbial processes. In other aspects, the invention provides methods for making products, including foods, beverages, and sweeteners (among others), by incorporating the mogrol glycosides produced according to the present disclosure.

In various aspects, the invention provides microbial strains and methods for making mogrol or mogrol glycoside(s). The invention involves a recombinant microbial host cell expressing a heterologous enzyme pathway catalyzing the conversion of isopentenyl pyrophosphate (IPP) and/or dimethylallyl pyrophosphate (DMAPP) to mogrol or mogrol glycoside(s). The microbial host cell in various embodiments may be prokaryotic (e.g., E. coli) or eukaryotic (e.g., yeast).

In various embodiments, the heterologous enzyme pathway comprises a farnesyl diphosphate synthase (FPPS) and a squalene synthase (SQS), which are recombinantly expressed. In various embodiments, the SQS comprises an amino acid sequence that is at least 70% identical to an amino acid sequence selected from SEQ ID NOS: 2 to 16, 166, and 167. In some embodiments, the SQS comprises an amino acid sequence that is at least 70% identical to SQS (SEQ ID NO: 11), which has high activity in E. coli.

In some embodiments, the host cell expresses one or more enzymes that produce mogrol from squalene. For example, the host cell may express one or more squalene epoxidase (SQE) enzymes, one or more triterpenoid cyclases, an epoxide hydrolase (EPH), one or more cytochrome P450 oxidase enzymes (CYP450), a non-heme iron-dependent oxygenases, and a cytochrome P450 reductases (CPR). As shown in FIG. 2, the heterologous pathway can proceed through several routes to mogrol, which may involve one or two epoxidations of the core substrate.

In some embodiments, the heterologous enzyme pathway comprises two squalene epoxidase (SQE) enzymes. For example, the heterologous enzyme pathway may comprise an SQE that produces 2,3-oxidosqualene. In some embodiments, the SQE will produce 2,3;22,23-dioxidosqualene, and this conversion can be catalyzed by the same SQE enzyme, or an enzyme that differs in amino acid sequence by at least one amino acid modification. For example, the squalene epoxidase enzymes may include at least two SQE enzymes each comprising (independently) an amino acid sequence that is at least 70% identical to any one of SEQ ID NOS: 17 to 39, 168 to 170, and 177 to 183.

In some embodiments, at least one SQE comprises an amino acid sequence that is at least 70% identical to SEQ ID NO: 39.

In some embodiments, the host cell comprises two squalene epoxidase enzymes that each comprise an amino acid sequence that is at least 70% identical to squalene epoxidase (SEQ ID NO: 39). For example, one of the SQE enzymes may have one or more amino acid modifications that improve specificity or productivity for conversion of 2,3-oxidosqualene to 2,3;22,23 dioxidosqualene, as compared to the enzyme having the amino acid sequence of SEQ ID NO: 39. In some embodiments, the amino acid modifications comprise one or more modifications at positions corresponding to the following positions of SEQ ID NO: 39: 35, 133, 163, 254, 283, 380, and 395. For example, the amino acid at the position corresponding to position 35 of SEQ ID NO: 39 may be arginine (e.g., H35R). The position corresponding to position 133 of SEQ ID NO: 39 may be glycine (e.g., N133G). The amino acid at the position corresponding to position 163 of SEQ ID NO: 39 may be alanine (e.g., F163A). The amino acid at the position corresponding to position 254 of SEQ ID NO: 39 may be phenylalanine (e.g., Y254F). The amino acid at the position corresponding to position 283 of SEQ ID NO: 39 may be leucine (e.g., M283L). The amino acid at the position corresponding to position 380 of SEQ ID NO: 39 may be leucine (e.g., V280L). The amino acid at the position corresponding to position 395 of SEQ ID NO: 39 may be tyrosine (e.g., F395Y).

In various embodiments, the heterologous enzyme pathway comprises a triterpene cyclase (TTC) enzyme. In some embodiments, where the microbial cell coexpresses FPPS, along with the SQS, SQE, and one or more triterpene cyclase enzymes, the microbial cell produces 2,3;22,23-dioxidosqualene. The 2,3;22,23-dioxidosqualene may be the substrate for downstream enzymes in the heterologous pathway. In some embodiments, the triterpene cyclase (TTC) comprises an amino acid sequence that is at least 70% identical to an amino acid sequence selected from SEQ ID NOS: 40 to 55 and 191 to 193. The TTC in various embodiments comprises an amino acid sequence that is at least 70% identical to the amino acid sequence of SEQ ID NO: 40.

In various embodiments, the heterologous enzyme pathway comprises at least two copies of a TTC enzyme gene, or comprises at least two enzymes having triterpene cyclase activity and converting 22,23-dioxidosqualene to 24,25-epoxycucurbitadienol. In such embodiments, product can be pulled to 24,25-epoxycucurbitadienol, with less production of cucurbitadienol. In some embodiments, the heterologous enzyme pathway comprises at least one TTC that comprises an amino acid sequence that is at least 70% identical to one of SEQ ID NO: 191, SEQ ID NO: 192, and SEQ ID NO: 193. For example, when co-expressed with SgCDS, these enzymes demonstrated improved production of 24,25-epoxycucurbitadienol compared to expression of SgCDS alone.

In some embodiments, the heterologous enzyme pathway comprises an epoxide hydrolase (EPH). The EPH may comprise an amino acid sequence that is at least 70% identical to amino acid sequence selected from SEQ ID NOS: 56 to 72, 184 to 190, and 212. In some embodiments, the EPH may employ as a substrate 24,25-epoxycucurbitadienol, for production of 24,25-dihydroxycucurbitadienol.

In some embodiments, the heterologous pathway comprises at least one EPH converting 24,25-epoxycucurbitadienol to 24,25-dihydroxycucurbitadienol, the at least one EPH comprising an amino acid sequence that is at least 70% identical to one of: SEQ ID NO: 189, SEQ ID NO: 58, SEQ ID NO: 184, SEQ ID NO: 185, SEQ ID NO: 187, SEQ ID NO: 188, SEQ ID NO: 190, and SEQ ID NO: 212.

In some embodiments, the heterologous pathway comprises one or more oxidases. The one or more oxidases may be active on cucurbitadienol or oxygenated products thereof as a substrate, adding (collectively) hydroxylations at C11, C24 and 25, thereby producing mogrol. Alternatively or in addition, the heterologous pathway may comprise one or more oxidases that oxidize C11 of C24,25 dihydroxycucurbitadienol to produce mogrol.

In some embodiments, at least one oxidase is a cytochrome P450 enzyme. Exemplary cytochrome P450 enzymes comprise an amino acid sequence that is at least 70% identical to an amino acid sequence selected from SEQ ID NOS: 73 to 91, 171 to 176, and 194 to 200.

In some embodiments, the microbial host cell expresses a heterologous enzyme pathway comprising a P450 enzyme having activity for oxidation at C11 of C24,25 dihydroxycucurbitadienol, to thereby produce mogrol. For example, in some embodiments, the cytochrome P450 comprises an amino acid sequence that is at least 70% identical to an amino acid sequence selected from SEQ ID NO: 194 and SEQ ID NO: 171.

In various embodiments, the microbial host cell expresses one or more electron transfer proteins selected from a cytochrome P450 reductase (CPR), flavodoxin reductase (FPR) and ferredoxin reductase (FDXR) sufficient to regenerate the one or more oxidases. Exemplary CPR proteins are provided herein as SEQ ID NOS: 92 to 99 and 201.

In some embodiments, the microbial host cell expresses SEQ ID NO: 194 or a derivative thereof, and SEQ ID NO: 98 or a derivative thereof. In some embodiments, the microbial host cell expresses SEQ ID NO: 171 or a derivative thereof, and SEQ ID NO: 201 or a derivative thereof.

In some embodiments, the heterologous enzyme pathway further comprises one or more uridine diphosphate-dependent glycosyltransferase (UGT) enzymes, thereby producing one or more mogrol glycosides. The mogrol glycoside may be pentaglycosylated, hexaglycosylated, or more, in some embodiments. In other embodiments, the mogrol glycoside has two, three, or four glucosylations. The one or more mogrol glycosides may be selected from Mog.II-E, Mog.III, Mog.III-A1, Mog.III-A2, Mog.III, Mog.IV, Mog.IV-A, siamenoside, Mog. V, and Mog. VI. In some embodiments, the host cell produces Mog. V or siamenoside.

In some embodiments, the host cell expresses a UGT enzyme that catalyzes the primary glycosylation of mogrol at C24 and/or C3 hydroxyl groups. In some embodiments, the UGT enzyme catalyzes a branching glycosylation, such as a beta 1,2 and/or beta 1,6 branching glycosylation at the primary C3 and C24 gluscosyl groups.

In some embodiments, at least one UGT enzyme comprises an amino acid sequence that is at least 70% identical to an amino acid sequence selected from SEQ ID NOS: 116 to 165, 202 to 210, 211, and 213 to 218.

For example, in some embodiments, the microbial cell expresses at least four UGT enzymes, resulting in glucosylation of mogrol at the C3 hydroxyl group, the C24 hydroxyl group, as well as a further 1,6 glucosylation at the C3 glucosyl group, and a further 1,6 glucosylation and a further 1,2 glucosylation at the C24 glucosyl group. The product of such glucosylation reactions is Mog. V.

In some embodiments, at least one UGT enzyme comprises an amino acid sequence having at least 70% sequence identity to one of SEQ ID NO: 164, 165, 138, 204 to 211, and 213 to 218. In some embodiments, the UGT enzyme is engineered to have higher glycosyltransferase productivity as compared to the wild type enzyme.

In various embodiments, the microbial strain expresses one or more UGT enzymes capable of primary glycosylation at C24 and/or C3 of mogrol. Exemplary UGT enzymes include UGT enzymes comprising: an amino acid sequence that is at least 70% identical to SEQ ID NO: 165, an amino acid sequence that is at least 70% identical to SEQ ID NO: 146, an amino acid sequence that is at least 70% identical to SEQ ID NO: 202, an amino acid sequence that is at least 70% identical to SEQ ID NO: 202, an amino acid sequence that is at least 70% identical to SEQ ID NO: 129, an amino acid sequence that is at least 70% identical to SEQ ID NO: 116, an amino acid sequence that is at least 70% identical to SEQ ID NO: 218, and amino acid sequence that is at least 70% identical to SEQ ID NO: 217.

In various embodiments, the microbial strain expresses one or more UGT enzymes capable of catalyzing a branching glycosylation of one or both primary glycosylations. Such UGT enzymes are summarized in Table 2.

In some embodiments, the microbial host cell has one or more genetic modifications that increase the production of UDP-glucose, the co-factor employed by UGT enzymes.

Mogrol glycosides can be recovered from the microbial culture. For example, mogrol glycosides may be recovered from microbial cells, or in some embodiments, are predominately available in the extracellular media, where they may be recovered or sequestered.

Other aspects and embodiments of the invention will be apparent from the following detailed disclosure.

### DESCRIPTION OF THE FIGURES

FIG. 1 shows the chemical structures of Mog. V, Mog. VI, Isomog. V, and Siamenoside. The type of glycosylation reaction is shown within each glucose moiety (e.g., C3 or C24 core glycosylation and the 1-2, 1-4, or 1-6 glycosylation additions).

FIG. 2 shows routes to Mog. V production in vivo. The enzymatic transformation required for each step is indicated, along with the type of enzyme required. Numbers in parentheses correspond to the chemical structures in FIG. 3. Abbreviations: FPP, farnesyl pyrophosphate; SQS, squalene synthase; SQE, squalene epoxidase; TTC, triterpene cyclase; EPH, epoxide hydrolase; CYP450, cytochrome P450 with reductase partner; UGTs, uridine diphosphate glycosyltransferases.

FIG. 3 depicts chemical structures of metabolites involved in Mog. V biosynthesis: (1) farnesyl pyrophosphate; (2) squalene; (3) 2,3-oxidosqualene; (4) 2,3;22,23-dioxidosqualene; (5) 24,25-epoxycucurbitadienol; (6) 24,25-dihydroxycucurbitadienol; (7) mogrol; (8) mogroside V; (9) cucurbitadienol.

FIG. 4 illustrates glycosylation routes to Mog. V. Bubble structures represent different mogrosides. White tetra-cyclic core represents mogrol. The numbers below each structure indicate the particular glycosylated mogroside. Black circles represent C3 or C24 glucosylations. Dark grey vertical circles represent 1,6-glucosylations. Light grey horizontal circles represent 1,2-glucosylations. Abbreviations: Mog, mogrol; sia, siamenoside.

FIG. 5 shows results for in vivo production of squalene in E. coli using different squalene synthases. The asterisk denotes a different plasmid construct and experiment run on a different day from the others shown. Legend: (1) SgSQS (SEQ ID NO:2), (2) AaSQS (SEQ ID NO: 11), (3) EsSQS (SEQ ID NO: 16), (4) EISQS (SEQ ID NO: 14), (5) FbSQS (SEQ ID NO: 166), (6) BbSQS (SEQ ID NO: 167).

FIG. 6 shows results for in vivo production of squalene, 2,3-oxidosqualene, and 2,3;22,23-dioxidosqualene using different squalene epoxidases. Legend: (A) SEQ ID NO: 2 and SEQ ID NO: 168; (B) SEQ ID NO: 11 and SEQ ID NO: 168; (C) SEQ ID NO: 2 and SEQ ID NO: 169; (D) SEQ ID NO: 11 and SEQ ID NO: 169; (E) SEQ ID NO: 2 and SEQ ID NO: 170; (F) SEQ ID NO: 2 and SEQ ID NO: 39; (G) SEQ ID NO: 11 and SEQ ID NO: 39.

FIG. 7 shows results for in vivo production of the cyclized triterpene product. Reactions involve an increasing number of enzymes expressed in an E. coli cell line having an overexpression of MEP pathway enzymes. The asterisks represent fermentation experiments incubated for a quarter of the time than the other experiments. As shown, co-expression of SQS (SEQ ID NO: 11), SQE (SEQ ID NO: 39), and TTC (SEQ ID NO: 40) (lane G) resulted in high production of the triterpenoid product, cucurbitadienol. Legends: Product 1 is squalene; Product 2 is 2,3-oxidosqualene; Product 3 is cucurbitadienol; (A) expression of SEQ ID NO: 2, (B) expression of SEQ ID NO: 11, (C) coexpression of SEQ ID NO: 2 and SEQ ID NO: SEQ ID NO: 17, (D) coexpression of SEQ ID NO: 2 and SEQ ID NO: 169; (E) coexpression of SEQ ID NO: 11 and SEQ ID NO: 169; (F) coexpression of SEQ ID NO: 2, SEQ ID NO: 17, and SEQ ID NO: 40; (G) coexpression of SEQ ID NO: 11, SEQ ID NO: 39, and SEQ ID NO: 40.

FIG. 8 shows results for SQE engineering to produce high titers of 2,3;22,23-dioxidosqualene. Expression of SQS (SEQ ID NO: 11), SQE (SEQ ID NO: 39), and TTC (SEQ ID NO: 40) whether on a bacterial artificial chromosome (BAC) or integrated, produce large amounts of cucurbitadienol. Point mutations in SQE (SEQ ID NO: 39) were screened to complement SQE to reduce levels of cucurbitadienol, with corresponding gain in titers of 2,3;22,23-dioxidosqualene. Two variants are shown in FIG. 8, SQE A4 (including H35R, F163A, M283L, V380L, and F395Y substitutions, SEQ ID NO: 203) and SQE C11 (including H35R, N133G, F163A, Y254F, V380L, and F395Y substitutions).

FIG. 9 shows production of 2,3;22,23 dioxidosqualene. Titers are plotted for each strain producing 2,3;22,23 dioxidosqualene. An engineered squalene epoxidase gene, SEQ ID NO: 203, was expressed in a strain producing 2,3 oxidosqualene via the squalene epoxidase of SEQ ID NO: 39. Strains were incubated for 48 hours before extraction. Lanes: (1) expression of SQE of SEQ ID NO: 39; (2) expression of SQE of SEQ ID NO: 39 and SEQ ID NO: 203.

FIG. 10 shows the coexpression of SQS, SQE, and TTC enzymes. CDS of SEQ ID NO: 40, when coexpressed with SQS (SEQ ID NO: 11), SQE (SEQ ID NO: 39), and SQE A4 (SEQ ID NO: 203) in E. coli, resulted in production of cucurbitadienol and 24,25-epoxycucurbitadienol. E. coli strains coexpressing SQS (SEQ ID NO: 11), SQE (SEQ ID NO: 39), SQE A4 (SEQ ID NO: 203), and CDS (SEQ ID NO: 40), with an additional TTC produced higher levels of 24,25-epoxycucurbitadienol. Legend: TTC1 is SEQ ID NO: 92, TTC2 is SEQ ID NO: 191, TTC3 is SEQ ID NO: 193, TTC4 is SEQ ID NO: 40.

FIG. 11 shows production of cucurbitadienol and 24,25-epoxycucurbitadienol. E. coli strains producing oxidosqualene and dioxidosqualene were complemented with CDS homologs and CAS genes engineered to produce cucurbitadienol. The ratio of 24,25-epoxycucurbitadienol to cucurbitadienol varies from 0.15 for Enzyme 1 (SEQ ID NO: 40) to 0.58 for Enzyme 2 (SEQ ID NO: 192), demonstrating improved substrate specificity toward the desired 24,25-epoxycucurbitadienol product for Enzyme 2. Enzyme 3 is SEQ ID NO: 219, and Enzyme 4 is SEQ ID NO: 220.

FIG. 12 shows the screening of EPH enzymes for hydration of 24,25-epoxycucurbitadienol to produce 24,25-dihydroxycucurbitadienol in E. coli strains coexpressing SQS (SEQ ID NO: 11), SQE (SEQ ID NO: 39), SQE A4 (SEQ ID NO: 203), and TTC (SEQ ID NO: 40). These fermentation experiments were performed at 30° C. for 72 hours in 96 well plates. Legend: EPH1 (SEQ ID NO: 186); EPH2 (SEQ ID NO: 212); EPH3 (SEQ ID NO: 190); EPH4 (SEQ ID NO: 187); EPH5 (SEQ ID NO: 184); EPH6 (SEQ ID NO: 185); EPH7 (SEQ ID NO: 188); EPH8 (SEQ ID NO: 189); and EPH9 (SEQ ID NO: 58).

FIG. 13(A-C) show the coexpression of SQS, SQE, TTC, EPH, and P450 enzymes to produce mogrol. An E. coli strain expressing SEQ ID NOS: 11, 39, 203 along with CDS, EPH, and P450 genes with a CPR resulted in production of mogrol and oxo-mogrol (FIG. 13A). These fermentation experiments were performed at 30° C. for 72 hours in 96 well plates. Mogrol production was validated by LC-QQQ mass spectrum analysis with a spiked authentic standard (FIG. 13B) and GC-FID chromatography versus an authentic standard (FIG. 13C). Legend: (1) coexpression of SEQ ID NO: 40, SEQ ID NO: 58, SEQ ID NO: 194), and SEQ ID NO: 98); (2) coexpression of SEQ ID NO: 40, SEQ ID NO: 58, SEQ ID NO: 197, and SEQ ID NO: 98; (3) SEQ ID NO: 40, SEQ ID NO: 58, SEQ ID NO: 171, and SEQ ID NO: 201.

FIG. 14 shows the screening of cytochrome P450s for oxidation at C11 of the 24,25-dihydroxycucurbitadienol-like molecule cucurbitadienol. Native anchor P450 enzymes shown are: (1) SEQ ID NO: 194, (2) SEQ ID NO: 197, (3) SEQ ID NO: 171, (4) SEQ ID NO: 74); and (5) SEQ ID NO: SEQ ID NO: 75. In some cases, the native transmembrane domain was replaced with the transmembrane domain from E. coli sohB (Anchor 3), E. coli zipA (Anchor 2), or bovine 17a (Anchor 1) to improve interaction with the E. coli membrane. Each P450 was coexpressed with either CPR SEQ ID NO: 98 or CPR (SEQ ID NO: 201), resulting in production of 11-hydroxycucurbitadienol. These fermentation experiments were performed at 30° C. for 72 hours in 96 well plates.

FIG. 15 shows production of products with oxidation at C11.

FIG. 16 shows Mog. V production using a combination of different enzymes. (A) Penta-glycosylated products are observed when UGTs of SEQ ID NO: 165, SEQ ID NO: 146, SEQ ID NO: 117, or SEQ ID NO: 164 are incubated together with mogrol as a substrate. Strains: (1) expresses SEQ ID NO: 165, (2) expresses SEQ ID NO: 146, (3) co-expresses SEQ ID NO: 165 and SEQ ID NO: 146, (4) co-expresses SEQ ID NO: 165, SEQ ID NO: 146, and SEQ ID NO: 117, (5) co-expresses SEQ ID NO: 165, SEQ ID NO: 146, SEQ ID NO: 117, and SEQ ID NO: 164. Mogroside substrates were incubated in Tris buffer containing magnesium chloride, beta-mercaptoethanol, UDP-glucose, single UGT, and a phosphatase. (B) Extracted ion chromatogram (EIC) for 1285.4 Da (mogroside V+H) of reactions containing SEQ ID NO: 165 and SEQ ID NO: 146, and either Enzyme 1 (SEQ ID NO: 117) or Enzyme 2 (SEQ ID NO: 164) when incubated with Mog.II-E. (C) Extracted ion chromatogram (EIC) for 1285.4 Da (mogroside V+H) of reactions containing SEQ ID NO: 165 and SEQ ID NO: 146 and either Enzyme 1 (SEQ ID NO: 117) or Enzyme 2 (SEQ ID NO: 164) when incubated with mogrol. Abbreviation: MogV, mogroside V.

FIG. 17 shows in vitro assays showing the conversion of mogroside substrates to more glycosylated products. Mogroside substrates were incubated in Tris buffer containing magnesium chloride, beta-mercaptoethanol, UDP-glucose, single UGT, and a phosphatase. The panels correspond to the use of different substrates: (A) mogrol; (B) Mog.I-A; (C) Mog.I-E; (D) Mog.II-E; (E) Mog.III; (F) Mog.IV-A; (G) Mog.IV; (H) siamenoside. Enzyme 1 (SEQ ID NO: 165), Enzyme 2 (SEQ ID NO: 146), Enzyme 3 (SEQ ID NO: 116), Enzyme 4 (SEQ ID NO: 117), and Enzyme 5 (SEQ ID NO: 164).

FIG. 18 shows the bioconversion of mogrol into mogroside-IA or mogroside-IIE. In the experiment, engineered E. coli strains were inoculated with 0.2 mM mogrol at 37° C. Product formation was examined after 48 hours. The values are reported relative to the empty vector control (the values reported are the detected compound minus the background level detected in the empty vector control). Products were measured on LC/MS-QQQ with authentic standards. Only Enzyme 1 shows formation of mogroside-IIE. Enzyme 1 to 5 are SEQ ID NOS: 202, 116, 216, 217, and 218 respectively.

FIG. 19A and FIG. 19B shows the bioconversion of Mog.IA (FIG. 19A) or Mog.IE (FIG. 18B) into Mog.IIE. Engineered E. coli strains (expressing either Enzyme 1, SEQ ID NO: 165; Enzyme 2, SEQ ID NO: 202; or Enzyme 3, SEQ ID NO: 116) were grown at 37° C. in fermentation media containing 0.2 mM Mog.IA (FIG. 19A) or Mog.IE (FIG. 19B). Product formation was measured after 48 hours using LC-MS/MS with authentic standards. Reported values are those in excess of the empty vector control.

FIG. 20 shows the production of Mog.III or siamenoside from Mog.II-E by engineered E. coli strains expressing Enzyme 1 (SEQ ID NO: 204), Enzyme 2 (SEQ ID NO: 138), or Enzyme 3 (SEQ ID NO: 206). Strains were grown at 37° C. in fermentation media containing 0.2 mM Mog.IA, and product formation was measured after 48 hours using LC-MS/MS with authentic standards.

FIG. 21 shows the in vitro production of Mog.IIA2 by cells expressing Enzyme 1 (SEQ ID NO: 205). 0.1 mM Mog.I-E was added, and reactions were incubated at 37° C. for 48 hr. Data was quantified by LC MS/MS with authentic standards of each compound.

FIG. 22 (A,B) shows production of Mog. V in E. coli. (A) Chromatogram indicating Mog. V production from engineered E. coli strains expressing SEQ ID NO: 11, SEQ ID NO: 39, SEQ ID NO: 203, SEQ ID NO: 40, SEQ ID NO: 189, SEQ ID NO: 199, SEQ ID NO: 202, SEQ ID NO: 165, and SEQ ID NO: 122. Strains were incubated at 30° C. for 72 hours before extraction. Mog. V production was verified by LC-QQQ spectrum analysis versus an authentic standard. (B) Chromatogram indicating Mog. V production from a biological sample with a spiked Mog. V authentic standard.

FIG. 23 shows bioconversion of mogroside-IIE to further glycosylated products using an engineered version of the UGT enzyme of SEQ ID NO: 164.

FIG. 24 shows bioconversion of Mog.IA to Mog.IIE with an engineered version of the UGT enzyme of SEQ ID NO: 165.

FIG. 25 shows bioconversion of Mog.IE to Mog.IIE with an engineered version of the UGT enzyme of SEQ ID NO: 217.

FIG. 26 is an amino acid alignment of CaUGT_1,6 and SgUGT94_289_3 using Clustal Omega (Version CLUSTAL O (1,2,4). These sequences share 54% amino acid identity.

FIG. 27 is an amino acid alignment of Homo sapiens squalene synthase (HsSQS) (NCBI accession NP_004453.3) and AaSQS (SEQ ID NO: 11) using Clustal Omega (Version CLUSTAL O (1.2.4)). HsSQS has a published crystal structure (PDB entry: 1EZF). These sequences share 42% amino acid identity.

FIG. 28 is an amino acid alignment of Homo sapiens squalene epoxidase (HsSQE) (NCBI accession XP_011515548) and MISQE (SEQ ID NO: 39) using Clustal Omega (Version CLUSTAL O (1.2.4)). HsSQE has a published crystal structure (PDB entry: 6C6N). These sequences share 35% amino acid identity.

### DETAILED DESCRIPTION OF THE INVENTION

The present invention, in various aspects and embodiments, provides microbial strains and methods for making mogrol and mogrol glycosides, using recombinant microbial processes. In other aspects, the invention provides methods for making products, including foods, beverages, and sweeteners (among others), by incorporating the mogrol glycosides produced according to the methods described herein. In still other aspects, the invention provides engineered UGT enzymes for glycosylating secondary metabolite substrates, such as mogrol or mogrosides.

As used herein, the terms “terpene or triterpene” are used interchangeably with the terms “terpenoid” or “triterpenoid,” respectively.

In various aspects, the invention provides microbial strains and methods for making the triterpenoid compound mogrol, or glycoside products thereof. The invention provides a recombinant microbial host cell expressing a heterologous enzyme pathway catalyzing the conversion of isopentenyl pyrophosphate (IPP) and/or dimethylallyl pyrophosphate (DMAPP) to one or more of mogrol or mogroside(s).

The microbial host cell in various embodiments may be prokaryotic or eukaryotic. In some embodiments, the microbial host cell is a bacterium, and which can be optionally selected from Escherichia spp., Bacillus spp., Corynebacterium spp., Rhodobacter spp., Zymomonas spp., Vibrio spp., and Pseudomonas spp. For example, in some embodiments, the bacterial host cell is a species selected from Escherichia coli, Bacillus subtilis, Corynebacterium glutamicum, Rhodobacter capsulatus, Rhodobacter sphaeroides, Zymomonas mobilis, Vibrio natriegens, or Pseudomonas putida. In some embodiments, the bacterial host cell is E. coli. Alternatively, the microbial cell may be a yeast cell, such as but not limited to a species of Saccharomyces, Pichia, or Yarrowia, including Saccharomyces cerevisiae, Pichia pastoris, and Yarrowia lipolytica.

The microbial cell will produce MEP or MVA products, which act as substrates for the heterologous enzyme pathway. The MEP (2-C-methyl-D-erythritol 4-phosphate) pathway, also called the MEP/DOXP (2-C-methyl-D-erythritol 4-phosphate/l-deoxy-D-xylulose 5-phosphate) pathway or the non-mevalonate pathway or the mevalonic acid-independent pathway refers to the pathway that converts glyceraldehyde-3-phosphate and pyruvate to IPP and DMAPP. The pathway, which is present in bacteria, typically involves action of the following enzymes: 1-deoxy-D-xylulose-5-phosphate synthase (Dxs), 1-deoxy-D-xylulose-5-phosphate reductoisomerase (IspC), 4-diphosphocytidyl-2-C-methyl-D-erythritol synthase (IspD), 4-diphosphocytidyl-2-C-methyl-D-erythritol kinase (IspE), 2C-methyl-D-erythritol 2,4-cyclodiphosphate synthase (IspF), 1-hydroxy-2-methyl-2-(E)-butenyl 4-diphosphate synthase (IspG), and isopentenyl diphosphate isomerase (IspH). The MEP pathway, and the genes and enzymes that make up the MEP pathway, are described in U.S. Pat. No. 8,512,988, which is hereby incorporated by reference in its entirety. For example, genes that make up the MEP pathway include dxs, ispC, ispD, ispE, ispF, ispG, ispH, idi, and ispA. In some embodiments, the host cell expresses or overexpresses one or more of dxs, ispC, ispD, ispE, ispF, ispG, ispH, idi, ispA, or modified variants thereof, which results in the increased production of IPP and DMAPP. In some embodiments, the triterpenoid (e.g., squalene, mogrol, or other intermediate described herein) is produced at least in part by metabolic flux through an MEP pathway, and wherein the host cell has at least one additional gene copy of one or more of dxs, ispC, ispD, ispE, ispF, ispG, ispH, idi, ispA, or modified variants thereof.

The MVA pathway refers to the biosynthetic pathway that converts acetyl-CoA to IPP. The mevalonate pathway, which will be present in yeast, typically comprises enzymes that catalyze the following steps: (a) condensing two molecules of acetyl-CoA to acetoacetyl-CoA (e.g., by action of acetoacetyl-CoA thiolase); (b) condensing acetoacetyl-CoA with acetyl-CoA to form hydroxymethylglutaryl-CoenzymeA (HMG-COA) (e.g., by action of HMG-COA synthase (HMGS)); (c) converting HMG-CoA to mevalonate (e.g., by action of HMG-COA reductase (HMGR)); (d) phosphorylating mevalonate to mevalonate 5-phosphate (e.g., by action of mevalonate kinase (MK)); (e) converting mevalonate 5-phosphate to mevalonate 5-pyrophosphate (e.g., by action of phosphomevalonate kinase (PMK)); and (f) converting mevalonate 5-pyrophosphate to isopentenyl pyrophosphate (e.g., by action of mevalonate pyrophosphate decarboxylase (MPD)). The MVA pathway, and the genes and enzymes that make up the MVA pathway, are described in U.S. Pat. No. 7,667,017, which is hereby incorporated by reference in its entirety. In some embodiments, the host cell expresses or overexpresses one or more of acetoacetyl-CoA thiolase, HMGS, HMGR, MK, PMK, and MPD or modified variants thereof, which results in the increased production of IPP and DMAPP. In some embodiments, the triterpenoid (e.g., mogrol or squalene) is produced at least in part by metabolic flux through an MVA pathway, and wherein the host cell has at least one additional gene copy of one or more of acetoacetyl-CoA thiolase, HMGS, HMGR, MK, PMK, MPD, or modified variants thereof.

In some embodiments, the host cell is a bacterial host cell engineered to increase production of IPP and DMAPP from glucose as described in U.S. Pat. Nos. 10,480,015 and 10,662,442, the contents of which are hereby incorporated by reference in their entireties. For example, in some embodiments the host cell overexpresses MEP pathway enzymes, with balanced expression to push/pull carbon flux to IPP and DMAP. In some embodiments, the host cell is engineered to increase the availability or activity of Fe—S cluster proteins, so as to support higher activity of IspG and IspH, which are Fe—S enzymes. In some embodiments, the host cell is engineered to overexpress IspG and IspH, so as to provide increased carbon flux to 1-hydroxy-2-methyl-2-(E)-butenyl 4-diphosphate (HMBPP) intermediate, but with balanced expression to prevent accumulation of HMBPP at an amount that reduces cell growth or viability, or at an amount that inhibits MEP pathway flux and/or terpenoid production. In some embodiments, the host cell exhibits higher activity of IspH relative to IspG. In some embodiments, the host cell is engineered to downregulate the ubiquinone biosynthesis pathway, e.g., by reducing the expression or activity of IspB, which uses IPP and FPP substrate.

In various embodiments, the heterologous enzyme pathway comprises a farnesyl diphosphate synthase (FPPS) and a squalene synthase (SQS), which are recombinantly expressed. In various embodiments, the SQS comprises an amino acid sequence that is at least 70% identical to an amino acid sequence selected from SEQ ID NOS: 2 to 16, 166, and 167.

By way of non-limiting example, the FPPS may be Saccharomyces cerevisiae farnesyl pyrophosphate synthase (ScFPPS) (SEQ ID NO: 1), or modified variants thereof. Modified variants may comprise an amino acid sequence that is at least 70% identical to SEQ ID NO: 1). For example, the FPPS may comprise an amino acid sequence that is at least 80%, or at least 85%, or at least 90%, or at least 95%, or at least 98%, or at least 99% identical to SEQ ID NO: 1. In some embodiments, the FPPS comprises an amino acid sequence having from 1 to 20 amino acid modifications or having from 1 to 10 amino acid modifications with respect to SEQ ID NO: 1, the amino acid modifications being independently selected from amino acid substitutions, deletions, and insertions. Numerous other FPPS enzymes are known in the art, and may be employed for conversion of IPP and/or DMAPP to farnesyl diphosphate in accordance with this aspect.

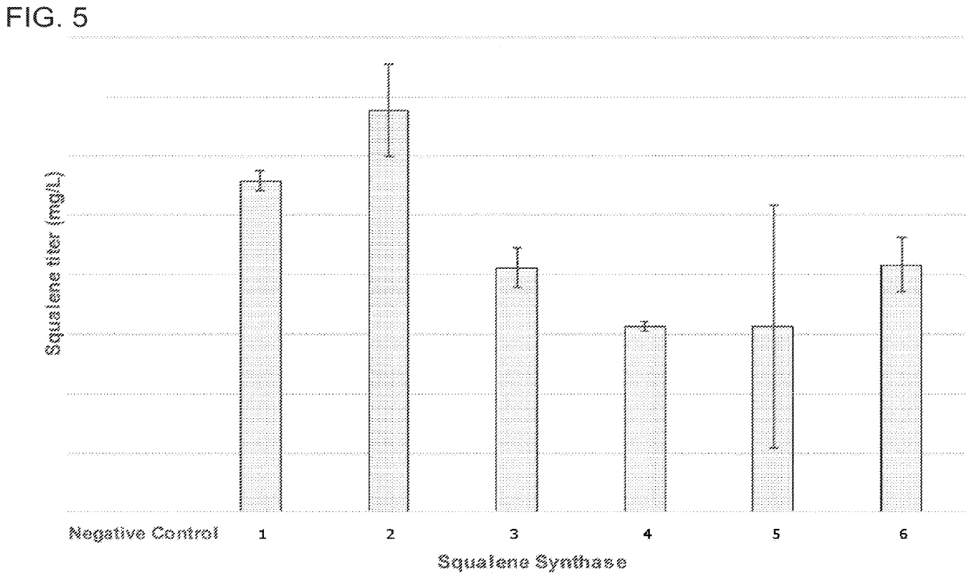

In some embodiments, the SQS comprises an amino acid sequence that is at least 70% identical to SEQ ID NO: 11. For example, the SQS may comprise an amino acid sequence that is at least 80%, or at least 85%, or at least 90%, or at least 95%, or at least 98%, or at least 99% identical to SEQ ID NO: 11. In some embodiments, the SQS comprises an amino acid sequence having from 1 to 20 amino acid modifications or from 1 to 10 amino acid modifications with respect to SEQ ID NO: 11, the amino acid modifications being independently selected from amino acid substitutions, deletions, and insertions. Amino acid modifications may be made to increase expression or stability of the enzyme in the microbial cell, or to increase productivity of the enzyme. As shown in FIG. 5, AaSQS has high activity in E. coli.

In some embodiments, the SQS comprises an amino acid sequence that is at least 70% identical to SEQ ID NO: 2. For example, the SQS may comprise an amino acid sequence that is at least 80%, or at least 85%, or at least 90%, or at least 95%, or at least 98%, or at least 99% identical to SEQ ID NO: 2. In some embodiments, the SQS comprises an amino acid sequence having from 1 to 20 amino acid modifications or from 1 to 10 amino acid modifications with respect to SEQ ID NO: 2, the amino acid modifications being independently selected from amino acid substitutions, deletions, and insertions. Amino acid modifications may be made to increase expression or stability of the enzyme in the microbial cell, or to increase productivity of the enzyme. As shown in FIG. 5, SgSQS has high activity in E. coli.

In some embodiments, the SQS comprises an amino acid sequence that is at least 70% identical to SEQ ID NO: 14. For example, the SQS may comprise an amino acid sequence that is at least 80%, or at least 85%, or at least 90%, or at least 95%, or at least 98%, or at least 99% identical to SEQ ID NO: 14. In some embodiments, the SQS comprises an amino acid sequence having from 1 to 20 amino acid modifications or from 1 to 10 amino acid modifications with respect to SEQ ID NO: 14, the amino acid modifications being independently selected from amino acid substitutions, deletions, and insertions. Amino acid modifications may be made to increase expression or stability of the enzyme in the microbial cell, or to increase productivity of the enzyme. As shown in FIG. 5, EISQS was active in E. coli.

In some embodiments, the SQS comprises an amino acid sequence that is at least 70% identical to SEQ ID NO: 16. For example, the SQS may comprise an amino acid sequence that is at least 80%, or at least 85%, or at least 90%, or at least 95%, or at least 98%, or at least 99% identical to SEQ ID NO: 16. In some embodiments, the SQS comprises an amino acid sequence having from 1 to 20 amino acid modifications or from 1 to 10 amino acid modifications with respect to SEQ ID NO: 16, the amino acid modifications being independently selected from amino acid substitutions, deletions, and insertions. Amino acid modifications may be made to increase expression or stability of the enzyme in the microbial cell, or to increase productivity of the enzyme. As shown in FIG. 5, EsSQS was active in E. coli.

In some embodiments, the SQS comprises an amino acid sequence that is at least 70% identical to SEQ ID NO: 166. For example, the SQS may comprise an amino acid sequence that is at least 80%, or at least 85%, or at least 90%, or at least 95%, or at least 98%, or at least 99% identical to SEQ ID NO: 166. In some embodiments, the SQS comprises an amino acid sequence having from 1 to 20 amino acid modifications or from 1 to 10 amino acid modifications with respect to SEQ ID NO: 166, the amino acid modifications being independently selected from amino acid substitutions, deletions, and insertions. Amino acid modifications may be made to increase expression or stability of the enzyme in the microbial cell, or to increase productivity of the enzyme. As shown in FIG. 5, FbSQS was active in E. coli.

In some embodiments, the SQS comprises an amino acid sequence that is at least 70% identical to SEQ ID NO: 167. For example, the SQS may comprise an amino acid sequence that is at least 80%, or at least 85%, or at least 90%, or at least 95%, or at least 98%, or at least 99% identical to SEQ ID NO: 167. In some embodiments, the SQS comprises an amino acid sequence having from 1 to 20 amino acid modifications or from 1 to 10 amino acid modifications with respect to SEQ ID NO: 167, the amino acid modifications being independently selected from amino acid substitutions, deletions, and insertions. Amino acid modifications may be made to increase expression or stability of the enzyme in the microbial cell, or to increase productivity of the enzyme. As shown in FIG. 5, BbSQS was active in E. coli.

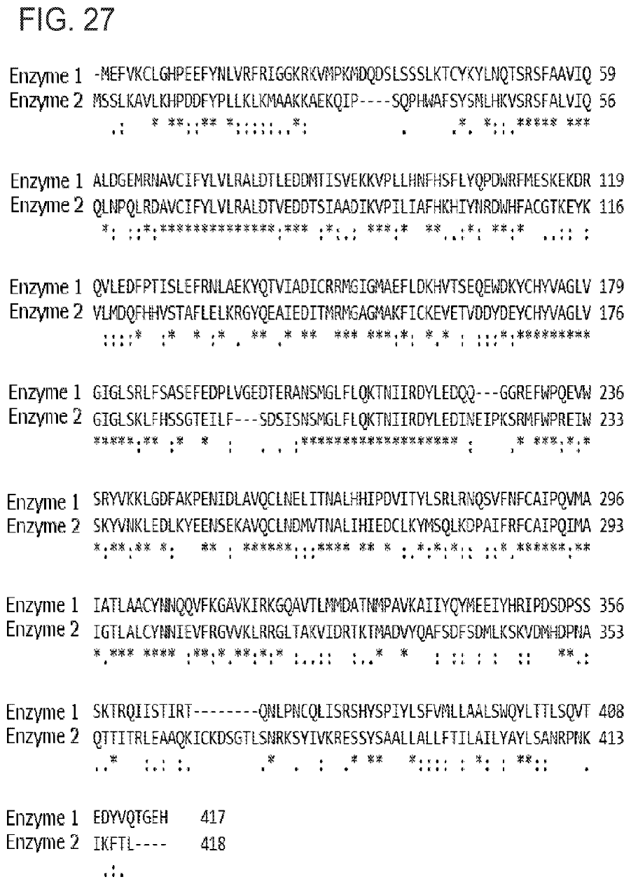

Amino acid modifications to the SQS enzyme can be guided by available enzyme structures and homology models, including those described in Aminfar and Tohidfar, In silico analysis of squalene synthase in Fabaceae family using bioinformatics tools, J. Genetic Engineer, and Biotech. 16 (2018) 739-747. The publicly available crystal structure for HsSQE (PDB entry: 6C6N) may be used to inform amino acid modifications. An alignment between AaSQS and HsSQS is shown in FIG. 27. The enzymes have 42% amino acid identity.

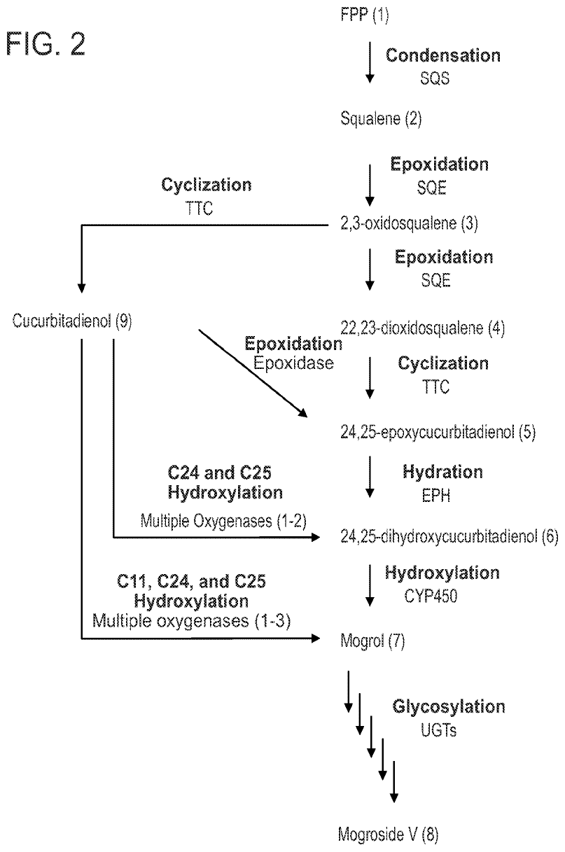

In some embodiments, the host cell expresses one or more enzymes that produce mogrol from squalene. For example, the host cell may express one or more squalene epoxidase (SQE) enzymes, one or more triterpenoid cyclases, one or more epoxide hydrolase (EPH) enzymes, one or more cytochrome P450 oxidases (CYP450), optionally one or more non-heme iron-dependent oxygenases, and one or more cytochrome P450 reductases (CPR). As shown in FIG. 2, the heterologous pathway can proceed through several routes to mogrol, which may involve one or two epoxidations of the core substrate. In some embodiments, the pathway proceeds through cucurbitadienol, and in some embodiments, does not involve a further epoxidation step. In some embodiments, cucurbitadienol intermediate is converted to 24,25-epoxycucurbitadienol (5) by one or or more epoxidase enzymes (such as that provided herein as SEQ ID NO: 221). In still other embodiments, the pathway largely proceeds through 2,3;24,25-dioxidosqualene, with only small or minimal production of cucurbitadienol intermediate. In some embodiments, one or more of SQE, CDS, EPH, CYP450, non-heme iron-dependent oxygenases, flavodoxin reductases (FPR), ferredoxin reductases (FDXR), and CPR enzymes are engineered to increase flux to mogrol.

In some embodiments, the heterologous enzyme pathway comprises two squalene epoxidase (SQE) enzymes. For example, the heterologous enzyme pathway may comprise an SQE that produces 2,3-oxidosqualene (intermediate (3) in FIG. 2). In some embodiments, the SQE will produce 2,3;22,23-dioxidosqualene (intermediate (4) in FIG. 2), and this conversion can be catalyzed by the same SQE enzyme, or an enzyme that differs in amino acid sequence by at least one amino acid modification. For example, the squalene epoxidase enzymes may include at least two SQE enzymes each comprising (independently) an amino acid sequence that is at least 70% identical to any one of SEQ ID NOS: 17 to 39, 168 to 170, and 177 to 183. By coexpression of an SQE enzyme engineered or screened for substrate specificity for 2,3-oxidosqualene, the di-epoxy intermediate can be produced, with low or minimal levels of cucurbitadienol. In these embodiments, P450 oxygenase enzymes hydroxylating C24 and C25 of the scaffold can be eliminated.

In some embodiments, the at least one SQE comprises an amino acid sequence that is at least 70%, or at least 80%, or at least 85%, or at least 90%, or at least 95%, or at least 98%, or at least 99% identical to SEQ ID NO: 39. For example, the SQE enzyme may comprise an amino acid sequence having from 1 to 20 amino acid modifications with respect to SEQ ID NO: 39, the amino acid modifications being independently selected from amino acid substitutions, deletions, and insertions.

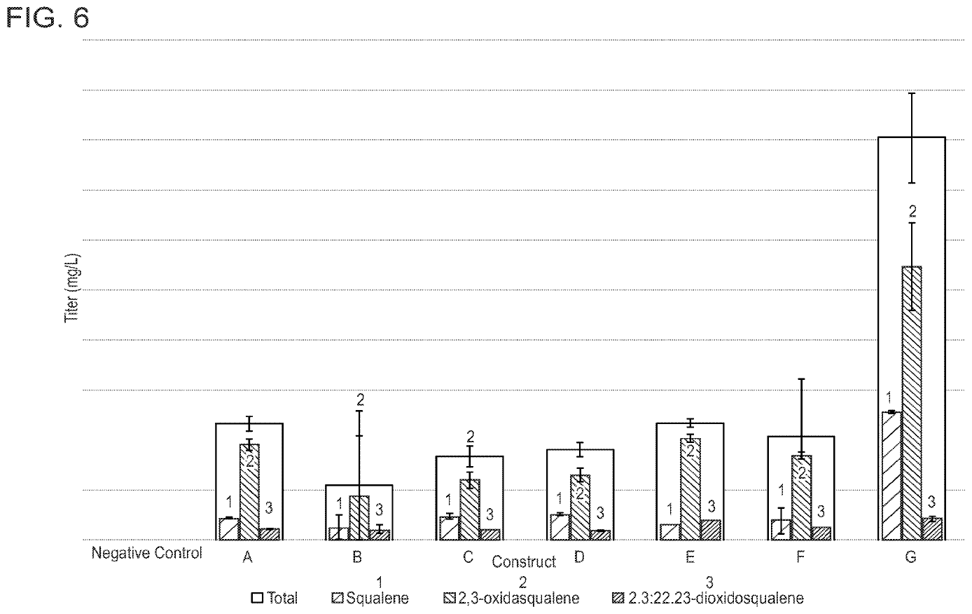

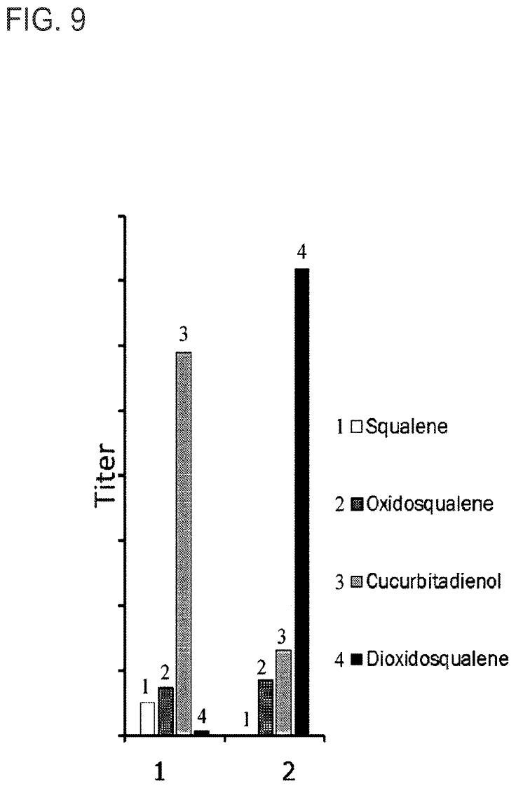

As shown in FIG. 6, MISQE has high activity in E. coli, particularly when coexpressed with AaSQS, where high levels of the single epoxylated product (2,3-oxidosqualene) were observed. Accordingly, coexpression of AaSQS (or an engineered derivative) with multiple copies of MISQE engineered as described above, has good potential for bioengineering of the mogrol pathway. See FIG. 9. Amino acid modifications may be made to increase expression or stability of the SQE enzyme in the microbial cell, or to increase productivity of the enzyme.

In some embodiments, the host cell comprises two squalene epoxidase enzymes that each comprise an amino acid sequence that is at least 70% identical to Methylomonas lenta squalene epoxidase (SEQ ID NO: 39). For example, one of the SQE enzymes may have one or more amino acid modifications that improve specificity or productivity for conversion of 2,3-oxidosqualene to 2,3;22,23 dioxidosqualene, as compared to the enzyme having the amino acid sequence of SEQ ID NO: 39. In some embodiments, the amino acid modifications comprise one or more (or in some embodiments, 2, 3, 4, 5, 6, or 7) modifications at positions corresponding to the following positions of SEQ ID NO: 39: 35, 133, 163, 254, 283, 380, and 395. For example, the amino acid at the position corresponding to position 35 of SEQ ID NO: 39 may be arginine or lysine (e.g., H35R). The position corresponding to position 133 of SEQ ID NO: 39 may be glycine, alanine, leucine, isoleucine, or valine (e.g., N133G). The amino acid at the position corresponding to position 163 of SEQ ID NO: 39 may be glycine, alanine, leucine, isoleucine, or valine (e.g., F163A). The amino acid at the position corresponding to position 254 of SEQ ID NO: 39 may be phenylalanine, alanine, leucine, isoleucine, or valine (e.g., Y254F). The amino acid at the position corresponding to position 283 of SEQ ID NO: 39 may be alanine, leucine, isoleucine, or valine (e.g., M283L). The amino acid at the position corresponding to position 380 of SEQ ID NO: 39 may be alanine, leucine, or glycine (e.g., V280L). The amino acid at the position corresponding to position 395 of SEQ ID NO: 39 may be tyrosine, serine, or threonine (e.g., F395Y). Exemplary SQE enzymes in these embodiments are at least 70%, or at least 80%, or at least 90%, or at least 95% identical to SEQ ID NO: 39, but comprise the following sets of amino acid substitutions: H35R, F163A, M283L, V380L, F395Y; or H35R, N133G, F163A, Y254F, V380L, and F395Y, in each case numbered according to SEQ ID NO: 39. For example, the host cell may express an SQE comprising the amino acid sequence of SEQ. ID NO: 203 (referred to herein as MISQE A4).

In still other embodiments, the squalene epoxidase comprises an amino acid sequence that is at least 70% identical to SEQ ID NO: 168). For example, the SQE may comprise an amino acid sequence that is at least 80%, or at least 85%, or at least 90%, or at least 95%, or at least 98%, or at least 99% identical to SEQ ID NO: 168. In various embodiments, the SQE comprises an amino acid sequence having from 1 to 20 amino acid modifications or from 1 to 10 amino acid modifications with respect to SEQ ID NO: 168, the amino acid modifications being independently selected from amino acid substitutions, deletions, and insertions. As shown in FIG. 6, BaESQE had good activity in E. coli. Amino acid modifications may be made to increase expression or stability of the enzyme in the microbial cell, or to increase productivity of the enzyme.

In some embodiments, the squalene epoxidase comprises an amino acid sequence that is at least 70% identical to SEQ ID NO: 169. For example, the SQE may comprise an amino acid sequence that is at least 80%, or at least 85%, or at least 90%, or at least 95%, or at least 98%, or at least 99% identical to SEQ ID NO: 169. In various embodiments, the SQE comprises an amino acid sequence having from 1 to 20 amino acid modifications or from 1 to 10 amino acid modifications with respect to SEQ ID NO: 169, the amino acid modifications being independently selected from amino acid substitutions, deletions, and insertions. As shown in FIG. 6, MsSQE had good activity in E. coli. Amino acid modifications may be made to increase expression or stability of the enzyme in the microbial cell, or to increase productivity of the enzyme.

In some embodiments, the squalene epoxidase comprises an amino acid sequence that is at least 70% identical to SEQ ID NO: 170. For example, the SQE may comprise an amino acid sequence that is at least 80%, or at least 85%, or at least 90%, or at least 95%, or at least 98%, or at least 99% identical to SEQ ID NO: 170. In various embodiments, the SQE comprises an amino acid sequence having from 1 to 20 amino acid modifications or from 1 to 10 amino acid modifications with respect to SEQ ID NO: 170, the amino acid modifications being independently selected from amino acid substitutions, deletions, and insertions. As shown in FIG. 6, MbSQE had good activity in E. coli. Amino acid modifications may be made to increase expression or stability of the enzyme in the microbial cell, or to increase productivity of the enzyme.

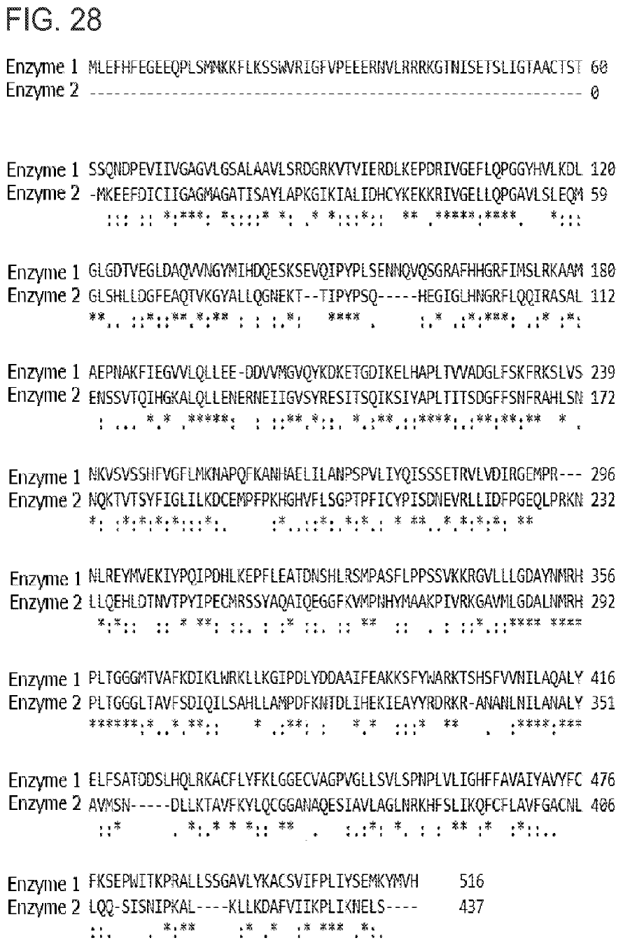

Amino acid modifications can be guided by available enzyme structures and homology models, including those described in Padyana A K, et al., Structure and inhibition mechanism of the catalytic domain of human squalene epoxidase, Nat. Comm. (2019) Vol. 10 (97): 1-10; or Ruckenstulh et al., Structure-Function Correlations of Two Highly Conserved Motifs in Saccharomyces cerevisiae Squalene Epoxidase, Antimicrob. Agents and Chemo. (2008) Vol. 52 (4): 1496-1499. FIG. 28 shows an alignment of HsSQE and MISQE, which is useful for guiding engineering of the enzymes for expression, stability, and productivity in microbial host cells. The two enzymes have 35% identity.

In various embodiments, the heterologous enzyme pathway comprises a triterpene cyclase (TTC). In some embodiments, where the microbial cell coexpresses FPPS, along with the SQS, SQE, and triterpene cyclase enzymes, the microbial cell produces 2,3;22,23-dioxidosqualene. The 2,3;22,23-dioxidosqualene may be the substrate for downstream enzymes in the heterologous pathway. In some embodiments, the triterpene cyclase (TTC) comprises an amino acid sequence that is at least 70%, or at least 80%, or at least 90%, or at least 95% identical to an amino acid sequence selected from SEQ ID NOS: 40 to 55, 191 to 193, and 219 to 220. The TTC in various embodiments comprises an amino acid sequence that is at least 70% identical to the amino acid sequence of SEQ ID NO: 40. In some embodiments, the TTC comprises an amino acid sequence that is at least 80%, or at least 85%, or at least 90%, or at least 95%, or at least 98%, or at least 99% identical to SEQ ID NO: 40. For example, the TTC may comprise an amino acid sequence having from 1 to 20 amino acid modifications with respect to SEQ ID NO: 40, the amino acid modifications being independently selected from amino acid substitutions, deletions, and insertions.

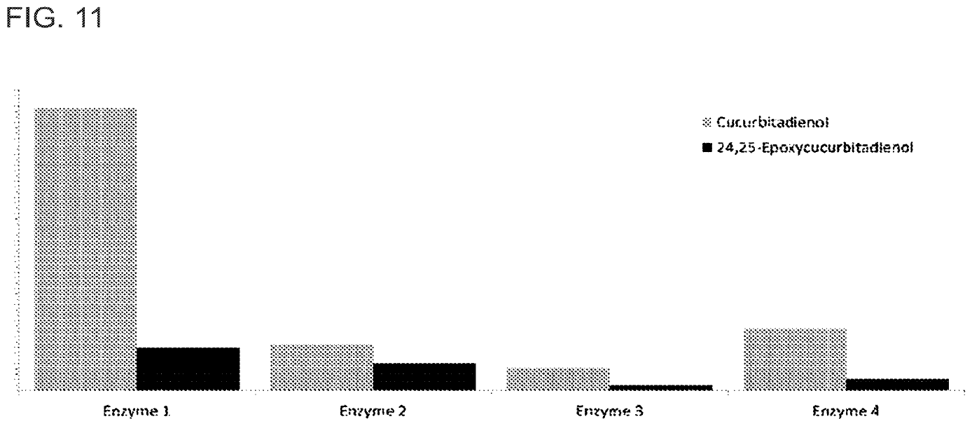

In some embodiments, the TTC comprises an amino acid sequence that is at least 80%, or at least 85%, or at least 90%, or at least 95%, or at least 98%, or at least 99% identical to SEQ ID NO: 192. For example, the TTC may comprise an amino acid sequence having from 1 to 20 amino acid modifications with respect to SEQ ID NO: 192, the amino acid modifications being independently selected from amino acid substitutions, deletions, and insertions. The enzyme defined by SEQ ID NO: 192 shows improved specificity toward production of 24,25-epoxycucurbitadienol (FIG. 11).

In various embodiments, the heterologous enzyme pathway comprises at least two copies of a TTC enzyme gene, or comprises at least two enzymes having triterpene cyclase activity and converting 22,23-dioxidosqualene to 24,25-epoxycucurbitadienol. In such embodiments, product can be pulled to 24,25-epoxycucurbitadienol, with less production of cucurbitadienol.

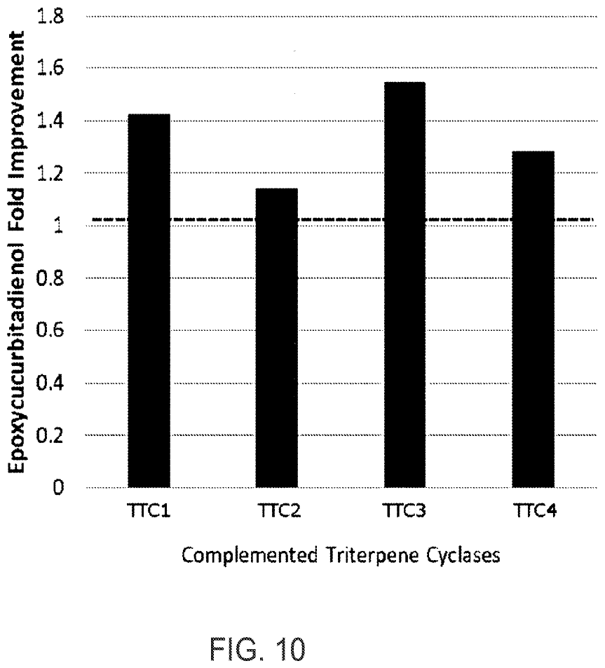

In some embodiments, the heterologous enzyme pathway comprises at least one TTC that comprises an amino acid sequence that is at least 70% identical to one of SEQ ID NO: 191, SEQ ID NO: 192, and SEQ ID NO: 193. These enzymes may be optionally co-expressed with SgCDS. These enzymes exhibit high production of 24,25-epoxycucurbitadienol. FIG. 10. Thus, in some embodiments, at least one TTC comprises an amino acid sequence that is at least 80%, or at least 85%, or at least 90%, or at least 95%, or at least 98%, or at least 99% identical to one of SEQ ID NOS: 191, 192, and 193. In some embodiments, the TTC comprises an amino acid sequence having from 1 to 20 amino acid modifications with respect to one of SEQ ID NOS: 191, 192, and 193, the amino acid modifications being independently selected from amino acid substitutions, deletions, and insertions.

Amino acid modifications may be made to increase expression or stability of the enzyme in the microbial cell, or to increase productivity of the enzyme. Amino acid modifications can be guided by available enzyme structures and homology models, including those described in Itkin M., et al., The biosynthetic pathway of the nonsugar, high-intensity sweetener mogroside V from Siraitia grosvenorii, PNAS (2016) Vol 113 (47): E7619-E7628. For example, the CDS may be modeled using the structure of human lanosterol synthase (oxidosqualene cyclase) (PDB 1W6K).

In various embodiments, cucurbitadienol (intermediate 9 in FIG. 2) is converted to 24,25-epoxycucurbitadienol (5) by one of more enzymes expressed in the host cell. For example, the heterologous pathway may comprise an enzyme having at least about 70%, or at least about 80%, or at least about 85%, or at least about 90%, or at least about 95%, or at least about 97%, 98%, or 99% sequence identity with SEQ ID NO: 221.

In some embodiments, the heterologous enzyme pathway comprises at least one epoxide hydrolase (EPH). The EPH may comprise an amino acid sequence that is at least 70% identical to amino acid sequence selected from SEQ ID NOS: 56 to 72, 184 to 190, and 212. In some embodiments, the EPH may employ as a substrate 24,25-epoxycucurbitadienol (intermediate (5) of FIG. 2), for production of 24,25-dihydroxycucurbitadienol (intermediate (6) of FIG. 2). In some embodiments, the EPH comprises an amino acid sequence that is at least 80%, or at least 85%, or at least 90%, or at least 95%, or at least 98%, or at least 99% identical to one of SEQ ID NOS: 56 to 72, 184 to 190, and 212. Thus, in some embodiments, the EPH comprises an amino acid sequence having from 1 to 20 amino acid modifications with respect to one of SEQ ID NOS: 56 to 72, 184 to 190, and 212, the amino acid modifications being independently selected from amino acid substitutions, deletions, and insertions.

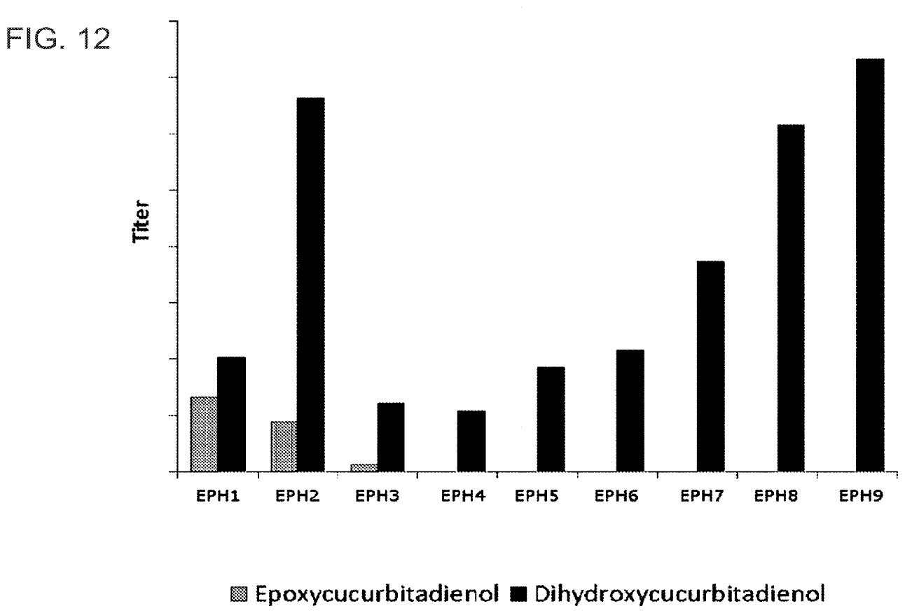

In some embodiments, the heterologous pathway comprises at least one EPH enzyme converting 24,25-epoxycucurbitadienol to 24,25-dihydroxycucurbitadienol, the at least one EPH enzyme comprising an amino acid sequence that is at least 70% identical to one of: SEQ ID NO: 189, SEQ ID NO: 58, SEQ ID NO: 184, SEQ ID NO: 185, SEQ ID NO: 187, SEQ ID NO: 188, SEQ ID NO: 190, and SEQ ID NO: 212. See FIG. 12. In some embodiments, the EPH enzyme comprises an amino acid sequence that is at least 80%, or at least 85%, or at least 90%, or at least 95%, or at least 98%, or at least 99% identical to one of SEQ ID NOS: 189, 58, 184, 185,187, 188, 190, and 212. For example, the EPH may comprise an amino acid sequence having from 1 to 20 amino acid modifications with respect to one of SEQ ID NOS: 189, 58, 184, 185, 187, 188, 190, and 212, the amino acid modifications being independently selected from amino acid substitutions, deletions, and insertions. Amino acid modifications may be made to increase expression or stability of the enzyme in the microbial cell, or to increase productivity of the enzyme.

In some embodiments, the heterologous pathway comprises one or more oxidases. The one or more oxidases may be active on cucurbitadienol or oxygenated products thereof as a substrate, adding (collectively) hydroxylations at C11, C24 and 25, thereby producing mogrol (see FIG. 2). Alternatively, the heterologous pathway may comprise one or more oxidases that oxidize C11 of C24,25 dihydroxycucurbitadienol to produce mogrol.

In some embodiments, at least one oxidase is a cytochrome P450 enzyme. Exemplary cytochrome P450 enzymes comprise an amino acid sequence that is at least 70% identical to an amino acid sequence selected from SEQ ID NOS: 73 to 91, 171 to 176, and 194 to 200. In some embodiments, at least one P450 enzyme comprises an amino acid sequence that is at least 80%, or at least 85%, or at least 90%, or at least 95%, or at least 98%, or at least 99% identical to one of SEQ ID NOS: 73 to 91, 171 to 176, and 194 to 200. For example, at least one cytochrome P450 enzyme comprises an amino acid sequence having from 1 to 20 amino acid modifications with respect to one of SEQ ID NOS: 73 to 91, 171 to 176, and 194 to 200, the amino acid modifications being independently selected from amino acid substitutions, deletions, and insertions.

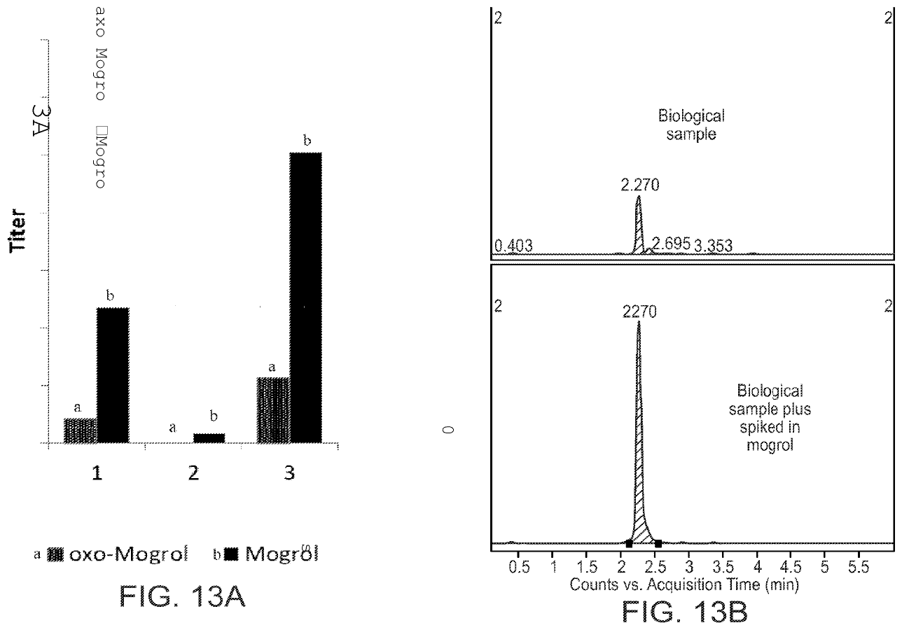

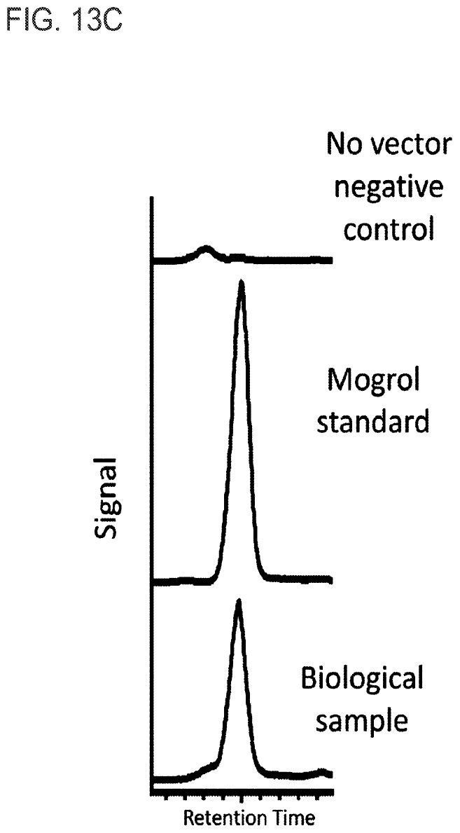

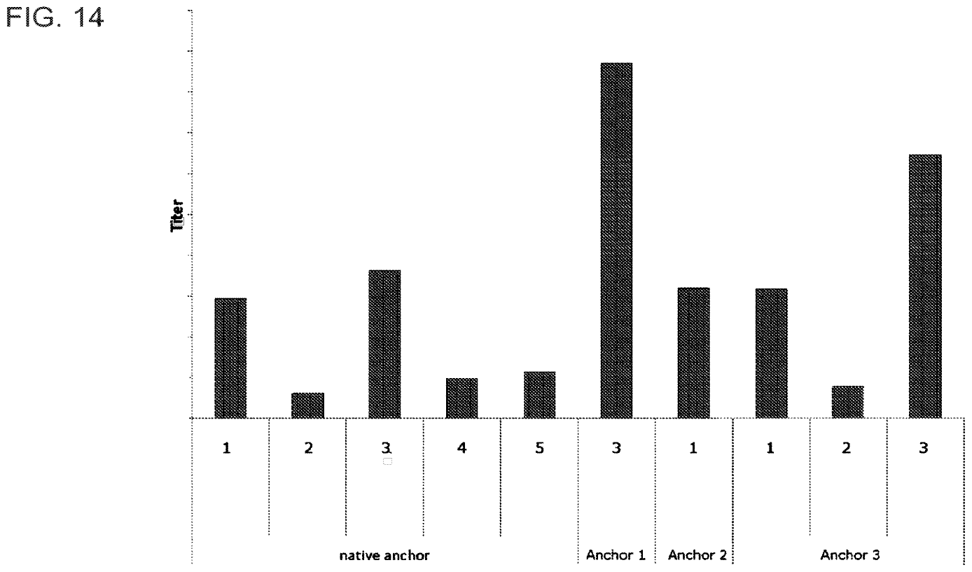

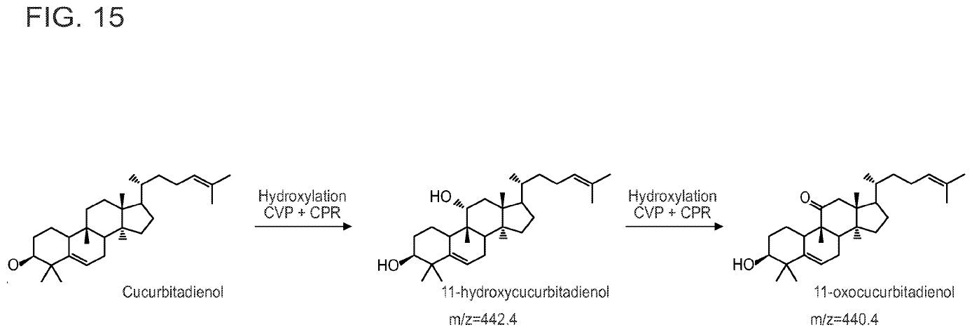

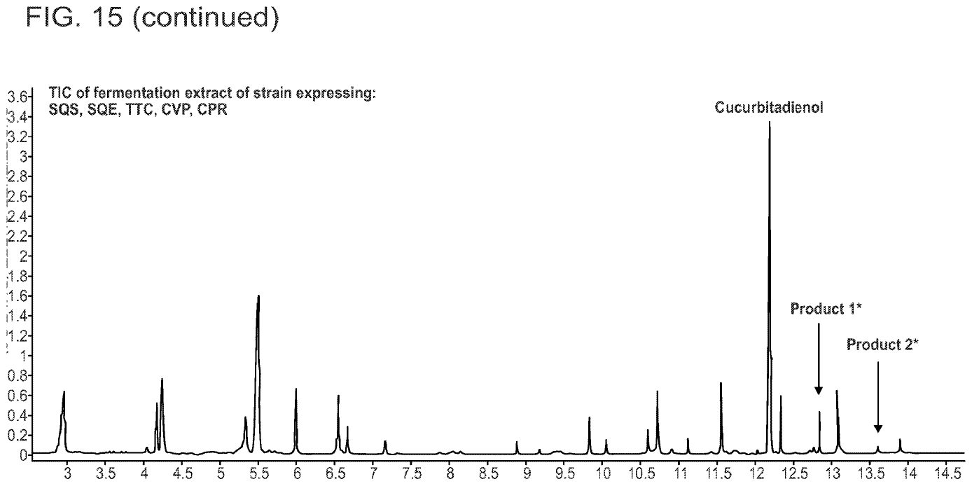

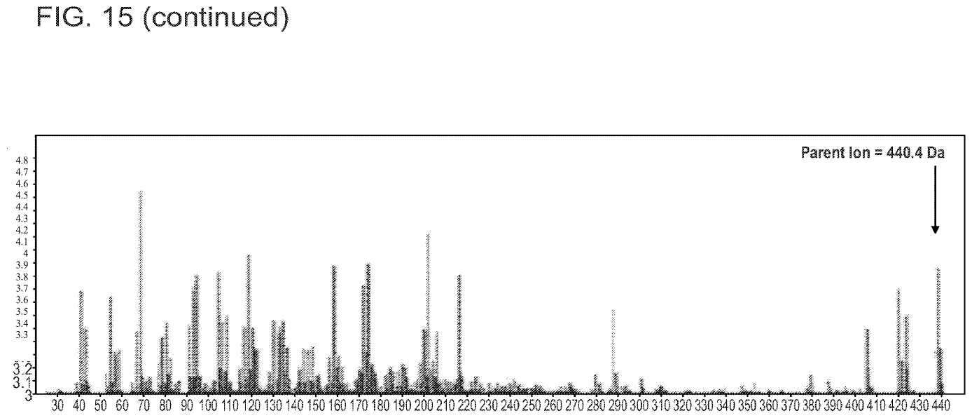

In some embodiments, the microbial host cell expresses a heterologous enzyme pathway comprising a P450 enzyme having activity for oxidation at C11 of C24,25 dihydroxycucurbitadienol, to thereby produce mogrol. For example, in some embodiments, the cytochrome P450 comprises an amino acid sequence that is at least 70% identical to an amino acid sequence selected from SEQ ID NO: 194 and SEQ ID NO: 171. See FIGS. 13A-C, FIG. 14, and FIG. 15. In some embodiments, the microbial host cell expresses a cytochrome P450 enzyme that comprises an amino acid sequence that is at least 80%, or at least 85%, or at least 90%, or at least 95%, or at least 98%, or at least 99% identical to one of SEQ ID NOS: 194 and 171. In some embodiments, at least one cytochrome P450 enzyme comprises an amino acid sequence having from 1 to 20 amino acid modifications with respect to one of SEQ ID NOS: 194 and 171, the amino acid modifications being independently selected from amino acid substitutions, deletions, and insertions.

In some embodiments, the cytochrome P450 enzyme has at least a portion of its transmembrane region substituted with a heterologous transmembrane region. For example, particularly in embodiments in which the microbial cell is a bacterium, the CYP450 and/or CPR is modified as described in US 2018/0251738, the contents of which are hereby incorporated by reference in their entireties. For example, in some embodiments, the CYP450 enzyme has a deletion of all or part of the wild type P450 N-terminal transmembrane region, and the addition of a transmembrane domain derived from an E. coli or bacterial inner membrane, cytoplasmic C-terminus protein. In some embodiments, the transmembrane domain is a single-pass transmembrane domain. In some embodiments, the transmembrane domain is a multi-pass (e.g., 2, 3, or more transmembrane helices) transmembrane domain. Exemplary transmembrane domains are derived from E. coli zipA or sohB. Alternatively, the P450 enzyme can employ its native transmembrane anchor, or the well-known bovine 17a anchor. See FIG. 14.

In some embodiments, the microbial host cell expresses a non-heme iron oxidase. Exemplary non-heme iron oxidases comprise an amino acid sequence that is at least 70% identical to an amino acid sequence selected from SEQ ID NOS: 100 to 115. In some embodiments, the non-heme iron oxidase comprises an amino acid sequence that is at least 80%, or at least 85%, or at least 90%, or at least 95%, or at least 98%, or at least 99% identical to one of SEQ ID NOS: 100 to 115.

In various embodiments, the microbial host cell expresses one or more electron transfer proteins selected from a cytochrome P450 reductase (CPR), flavodoxin reductase (FPR) and ferredoxin reductase (FDXR) sufficient to regenerate the one or more oxidases. Exemplary CPR proteins are provided herein as SEQ ID NOS: 92 to 99 and 201.

In some embodiments, the microbial host cell expresses a cytochrome P450 reductase, and which may comprise an amino acid sequence that is at least 70%, or at least 80%, or at least 85%, or at least 90%, or at least 95%, or at least 98%, or at least 99% identical to one of SEQ ID NOS: 92 to 99 and 201. For example, in some embodiments, the microbial host cell expresses SEQ ID NO: 194 or a derivative thereof (as described above), and SEQ ID NO: 98 or a derivative thereof (i.e., having at least 70%, at least 80%, or at least 90% sequence identity thereto). In some embodiments, the microbial host cell expresses SEQ ID NO: 171 or a derivative thereof (as described above), and SEQ ID NO: 201 or a derivative thereof (i.e., having at least 70%, at least 80%, or at least 90% sequence identity thereto).

In various embodiments, the heterologous enzyme pathway produces mogrol, which may be an intermediate for downstream enzymes in the heterologous pathway, or in some embodiments is recovered from the culture. Mogrol may be recovered from host cells in some embodiments, and/or can be recovered from the culture media.

In some embodiments, the heterologous enzyme pathway further comprises one or more uridine diphosphate-dependent glycosyltransferase (UGT) enzymes, thereby producing one or more mogrol glycosides (or “mogrosides”). The mogrol glycoside may be pentaglycosylated, hexaglycosylated, or more (e.g., 7, 8, or 9 glycosylations), in some embodiments. In other embodiments, the mogrol glycoside has two, three, or four glucosylations. The one or more mogrol glycosides may be selected from Mog.II-E, Mog.III, Mog.III-A1, Mog.III-A2, Mog.III, Mog.IV, Mog.IV-A, siamenoside, isomog. V, Mog. V, or Mog. VI. In some embodiments, the host cell produces Mog. V or siamenoside.

In some embodiments, the host cell expresses a UGT enzyme that catalyzes the primary glycosylation of mogrol at C24 and/or C3 hydroxyl groups. In some embodiments, the UGT enzyme catalyzes a branching glycosylation, such as a beta 1,2 and/or beta 1,6 branching glycosylation at the primary C3 and C24 gluscosyl groups. UGT enzymes observed to catalyze primary glycosylation of C24 and/or C3 hydroxyl groups are summarized in Table 1. UGT enzymes observed to catalyze various branching glycosylation reactions are summarized in Table 2.

In some embodiments, at least one UGT enzyme comprises an amino acid sequence that is at least 70% identical to an amino acid sequence selected from SEQ ID NOS: 116 to 165, 202 to 210, 211, and 213 to 218. For example, in some embodiments, the UGT enzyme comprises an amino acid sequence that is at least 80%, or at least 85%, or at least 90%, or at least 95%, or at least 98%, or at least 99% identical to one of SEQ ID NOS: 116 to 165, 202 to 210, 211, and 213 to 218. Thus, at least one UGT enzyme comprises an amino acid sequence having from 1 to 20 amino acid modifications with respect to one of SEQ ID NOS: 116 to 165, 202 to 210, 211, and 212 to 218, the amino acid modifications being independently selected from amino acid substitutions, deletions, and insertions.

For example, in some embodiments, the microbial cell expresses at least four UGT enzymes, resulting in glucosylation of mogrol at the C3 hydroxyl group, the C24 hydroxyl group, as well as a further 1,6 glucosylation at the C3 glucosyl group, and a further 1,6 glucosylation and a further 1,2 glucosylation at the C24 glucosyl group. The product of such glucosylation reactions is Mog. V.

In some embodiments, at least one UGT enzyme comprises an amino acid sequence having at least 70% sequence identity to one of SEQ ID NO: 164, 165, 138, 204 to 211, and 213 to 218.

In some embodiments, at least one UGT enzyme comprises an amino acid sequence that is at least 70% identical to UGT85C1 (SEQ ID NO: 165). UGT85C1 exhibits primary glycosylation at the C3 and C24 hydroxyl groups. Thus, in some embodiments, at least one UGT enzyme comprises an amino acid sequence that is at least 80%, or at least 85%, or at least 90%, or at least 95%, or at least 98%, or at least 99% identical to SEQ ID NO: 165. The at least one UGT enzyme may comprise an amino acid sequence having from 1 to 20 amino acid modifications with respect to SEQ ID NO: 165, the amino acid modifications being independently selected from amino acid substitutions, deletions, and insertions. Exemplary amino acid substitutions include substitutions at positions 41 (e.g., L41F or L41Y), 49 (e.g., D49E), and 127 (e.g., C127F or C127Y).

In some embodiments, at least one UGT enzyme comprises an amino acid sequence that is at least 70% identical to SEQ ID NO: 164, which exhibits activity for adding branching glycosylations, both 1-2 and 1-6 branching glycosylations. In various embodiments, at least one UGT enzyme comprises an amino acid sequence that is at least 80%, or at least 85%, or at least 90%, or at least 95%, or at least 98%, or at least 99% identical to SEQ ID NO: 164. In exemplary embodiments, at least one UGT enzyme comprises an amino acid sequence having from 1 to 20 amino acid modifications with respect to SEQ ID NO: 164, the amino acid modifications being independently selected from amino acid substitutions, deletions, and insertions. Exemplary amino acid substitutions are shown in Table 3. Exemplary amino acid substitutions include substitutions at one or more positions selected from 150 (e.g., S15OF, S150Y), 147 (e.g., T147L, T147V, T147I, and T147A), 207 (e.g., N207K or N207R), 270 (e.g., K270E or K270D), 281 (V281L or V281I), 354 (e.g., L354V or L3541), 13 (e.g., L13F or L13Y), 32 (T32A or T32G or T32L), and 101 (K101A or K101G), with respect to SEQ ID NO: 164. An exemplary engineered UGT enzyme comprises the amino acid substitutions T147L and N207K, with respect to SEQ ID NO: 164.

In some embodiments, at least one UGT enzyme comprises an amino acid sequence that is at least 70% identical to SEQ ID NO: 138, which exhibits an activity to catalyze 1-6 branching glycosylations. In some embodiments, at least one UGT enzyme comprises an amino acid sequence that is at least 80%, or at least 85%, or at least 90%, or at least 95%, or at least 98%, or at least 99% identical to SEQ ID NO: 138. In exemplary embodiments, at least one UGT enzyme comprises an amino acid sequence having from 1 to 20 amino acid modifications with respect to SEQ ID NO: 138, the amino acid modifications being independently selected from amino acid substitutions, deletions, and insertions.

In some embodiments, at least one UGT enzyme comprises an amino acid sequence that is at least 70% identical to SEQ ID NO: 204, which catalyzes 1-6 branching glycosylation, particularly at the C3 primary glucosylation. For example, at least one UGT enzyme may comprise an amino acid sequence that is at least 80%, or at least 85%, or at least 90%, or at least 95%, or at least 98%, or at least 99% identical to SEQ ID NO: 204. In exemplary embodiments, at least one UGT enzyme comprises an amino acid sequence having from 1 to 20 amino acid modifications with respect to SEQ ID NO: 204, the amino acid modifications being independently selected from amino acid substitutions, deletions, and insertions.

In some embodiments, at least one UGT enzyme comprises an amino acid sequence that is at least 70% identical to SEQ ID NO: 205, which catalyzes 1-6 branching glycosylation, including at both the C3 and C24 primary glucosylations. For example, at least one UGT enzyme may comprise an amino acid sequence that is at least 80%, or at least 85%, or at least 90%, or at least 95%, or at least 98%, or at least 99% identical to SEQ ID NO: 205. In exemplary embodiments, at least one UGT enzyme comprises an amino acid sequence having from 1 to 20 amino acid modifications with respect to SEQ ID NO: 205, the amino acid modifications being independently selected from amino acid substitutions, deletions, and insertions.

In some embodiments, at least one UGT enzyme comprises an amino acid sequence that is at least 70% identical to SEQ ID NO: 206, which catalyzes 1-2 and 1-6 branching glycosylations. For example, at least one UGT enzyme may comprise an amino acid sequence that is at least 80%, or at least 85%, or at least 90%, or at least 95%, or at least 98%, or at least 99% identical to SEQ ID NO: 206. In exemplary embodiments, at least one UGT enzyme comprises an amino acid sequence having from 1 to 20 amino acid modifications with respect to SEQ ID NO: 206, the amino acid modifications being independently selected from amino acid substitutions, deletions, and insertions.

In some embodiments, at least one UGT enzyme comprises an amino acid sequence that is at least 70% identical to SEQ ID NO: 207, which catalyzes 1-6 branching glycosylations of the primary glucosylations. For example, at least one UGT enzyme may comprise an amino acid sequence that is at least 80%, or at least 85%, or at least 90%, or at least 95%, or at least 98%, or at least 99% identical to SEQ ID NO: 207. In exemplary embodiments, at least one UGT enzyme comprises an amino acid sequence having from 1 to 20 amino acid modifications with respect to SEQ ID NO: 207, the amino acid modifications being independently selected from amino acid substitutions, deletions, and insertions.

In some embodiments, at least one UGT enzyme comprises an amino acid sequence that is at least 70% identical to SEQ ID NO: 208, which catalyzes 1-2 and 1-6 branching glycosylations. For example, at least one UGT enzyme may comprise an amino acid sequence that is at least 80%, or at least 85%, or at least 90%, or at least 95%, or at least 98%, or at least 99% identical to SEQ ID NO: 208. In exemplary embodiments, at least one UGT enzyme comprises an amino acid sequence having from 1 to 20 amino acid modifications with respect to SEQ ID NO: 208, the amino acid modifications being independently selected from amino acid substitutions, deletions, and insertions.

In some embodiments, at least one UGT enzyme comprises an amino acid sequence that is at least 70% identical to SEQ ID NO: 209, which catalyzes 1-6 branching glycosylations of the primary glucosylations. For example, at least one UGT enzyme may comprise an amino acid sequence that is at least 80%, or at least 85%, or at least 90%, or at least 95%, or at least 98%, or at least 99% identical to SEQ ID NO: 209. In exemplary embodiments, at least one UGT enzyme comprises an amino acid sequence having from 1 to 20 amino acid modifications with respect to SEQ ID NO: 209, the amino acid modifications being independently selected from amino acid substitutions, deletions, and insertions.

In some embodiments, at least one UGT enzyme comprises an amino acid sequence that is at least 70% identical to SEQ ID NO: 210), which catalyzes 1-6 branching glycosylations of the primary glucosylations. For example, at least one UGT enzyme may comprise an amino acid sequence that is at least 80%, or at least 85%, or at least 90%, or at least 95%, or at least 98%, or at least 99% identical to SEQ ID NO: 210. In exemplary embodiments, at least one UGT enzyme comprises an amino acid sequence having from 1 to 20 amino acid modifications with respect to SEQ ID NO: 210, the amino acid modifications being independently selected from amino acid substitutions, deletions, and insertions.

In some embodiments, at least one UGT enzyme comprises an amino acid sequence that is at least 70% identical to SEQ ID NO: 211, which catalyzes 1-2 branching glycosylation of the C24 primary glucosylation. For example, at least one UGT enzyme may comprise an amino acid sequence that is at least 80%, or at least 85%, or at least 90%, or at least 95%, or at least 98%, or at least 99% identical to SEQ ID NO: 211. In exemplary embodiments, at least one UGT enzyme comprises an amino acid sequence having from 1 to 20 amino acid modifications with respect to SEQ ID NO: 210, the amino acid modifications being independently selected from amino acid substitutions, deletions, and insertions.

In some embodiments, at least one UGT enzyme comprises an amino acid sequence that is at least 70% identical to SEQ ID NO: 213, which catalyzes 1-6 branching glycosylation of the primary glucosylation at C24. For example, at least one UGT enzyme may comprise an amino acid sequence that is at least 80%, or at least 85%, or at least 90%, or at least 95%, or at least 98%, or at least 99% identical to SEQ ID NO: 213. In exemplary embodiments, at least one UGT enzyme comprises an amino acid sequence having from 1 to 20 amino acid modifications with respect to SEQ ID NO: 213, the amino acid modifications being independently selected from amino acid substitutions, deletions, and insertions.

In some embodiments, at least one UGT enzyme comprises an amino acid sequence that is at least 70% identical to SEQ ID NO: 214, which catalyzes primary glucosylation at C24. For example, at least one UGT enzyme may comprise an amino acid sequence that is at least 80%, or at least 85%, or at least 90%, or at least 95%, or at least 98%, or at least 99% identical to SEQ ID NO: 214. In exemplary embodiments, at least one UGT enzyme comprises an amino acid sequence having from 1 to 20 amino acid modifications with respect to SEQ ID NO: 214, the amino acid modifications being independently selected from amino acid substitutions, deletions, and insertions.

In some embodiments, at least one UGT enzyme comprises an amino acid sequence that is at least 70% identical to SEQ ID NO: 215, which catalyzes 1-6 branching glucosylation at C24. For example, at least one UGT enzyme may comprise an amino acid sequence that is at least 80%, or at least 85%, or at least 90%, or at least 95%, or at least 98%, or at least 99% identical to SEQ ID NO: 215. In exemplary embodiments, at least one UGT enzyme comprises an amino acid sequence having from 1 to 20 amino acid modifications with respect to SEQ ID NO: 215, the amino acid modifications being independently selected from amino acid substitutions, deletions, and insertions.

In still other embodiments, at least one UGT enzyme comprises an amino acid sequence that is at least 70% identical to SEQ ID NO: 146, which provides for glucosylation of the C24 hydroxyl of mogrol or Mog. 1E. In some embodiments, at least one UGT enzyme comprises an amino acid sequence that is at least 80%, or at least 85%, or at least 90%, or at least 95%, or at least 98%, or at least 99% identical to SEQ ID NO: 146. In some embodiments, at least one UGT enzyme comprises an amino acid sequence having from 1 to 20 or from 1 to 10 amino acid modifications with respect to SEQ ID NO: 146, the amino acid modifications being independently selected from amino acid substitutions, deletions, and insertions. Amino acid modifications may be made to increase expression or stability of the enzyme in the microbial cell, or to increase productivity of the enzyme for particular substrates.

In some embodiments, at least one UGT enzyme comprises an amino acid sequence that is at least 70% identical to SEQ ID NO: 202, which catalyzes primary glycosylation at the C3 and C24 hydroxyl. For example, at least one UGT enzyme may comprise an amino acid sequence that is at least 80%, or at least 85%, or at least 90%, or at least 95%, or at least 98%, or at least 99% identical to SEQ ID NO: 202. In exemplary embodiments, at least one UGT enzyme comprises an amino acid sequence having from 1 to 20 amino acid modifications with respect to SEQ ID NO: 202, the amino acid modifications being independently selected from amino acid substitutions, deletions, and insertions.

In some embodiments, at least one UGT enzyme comprises an amino acid sequence that is at least 70% identical to SEQ ID NO: 218, which catalyzes primary glycosylation at the C24 hydroxyl. For example, at least one UGT enzyme may comprise an amino acid sequence that is at least 80%, or at least 85%, or at least 90%, or at least 95%, or at least 98%, or at least 99% identical to SEQ ID NO: 218. In exemplary embodiments, at least one UGT enzyme comprises an amino acid sequence having from 1 to 20 amino acid modifications with respect to SEQ ID NO: 218, the amino acid modifications being independently selected from amino acid substitutions, deletions, and insertions.

In some embodiments, at least one UGT enzyme comprises an amino acid sequence that is at least 70% identical to SEQ ID NO: 217, which catalyzes primary glycosylation at the C24 hydroxyl. For example, at least one UGT enzyme may comprise an amino acid sequence that is at least 80%, or at least 85%, or at least 90%, or at least 95%, or at least 98%, or at least 99% identical to SEQ ID NO: 217. In exemplary embodiments, at least one UGT enzyme comprises an amino acid sequence having from 1 to 20 amino acid modifications with respect to SEQ ID NO: 217, the amino acid modifications being independently selected from amino acid substitutions, deletions, and insertions. Exemplary amino acid substitutions include substitutions at one or more positions (with respect to SEQ ID NO: 17) selected from 74 (e.g., A74E or A74D), 91 (191F or 191Y), 101 (e.g., H101P), 241 (e.g., Q241E or Q241D), and 436 (e.g., 1436L or I436A). In some embodiments, the UGT enzyme comprises the following amino acid substitutions with respect to SEQ ID NO: 217: A74E, I91F, and H101P.

In some embodiments, at least one UGT enzyme comprises an amino acid sequence that is at least 70% identical to SEQ ID NO: 216, which catalyzes primary glycosylation at the C24 hydroxyl. For example, at least one UGT enzyme may comprise an amino acid sequence that is at least 80%, or at least 85%, or at least 90%, or at least 95%, or at least 98%, or at least 99% identical to SEQ ID NO: 216. In exemplary embodiments, at least one UGT enzyme comprises an amino acid sequence having from 1 to 20 amino acid modifications with respect to SEQ ID NO: 216, the amino acid modifications being independently selected from amino acid substitutions, deletions, and insertions.

In some embodiments, at least one UGT enzyme comprises an amino acid sequence that is at least 70% identical to SEQ ID NO: 117, SEQ ID NO: 210, or SEQ ID NO: 122. For example, the enzyme defined by SEQ ID NO: 117 catalyzes branching glycosylations. In some embodiments, at least one UGT enzyme comprises an amino acid sequence that is at least 80%, or at least 85%, or at least 90%, or at least 95%, or at least 98%, or at least 99% identical to SEQ ID NO: 117, SEQ ID NO: 210, or SEQ ID NO: 122. In some embodiments, at least one UGT enzyme comprises an amino acid sequence having from 1 to 20 amino acid modifications with respect to SEQ ID NO: 117, 210, or 122, the amino acid modifications being independently selected from amino acid substitutions, deletions, and insertions.

In some embodiments, the microbial cell expresses at least one UGT enzyme capable of catalyzing beta 1,2 addition of a glucose molecule to at least the C24 glucosyl group (e.g., of Mog.IVA). Exemplary UGT enzymes in accordance with these embodiments include SEQ ID NO: 117, SEQ ID NO: 147, SEQ ID NO: 148, SEQ ID NO: 149, SEQ ID NO: 150, or SEQ ID NO: 163, or derivatives thereof. Derivatives include enzymes comprising amino acid sequence that are least 70% identical to one or more of SEQ ID NO: 117, SEQ ID NO: 147, SEQ ID NO: 148, SEQ ID NO: 149, SEQ ID NO: 150, and SEQ ID NO: 163. In some embodiments, the UGT enzyme catalyzing beta 1,2 addition of a glucose molecule to at least the C24 glucosyl group comprises an amino acid sequence that is at least 80%, or at least 85%, or at least 90%, or at least 95%, or at least 98%, or at least 99% identical to one or more of SEQ ID NO: 117, SEQ ID NO: 147, SEQ ID NO: 148, SEQ ID NO: 149, SEQ ID NO: 150, and SEQ ID NO: 163. In some embodiments, at least one UGT enzyme comprises an amino acid sequence having from 1 to 20 or having from 1 to 10 amino acid modifications with respect to SEQ ID NO: 117, SEQ ID NO: 147, SEQ ID NO: 148, SEQ ID NO: 149, SEQ ID NO: 150, and SEQ ID NO: 163, the amino acid modifications being independently selected from amino acid substitutions, deletions, and insertions. Amino acid modifications may be made to increase expression or stability of the enzyme in the microbial cell, or to increase productivity of the enzyme for particular substrates.

In some embodiments, at least one UGT enzyme is a circular permutant of a wild-type UGT enzyme, optionally having amino acid substitutions, deletions, and/or insertions with respect to the corresponding position of the wild-type enzyme. Circular permutants can provide novel and desirable substrate specificities, product profiles, and reaction kinetics over the wild-type enzymes. A circular permutant retains the same basic fold of the parent enzyme, but has a different position of the N-terminus (e.g., “cut-site”), with the original N- and C-termini connected, optionally by a linking sequence. For example, in the circular permutants, the N-terminal Methionine is positioned at a site in the protein other than the natural N-terminus. UGT circular permutants are described in US 2017/0332673, which is hereby incorporated by reference in its entirety. In some embodiments, at least one UGT enzyme is a circular permutant of a UGT enzyme described herein, such as but not limited to SEQ ID NO: 146, SEQ ID NO: 164, or SEQ ID NO: 165, SEQ ID NO: 117, SEQ ID NO: 147, SEQ ID NO: 148, SEQ ID NO: 149, SEQ ID NO: 150, SEQ ID NO: 163, SEQ ID NO: 202, SEQ ID NO: 216, SEQ ID NO: 217, and SEQ ID NO: 218. In some embodiments, the circular permutant further has one or more amino acid modifications (e.g., amino acid substitutions, deletions, and/or insertions) with respect to the parent UGT enzyme. In these embodiments, the circular permutant will have at least about 70%, or at least about 80%, or at least about 90%, or at least about 95%, or at least about 98% identity to the parent enzyme, when the corresponding amino acid sequences are aligned (i.e., without regard to the new N-terminus of the circular permutant). An exemplary circular permutant for use according to some embodiments is SEQ ID NO: 206.

In some embodiments, the microbial host cell expresses at least three UGT enzymes: a first UGT enzyme catalyzing primary glycosylation at the C24 hydroxyl of mogrol, a second UGT enzyme catalyzing primary glycosylation at the C3 hydroxyl of mogrol, and a third UGT enzyme catalyzing one or more branching glycosylation reactions. In some embodiments, the microbial host cell expresses one or two UGT enzymes catalyzing beta 1,2 and/or beta 1,6 branching glycosylations of the C3 and/or C24 primary glycosylations. For example, the UGT enzymes may comprise three or four UGT enzymes selected from:
SEQ ID NO: 165 or a derivative thereof;
SEQ ID NO: 146 or a derivative thereof;
SEQ ID NO: 214 or a derivative thereof;
SEQ ID NO: 129 or a derivative thereof;
SEQ ID NO: 164 or a derivative thereof;
SEQ ID NO: 116 or a derivative thereof;
SEQ ID NO: 202 or a derivative thereof;
SEQ ID NO: 218 or a derivative thereof;
SEQ ID NO: 217 or a derivative thereof;
SEQ ID NO: 138 or a derivative thereof;
SEQ ID NO: 204 or a derivative thereof;
SEQ ID NO: 205 or a derivative thereof;
SEQ ID NO: 207 or a derivative thereof;
SEQ ID NO: 208 or a derivative thereof;
SEQ ID NO: 209 or a derivative thereof;
SEQ ID NO: 11 or a derivative thereof;
SEQ ID NO: 215 or a derivative thereof;
SEQ ID NO: 213 or a derivative thereof;
SEQ ID NO: 206 or a derivative thereof;
SEQ ID NO: 122 or a derivative thereof; and
SEQ ID NO: 210) or a derivative thereof. Derivatives have sequence identity to the reference enzyme as described herein.

In some embodiments, the microbial host cell has one or more genetic modifications that increase the production of UDP-glucose, the co-factor employed by UGT enzymes. These genetic modifications may include one or more, or two or more (or all) of AgalE, AgalT, AgalK, AgalM, AushA, Aagp, Apgm, duplication of E. coli galU, expression of Bacillus subtillus UGPA, and expression of Bifidobacterium adolescentis SPL.

Mogrol glycosides can be recovered from the microbial culture. For example, mogrol glycosides may be recovered from microbial cells, or in some embodiments, are predominately available in the extracellular media, where they may be recovered or sequestered.

In various embodiments, the reaction is performed in a microbial cell, and UGT enzymes are recombinantly expressed in the cell. In some embodiments, mogrol is produced in the cell by a heterologous mogrol synthesis pathway, as described herein. In other embodiments, mogrol or mogrol glycosides (such as a monkfruit extract) are fed to the cells for glycosylation. In still other embodiments, the reaction is performed in vitro using purified UGT enzyme, partially purified UGT enzyme, or recombinant cell lysates.

As described herein, the microbial host cell can be prokaryotic or eukaryotic, and is optionally a bacterium selected from Escherichia coli, Bacillus subtilis, Corynebacterium glutamicum, Rhodobacter capsulatus, Rhodobacter sphaeroides, Zymomonas mobilis, Vibrio natriegens, or Pseudomonas putida. In some embodiments, the microbial cell is a yeast selected from a species of Saccharomyces, Pichia, or Yarrowia, including Saccharomyces cerevisiae, Pichia pastoris, and Yarrowia lipolytica. In some embodiments, the microbial host cell is E. coli.

The bacterial host cell is cultured to produce the triterpenoid product (e.g., mogroside). In some embodiments, carbon substrates such as C1, C2, C3, C4, C5, and/or C6 carbon substrates are employed for the production phase. In exemplary embodiments, the carbon source is glucose, sucrose, fructose, xylose, and/or glycerol. Culture conditions are generally selected from aerobic, microaerobic, and anaerobic.

In various embodiments, the bacterial host cell may be cultured at a temperature between 22° C. and 37° C. While commercial biosynthesis in bacteria such as E. coli can be limited by the temperature at which overexpressed and/or foreign enzymes (e.g., enzymes derived from plants) are stable, recombinant enzymes may be engineered to allow for cultures to be maintained at higher temperatures, resulting in higher yields and higher overall productivity. In some embodiments, the culturing is conducted at about 22° C. or greater, about 23° C. or greater, about 24° C. or greater, about 25° C. or greater, about 26° C. or greater, about 27° C. or greater, about 28° C. or greater, about 29° C. or greater, about 30° C. or greater, about 31° C. or greater, about 32° C. or greater, about 33° C. or greater, about 34° C. or greater, about 35° C. or greater, about 36° C. or greater, or about 37° C.

In some embodiments, the bacterial host cells are further suitable for commercial production, at commercial scale. In some embodiments, the size of the culture is at least about 100 L, at least about 200 L, at least about 500 L, at least about 1,000 L, or at least about 10,000 L, or at least about 100,000 L, or at least about 500,000 L, or at least about 600,000 L. In an embodiment, the culturing may be conducted in batch culture, continuous culture, or semi-continuous culture.

In various embodiments, methods further include recovering the product from the cell culture or from cell lysates. In some embodiments, the culture produces at least about 100 mg/L, or at least about 200 mg/L, or at least about 500 mg/L, or at least about 1 g/L, or at least about 2 g/L, or at least about 5 g/L, or at least about 10 g/L, or at least about 20 g/L, or at least about 30 g/L, or at least about 40 g/L of the terpenoid or terpenoid glycoside product.

In some embodiments, the production of indole (including prenylated indole) is used as a surrogate marker for terpenoid production, and/or the accumulation of indole in the culture is controlled to increase production. For example, in various embodiments, accumulation of indole in the culture is controlled to below about 100 mg/L, or below about 75 mg/L, or below about 50 mg/L, or below about 25 mg/L, or below about 10 mg/L. The accumulation of indole can be controlled by balancing protein expression and activity using the multivariate modular approach as described in U.S. Pat. No. 8,927,241 (which is hereby incorporated by reference), and/or is controlled by chemical means.

Other markers for efficient production of terpene and terpenoids, include accumulation of DOX or ME in the culture media. Generally, the bacterial strains may be engineered to accumulate less of these chemical species, which accumulate in the culture at less than about 5 g/L, or less than about 4 g/L, or less than about 3 g/L, or less than about 2 g/L, or less than about 1 g/L, or less than about 500 mg/L, or less than about 100 mg/L.

The optimization of terpene or terpenoid production by manipulation of MEP pathway genes, as well as manipulation of the upstream and downstream pathways, is not expected to be a simple linear or additive process. Rather, through combinatorial analysis, optimization is achieved through balancing components of the MEP pathway, as well as upstream and downstream pathways. Indole (including prenylated indole) accumulation and MEP metabolite accumulation (e.g., DOX, ME, MEcPP, and/or farnesol) in the culture can be used as surrogate markers to guide this process.

For example, in some embodiments, the bacterial strain has at least one additional copy of dxs and idi expressed as an operon/module; or dxs, ispD, ispF, and idi expressed as an operon or module (either on a plasmid or integrated into the genome), with additional MEP pathway complementation described herein to improve MEP carbon. For example, the bacterial strain may have a further copy of dxr, and ispG and/or ispH, optionally with a further copy of ispE and/or idi, with expressions of these genes tuned to increase MEP carbon and/or improve terpene or terpenoid titer. In various embodiments, the bacterial strain has a further copy of at least dxr, ispE, ispG and ispH, optionally with a further copy of idi, with expressions of these genes tuned to increase MEP carbon and/or improve terpene or terpenoid titer.

Manipulation of the expression of genes and/or proteins, including gene modules, can be achieved through various methods. For example, expression of the genes or operons can be regulated through selection of promoters, such as inducible or constitutive promoters, with different strengths (e.g., strong, intermediate, or weak). Several non-limiting examples of promoters of different strengths include Trc, T5 and T7. Additionally, expression of genes or operons can be regulated through manipulation of the copy number of the gene or operon in the cell. In some embodiments, expression of genes or operons can be regulated through manipulating the order of the genes within a module, where the genes transcribed first are generally expressed at a higher level. In some embodiments, expression of genes or operons is regulated through integration of one or more genes or operons into the chromosome.

Optimization of protein expression can also be achieved through selection of appropriate promoters and ribosomal binding sites. In some embodiments, this may include the selection of high-copy number plasmids, or single-, low- or medium-copy number plasmids. The step of transcription termination can also be targeted for regulation of gene expression, through the introduction or elimination of structures such as stem-loops.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA. The heterologous DNA is placed under operable control of transcriptional elements to permit the expression of the heterologous DNA in the host cell.

In some embodiments, endogenous genes are edited, as opposed to gene complementation. Editing can modify endogenous promoters, ribosomal binding sequences, or other expression control sequences, and/or in some embodiments modifies trans-acting and/or cis-acting factors in gene regulation. Genome editing can take place using CRISPR/Cas genome editing techniques, or similar techniques employing zinc finger nucleases and TALENs. In some embodiments, the endogenous genes are replaced by homologous recombination.

In some embodiments, genes are overexpressed at least in part by controlling gene copy number. While gene copy number can be conveniently controlled using plasmids with varying copy number, gene duplication and chromosomal integration can also be employed. For example, a process for genetically stable tandem gene duplication is described in US 2011/0236927, which is hereby incorporated by reference in its entirety.

The terpene or terpenoid product can be recovered by any suitable process. For example, the aqueous phase can be recovered, and/or the whole cell biomass can be recovered, for further processing. The production of the desired product can be determined and/or quantified, for example, by gas chromatography (e.g., GC-MS). The desired product can be produced in batch or continuous bioreactor systems.

The similarity of nucleotide and amino acid sequences, i.e. the percentage of sequence identity, can be determined via sequence alignments. Such alignments can be carried out with several art-known algorithms, such as with the mathematical algorithm of Karlin and Altschul (Karlin & Altschul (1993) Proc. Natl. Acad. Sci. USA 90:5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80). The grade of sequence identity (sequence matching) may be calculated using e.g. BLAST, BLAT or BlastZ (or BlastX). A similar algorithm is incorporated into the BLASTN and BLASTP programs of Altschul et al (1990) J. Mol. Biol. 215:403-410. BLAST polynucleotide searches can be performed with the BLASTN program, score=100, word length=12.

BLAST protein searches may be performed with the BLASTP program, score=50, word length=3. To obtain gapped alignments for comparative purposes, Gapped BLAST is utilized as described in Altschul et al (1997) Nucleic Acids Res. 25:3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups:
(1) hydrophobic: Met, Ala, Val, Leu, Ile;
(2) neutral hydrophilic: Cys, Ser, Thr; Asn, Gin;
(3) acidic: Asp, Glu;
(4) basic: His, Lys, Arg;
(5) residues that influence chain orientation: Gly, Pro; and
(6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. Some preferred conservative substitutions within the above six groups are exchanges within the following sub-groups: (i) Ala, Val, Leu and Ile; (ii) Ser and Thr; (ii) Asn and Gln; (iv) Lys and Arg; and (v) Tyr and Phe.

As used herein, “non-conservative substitutions” are defined as exchanges of an amino acid by another amino acid listed in a different group of the six standard amino acid groups (1) to (6) shown above.

Modifications of enzymes as described herein can include conservative and/or non-conservative mutations. In some embodiments, an Alanine is substituted or inserted at position 2, to increase stability.

In some embodiments “rational design” is involved in constructing specific mutations in enzymes. Rational design refers to incorporating knowledge of the enzyme, or related enzymes, such as its reaction thermodynamics and kinetics, its three dimensional structure, its active site(s), its substrate(s) and/or the interaction between the enzyme and substrate, into the design of the specific mutation. Based on a rational design approach, mutations can be created in an enzyme which can then be screened for increased production of a terpene or terpenoid relative to control levels. In some embodiments, mutations can be rationally designed based on homology modeling. As used herein, “homology modeling” refers to the process of constructing an atomic resolution model of one protein from its amino acid sequence and a three-dimensional structure of a related homologous protein.

In other aspects, the invention provides a method for making a product comprising a mogrol glycoside. The method comprises producing a mogrol glycoside in accordance with this disclosure, and incorporating the mogrol glycoside into a product. In some embodiments, the mogrol glycoside is siamenoside, Mog. V, Mog. VI, or Isomog. V. In some embodiments, the product is a sweetener composition, flavoring composition, food, beverage, chewing gum, texturant, pharmaceutical composition, tobacco product, nutraceutical composition, or oral hygiene composition.

The product may be a sweetener composition comprising a blend of artificial and/or natural sweeteners. For example, the composition may further comprise one or more of a steviol glycoside, aspartame, and neotame. Exemplary steviol glycosides comprises one or more of RebM, RebB, RebD, RebA, RebE, and RebI.

Non-limiting examples of flavors for which the products can be used in combination include lime, lemon, orange, fruit, banana, grape, pear, pineapple, mango, bitter almond, cola, cinnamon, sugar, cotton candy and vanilla flavors. Non-limiting examples of other food ingredients include flavors, acidulants, and amino acids, coloring agents, bulking agents, modified starches, gums, texturizers, preservatives, antioxidants, emulsifiers, stabilizers, thickeners and gelling agents.

Mogrol glycosides obtained according to this invention may be incorporated as a high intensity natural sweetener in foodstuffs, beverages, pharmaceutical compositions, cosmetics, chewing gums, table top products, cereals, dairy products, toothpastes and other oral cavity compositions, etc.

Mogrol glycosides obtained according to this invention can be used in combination with various physiologically active substances or functional ingredients. Functional ingredients generally are classified into categories such as carotenoids, dietary fiber, fatty acids, saponins, antioxidants, nutraceuticals, flavonoids, isothiocyanates, phenols, plant sterols and stanols (phytosterols and phytostanols); polyols; prebiotics, probiotics; phytoestrogens; soy protein; sulfides/thiols; amino acids; proteins; vitamins; and minerals. Functional ingredients also may be classified based on their health benefits, such as cardiovascular, cholesterol-reducing, and anti-inflammatory.

Mogrol glycosides obtained according to this invention may be applied as a high intensity sweetener to produce zero calorie, reduced calorie or diabetic beverages and food products with improved taste characteristics. It may also be used in drinks, foodstuffs, pharmaceuticals, and other products in which sugar cannot be used. In addition, highly purified target mogrol glycoside(s), particularly, Mog. V, Mog. VI, or Isomog. V, can be used as a sweetener not only for drinks, foodstuffs, and other products dedicated for human consumption, but also in animal feed and fodder with improved characteristics.

Examples of products in which mogrol glycoside(s) may be used as a sweetening compound include, but are not limited to, alcoholic beverages such as vodka, wine, beer, liquor, and sake, etc.; natural juices; refreshing drinks; carbonated soft drinks; diet drinks; zero calorie drinks; reduced calorie drinks and foods; yogurt drinks; instant juices; instant coffee; powdered types of instant beverages; canned products; syrups; fermented soybean paste; soy sauce; vinegar; dressings; mayonnaise; ketchups; curry; soup; instant bouillon; powdered soy sauce; powdered vinegar; types of biscuits; rice biscuit; crackers; bread; chocolates; caramel; candy; chewing gum; jelly; pudding; preserved fruits and vegetables; fresh cream; jam; marmalade; flower paste; powdered milk; ice cream; sorbet; vegetables and fruits packed in bottles; canned and boiled beans; meat and foods boiled in sweetened sauce; agricultural vegetable food products; seafood; ham; sausage; fish ham; fish sausage; fish paste; deep fried fish products; dried seafood products; frozen food products; preserved seaweed; preserved meat; tobacco; medicinal products; and many others.

During the manufacturing of products such as foodstuffs, drinks, pharmaceuticals, cosmetics, table top products, and chewing gum, the conventional methods such as mixing, kneading, dissolution, pickling, permeation, percolation, sprinkling, atomizing, infusing and other methods may be used.

As used in this specification and the appended claims, the singular forms “a”, “an” and “the” include plural referents unless the content clearly dictates otherwise. For example, reference to “a cell” includes a combination of two or more cells, and the like.

As used herein, the term “about” in reference to a number is generally taken to include numbers that fall within a range of 10% in either direction (greater than or less than) of the number.

### Examples

The biosynthesis of mogrosides in fruit involves a number of consecutive glycosylations of the aglycone mogrol to the final sweet products, including mogroside V (Mog. V). Mog. V has a sweetening capacity that is about 250 times that of sucrose (Kasai et al., Agric Biol Chem (1989)). Mogrosides are reported to have health benefits as well (Li et al., Chin J Nat Med (2014)).

A variety of factors are promoting a surge in interest in mogrosides and monkfruit in general, including an explosion in demand for natural sweeteners, difficulties in scalable sourcing of the current lead natural sweetener, rebaudioside M (RebM) from the Stevia plant, the superior taste performance of Mog. V relative to other natural and artificial sweetener products on the market, and the medicinal potential of the plant and fruit.

Purified Mog. V has been approved as a high-intensity sweetening agent in Japan (Jakinovich et al., Journal of Natural Products (1990)) and the extract has gained GRAS status in the USA as a non-nutritive sweetener and flavor enhancer (GRAS 522). Extraction of mogrosides from the fruit can yield a product of varying degrees of purity, often accompanied by undesirable aftertaste. In addition, yields of mogroside from cultivated fruit are limited due to low plant yields and particular cultivation requirements of the plant. Mogrosides are present at ˜1% in the fresh fruit and ˜4% in the dried fruit. Mog. V is the main component, with a content of 0.5%-1.4% in the dried fruit. Moreover, purification difficulties limit purity for Mog. V, with commercial products from plant extracts being standardized to ˜50% Mog. V. A pure Mog. V product is desirable to avoid off flavors, and will be easier to formulate into products, since Mog. V has good solubility potential. It is therefore advantageous to produce sweet mogroside compounds, such as but not limited to Mog. V, via biotechnological processes.

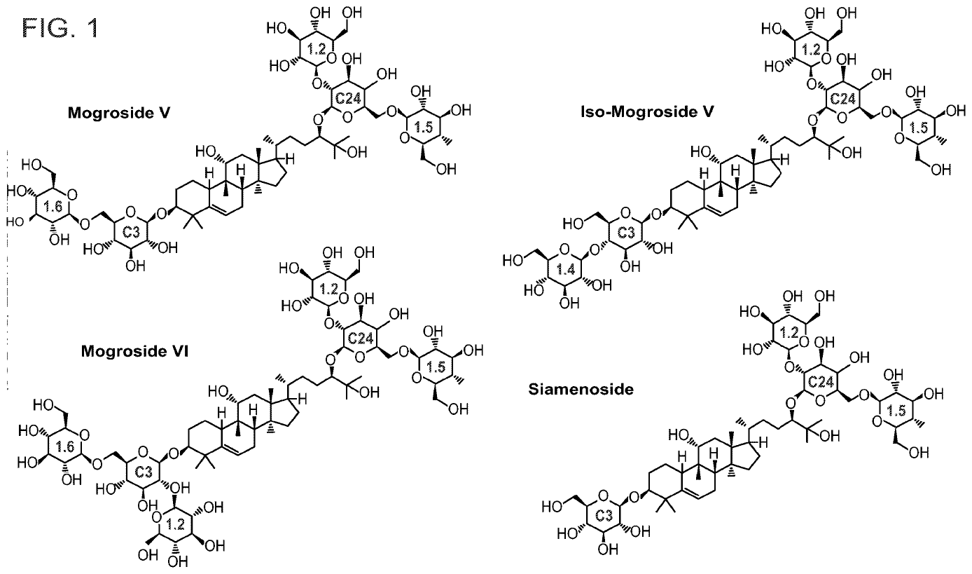

FIG. 1 shows the chemical structures of Mog. V, Mog. VI, Isomog. V, and Siamenoside. Mog. V has five glucosylations with respect to the mogrol core, including glucosylations at the C3 and C24 hydroxyl groups, followed by 1-2, 1-4, and 1-6 glucosyl additions. These glucosylation reactions are catalyzed by uridine diphosphate-dependent glycosyltransferase enzymes (UGTs).

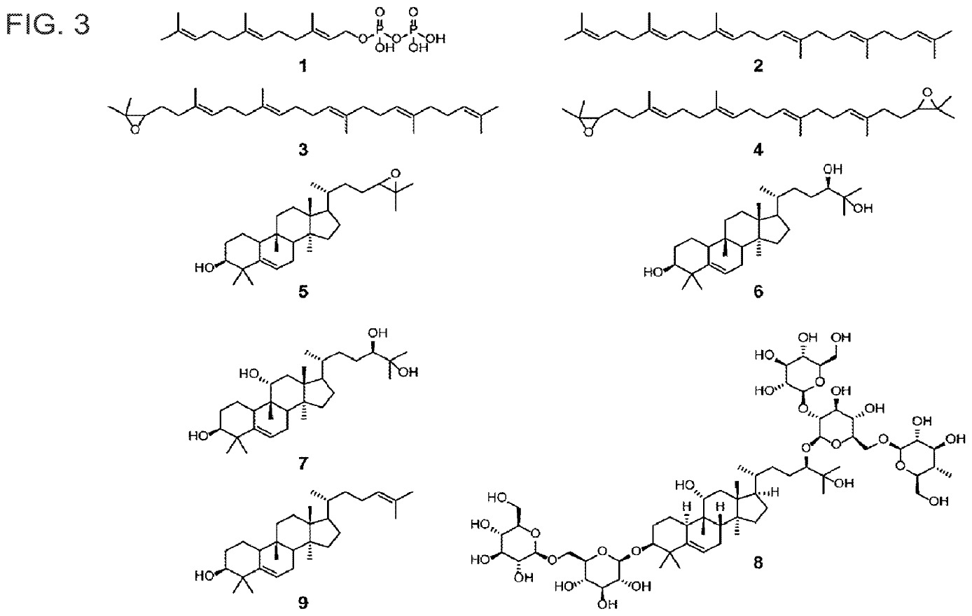

FIG. 2 shows routes to Mog. V production in vivo. The enzymatic transformation required for each step is indicated, along with the type of enzyme required. Numbers in parentheses correspond to the chemical structures in FIG. 3, namely: (1) farnesyl pyrophosphate; (2) squalene; (3) 2,3-oxidosqualene; (4) 2,3;22,23-dioxidosqualene; (5) 24,25-epoxycucurbitadienol; (6) 24,25-dihydrooxycucurbitadienol; (7) mogrol; (8) mogroside V; (9) cucurbitadienol.

Mogrosides can be produced by biosynthetic fermentation processes, as illustrated in FIG. 2, using microbial strains that produce high levels of methylerythritol 4-phosphate (MEP) pathway products, along with heterologous expression of mogrol biosynthesis enzymes and UGT enzymes that direct glucosylation reactions to Mog. V, or other desired mogroside compound. For example, in bacteria such as E. coli, isopentenyl pyrophosphate (IPP) and dimethylallyl pyrophosphate (DMAPP) can be produced from glucose, and are converted to farnesyl diphosphate (FPP) (1) by recombinant farnesyl diphosphate synthase (FPPS). FPP is converted to squalene (2) by a condensation reaction catalyzed by squalene synthase (SQS). Squalene is converted to 2,3-oxidosqualene (3) by an epoxidation reaction catalyzed by a squalene epoxidase (SQE). The pathway can proceed to 22,23-dioxidosqualene (4) by further epoxidation followed by cyclization to 24,25-epoxycucurbitadienol (5) by a triterpene cyclase, and then hydration of the remaining epoxy group to 24,25-dihydroxycucurbitadienol (6) by an epoxide hydrolase. A further hydroxylation catalyzed by a P450 oxidase produces mogrol (7).

The pathway can alternatively proceed by cyclization of (3) to produce cucurbitadienol (9), followed by epoxidation to (5), or multiple hydroxylations of cucurbitadienol to 24,25-dihydroxycucurbitadienol (6), or to mogrol (7).

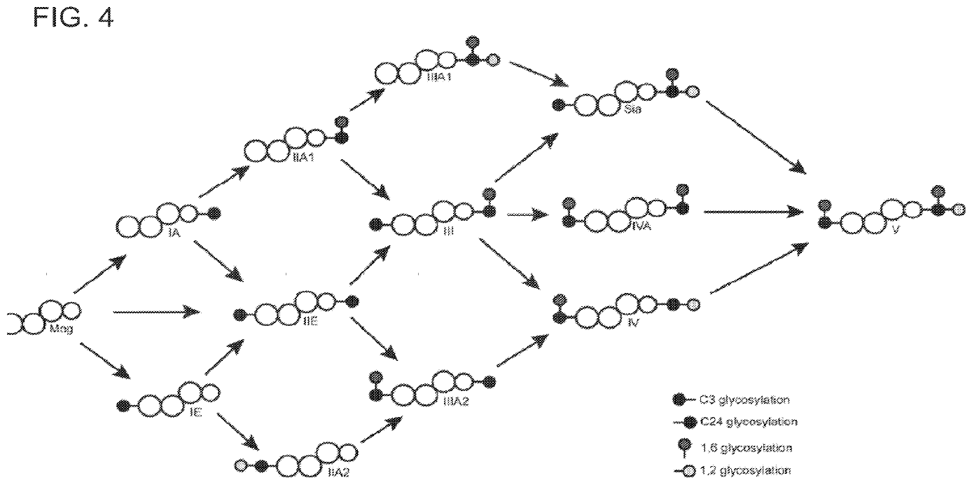

FIG. 4 illustrates glucosylation routes to Mog. V. Glucosylation of the C3 hydroxyl produces Mog.I-E, or glucosylation of the C24 hydroxyl produces Mog.I-A1. Glucosylation of Mog.I-A1 at C3 or glucosylation of Mog.I-E1 at C24 produces Mog.II-E. Further 1-6 glucosylation of Mog.II-E at C3 produces Mog.III-A2. Further 1-6 glucosylation at C24 of Mog.IIE produces Mog.III. 1-2 glucosylation of Mog.III-A2 at C24 produces Mog.IV, and then to Mog. V with a further 1-6 glucosylation at C24. Alternatively, glucosylations may proceed through Mog.III, with a 1-6 glucosylation at C3 and a 1-2 glucosylation at C24, or through Siamenoside or Mog.IV with 1-6 glucosylations.

While biosynthetic enzymes from monkfruit (Siraitia grosvenorii) have been identified for production of mogrol (See, WO 2016/038617 and US 2015/0322473, which are hereby incorporated by reference in their entireties), many of these enzymes lack the productivity or physical properties desired for overexpression in microbial hosts, particularly for fermentation approaches that operate at higher temperatures than the natural climate of the plant. Accordingly, alternative or engineered enzymes are desired to improve production of mogrol using microbial fermentation, with mogrol acting as the substrate for glucosylation to produce Mog. V or other target mogroside.

Using an E. coli strain that produces high levels of the MEP pathway products IPP and DMAPP (see US 2018/0245103 and US 2018/0216137, which are hereby incorporated by reference), and with overexpression of ScFPPS, enzymes were screened for their ability to convert FPP to squalene (SQS activity), as well epoxidation of squalene to produce 2,3-oxidosqualene (SQE activity). The 2,3-oxidosqualene intermediate can by cyclized by a triterpene cyclase, such as CDS from Siraitia grosvenorii. As demonstrated in FIG. 5, several enzymes were identified with good activity in E. coli. In particular, SEQ ID NO: 11 showed high activity in E. coli at 37° C. culture conditions.

As shown in FIG. 6, co-expression of SQS (SEQ ID NO: 11) and SQE (SEQ ID NO: 39) in E. coli provided a substantial gain in titer of the 2,3-oxidosqualene intermediate. Other SQE enzymes were active in E. coli.

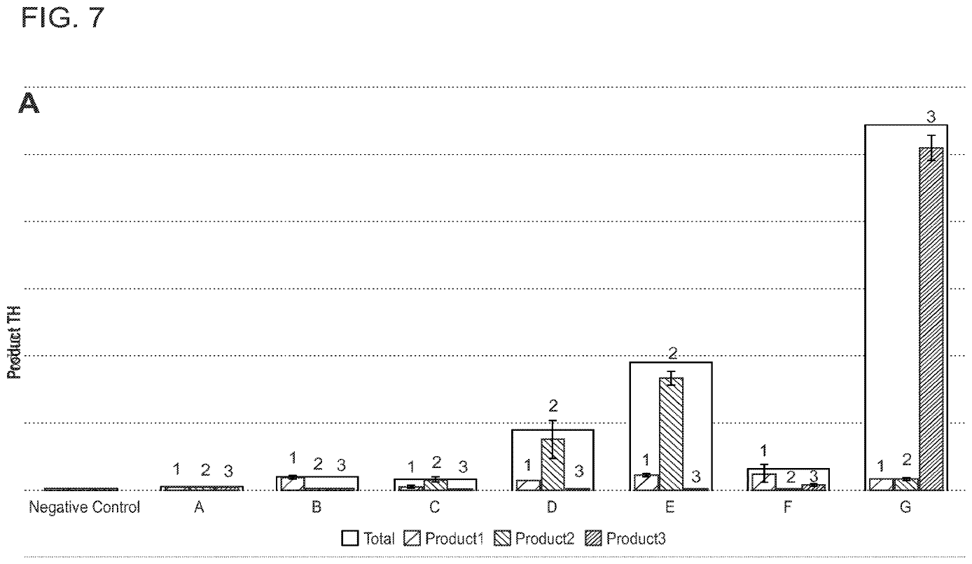

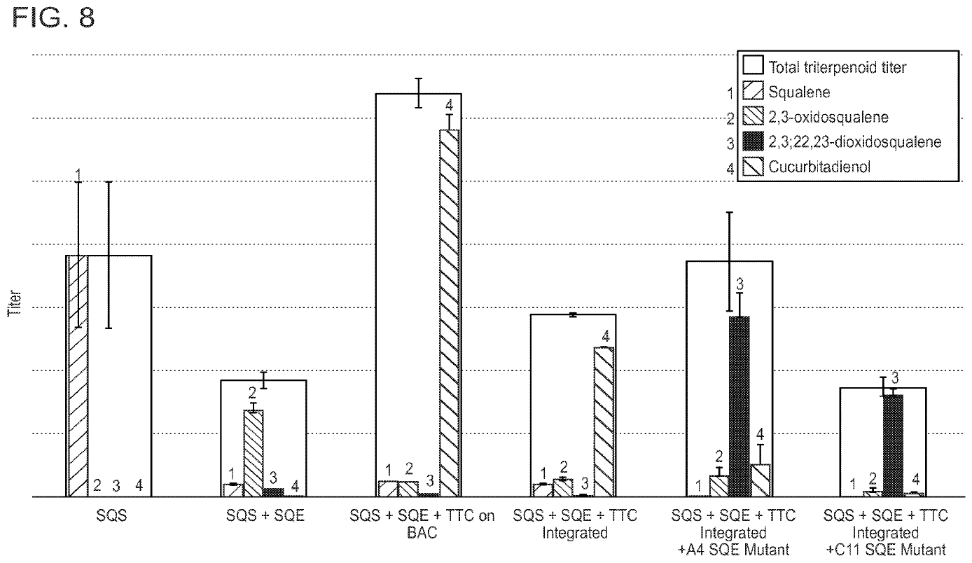

FIG. 7 shows coexpression of SQS, SQE, and TTC enzymes. CDS (or triterpene cyclase, or “TTC”) (SEQ ID NO: 40), when coexpressed with SQS (SEQ ID NO: 11) and SQE (SEQ ID NO: 39), resulted in high production of the triterpenoid product, cucurbitadienol (Product 3). These fermentation experiments were performed at 37° C. for 48 to 120 hours. FIG. 8 shows results for SQE engineering to produce high titers of 2,3;22,23-dioxidosqualene. Expression of SQS, SQE, and TTC whether on a bacterial artificial chromosome (BAC) or integrated, produce large amounts of cucurbitadienol. Point mutations in SQE (SEQ ID NO: 39) were screened to complement SQE (SEQ ID NO: 39) to reduce levels of cucurbitadienol, with corresponding gain in titers of 2,3;22,23-dioxidosqualene. Two SQE mutants are shown in FIG. 8, SQE A4 and SQE C11. By complementing SQE (SEQ ID NO: 39) with a second engineered version with higher specificity/activity for 2,3-oxidosqualene, titers can be pushed toward 2,3;22,23-dioxidosqualene, as opposed to cucurbitadienol. This concept is demonstrated further in FIG. 9. SQE A4 (SEQ ID NO: 203) was co-expressed with SQE (SEQ ID NO: 39), SQS (SEQ ID NO: 11), and TTC (SEQ ID NO: 40). These fermentation experiments were performed at 37° C. for 48 hours in 96 well plates. Titers were plotted for each strain producing 2,3;22,23 dioxidosqualene. As shown in FIG. 9, the strain expressing SQE A4 (SEQ ID NO: 203) produced much more 2,3;22,23 dioxidosqualene.

FIG. 10 shows the coexpression of SQS, SQE, and TTC enzymes. TTC (SEQ ID NO: 40), when coexpressed with SQS (SEQ ID NO: 11), SQE (SEQ ID NO: 39), and SQE A4 (SEQ ID NO: 203) in E. coli, resulted in production of cucurbitadienol and 24,25-epoxycucurbitadienol. Candidate enzymes for an additional or alternative TTC include SEQ ID NO: 40, SEQ ID NO: 191, SEQ ID NO: 192, and SEQ ID NO: 193. Each candidate TTC enzyme was expressed in this strain and screened for production of 24,25-epoxy-cucurbitadienol. These fermentation experiments were performed at 30° C. for 72 hours in 96 well plates. 24,25-epoxy-cucurbitadienol production was verified by GC-MS spectrum analysis. Concentrations were plotted relative to production of 24,25-epoxy-cucurbitadienol from an E. coli strain expressing SEQ ID NO: 40 as the only cyclase. As shown in FIG. 10, E. coli strains coexpressing SQS (SEQ ID NO: 11), SQE (SEQ ID NO: 39), SQE A4 (SEQ ID NO: 203), and TTC (SEQ ID NO: 40), with an additional TTC, produced higher levels of 24,25-epoxycucurbitadienol.

FIG. 11 shows substrate specificity for production of cucurbitadienol and 24,25-epoxycucurbitadienol with candidate TTC enzymes. Engineered E. coli strains producing oxidosqualene and dioxidosqualene were complemented with CDS homologs and CAS genes engineered for cucurbitadienol production. Strains were incubated at both 30° C. for 72 hours before extraction. The ratio of 24,25-epoxycucurbitadienol to cucurbitadienol varies from 0.15 for Enzyme 1 (SEQ ID NO: 40) to 0.58 for Enzyme 2 (SEQ ID NO: 192), pointing to improved substrate specificity toward the desired 24,25-epoxycucurbitadienol product for Enzyme 2.

FIG. 12 shows the screening of EPH enzymes for hydration of epoxycucurbitadienol to produce 24,25-dihydroxycucurbitadienol in E. coli strains coexpressing SQS (SEQ ID NO: 11), SQE (SEQ ID NO: 39), SQE A4 (SEQ ID NO: 203), and TTC (SEQ ID NO: 40). EPH homologs were expressed in a strain producing 24,25-epoxycucurbitadienol for production of 24,25-dihydroxycucurbitadienol. Candidate EPH enzymes for this reaction include SEQ ID NO: 184, SEQ ID NO: 185, SEQ ID NO: 186, SEQ ID NO: 212, SEQ ID NO: 187, SEQ ID NO: 188, SEQ ID NO: 189, and SEQ ID NO: 190. These fermentation experiments were performed at 30° C. for 72 hours in 96 well plates. 24,25-dihydroxycucurbitadienol production was verified by GC-MS spectrum analysis. Titers were plotted for each strain producing 24,25-dihydroxycucurbitadienol. As shown in FIG. 12, the E. coli strains expressing the EPHs were able to produce 24,25-dihydroxycucurbitadienol. ToEPH and SgEPH3 in particular demonstrated high activity in E. coli.

FIG. 13A-C shows the coexpression of SQS, SQE, TTC, EPH, and P450 enzymes to produce mogrol. E. coli strains were constructed that express SQS (SEQ ID NO: 11), SQE (SEQ ID NO: 39), SQE A4 (SEQ ID NO: 203), TTC (SEQ ID NO: 40), EPH (SEQ ID NO: 58), and a P450 selected from SEQ ID NO: 194, SEQ ID NO: 197, and SEQ ID NO: 171, together with a cytochrome P450 reductase (9SEQ ID NO: 98 or SEQ ID NO: 201). These fermentation experiments were performed at 30° C. for 72 hours in 96 well plates. Mogrol production was verified by LC-QQQ spectrum analysis. As shown in FIG. 13A, the expression of SQS (SEQ ID NO: 11), SQE (SEQ ID NO: 39), SQE A4 (SEQ ID NO: 203), TTC (SEQ ID NO: 40), EPH (SEQ ID NO: 58), and the P450s SEQ ID NO: 194, SEQ ID NO: 197, and SEQ ID NO: 171 resulted in production of mogrol and oxo-mogrol. As shown in FIG. 13B and FIG. 13C, mogrol production was validated by LC-QQQ mass spectrum analysis using spiked authentic standard (FIG. 13B) and GC-FID chromatography versus an authentic standard (FIG. 13C), respectively.

FIG. 14 shows the screening of cytochrome P450s for oxidation at C11 of the 24,25-dihydroxycucurbitadienol-like molecule cucurbitadienol. In many cases, the native transmembrane domain was replaced with the transmembrane domain from E. coli sohB (SEQ ID NO: 195, SEQ ID NO: 198, and SEQ ID NO: 199), E. coli zipA (SEQ ID NO: 196), or bovine 17a (e.g. SEQ ID NO: 200) to improve interaction with the E. coli membrane. Each P450 was coexpressed with either SEQ ID NO: 201 or SEQ ID NO: 98, resulting in production of 11-hydroxycucurbitadienol. These fermentation experiments were performed at 30° C. for 72 hours in 96 well plates. 11-hydroxy-cucurbitadienol production was verified by GC-MS. Concentrations were plotted for strains producing 11-hydroxycucurbitadienol. As shown in FIGS. 14 and 15, the strains disclosed herein were capable of production of 11-hydroxy-cucurbitadienol.

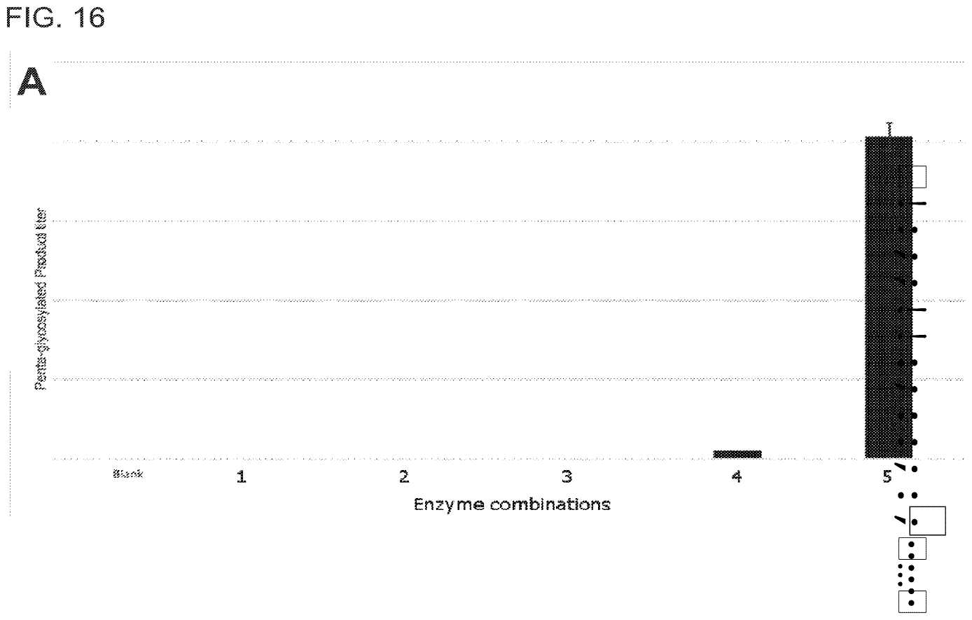

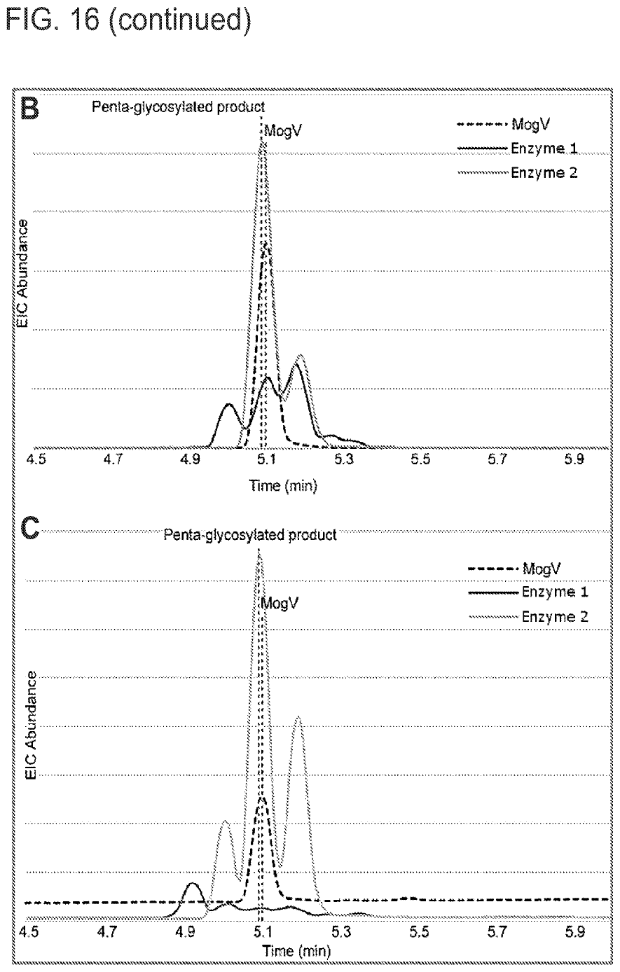

Mogrol was used as a substrate for in vitro glucosylation reactions with candidate UGT enzymes, to identify candidate enzymes that provide efficient glucosylation of mogrol to Mog. V. Reactions were carried out in 50 mM Tris-HCl buffer (pH 7.0) containing beta-mercaptoethanol (5 mM), magnesium chloride (400 μM), substrate (200 μM), UDP-glucose (5 mM), and a phosphatase (1 U). Results are shown in FIG. 16A. Mog. V product is observed when the UGT enzymes of SEQ ID NO: 165, SEQ ID NO: 146, and SEQ ID NO: 117 are incubated together. A penta-glycosylated product is formed when the UGT enzymes of SEQ ID NO: 165, SEQ ID NO: 146, and SEQ ID NO: 164) are incubated together. FIG. 16B, Extracted ion chromatogram (EIC) for 1285.4 Da (mogroside V+H) of reactions containing enzymes of SEQ ID NO: 165+SEQ ID NO: 146 and either SEQ ID NO: 117 (solid dark grey line) or SEQ ID NO: 164 (light grey line) when incubated with Mog.II-E. FIG. 16C, Extracted ion chromatogram (EIC) for 1285.4 Da (mogroside V+H) of reactions containing enzymes of SEQ ID NO: 165+SEQ ID NO: 146 and either SEQ ID NO: 117 (solid dark grey line) or SEQ ID NO: 164 (light grey line) when incubated with mogrol.

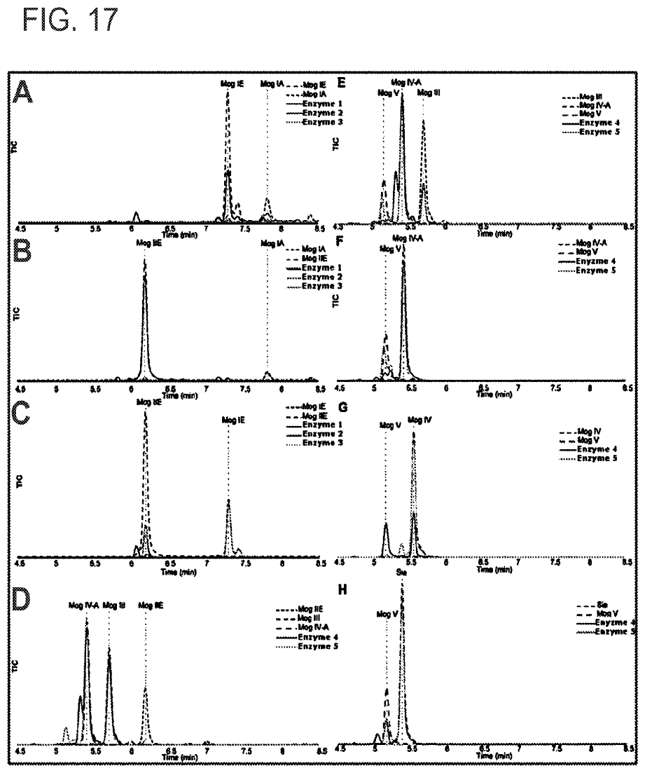

FIG. 4 and FIG. 17 show additional glycosyltransferase activities observed on particular substrates. Coexpression of UGT enzymes can be selected to move product to the desired mogroside product.

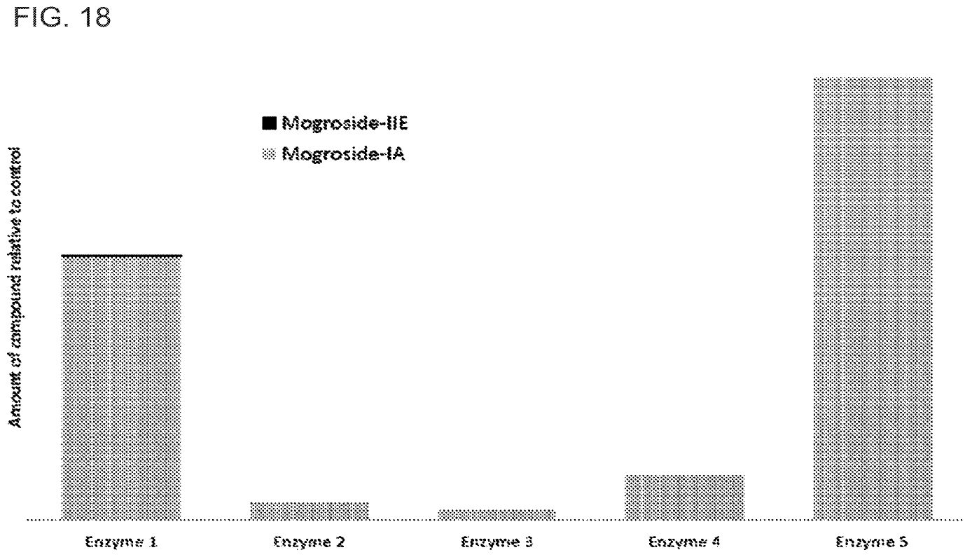

FIG. 18 shows the bioconversion of mogrol into mogroside intermediates. Engineered E. coli strains (see US 2020/0087692, which is hereby incorporated by reference in its entirety) expressing UGT enzymes were incubated in 96-well plates with 0.2 mM mogrol. Product formation was examined after 48 hours. Reported values are those in excess of the empty vector control. Products were measured on LC-MS/MS with authentic standards. Only Enzyme 1 shows formation of Mog.IIE. Enzymes 1 to 5 are SEQ ID NOS: 202, 116, 216, 217, and 218, respectively.

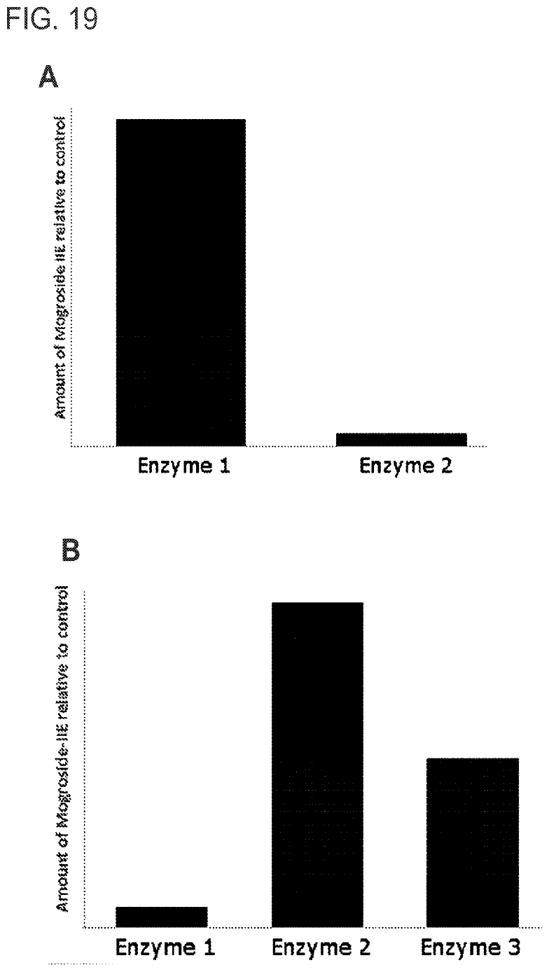

FIG. 19A and FIG. 19B shows the bioconversion of Mog.IA (FIG. 19A) or Mog.IE (FIG. 19B) into Mog.IIE. In the experiment, engineered E. coli strains (as above) expressing UGT enzymes, SEQ ID NO: 165, SEQ ID NO: 202, or SEQ ID NO: 116 were incubated in fermentation media containing 0.2 mM Mog.IA (FIG. 19A) or Mog.IE (FIG. 19B) in 96-well plates at 37° C. Product formation was examined after 48 hours. Products were measured on LC-MS/MS with authentic standards. The values of Mog.IIE levels in excess of the empty vector control were calculated. As shown in FIG. 19A, SEQ ID NO: 165 and SEQ ID NO: 202 were able to catalyze bioconversion of Mog.IA into Mog.IIE. Similarly, as shown in FIG. 19B, SEQ ID NO: 165, SEQ ID NO: 202, and SEQ ID NO: 116 were able to catalyze the bioconversion of Mog.IE into Mog.IIE.

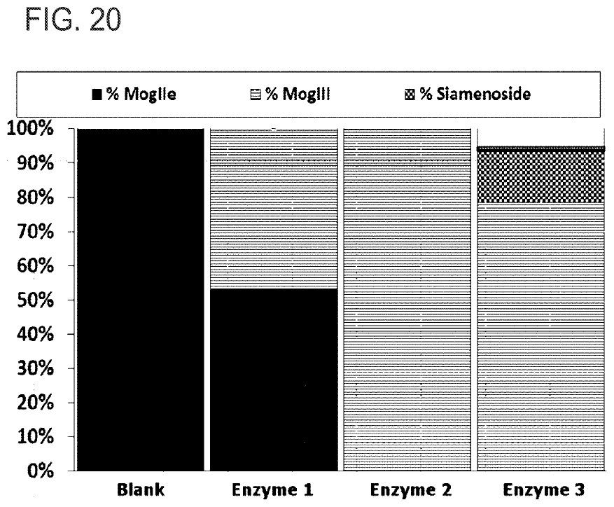

FIG. 20 shows the production of Mog.III or siamenoside from Mog.II-E. In the experiment, engineered E. coli strains expressing UGT enzymes SEQ ID NO: 204, SEQ ID NO: 138 or SEQ ID NO: 206 were grown in fermentation media containing 0.1 mM Mog.II-E at 37° C. for 48 hr. Products were quantified by LCMS/MS with authentic standards of each compound. As shown in FIG. 20, all strains were able to catalyze bioconversion of Mog.IIE to Mog.III. In addition, MbUGT1,2.2 also showed production of substantial amounts of siamenoside.

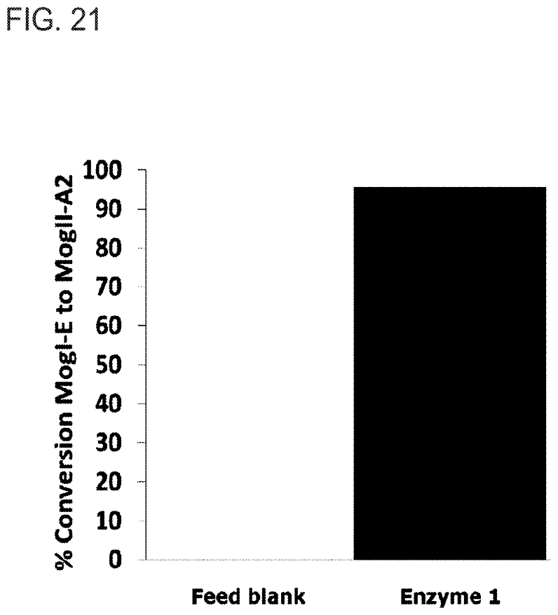

FIG. 21 shows the production of Mog.II-A2. 0.1 mM Mog.I-E was fed in vitro. In the experiment, engineered E. coli strains expressing UGT enzyme SEQ ID NO: 205 were incubated at 37° C. for 48 hr. Products were quantified by LC-MS/MS with authentic standards of each compound. As shown in FIG. 21, SEQ ID NO: 205 is able to catalyze bioconversion of Mog.IE to Mog.II-A2.

A summary of observed primary glycosylation reactions at C3 and C24 hydroxyls of mogrol are provided in Table 1. Specifically, 0.2 mM mogrol was fed to cells expressing various UGT enzymes. Reactions were incubated at 37° C. for 48 hrs. Products were quantified by LCMS/MS with authentic standards of each compound.

TABLE 1

| UGT | C3 O-Glucosylation | C24 O-Glucosylation |
|---|---|---|
| SEQ ID NO: 165 | Yes | Yes |
| SEQ ID NO: 146 | No | Yes |
| SEQ ID NO: 214 | No | Yes |
| SEQ ID NO: 202 | Yes | Yes |
| SEQ ID NO: 129 | Yes | No |
| SEQ ID NO: 116 | Yes | Yes |
| SEQ ID NO: 218 | No | Yes |
| SEQ ID NO: 216 | No | Yes |
| SEQ ID NO: 217 | No | Yes |

A summary of branched glycosylation reactions are provided in Table 2. 0.2 mM Mog.IIE or Mog.IE was fed to cells expressing various UGT enzymes. Reactions were incubated at 37° C. for 48 hr. Products were quantified by LC-MS/MS with authentic standards of each compound. “Indirect” evidence means that consumption of substrate was observed.

TABLE 2

| Name | C3 1-2 | C3 1-6 | C24 1-2 | C24 1-6 |
|---|---|---|---|---|
| SEQ ID NO: 205 | No | Yes | No | Yes |
| SEQ ID NO: 204 | No | Yes | No | No |
| SEQ ID NO: 122 | No | Yes | Yes | Yes |
| SEQ ID NO: 211 | No | No | Yes | No |
| SEQ ID NO: 138 | No | Yes | No | Yes |
| SEQ ID NO: 207 | No | Yes | No | Yes |
| SEQ ID NO: 209 | No | Yes | No | Yes |
| SEQ ID NO: 208 | Yes | Yes | Yes | Yes |
| | (Indirect) | | (Indirect) | (Indirect) |
| SEQ ID NO: 206 | Yes | Yes | Yes | Yes |
| | (Indirect) | (Indirect) | | |
| SEQ ID NO: 164 | No | Yes | Yes | Yes |
| SEQ ID NO: 210 | No | Yes | No | Yes |
| SEQ ID NO: 215 | No | No | No | Yes |
| SEQ ID NO: 213 | No | No | No | Yes |

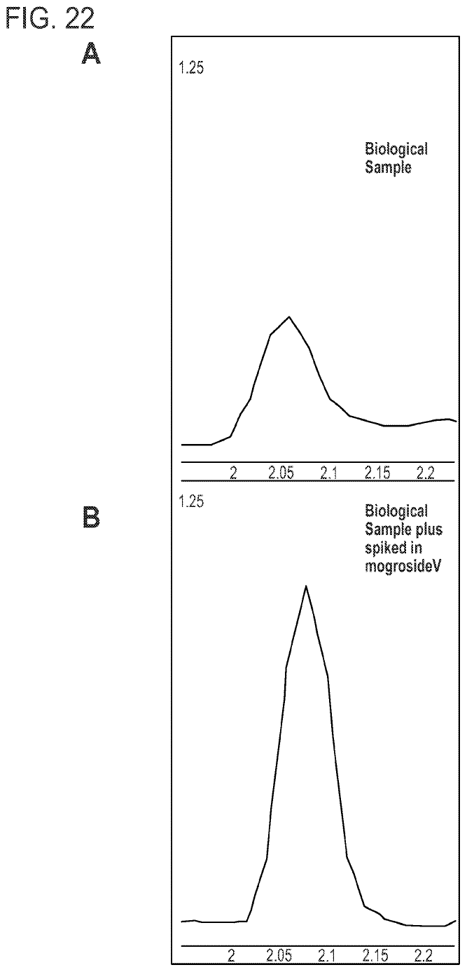

An exemplary E. coli strain producing Mog. V was created by expressing the following enzymes in an E. coli strain engineered to produce high levels of MEP pathway products: SQS (SEQ ID NO: 11), SQE (SEQ ID NO: 39), SQE A4 (SEQ ID NO: 203), TTC (SEQ ID NO: 40), EPH (SEQ ID NO: 189), sohB_CppCYP (SEQ ID NO: 199), AtUGT73C3 (SEQ ID NO: 202), UGT85C1 (SEQ ID NO: 165), and UGT94-289-1 (SEQ ID NO: 122). Production of Mog. V is demonstrated in FIG. 22A, B. Strains were incubated at 30° C. for 72 hours before extraction. Mog. V production was verified by LC-QQQ spectrum analysis versus an authentic standard FIG. 22A. FIG. 22B shows a chromatogram indicating Mog. V production from a biological sample with a spiked Mog. V authentic standard.

Biosynthesis enzymes can be further engineered for expression and activity in microbial cells, using known structures and primary sequences.

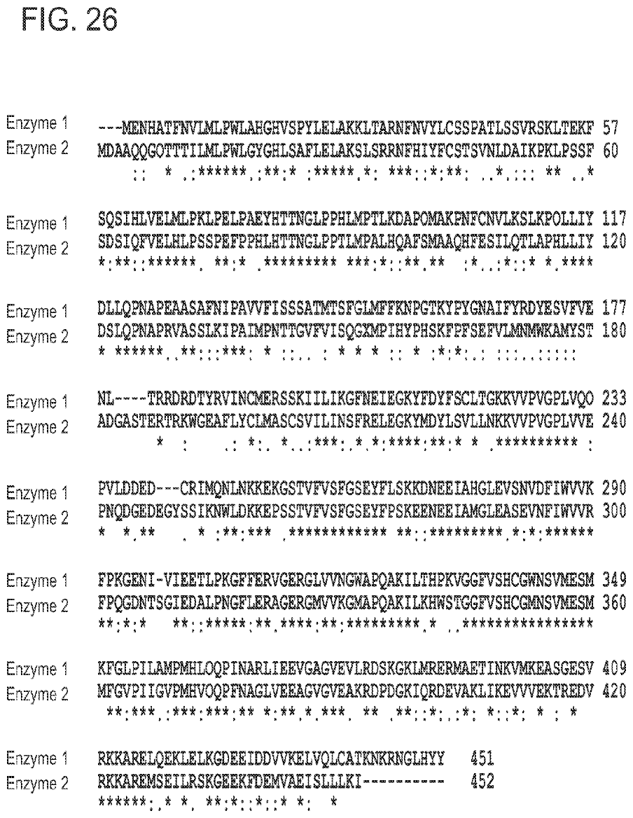

FIG. 26 is an amino acid alignment of CaUGT_1,6 and SgUGT94_289_3 using Clustal Omega (Version CLUSTAL O (1,2,4). These sequences share 54% amino acid identity. Coffea arabica UGT_1,6 is predicted to be a beta-D-glucosyl crocetin beta 1,6-glucosyltransferase-like (XP_027096357.1). Together with known UGT structures and primary sequences, CaUGT_1,6 can be further engineered for microbial expression and activity, including engineering of a circular permutant.

FIG. 27 is an amino acid alignment of Homo sapiens squalene synthase (HsSQS) (NCBI accession NP_004453.3) and AaSQS (SEQ ID NO: 11) using Clustal Omega (Version CLUSTAL O (1.2.4)). HsSQS has a published crystal structure (PDB entry: 1EZF). These sequences share 42% amino acid identity.

FIG. 28 is an amino acid alignment of Homo sapiens squalene epoxidase (HsSQE) (NCBI accession XP_011515548) and MISQE (SEQ ID NO: 39) using Clustal Omega (Version CLUSTAL O (1.2.4)). HsSQE has a published crystal structure (PDB entry: 6C6N). These sequences share 35% amino acid identity.

The UGT enzyme of SEQ ID NO: 164 was engineered for improved glysoylation activity. Various amino acid substitutions were made to the enzyme, as informed by in silico analysis. The following amino acid substitutions in Table 3 were tested for further glycosylation of mog. IIE.

TABLE 3

| | Fold Improviment in UDP-Glucose |
|---|---|
| Substitution | Transferred |
| G150F | 13.2 |
| T147L | 13.0 |
| N207K | 10.9 |
| K270E | 10.0 |
| V281L | 9.1 |
| L354V | 8.6 |
| L13F | 7.5 |
| T32A | 5.6 |
| K101A | 5.3 |
| C219E | 4.9 |
| V281Q | 4.6 |
| S43T | 4.6 |
| M394V | 4.6 |
| E74G | 4.5 |
| K270P | 4.1 |
| T256V | 3.9 |
| V175K | 3.9 |
| N283G | 3.4 |
| D285P | 3.3 |
| A377V | 3.2 |
| F217L | 3.1 |
| K204R | 3.1 |
| T303A | 3.0 |
| D95K | 2.9 |
| S141I | 2.7 |
| K270T | 2.7 |
| V281A | 2.5 |
| A166 del. | 2.2 |
| G205S | 2.1 |
| N333S | 2.0 |
| K270M | 2.0 |
| F132L | 2.0 |
| L40F | 1.9 |
| A166K | 1.9 |
| V281K | 1.8 |
| R185S | 1.7 |
| F8L | 1.7 |
| F258Y | 1.7 |
| N35G | 1.7 |
| N133G | 1.7 |
| A77P | 1.6 |
| N207Y | 1.6 |
| K386D | 1.6 |
| Y163F | 1.5 |
| N399R | 1.5 |
| H18Y | 1.5 |
| A166S | 1.3 |
| K101E | 1.3 |
| Q418K | 1.3 |
| I191V | 1.3 |
| R182S | 1.2 |
| K101Q | 1.2 |
| S142F | 1.2 |
| T46N | 1.2 |
| T159E | 1.2 |
| T55P | 1.2 |
| K160D | 1.2 |
| T7K | 1.2 |
| A166T | 1.1 |

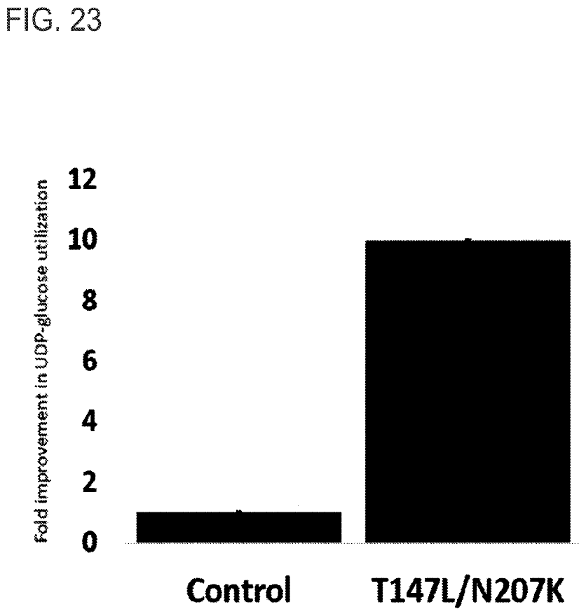

An engineered UGT enzyme based on SEQ ID NO: 164 was prepared having substitutions T147L and N207K. The bioconversion of Mog.IIE to further glycosylated products is shown in FIG. 23. In the experiment, engineered E. coli strains expressing the engineered CaUGT_1,6 were inoculated with Mog.IIE substrate at 37° C. Product formation was examined after 48 hours. Products were measured on LC/MS-QQQ with authentic standards.

The UGT enzyme of SEQ ID NO: 165 was engineered for improved glysoylation activity. The following amino acid substitutions were identified as improving bioconversion of Mog.IA to Mog.IIE (Table 4):

TABLE 4

| | Fold Improvement in Mog.IA to Mog.IIE |
|---|---|
| Substitution | Bioconversion |
| CTL | 1 |
| L41F | 1.29 |
| D49E | 1.36 |
| C127F | 1.48 |

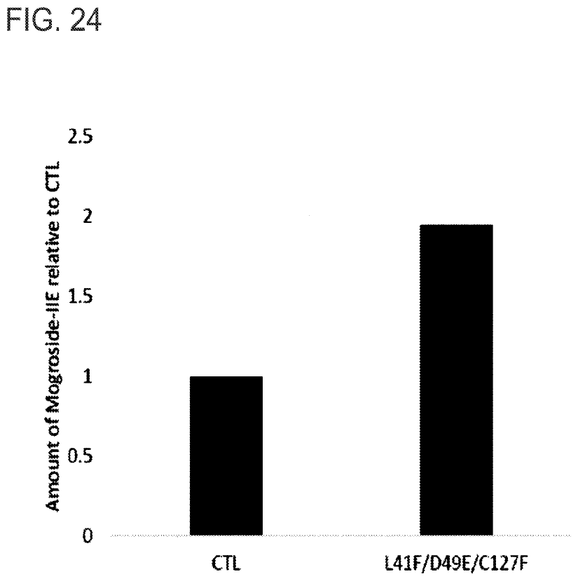

An engineered UGT enzyme based on 85C1 was prepared having substitutions L41F, D49E, and C127F. The bioconversion of Mog.IA to Mog.IIE is shown in FIG. 24. In the experiment, engineered E. coli strains expressing the engineered 85C1 were inoculated with Mog.IA substrate at 37° C. Product formation was examined after 48 hours. Products were measured on LC/MS-QQQ with authentic standards. FIG. 24 shows the fold improvement of the engineered version compared to the control (85C1).

The UGT enzyme of SEQ ID NO: 217 (UGT73F24) was engineered for improved glysoylation activity. The following amino acid substitutions were identified as improving bioconversion of Mog.IE to Mog.IIE with UGT73F24 (Table 5):

TABLE 5

| | Fold Improvement in Mog.IE to Mog.IIE |
|---|---|
| Substitution | Production |
| CTL | 1 |
| A74E | 1.88 |
| I91F | 2.01 |
| H101P | 2.38 |
| Q241E | 1.31 |
| I436L | 1.09 |

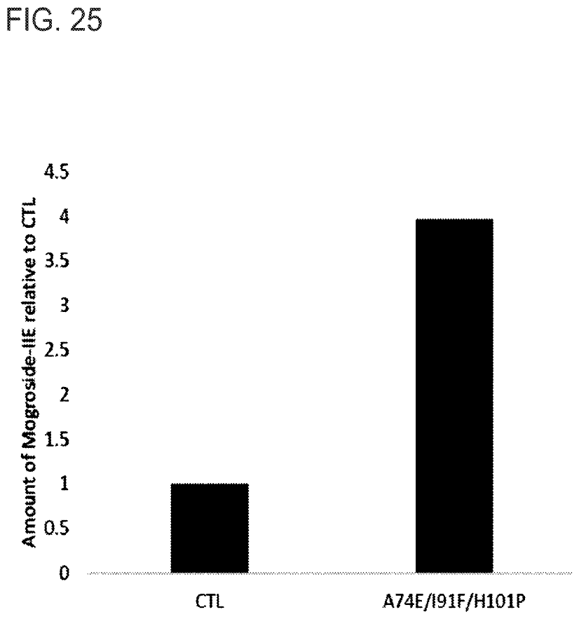

An engineered UGT enzyme based on UGT73F24 was prepared having substitutions A74E, I91F, and H101P. The bioconversion of Mog.IE to Mog.IIE is shown in FIG. 25. In the experiment, engineered E. coli strains expressing the engineered UGT73F24 were inoculated with Mog.IE substrate at 37° C. Product formation was examined after 48 hours. Products were measured on LC/MS-QQQ with authentic standards. FIG. 25 shows the fold improvement of the engineered version compared to the control (73F24).

SEQUENCES
Farnesyl Pyrophosphate Synthase (FPPS)
Saccharomyces cerevisiae FPPS
(SEQ ID NO: 1)
MASEKEIRRERFLNVFPKLVEELNASLLAYGMPKEACDWYAHSLNYNTPGGKLNRCLSVVDTYA
ILSNKTVEQLGQEEYEKVAILGWCIELLQAYFLVADDMMDKSITRRGQPCWYKVPEVGEIAIND
AFMLEAAIYKLLKSHFRNEKYYIDITELFHEVTFQTELGQLMDLITAPEDKVDLSKFSLKKHSF
IVTFKTAYYSFYLPVALAMYVAGITDEKDLKQARDVLIPLGEYFQIQDDYLDCFGTPEQIGKIG
TDIQDNKCSWVINKALELASAEQRKTLDENYGKKDSVAEAKCKKIFNDLKIEQLYHEYEESIAK
DLKAKISQVDESRGFKADVLTAFLNKVYKRSK
Squalene Synthase (SQS)
Siraitia grosvenorii SQSa
(SEQ ID NO: 2)
MGSLGAILRHPDDFYPLLKLKMAARHAEKQIPPEPHWGFCYTMLHKVSRSFALVIQQLAPELRN
AICIFYLVLRALDTVEDDTSIQTDIKVPILKAFHCHIYNRDWHFSCGTKDYKVLMDQFHHVSTA
FLELGKGYQEATEDITKRMGAGMAKFICKEVETVDDYDEYCHYVAGLVGLGLSKLFHASDLEDL
APDSLSNSMGLLLQKTNIIRDYLEDINEIPKSRMFWPREIWGKYADKLEDFKYEENSVKAVQCI
NDLVTNALNHVEDCLKYMSNLRDLSIFRFCAIPQIMAIGTLALCYNNVEVFRGVVKMRRGLTAK
VIDRTQTMADVYGAFFDFSVMLKAKVNSSDPNATKTLSRIEAIQKTCEQSGLLNKRKLYAVKSE
PMFNPTLIVILFSLLCIILAYLSAKRIPANQPV
Siraitia grosvenorii SQSb
(SEQ ID NO: 3)
MGSLGAILRHPDDFYPLLKLKMAARHAEKQIPPEPHWGFCYTMLHKVSRSFALVIQQLAPELRN
AICIFYLVLRALDTVEDDTSIQTDIKVPILKAFHCHIYNRDWHFSCGTKDYKVLMDQFHHVSTA
FLELGKGYQEAIEDITKRMGAGMAKFICKEVETVDDYDEYCHYVAGLVGLGLSKLFHASDLEDL
APDSLSNSMGLLLQKTNIIRDYLEDINEIPKSRMFWPREIWGKYADKLEDFKYEENSVKAVQCL
NDLVTNALNHVEDCLKYMSNLRDLSIFRFCAIPQIMAIGTLALCYNNVEVFRGVVKMRRGLTAK
VIDRTQTMADVYGAFFDFSVMLKAKVNNSDPNATKTLSRIEAIQKTCEQSGLLNKRKLYAVKSE
PMFNPTLIVILFSLLCIILAYLSAKRLPANQPV
Cucumis sativus
(SEQ ID NO: 4)
MGSLGAILKHPDDFYPLLKLKIAARHAEKQIPPEPHWGFCYTMLHKVSRSFALVIQQLKPELRN
AVCIFYLVLRALDTVEDDTSIQTDIKVPILKAFHCHIYNRDWHFSCGTKDYKVLMDEFHHVSTA
FLELGKGYQEAIEDITKRMGAGMAKFICKEVETVDDYDEYCHYVAGLVGLGLSKLFHAAELEDL
APDSLSNSMGLFLQKTNIIRDYLEDINEIPKSRMFWPREIWGKYADKLEDFKYEENSVKAVQCL
NDLVTNALNHVEDCLKYMSNLRDLSIFRFCAIPQIMAIGTLALCYNNVEVFRGVVKMRRGLTAK
VIDRTKTMADVYGAFFDFSVMLKAKVNSNDPNASKTLSRIEAIQKTCKQSGILNRRKLYVVRSE
PMFNPAVIVILFSLLCIILAYLSAKRLPANQSV
Cucumis melo
(SEQ ID NO: 5)
MGSLGAILKHPDDFYPLLKLKMAARHAEKQIPPESHWGFCYTMLHKVSRSFALVIQQLKPELRN
AVCIFYLVLRALDTVEDDTSIQTDIKVPILKAFHCHIYNRDWHFSCGTKDYKVLMDEFHHVSTA
FLELGKGYQEAIEDITKRMGAGMAKFICKEVETVDDYDEYCHYVAGLVGLGLSKLFHAAELEDL
APDSLSNSMGLFLQKTNIIRDYLEDINEIPKSRMFWPREIWGKYADKLEDFKYEENSVKAVQCL
NDLVTNALNHVEDCLKYMSNLRDLSIFRFCAIPQIMAIGTLALCYNNVEVFRGVVKMRRGLTAK
VIDRTKTMADVYGAFFDESVMLKAKVNSNDPNASKTLSRIEAIQQTCQQSGLMNKRKLYVVRSE
PMYNPAVIVILFSLLCIILAYLSAKRLPANQSV
Cucumis melo
(SEQ ID NO: 6)
MGSLGAILKHPDDFYPLLKLKMAARHAEKQIPPESHWGFCYTMLHKVSRSFALVIQQLKPELRN
AVCIFYLVLRALDTVEDDTSIQTDIKVPILKAFHCHIYNRDWHFSCGTKDYKVLMDEFHHVSTA
FLELGKGYQEAIEDITKRMGAGMAKFICKEVETVDDYDEYCHYVAGLVGLGLSKLFHAAELEDL
APDSLSNSMGLFLQKTNIIRDYLEDINEIPKSRMFWPREIWGKYADKLEDFKYEENSVKAVQCI
NDLVTNALNHVEDCPKYMSNLRDLSIFRFCAIPQIMAIGTLALCYNNVEVFRGVVEMRRGLTAK
VIDRTKTMADVYGAFFDFSVMLKAKVNSNDPNASKTLSRIEAIQQTCQQSGLMNKRKLYVVRSE
PMYNPAVIVILFSLLCIILAYLSAKRLPANQSV
Cucurbita moschata
(SEQ ID NO: 7)
MGSLGAILRHPDDIYPLLKLKMAARHAEKQIPPESHWGFCYTMLHKVSRSFALVIQQLKPELRN
AVCIFYLVLRALDTVEDDTSIQTDIKVPILKAPHCHIYNRDWHFSCGTKDYKVLMDEFHHVSTA
FLELGRGYQEAIEDITKRMGAGMAKFICKEVETVEDYDEYCHYVAGLVGLGLSKLFHASKSENL
APDSLSNSMGLFLQKTNIIRDYLEDINEIPKSRMFWPREIWSKYADKLEDFKYEKNSVKAVQCL
NDLVTNALTHVEDCLEYMSNLKDLSIFRFCAIPQIMAIGTLALCYNNVDVFRGVVKMRRGLTAK
VIYRTKTMADVYGAFFDFSVMLKAKVNSSDPNASKTLTRIEAIQKTCKQSGLLNKRELYAVRSE
PMCNPAAIVVLFSLLCIILAYLSAKLLPANQPV
Sechium edule
(SEQ ID NO: 8)
MGSLGAILSHPDDLYPLLKLKMAAKHAEKQIPPDPHWGFCFSMLHKVSRSFALVIQQLKPELRN
AVCIFYLVLRALDTVEDDTGIHPDIKVPILQAFHCHIYNRDWHFSCGTKHYKVLMDEFHHVSTA
FLELGKGYQEAIEDVTERMGAGMAKFICKEVETVDDYDEYCHYVAGLVGLGLSKLFHAAELEDL
APDSLSNSMGLFLQKTNIIRDYLEDINEIPKSRMFWPREIWNKYADKLEDFKYEENSVKAVQCL
NDLVTNALNHVEDCLKYMSNLKDLSTFRFCAIPQIMAIGTLALCYDNVEVFRGVVKMRRGLTAK
IIDRTKKIADVYGAFFDFSVMLKAKVNSSDPNAAKTLSRIEAIEKTCKESGLLNKRKLYVIRSE
PLFNPAVLVILFSLICILLAYLSAKRLPANQPV
Panax quinquefolius
(SEQ ID NO: 9)
MGSLGAILKHPDDFYPLLKLKFAARHAEKQIPPEPHWAFCYSMLHKVSRSFGLVIQQLGPQLRD
AVCIFYLVLRALDTVEDDTSIPTEVKVPILMAFHRHIYDKDWHFSCGTKEYKVLMDEFHHVSNA
FLELGSGYQEAIEDITMRMGAGMAKFICKEVETIDDYDEYCHYVAGLVGLGLSKLFHASGAEDL
ATDSLSNSMGLFLQKTNIIRDYLEDINEIPKSRMFWPRQIWSKYVDKLEDLKYEENSAKAVQCI
NDMVTDALVHAEDCLKYMSDLRDPAIFRFCAIPQIMAIGTLALCFNNTQVFRGVVKMRRGLTAK
VIDRTKTMSDVYGAFFDFSCLLKSKVDNNDPNATKTLSRLEAIQKTCKESGTLSKRKSYIIESE
SGHNSALIAIIFIILAILYAYLSSNLLLNKQ
Malus domestica
(SEQ ID NO: 10)
MGALSTMLKHPDDIYPLLKLKIASRQIEKQIPAEPHWAFCYTMLQKVSRSFALVIQQLGTELRN
AVCLFYLVLRALDTVEDDTSVATDVKVPILLAFHRHIYDPDWHFACGTNNYKVLMDEFHHVSTA
FLELGTGYQEAIEDITKRMGAGMAKFILKEVETIDDYDEYCHYVAGLVGLGLSKLFHAAGKEDL
ASDSLSNSMGLFLQKTNIIRDYLEDINEIPKSRMFWPRQIWSKYVNKLEDLKYEENSEKAVQCL
NDMVTNALIHMEDCLKYMAALRDPAIFKFCAIPQIMAIGTLALCYNNIEVFRGVVKMRRGLTAK
VIDRTKSMDDVYGAFFDFSSILKSKVDKNDPNATKTLSRVEAVQKLCRDSGALSKRKSYIANRE
QSYNSTLIVALFIILAIIYAYLSASPRI
Artemisia annua
(SEQ ID NO: 11)
MSSLKAVLKHPDDFYPLLKLKMAAKKAEKQIPSQPHWAFSYSMLHKVSRSFALVIQQLNPQLRD
AVCIFYLVLRALDTVEDDTSIAADIKVPILIAFHKHIYNRDWHFACGTKEYKVLMDQFHHVSTA
FLELKRGYQEAIEDITMRMGAGMAKFICKEVETVDDYDEYCHYVAGLVGIGLSKLFHSSGTEIL
FSDSISNSMGLFLQKTNIIRDYLEDINEIPKSRMFWPREIWSKYVNKLEDLKYEENSEKAVQCL
NDMVTNALIHIEDCLKYMSQLKDPAIFRFCAIPQIMAIGTLALCYNNIEVFRGVVKLRRGLTAK
VIDRTKTMADVYQAFSDFSDMLKSKVDMHDPNAQTTITRLEAAQKICKDSGTLSNRKSYIVKRE
SSYSAALLALLFTILAILYAYLSANRPNKIKFTL
Glycine soja
(SEQ ID NO: 12)
MDQRSEDEFYPLLKLKIVARNAEKQIPPEPHWAFCYTMLHKVSRSFALVIQQLGIELRNAVCIF
YLVLRALDTVEDDTSIETDVKVPILIAFHRHIYDRDWHFSCGTKEYKVLMGQFHHVSTAFLELG
KNYQEAIEDITKRMGAGMAKFICKEVETIDDYDEYCHYVAGLVGLGLSKLFHASGSEDLAPDDL
SNSMGLFLQKTNIIRDYLEDINEIPKSRMFWPRQIWSEYVNKLEDLKYEENSVKAVQCLNDMVT
NALMHAEDCLTYMAALRDPPIFRFCAIPQIMAIGTLALCYNNIEVFRGVVKMRRGLTAKVIDRT
KTMADVYGAFFDFASMLEPKVDKNDPNATKTLSRLEAIQKTCRESGLLSKRKSYIVNDESGYGS
TMIVILVIMVSIIFAYLSANHHNS
Diospyros kaki
(SEQ ID NO: 13)
MGSLAAMLRHPDDVYPLVKLKMAARHAEKQIPPEPHWAFCYTMLHKVSRSFGLVIQQLGTELRN
AVCIFYLVLRALDTVEDDTSIATEVKVPILLAFHHHIYDRDWHFSCGTREYKVLMDEFHHVSTA
FLELGKGYQEAIEDITMRMGAGMAKFICKEVETIDDYDEYCHYVAGLVGLGLSKLFHASGLEDL
APDSLSNSMGLFLQKTNIIRDYLEDINEIPKSRMFWPRQIWSKYVNKLEDLKYEKNSVKSVQCL
NDMVTNALIHVDDCLKYMSALRDPAIFRFCAIPQIMAIGTLALCYNNIEVFRGVVKMRRGLTAK
VIDQTKTISDVYGAFFDFSCMLKSKVEKNDPNSTKTLSRIEAIQKTCRESGTLSKRKSYILRSK
RTHNSTLIFVLFIILAILFAYLSANRPPINM
Euphorbia lathyris
(SEQ ID NO: 14)
MGSLGAILKHPDDFYPLLKLKMAAKHAEKQIPAQPHWGFCYSMLHKVSRSFSLVIQQLGTELRD
AVCIFYLVLRALDTVEDDTSIPTDVKVPILIAFHKHIYDPEWHFSCGTKEYKVLMDQIHHLSTA
FLELGKSYQEAIEDITKKMGAGMAKFICKEVETVDDYDEYCHYVAGLVGLGLSKLFDASGFEDL
APDDLSNSMGLFLQKTNIIRDYLEDINEIPKSRMFWPRQIWSKYVNKLEDLKYEENSVKAVQCL
NDMVTNALIHMDDCLKYMSALRDPAIFRFCAIPQIMAIGTLALCYNNVEVFRGVVKMRRGLTAK
VIDRTRTMADVYRAFFDFSCMMKSKVDRNDPNAEKTLNRLEAVQKTCKESGLLNKRRSYINESK
PYNSTMVILLMIVLAIILAYLSKRAN
Camellia oleifera
(SEQ ID NO: 15)
MGSLGAILKHPDDFYPLMKLKMAARRAEKNIPPEPHWGFCYSMLHKVSRSFALVIQQLDTELRN
AVCIFYLVLRALDTVEDDTSIATEVKVPILMAFHRHIYDRDWHFSCGTKEYKVLMDEFHHVSTA
FSELGRGYQEAIEDITMRMGAGMAKFICKEVETIDDYDEYCHYVAGLVGLGLSKLFHASGSEDL
ASDSLSNSMGLFLQVFLLTCIKTNIIRDYLEDINEIPKSRMFWPRQIWSKYVNKLEDLKDKENS
VKAVECLNDMVTNALIHVEDCLTYMSALRDPSIFRFCAIPQIMAIGTLALCYNNIEVFRGVVKM
RRGLTAKVIDRTKTMSDVYGGFFDFSCMLKSKVNKSDPNAMKALSRLEAIQKICRESGTLNKRK
SYIIKSEPRYNSTLVFVLFIILAILFAYL
Eleutherococcus senticosus
(SEQ ID NO: 16)
MGSLGAILKHPDDFYPLLKLKFAARHAEKQIPPEPHWAFCYSMLHKVSRSFGLVIQQLDAQLRD
AVCIFYLVLRALDTVEDDTSIPTEVKVPILMAFHRHIYDKDWHFSCGTKEYKVLMDEFHHVSNA
FLELGSGFQEAIEDITMRMGAGMAKFICKEVETIDDYDEYCHYVAGLVGLGLSKLFHASGAEDL
ATDSLSNSMGLFLQKTNIIRDYLEDINEIPKSRMFWPRQIWSKYVDKLENLKYEENSAKAVQCL
NDMVTNALLHAEDCLKYMSNLRDPAIFRFCAIPQIMAIGTLALCFNNIQVFRGVVKMRRGLTAK
VIDRTKTMSDVYGAFFDFSCLLKSKVDNNDPNATKTLSRLEAIQKTCKESGTLSKRKSYIIESK
SAHNSALIAIIFIILAILYAYLSSNLPNNQ
Flavobacteriales bacterium
(SEQ ID NO: 166)
MLNNSLFSRLEEIPALLKLKLGSKDYYKNNNSETLTCDNLRYCFDTLNKVSRSFATVIKQLPNE
LGNNVCVFYLILRALDSIEDDMNLPKELKIKLLREFHKKNYESGWNISGVGDKKEHVELLENYD
KVIQSFLAIDQKNQLIITDICRKVGAGMANFVKAEIESVEDYNLYCHHVAGLVGIGLSRMFISS
GLENDDFLNQDEISNSMGLFLQKTNIVRDYREDLDEGRMFWPKDIWHVYGSKINDFAINPTHDQ
SVLCLNHMLNNALTHATDCLAYLKHLRNENIFKFCAIPQVMAMATLCKIYSNPDVFIKNVKIRK
GLAAKLILNTTSMDEVIKVYKDMLLVIESKISSDNNPVSAETIQLLKQIREYFNDETLIVRKIA
Bacteroidetes bacterium
(SEQ ID NO: 167)
MLNSSLFSRLEEIPALLKLKLGSINNYKNNNSENLTSKNLRYCFDTLNKVSRSFASVIKQLPNE
LMVNVCLFYLILRALDSIEDDMNLPKDFKINLLREFLDKNYEPGWKISGVGDKKEYVELLENYD
KVIQVFLDIDPKNQLIITDICRKMGAGMAHFVEAEINSVKDYNLYCYHVAGLVGIGLSKMFLAS
GLENCDYLNQEEISSSMGLFLQKTNIVRDYKEDMEENRIFWPKEIWRTYASKFSDFSINPQHET
SISCLNHMVNDALGHVIDCLEYLRHLRNENIFKFCAIPQVMAMATLCKVYNNPDVFIKTVKIRK
GLAAKLILNTTSMDEVIKVYKGLLLDIENKIPLHNPTSDETLRLIKNIRSYCNNETMVVSKTA
Squalene Epoxidase
Siraitia grosvenorii SQE1
(SEQ ID NO: 17)
MVDQCALGWILASALGLVIALCFFVAPRRNHRGVDSKERDECVQSAATTKGECRFNDRDVDVIV
VGAGVAGSALAHTLGKDGRRVHVIERDLTEPDRIVGELLQPGGYLKLIELGLQDCVEEIDAQRV
YGYALFKDGKNTRLSYPLENFHSDVSGRSFHNGRFIQRMREKAASLPNVRLEQGTVTSLLEEKG
TIKGVQYKSKNGEEKTAYAPLTIVCDGCFSNLRRSLCNPMVDVPSYFVGLVLENCELPFANHGH
VILGDPSPILFYQISRTEIRCLVDVPGQKVPSIANGEMEKYLKTVVAPQVPPQIYDSFIAAIDK
GNIRTMPNRSMPAAPHPTPGALLMGDAFNMRHPLTGGGMTVALSDIVVLRNLLKPLKDLSDAST
LCKYLESFYTLRKPVASTINTLAGALYKVFCASPDQARKEMRQACFDYLSLGGIFSNGPVSLLS
GLNPRPLSLVLHFFAVAIYGVGRLLLPFPSVKGIWIGARLIYSASGIIFPIIRAEGVRQMFFPA
TVPAYYRSPPVFKPIV
Siraitia grosvenorii SQE2
(SEQ ID NO: 18)
MVDQCALGWILASVLGAAALYFLFGRKNGGVSNERRHESTKNIATTNGEYKSSNSDGDIIIVGA
GVAGSALAYTLGKDGRRVHVIERDLTEPDRIVGELLQPGGYLKLTELGLEDCVDDIDAQRVYGY
ALFKDGKDTRLSYPLEKFHSDVAGRSFHNGRFIQRMREKAASLPKVSLEQGTVTSLLEENGIIK
GVQYKTKTGQEMTAYAPLTIVCDGCFSNLRRSLCNPKVDVPSCFVGLVLENCDLPYANHGHVIL
ADPSPILFYRISSTEIRCLVDVPGQKVPSISNGEMANYLKNVVAPQIPSQLYDSFVAATDKGNI
RTMPNRSMPADPYPTPGALLMGDAFNMRHPLTGGGMTVALSDVVVLRDLLKPLRDLNDAPTLSK
YLEAFYTLRKPVASTINTLAGALYKVFCASPDQARKEMRQACFDYLSLGGIFSNGPVSLLSGLN
PRPISLVLHFFAVAIYGVGRLLIPFPSPKRVWIGARIISGASAIIFPIIKAEGVRQMFFPATVA
AYYRAPRVVKGR
Momordica charantia
(SEQ ID NO: 19)
MVDECALGWILAAALGAVIALCLEVAPKTNNQDGGVDSKATPECVQTTNGECRSDGDSDVIIVG
AGVAGSALAHTLGKDGRRVHVIERDLTEPDRIVGELLQPGGYLKLIELGLADCVEEIDAQRVYG
YALFKDGKNTRLSYPLEKFHSDVSGRSFHNGRFIQRMREKADSLPNVRLEQGTVTSLLEEKGTI
KGVQYKSKDGKEKTAYAPLTIVCDGCFSNLRRSLCNPMVDVPSCFVGLVLENCQLPFANHGHVV
LGDPSPILFYPISSTEIRCLVDVPGQKVPSISNGEMEKYLKTVVAPQVPPQIYDAFIAAIDKGN
IRTMPNRSMPAAPHPTPGALLMGDAFNMRHPLTGGGMTVALSDIVVLRNLLKPLKDLHDAPTLC
KYLESFYTLRKPVASTINTLAGALYKVFCASPDQARKEMRQACFDYLSLGGMFSNGPVSLLSGL
NPRPLSLVLHFFAVAIYGVGRLLFPFPSPKGIWIGARLIYSASGIIFPIIKAEGVRQMFFPATV
PAYYRSPPALKPVA
Cucurbita maxima
(SEQ ID NO: 20)
MVDYCAFGWILAAVLGLAIALSFFVSPRRNRRGGADSTPRSEGVRSSSTTNGECRSVDGDADVI
IVGAGVAGSALAHTLGKDGRLVHVIERDLTEPDRIVGELLQPGGYLKLIELGLQDCVEEIDAQK
VYGYALFKDGKNTQLSYPLEKFQSDVSGRSFHNGRFIQRMREKAASLPNVRLEQGTVTSLLEEK
GTIKGVQYKSKNGEEKTAYAPLTIVCDGCFSNLRRSLCKPMVDVPSCFVGLVLENCQLPFANHG
HVVLGDPSPILFYPISSTEIRCLVDVPGQKIPSISNGEMEKYLKTIVAPQVPPQIHDAFIAAID
KGNIRTMPNRSMPAAPQPTPGALLMGDAENMRHPLTGGGMIVALSDIVVLRNLLKPLKDLNDAL
TLCKYLESFYTLRKPVASTINTLAGALYKVFCASPDQARKEMRQACFDYLSLGGIFSNGPVSLL
SGLNPRPLSLVLHFFAVAIYGVGRLLLPFPSPKGIWIGARLVYSASGIIFPIIKAEGVRQMFFP
ATVPAYYRSPPVHKSIA
Cucurbita moschata
(SEQ ID NO: 21)
MVDYCAFGWILAAVLGLAIALSFFVSPRRNRRGGADSTPRSEGVRSSSTTNGECRSVDCDADVI
IVGAGVAGSALAHTLGKDGRLVHVIERDLTEPDRIVGELLQPGGYLKLIELGLQDCVEEIDAQK
VYGYALFKDGKNTQLSYPLEKFQSDVSGRSFHNGRFIQRMREKAASLPNVRLEQGTVTSLLEEK
GTIKGVQYKSENGEEKTAHAPLTTVCDGCFSNLRRSLCKPMVDVPSCFVGLVLENCQLPFANHG
HVVLGDPSPILFYPISSTEIRCLVDVPGQKVPSISNGEMEKYLKTIVAPQVPPQIHDAFIAAID
KGNIRTMPNRSMPAAPQPTPGALLMGDAFNMRHPLTGGGMTVALSDIVVLRNLLKPLKDLNDAP
TLCKYLESFYTLRKPVASTINTLAGALYKVFCASPDQARKEMRQACFDYLSLGGIFSNGPVSLL
SGLNPRPLSLVLHFFAVAIYGVGRLLLPFPSPKGIWIGARLVYSASGIIFPIIKAEGVRQMFFP
ATVPAYYRSPPVIKTIA
Cucurbita moschata
(SEQ ID NO: 22)
MMVDHCAFAWILDVVLGLVVAVTFFVAAPRRNRRGGTDSTASKDCVISTAIANGECKPDDADAE
VIIVGAGVAGSALAYTLGKDGRRVHVIERDLTEPDRIVGEFLQPGGYLKLIELGLGDCVEEIDA
QKLYGYALFKDGKNTRVSYPLGNFHSDVSGRSFHNGRFIQRMREKAASLPNVRLEQGTVTSLLE
TKGTIKGVQYKSKNGEEKTAYAPLTIVCDGCFSNLRRSLCKPMVDVPSCFVGLVLENCQLPFAN
HGHVVLGDPSPILFYPISSTEIRCLVDVPGQKVPSISNGDMEKYLKTVVAPQVPPQIHDAFIAA
IEKGNVRTMPNRSMPAAPHPTPGALLMGDAFNMRHPLTGGGMTVALSDIVVLRNLLKPLKDLND
ASTLCKYLESFYTLRKPVASTINTLAGALYKVFCASPDQARKEMRQACFDYLSLGGVFSNGPIS
LLSGLNPRPSSLVLHFFAVAIYGVGRLLLPFPSLKGIWIGARLIYSASGIILPIIKAEGVRQMF
FPATVPAYYRSPPVHKPIT
Cucumis sativus
(SEQ ID NO: 23)
MVDHCTFGWIFSAFLAFVIAFSFFLSPRKNRRGRGTNSTPRRDCLSSSATTNGECRSVDGDADV
IIVGAGVAGSALAHTLGKDGRRVHVIERDLTEPDRIVGELLQPGGYLKLIELGLQDCVEEIDAQ
KVYGYALFKDGKSTRLSYPLENFQSDVSGRSFHNGRFIQRMREKAAFLPNVRLEQGTVTSLLEE
KGTITGVQYKSKNGEQKTAYAPLTIVCDGCFSNLRRSLCNPMVDVPSCFVGLVLENCQLPYANL
GHVVLGDPSPILFYPISSTEIRCLVDVPGQKVPSISNGEMEKYLKTVVAPQVPPQIHDAFIAAI
EKGNIRTMPNRSMPAAPQPTPGALLMGDAFNMRHPLTGGGMTVALSDIVVLRNLLKPLKDLNDA
PTLCKYLESEYTLRKPVASTINTLAGALYKVFCASSDQARKEMRQACFDYLSLGGIESNGPVSL
LSGLNPRPLSLVLHFFAVAIYGVGRLLLPFPSPKGIWIGARLVYSASGIIFPIIKAEGVRQMFF
PATVPAYYRTPPVFNS
Cucumis melo
(SEQ ID NO: 24)
MVDHCAFGWIFSALLAFPIALSLFLSPWRNRRVRGTDSTPRSASVSSSATTNGECRSVDGDADV
VIVGAGVAGSALAHTLGKDGRRVHVIERDLTEPDRIVGELLQPGGYLKLIELGLQDCVEEIDAQ
KVYGYALFKDGKNTRLSYPLENFHSDVSGRSFHNGRFTQRMREKAASLPNVRLEQGTVTSLLEE
KGTITGVQYKSKNGEQKTAYAPLTIVCDGCFSNLRRSLCTPMVDVPSYFVGLVLENCQLPYANL
GHVVLGDPSPILFYPISSTEIRCLVDVPGQKVPSISNGEMEKYLKTVVAPQVPPQIHDAFIAAI
EKGNTRTMPNRSMPAAPQPTPGALLMGDAFNMRHPLTGGGMTVALSDIVVLRNLLKPLKDLNDA
PTLCKYLESFYTLRKPVASTINTLAGALYKVFCASPDQARKEMRQACFDYLSLGGIFSNGPVSL
LSGLNPRPLSLVLHFFAVAIYGVGRLLLPFPSLKGIWIGARLVYSASGIIFPIIKAEGVRQMFF
PATVPAYYRTPPVLNS
Cucurbita maxima
(SEQ ID NO: 25)
MMVEHCAYGWILAAVLGLVVAVTFFVAVPRRNRRGGTDSTASKDCVISPAIANGECEPEDADAD
ADVIIVGAGVAGSALAHTLGKDGRRVHVIERDLTEPDRIVGEFLQPGGHLKLIELCLGDCVEEI
DAQKLYGYALFKDGKNTRVSYPLGNFHSDVSGRSFHNGRFIQRMREKAASLPNVRLEQGTVTSL
LEKKGTIKGVQYKSKNGEEKTAYAPLTIVCDGCFSNLRRSLCKPMVDVPSCFVGLVLENCRLPF
ANHGHVVLGDPSPILFYPISSTEIRCLVDVPGQKVPSIPNGDMEKYLKTVVAPQVPPQIHDAFI
AAIEKGNIRTMPNRSMPAAPHPTPGALLMGDAFNMRHPLTGGGMTVALSDIVVLRNLLKPLKDL
NDAPTLCKYLESYYTLRKPVASTINTLAGALYKVFCASPDQARKEMRQACFDYLSLGGVFSNGP
ISLLSGLNPRPSCLVLHFFAVAIYGVGRLLLPFPSLKGIWIGARLIYSASGIILPIIKAEGVRQ
MFFPATVPAYYRSPPVHKPIT
Ziziphus jujube
(SEQ ID NO: 26)
MLDQCPLGWILASVLGLFVLCNLIVKNRNSKASLEKRSECVKSIATTNGECRSKSDDVDVIIVG
AGVAGSALAHTLGKDGRRLHVIERDLTEPDRIVGELLQPGGYLKLIELGLQDCVEEIDAQRVFG
YALFKDGKDTRLSYPLEKFHSDVSGRSFHNGRFIQRMREKSASLPNVRLEQGTVTSLLEEKGTI
KGVQYKTKTGQELTAFAPLTIVCDGCFSNLRRSLCNPKVDVPSCFVGLVLENCELPYANHGHVI
LADPSPILFYPISSTEVRCLVDVPGQKVPSISNGEMARYLKSVVAPQIPPQIYDAFIAAVDKGN
IRTMPNRSMPASPFPTPGALLMGDAFNMRHPLTGGGMTVALSDIVVLRDLLKPLGDLNDAATLC
KYLESFYTLRKPVASTINTLAGALYKVFCASPDQARKEMRQACFDYLSLGGIFSTGPVSLLSGL
NPRPLSLVLHFFAVAIYGVGRLLLPFPSPKRIWIGARLISGASGIIFPIIKAEGVRQMFFPATV
PAYYRAAPVE
Morus alba
(SEQ ID NO: 27)
MADPYTMGWILASLLGLFALYYLFVNNKNHREASLQESGSECVKSVAPVKGECRSKNGDADVII
VGAGVAGSALAHTLGKDGRRVHVIERDLAEPDRIVGELLQPGGYLKLIELGLQDCVEEIDSQRV
YGYALFKDGKDTRLSYPLEKFHSDVSGRSFHNGRFIQRMREKAASLPNVQLEQGTVTSLLEENG
TIKGVQYKTKTGQELTAYAPLTIVCDGCFSNLRRSLCIPKVDVPSCFVGLVLENCNLPYANHGH
VVLADPSPILFYPISSTEVRCLVDVPGQKVPSISNGEMAKYLKTVVASQIPPQIYDSFVAAVDK
GNIRTMPNRSMPAAPHPTPGALLMGDAFNMRHPLTGGGMTVALSDIVVLRDLLKPLRDLNDSVT
LCKYLESFYTLRKPVASTINTLAGALYKVFCASPDQARKEMREACFDYLSLGGVFSEGPVSLLS
GLNPRPLSLVCHFFAVAIYGVGRLLLPFPSPKRLWIGARLISGASGIIFPIIRAEGVRQMFFPA
TIPAYYRAPRPN
Juglans regia (JrSQE1)
(SEQ ID NO: 28)
MVDPYALGWSFASVLMGLVALYILVDKKNRSRVSSEARSEGVESVTTTTSGECRLTDGDADVII
VGAGVAGSALAHTLGKDGRRVHVIERDLTEPDRIVGELLQPGGYLKLIELGLEDCVEDIDAQRV
FGYALFKDGKNTRLSYPLEKFHSDVSGRSFHNGRFIQRMREKAASLLNVRLEQGTVTSLLEENG
TVKGVQYKTKDGNELTAHAPLTIVCDGCFSNLRRSLCNPQVDVPSSFVGLVLENCELPYANHGH
VILADPSPILFYPISSTEVRCLVDVPGKKVPSIANGEMEKYLKNMVAPQLPPEIYDSFVAAVDR
GNIRTMPNRSMPAAPHPTPGALLMGDAFNMRHPLTGGGMTVALSDIVVLRDLLKPLRDLNDAPT
LCKYLESFYTLRKPVASTINTLAGALYKVFCASPDRARKEMRQACFDYLSLGGVFSMGPVSLLS
GLNPRPLSLVLHFFAVAVYGVGRLLVPFPSPSRIWIGARLISGASAIIFPIIKAEGVRQMFFPA
TVPAYYRAPPVKRDH
Cucumis melo
(SEQ ID NO: 29)
MVDQCALGWILASVLGASALYLLFGKKNCGVLNERRRESLKNIATTNGECKSSNSDGDIIIVGA
GVAGSALAYTLAKDGRQVHVIERDLSEPDRIVGELLQPGGYLKLTELGLEDCVDDIDAQRVYGY
ALFKDGKDTRLSYPLEKFHSDVSGRSFHNGRFIQRMREKAASLPNVRLEQGTVTSLLEENGTIK
GVQYKNKSGQEMTAYAPLTIVCDGCFSNLRRSLCNPKVDVPSCFVGLILENCDLPYANHGHVII
ADPSPILFYPISSTEIRCLVDVPGQKVPSISNGEMANYLKNVVAPQIPPQLYNSFIAAIDKGNI
RTMPNRSMPADPYPTPGALLMGDAFNMRHPLTGGGMTVALSDIVVLRDLLKPLRDLNDAPTLCK
YLEAFYTLRKPVASTINTLAGALYKVFCASPDQARKEMRQACFDYLSLGGIFSNGPVSLLSGLN
PRPLSLVLHFFAVAIYGVGRLLIPFPSPKRVWIGARLISGASAIIFPIIKAEGVRQMFFPKTVA
AYYRAPPVVRER
Cucumis sativus
(SEQ ID NO: 30)
MVDQCALGWILASVLGASALYLLFGKKNCGVSNERRRESLKNIATTNGECKSSNSDGDIIIVGA
GVAGSALAYTLAKDGRQVHVIERDLSEPDRIVGELLQPGGYLKLTELGLEDCVDEIDAQRVYGY
ALFKDGKDTRLSYPLEKFHSDVSGRSFHNGRFIQRMREKAASLPNVRLEQGTVTSLLEENGTIR
GVQYKNKSGQEMTAYAPLTIVCDGCFSNLRRSLCNPKVDVPSCFVGLILENCDLPHANHGHVIL
ADFSPILFYPISSTEIRCLVDVPGQKVPSISNGEMANYLKNVVAPQIPPQLYNSFIAAIDKGMI
RTMPNRSMPADPYPTPGALLMGDAFNMRHPLTGGGMTVALSDIVVLRDLLKPLRDLNDAPTLCK
YLEAFYTLRKPVASTINTLAGALYKVFCASPDQARKEMRQACFDYLSLGGIFSNGPVSLLSGLN
PRPLSLVLHFFAVAIYGVGRLLIPFPSPKRVWIGARLISGASAIIFPIIKAEGVRQMFFPKTVA
AYYRAPPIVRER
Juglans regia (JrSQE2)
(SEQ ID NO: 31)
MVDQYALGLILASVLGFVVLYNLMAKKNRIRVSSEARTEGVQTVITTTNGECRSIEGDVDVIIV
GAGVAGSALAHTLGKDGRKVHVIERDLSEPDRIVGELLQPGGYLKLVELGLQDSVEDIDAQRVF
GYALFKDGKNTRLSYPLEKFHSDVSGRSFHNGRFIQRMREKAASLPNIRLEQGTVTSLLEENGT
IKGVQYKTKDGKELAAHAPLTIVCDGCFSNLRRSLCNPQVDVPSSFVGLVLENCELPYANHGHV
VLADPSPILFYPISSTEVRCLVDVPGQKVPSISNGEMAKYLKTMVAPQVPPEIYDSFVAAVDRG
NIRTMPNRSMPAAPQPTPGALLMGDAFNMRHPLTGGGMTVALSDIVVLRDLLRPLRDLNDAPTL
CKYLESFYTLRKPVASTINTLAGALYKVFCASPDRARNEMRQACFDYLSLGGVFSTGPVSLLSG
LNPRPLSLVLHFFAVAVYGVGRLLVPFPSPSRMWIGARLISGASAIIFPIIKAEGVRQMFFPAT
VPAYYRAPPVNCQARSLKPDALKGL
Theobroma cacao
(SEQ ID NO: 32)
MADSYVWGWILGSVMTLVALCGVVLKRRKGSGISATRTESVKCVSSINGKCRSADGSDADVIIV
GAGVAGSALAHTLGKDGRRVHVIERDLTEPDRIVGELLQPGGYLKLIELGLEDCVEEIDAQQVF
GYALFKDGKHTRLSYPLEKFHSDVSGRSFHNGRFIQRMREKSASLPNVRLEQGTVTSLLEEKGT
IRGVQYKTKDGRELTAFAPLTIVCDGCFSNLRRSLCNPKVDVPSCFVGLVLENCNLPYSNHGHV
ILADPSPILFYPISSTEVRCLVDVPGQKVPSIANGEMANYLKTIVAPQVPPEIYNSFVAAVDKG
NIRTMPNRSMPAAPYPTPGALLMGDAFNMRHPLTGGGMTVALSDIVVLRDLLRPLRDLNDAPTL
CKYLESEYTLRKPIASTINTLAGALYKVFCASPDQARKEMRQACFDYLSLGGVFSTGPISLLSG
LNPRPVSLVLHFFAVAIYGVGRLLLPFPSPKRIWIGARLISGASGIIFPIIKAEGVRQMFFPAT
VPAYYRAPPVE
Cucurbita moschata
(SEQ ID NO: 33)
MMVDHCAFAWILDVVLGLVVAVTFFVAAPRRNRRGGTDSTASKDCVISTAIANGECKPDDADAE
VIIVGAGVAGSALAYTLGKDGRRVHVIERDLTEPDRIVGEFLQPGGYLKLIELGLGDCVEEIDA
QKLYGYALFKDGKNTRVSYPLGNFHSDVSGRSFHNGRFIQRMREKAASLPNVRLEQGTVTSLLE
TKGTTKGVQYKSKNGEEKTAYAPLTIVCDGCFSNLRRSLCKPMVDVPSCFVGLVLENCQLPFAN
HGHVVLGDPSPILFYPISSTEIRCLVDVPGQKVPSISNGDMEKYLKTVVAPQVPPQIHDAFIAA
IEKGNVRTMPNRSMPAAPHPTPGALLMGDAFNMRHPLTGGGMTVALSDIVVLRNLLKPLKDLND
ASTLCKYLESFYTLRKPVASTINTLAGALYKVFCASPDQARKEMRQACFDYLSLGGVFSNGPIS
LLSGLNPRPSSLVLHFFAVAIYGVGRLLLPFPSLKGIWIGARLIYSASGIILPIIKAEGVRQMF
FPATVPAYYRSPPVHKPIT
Phaseolus vulgaris
(SEQ ID NO: 34)
MLDTYVFGWIICAALSVFVIRNFVFAGKKCCASSETDASMCAENITTAAGECRSSMRDGEFDVL
IVGAGVAGSALAYTLGKDGRQVLVIERDLSEPDRIVGELLQPGGYLKLIELGLEDCVDKIDAQQ
VFGYALFKDGKHIRLSYPLEKFHSDVAGRSFHNGRFIQRMREKAASLPNVRLEQGTVTSLLEEK
GVIKGVQYKTKDSQELSVCAPFTIVCDGCFSNLRRSLCDPKVDVPSCFVGLVLENCELPCANHG
HVILGEPSPVLFYPISSTEIRCLVDVPGQKVPSISNGEMAKYLKTVIAPQVPHELHNAFIAAVD
KGSIRTMPNRSMPAAPYPTPGALLMGDAFNMRHPLTGGGMTVALSDIVVLRNLLRPLRDLNDAP
SLCKYLESFYTLRKPVASTINTLAGALYKVFCASSDPARKEMRQACFDYLSLGGQFSEGPISLL
SGLNPRPLTLVLHFFAVATYGVGRLLLPFPSPKRMWIGLRLISSASGIIMPIIKAEGVRQMFFP
ATVPAYYRNPPAA
Hevea brasiliensis
(SEQ ID NO: 35)
MKMADHYLLGWILASVMGLFAFYYIVYLLVKPEEDNNRRSLPQPRSDFVKTMTATNGECRSDDD
SDVDVIIVGAGVAGAALAHTLGKDGRRVHVIERDLTEPDRIVGELLQPGGYLKLIELGLEDCVE
EIDAQRVFGYALFKDGKHTQLAYPLEKFHSEVAGRSFHNGRFIQRMREKAASLPSVKLEQGTVT
SLLEEKGTIKGVLYKTKTGEELTAFAPLTIVCDGCFSNLRRSLCNPKVDVPSCFVGLVLENCRL
PYANNGHVILADPSPILFYPISSTEVRSLVDVPGQKVPSVSSGEMANYLKNVVAPQVPPEIYDS
FVAAVDKGNIRTMPNRSMPASPYPTPGALLMGDAFNMRHPLTGGGMTVALSDIVVLRDLLKPLR
DLHDAPTLCRYLESFYTLRKPVASTINTLAGALYKVFCASPDEARKEMRQACFDYLSLGGVFST
GPVSLLSGLNPRPLSLVLHFFAVAIYGVGRLLLPFPSPHRIWVGARLISGASGIIFPIIKAEGV
RQMFFPATVPAYYRAPPIKCN
Sorghum bicolor
(SEQ ID NO: 36)
MAAAAAAASGVGFQLIGAAAATLLAAVLVAAVLGRRRRRARPQAPLVEAKPAPEGGCAVGDGRT
DVIIVGAGVAGSALAYTLGKDGRRVHVIERDLTEPDRIVGELLQPGGYLKLIELGLEDCVEEID
AQRVLGYALFKDGRNTKLAYPLEKFHSDVAGRSFHNGRFTQRMRQKAASLPNVQLEQGTVTSLL
EENGTVKGVQYKTKSGEELKAYAPLTIVCDGCFSNLRRALCSPKVDVPSCFVGLVLENCQLPHP
NHGHVILANPSPILFYPISSTEVRCLVDVPGQKVPSIASGEMANYLKTVVAPQIPPEIYDSFIA
AIDKGSIRTMPNRSMPAAPHPTPGALLMGDAFNMRHPLTGGGMTVALSDIVVLRNLLKPLHNLH
DASSLCKYLESFYTLRKPVASTINTLAGALYKVFSASPDQARNEMRQACFDYLSLGGVFSNGPI
ALLSGLNPRPLSLVAHFFAVAIYGVGRLMLPLPSPKRMWIGARLISGACGIILPIIKAEGVRQM
FFPATVPAYYRAAPMGE
Zea mays
(SEQ ID NO: 37)
MRKNLEEAGCAVSDGGTDVIIVGAGVAGSALAYTLGKDGRRVHVIERDLTEPDRIVGELLQPGG
YLKLIELGLQDCVEEIDAQRVLGYALFKDGRNTKLAYPLEKFHSDVAGRSFHNGRFIQRMRQKA
ASLPNVQLEQGTVTSLLEENGTVKGVQYKTKSGEELKAYAPLTIVCDGCFSNLRRALCSPKVDV
PSCFVGLVLENCQLPHPNEGHVILANPSPILFYPISSTEVRCLVDVPCQKVPSIATGEMANYLK
TVVAPQIPPEIYDSFIAAIDKGSIRTMPNRSMPAAPHPTPGALLMGDAFNMRHPLTGGGMTVAL
SDIVVLRNLLKPLRNLHDASSLCKYLESFYTLRKPVASTINTLAGALYKVFSASPDQARNEMRQ
ACFDYLSLGGVFSNGPIALLSGLNPRPLSLVAHFFAVAIYGVGRLMLPLPSPKRMWIGARLISG
ACGIILPIIKAEGVRQMFFPATVPAYYRAAPTGEKA
Medicago sativa
(SEQ ID NO: 38)
MDLYNIGWILSSVLSLFALYNLIFSGKRNYHDVNDKVKDSVTSTDAGDIQSEKLNGDADVIIVG
AGIAGAALAHTLGKDGRRVHIIERDLSEPDRIVGELLQPGGYLKLVELGLQDCVDNIDAQRVFG
YALFKDGKHTRLSYPLEKFHSDVSGRSFHNGRFIQRMREKAASLPNVNMEQCTVISLLEEKGTI
KGVQYKNKDGQALTAYAPLTIVCDGCFSNLRRSLCNPKVDNPSCFVGLILENCELPCANHGHVI
LGDPSPILFYPISSTEIRCLVDVPGTKVPSISNGDMTKYLKTTVAPQVPPELYDAFIAAVDKGN
IRTMPNRSMPADPRPTPGAVLMGDAFNMRHPLTGGGMTVALSDIVVLRNLLKPMRDLNDAPTLC
KYLESFYTLRKPVASTINTLAGALYKVFSASPDEARKEMRQACFDYLSLGGLFSEGPISLLSGL
NPRPLSLVLHFFAVAVFGVGRLLLPFPSPKRVWIGARLLSGASGIILPIIKAEGIRQMFFPATV
PAYYRAPPVNAF
Methylomonas lenta
(SEQ ID NO: 39)
MKEEFDICIIGAGMAGATISAYLAPKGIKIALIDHCYKEKKRIVGELLQPGAVLSLEQMGLSHL
LDGFEAQTVKGYALLQGNEKTTIPYPSQHEGIGLHNGRFLQQIRASALENSSVTQIHGKALQLL
ENERNEIIGVSYRESITSQIKSIYAPLTITSDGFFSNFRAHLSNNQKTVTSYFIGLILKDCEMP
FPKHGHVFLSGPTPFICYPISDNEVRLLIDFPGEQLPRKNLLQEHLDTNVTPYIPECMRSSYAQ
AIQEGGFKVMPNHYMAAKPIVRKGAVMLGDALNMRHPLTGGGLTAVFSDIQILSAHLLAMPDFK
NTDLIHEKIEAYYRDRKRANANLNILANALYAVMSNDLLKTAVFKYLQCGGANAQESIAVLAGL
NRKHFSLIKQFCFLAVFGACNLLQQSISNIPKALKlLKDAFVIIKPLIKNELS
Bathymodiolus azoricus Endosymbiont
(SEQ ID NO: 168)
MHTTSEHNDLFDICIVGAGMAGATIATYLAPRGIKIALIDRDYAEKRRIVGELLQPGAVQTLKK
MGLEHLLEGFDAQPIYGYALFNKDCEFSIEYNQDKSTNYRGVGLHNGRFLQKIREDALKQPSIT
QIHGTVSELIEDENHVVTGVKYKEKYTRELKTVNAKLTITSDGFFSSFRKDLTNNVKTVTSFFV
GIILKDCELPYPHHGHVFLSAPTPFICYPISSTESRLLIDFPGDQAPKKEAVKHHIENNVIPFL
PKEFRLCLDQALRENDYKIMPNHYMPAKPVLKKGVVLLGDALNMRHPITGGGLTAVFNDVYLLS
THLLAMPDFNDTKLIHSKVNLYYNDRYHANTNVNIMANALYGVMSNDLLKQSVFEYLRKGGDNS
GGPISLLAGLNRNPTILIKHFFSVALLCLRNLFKAHKMSLTNAFYVIKDAFCIIVPLAINELRP
SSFLKKNIHN
Methyloprofundus sediment
(SEQ ID NO: 169)
MNTSPEHNDLFDICIVGVGMAGATIAAYLAPRGLKIALIDREYTEKRRIVGELLQPGAVQTLKK
MGLEHLLEGFDAQPIYGYALFNNDKEFSISYNSDDSTEYHGVGLHNGRFLQKIREDVFKNETVT
QIHGTVSELIEDKKGVVKGVTYREKHTREYKTVKAKLTVTSDGFFSNFRKDLSNNVKTVTSFFI
GLVLNDCNLPFPNHGHVFLSAPTPFICYPISSTETRLLIDYPGDKAPKKDEIREHILNKVAPFL
PEEFKECFANAMEDDDFKVMPNHYMPAKPVLKEGAVLLGDALNMRHPLTGGGLTAVFNDVYLLS
THLLAMPDFNDPKLLHEKLELYYQDRYHANTNVNIMANALYGVMSNDLLKQGVFEYLRKGGDNS
GGPITLLAGLNRNPTLLIKHFFSVAFLCICNLSGNNKMNFTNVFRVMKDAFCIIKPLAVNELRP
SSFYKKNIQL
Methylomicrobium buryatense
(SEQ ID NO: 170)
MESNFDICIIGAGMAGATIAAYLAPKGINIALIDHCYKEKKRIVGELLQPGAVLSLEQLGLGHL
LDGIDAQPVEGYALLQGNEQTTIPYPSPNHGMGLHNGRFLQQIRASALQNSSVTQIQGKALSLL
ENEQNEIIGVNYRDSVSNEIKSIYAPLTITSDGFFSNFRELLSNNEKTVTSYFIGLILKDCEIP
VPKHGHVFLSGPTPFICYPISSNEVRLLIDFPGGQFPRKAFLQAHLETNVTPYIPEGMQTSYRH
ALQEDRLKVMPNHYMAAKPKIRKGAVMLGDALNMRHPLTGGGLTAVFSDIEILSGHLLAMPDFN
NNDLIYQKIEAYYRDRQYANANLNILANALYGVMSNELLKNSVFKYLQRGGVNAKESIAILAGL
NKNHYSLMKQFFFVALFGAYTLVRENITNLPKATKILSDALTIIKPLAKNELSLVCIFSDYFKR
Ononis spinosa SQE1
(SEQ ID NO: 177)
MVDPYAVGWIICSLTTIVALYNFVFYRQNRSDKTTPTTTENITTATGDCRSLNPNGDVDIVIVG
AGVAGSALAYTLGKDGRRVLVIERDLNEPDRIVGELLQPGGYLKLIELGLEDCVEKIDAQQVFG
YALFKDGKHTRLSYPLEKFHSDIAGRSFHNGRFIQRMREKAASLPNVQLVQGTVTSLLEENGTI
KGVQYKTKDAQELSACAPLTIVCDGCFSNLRRNLCNPKVEVPSCFVGLVLENCELPCANHGHVI
LGDPSPVLFYPISSTEIRCLVDVPGQKVPSISNGEMAKYLKEVVAPQVPPELHDAFIAAVDKGN
IRTMPNRSMPAAPYPTPGALLMGDAFNMRHPLTGGGMTVALSDIVVLRNLLKPLRDLNDAPSLC
KYLESFYTLRKPVASTINTLAGALYKVFCASPDPARKEMRQACFDYLSLGGLFSEGPVSLLSGL
NPRPLSLVLHFFAVAIYGVGRLLLPFPSPKRIWIGVRLIASASGIILPIIKAEGIRQMFFPATV
PAYYRTPPAA
Ononis spinosa SQE2
(SEQ ID NO: 178)
MDLYLLGWILSSVLSLFALYCLVFDGNRSRANAEKQIQRGYSVTTDAGDVKSEKLNGDADVIIV
GAGIAGAALAHTLGKDGRRVRVIERDLSEPDRIVGELLQPGGYLKLVELGLADCVDNIDAQKVF
GYALFKDGKHTRLSYPLEKFHADVSGRSFHNGRFIQRMREKAASLLNVNLEQGTVTSLLEEKGT
IKGVQYKNKDGQELTAYAPLTIVCDGCFSNLRRSLCNPKVDNPSCFVGLVLENCELPCANHGHV
ILGDPSPILFYPISSTEIRCLVDVPGQKVPSISNGDMTKYLKLTVAPQVPPELYDAFIAAVDKG
NIRTMPNKSMPADPCPTPGAVLMGDAFNMRHPLTGGGMTVALSDIVVLRNLLRPLRDLNDAPAL
CKYLESFYTLRKPVASTINTLAGALYKVFSSSPDQARREMRQACFDYLSLGGLFSEGPISLLSG
LNPRPLSLVLHFFAVAVFGVGRLLLPFPSPKRVWIGARLLSAASGIILPIIKAEGIRQMFFPVT
VPAYYRAPPTSQE
Medicago truncatula SQE1
(SEQ ID NO: 179)
MIDPYGFGWITCTLITLAALYNFLFSRKNHSDSTTTENITTATGECRSFNPNGDVDIIIVGAGV
AGSALAYTLGKDGRRVLIIERDLNEPDRIVGELLQPGGYLKLIELGLDDCVEKIDAQKVFGYAL
FKDGKHTRLSYPLEKFHSDIAGRSFHNGRFILRMREKAASLPNVRLEQGTVTSLLEENGTIKGV
QYKTKDAQEFSACAPLTIVCDGCFSNLRRSLCNPKVEVPSCFVGLVLENCELPCADHGHVILGD
PSPVLFYPISSTEIRCLVDVPGQKVPSISNGEMAKYLKTVVAPQVPPELHAAFIAAVDKGHIRT
MPNRSMPADPYPTPGALLMGDAFNMRHPLTGGGMTVALSDIVVLRNLLKPLRDLNDASSLCKYL
ESFYTLRKPVASTINTLAGALYKVFCASPDPARKEMRQACFDYLSLGGLFSEGPVSLLSGLNPC
PLSLVLHFFAVAIYGVGRLLLPFPSPKRLWIGIRLIASASGIILPIIKAEGIRQMFFPATVPAY
YRAPPDA
Medicago truncatula SQE2
(SEQ ID NO: 180)
MDLYNIGWILSSVLSLFALYNLIFAGKKNYDVNEKVNQREDSVTSTDAGEIKSDKLNGDADVII
VGAGIAGAALAHTLGKDGRRVHIIERDLSEPDRIVGELLQPGGYLKLVELGLQDCVDNIDAQRV
FGYALFKDGKETRLSYPLEKFHSDVSGRSFHGRFIQRMREKAASLPNVNMEQGTVISLLEEKGT
IKGVQYKNKDGQALTAYAPLTIVCDGCFSNLRRSLCNPKVDNPSCFVGLILENCELPCANHGHV
ILGDPSPILFYPISSTEIRCLVDVPGTKVPSISNGDMTKYLKTTVAPQVPPELYDAFIAAVDKG
NIRTMPNRSMPADPRPTPGAVLMGDAFNMRHPLTGGGMTVALSDIVVLRNLLKPMRDLNDAPTL
CKYLESFYTLRKPVASTINTLAGALYKVFSASPDEARKEMRQACFDYLSLGGLFSEGPISLLSG
LNPRPLSLVLHEFAVAVFGVGRLLLPFPSPKRVWIGARLLSGASGIILPIIKAEGIRQMFFPAT
VPAYYRAPPVNAF
Hypholoma sublateritium SQE
(SEQ ID NO: 181)
MSKSRSNYDVIIVGAGIAGCALAHGLSTLSRATPLRIAIVERSLAEPDRIVGELLQPGGVMALQ
RLGMEGCLEGIDAVKVHGYCVVENGTSVHIPYPGVHEGRSFHHGRFIMKLREAARAARGVELVE
ATVTELIPREGGKGIAGVRVARKGKDGEEDTTEALGAALVVVADGCFSNFRAAVMGGAAVKPET
KSHFVGAILKDARLPIPNHGTVALVKGFGPVLLYQISEHDTRMLVDVKAPLPADLKVCAHILSN
IVPQLPAALHLPIQRALDAERLRRMPNSFLPPVEQGATRGAVLVGDAWNMRHPLTGGGMTVALN
DVCCLRDLLGSVGDLGDWRQVASTVNILSVALYDLFGADGELQVLRTGCFKYFERGGDCIDGPV
SLLSGIAPSPMLLAYHFFSVAFYSIYVIAVGAQNGSAKQVLAVPGALQYPALCVKGLRVFYTAC
VVFGPLLWTELRW
Hypholoma sublateritium SQE2
(SEQ ID NO: 182)
MHPTHYDVVIVGAGVAGSSLAHALATLPREKPLQIALIERSFEEPDRIVGELLQPGGVDALKTL
KMTSSVEGIDAITVTGYILVESGDMVRTPYPKGKEGRSFHHGRFIMGLRRVALENPNVHPIEAT
AADLIECPCTGQVIGVRATSKTAPAPSSIDAQQTPPAPFSVYGDLVIVADGCFSNFRNVVMGKA
ACKATTKSYFVGTILKDAVLPVAGHGTVILPQGSGPVLLYQISEHDTRMLIDIQHPLPSDLRAH
ILTNILPQLPASIQGVVSDAPTKDRIRRMPNSFLPSVQQGSPLSKKGVILLGDSWNMRHPLTGG
GMTVALNDVVYLRSIFASIQNLDDWDEIRYALRHWHWGRKPLSSTINILSGTLYGLFEKDDDDY
RATRKGCFKYFQLGGKCIDDPVSLLSGLSPSPTLLSSHFFAVTLYAIWVVFTHPRVGSSMSANP
ADVKRVYDIPSADEYPQLTLKGIRMFSQACGVFLPVLWSEIRWWAPCESS
Hypholoma sublateritium SQE3
(SEQ ID NO: 183)
MSKSRSNYDVIIVGAGIAGCALAHGLSTLSRATPLRIAIVERSLAEPDRIVGELLQPGGVMALQ
RLGMEGCLEGIDAVKVHGYCVVENGTSVHIPYPGVHEGRSFHHGRFIMKLREAARAARGVELVE
ATVTELIPREGGKGIAGVRVARKGKDGEEDTTEALGAALVVVADGCFSNFRAAVMGGAAVKPET
KSHFVGAILKDARLPIPNHGTVALVKGFGPVLLYQISEHDTRMLVDVKAPLFADLKAHILSNIV
PQLPAALHLPIQRALDAERLRRMPNSFLPPVEQGATRGAVLVGDAWNMRHPLTGGGMTVALNDV
VVLRDLLGSVGDLGDWRQVRRALHRWHWDRKPLASTVNILSVALYDLFGADGEELQVLRTGCFK
YFERGGDCIDGPVSLLSGIAPSPMLLAYHFFSVAFYSIYVMFAHPQPVAQSKAVGAQNGSAKQV
LAVPGALQYPALCVKGLRVFYTACVVFGPLLWTELRWWTAAEASRGRLLVMSLVPLLLLLGAAN
YGIPGMGLLGVL
MlSQE A4
(SEQ ID NO: 203)
MAKEEFDICIIGAGMAGATISAYLAPKGIKIALIDRCYKEKKRIVGELLQPGAVLSLEQMGLSH
LLDGFEAQTVKGYALLQGNEKTTIPYPSQHEGIGLHNGRFLQQIRASALENSSVTQIHGKALQL
LENERNEIIGVSYRESITSQIKSIYAPLTITSDGFASNFRAHLSNNQKTVTSYFIGLILKDCEM
PFPKHGHVFLSGPTPFICYPISDNEVRLLIDFPGEQLPRKNLLQEHLDTNVTPYIPECMRSSYA
QAIQEGGFKVMPNHYMAAKPIVRKGAVLLGDALNMRHPLTGGGLTAVFSDIQILSAHLLAMPDF
KNTDLIHEKIEAYYRDRKRANANLNILANALYAVMSNDLLKTAVFRYLQCGGANAQESTALLAG
LNRKHFSLIKQYCFLAVFGACNLLQQSISNIPKALKLLKDAFVIIKPLIKNELS
Cucurbitadienol Synthase (CDS), Triterpene Synthase (TTP)
Siraitia grosvenorii CDS
(SEQ ID NO: 40)
MWRLKVGAESVGENDEKWLKSISNHLGRQVWEFCPDAGTQQQLLQVHKARKAFHDDRFHRKQSS
DLFITIQYGKEVENGGKTAGVKLKEGEEVRKEAVESSLERALSFYSSIQTSDGNWASDLGGPMF
LLPGLVIALYVTGVLNSVLSKHHRQEMCRYVYNHQNEDGGWGLHIEGPSTMFGSALNYVALRLL
GEDANAGAMPKARAWILDHGGATGITSWGKLWLSVLGVYEWSGNNPLPPEFWLFPYFLPFHPGR
MWCHCRMVYLPMSYLYGKRFVGPITPIVLSLRKELYAVPYHEIDWNKSRNTCAKEDLYYPHPKM
QDILWGSLHHVYEPLFTRWPAKRLREKALQTAMQHIHYEDENTRYICLGPVNKVLNLLCCWVED
PYSDAFKLHLQRVHDYLWVAEDGMKMQGYNGSQLWDTAFSIQAIVSTKLVDNYGPTLRKAHDFV
KSSQIQQDCPGDPNVWYRHIHKGAWPFSTRDHGWLISDCTAEGLKAALMLSKLPSETVGESLER
NRLCDAVNVLLSLQNDNGGFASYELTRSYPWLELINPAETFGDIVIDYPYVECTSATMEALTLE
KKLHPGHRTKEIDTAIVRAANFLENMQRTDGSWYGCWGVCFTYAGWFGIKGLVAAGRTYNNCLA
IRKACDFLLSKELPGGGWGESYLSCQNKVYTNLEGNRPHLVNTAWVLMALIEAGQAERDPTPLH
RAARLLINSQLENGDFPQQEIMGVFNKNCMITYAAYRNIFPIWALGEYCHRVLTE
Momordica charantia
(SEQ ID NO: 41)
MWRLKVGAESVGENDEKWVKSISNHLGRQVWEFCPDAGTPQQLLQIEKARKAFQDNRFHRKQTS
DLLVSIQCEKGTTNGARVPGTKLKEGEEVRKEAVKSTLERALSFYSSIQTSDGNWASDLGGPME
LLPGLVIALCVTGALNSVLSKHHRQEMCRYLYNHQNEDGGWGLHIESPSTMFGSALNYVALRLL
GEDADGGEGRAMTKARAWILGHGGATAITSWGKLWLSVLGVYEWSGNNPLPPEFWLLPYFLPFE
PGRMWCHCRMVYLPMSYLYGKRFVGPITPVVLSLRKELYTVPYHEIDWNKSRNTCAKEDLYYPH
SKMQDILWGSIHHMYEPLFTHWPAKRLREKALKTAMQHIHYEDENTRYICLGPVNKVLNMLCCW
VEDPYSEAFKLHLQRVHDYLWVAEDGMKMQGYNGSQLWDTAFSVQAIISTKLVDNYGPTLRKAH
DYVKNSQIQQDCPGEPNVWFRHIHKGAWPFSTRDHGWLISDCTAEGLKASLMLSKLPSETVGEP
LERNRLCDAVNVLLSLQNDNGGFASYELTRSYPWLELINPAETFGDIVIDYPYVECTSATMEAL
ALFKKLHPGHRTKEIDTAIARAADFLENMQRTDGSWYGCWGVCETYAGWFGIKGLVAAGRAYSN
CLAIRKACDFLLSKELPGGGWGESYLSCQNKVYTNLEGNRPHLVNTAWVLMALIEAGQGERDPA
PLHRAARLLINSQLENGDFPQEEIMGVFNKNCMITYAAYRNIFPIWALGEYCHRVLTE
Cucurbita maxima
(SEQ ID NO: 42)
MWRLKVGAESVGEKDEKKVKSVSNKLGRQVWEFCADAAADTPHQLLQIQMARNHFHHNRFHRKC
SSDLFLAIQYEKEIAKGAKGGAVKVKEGSEVGKEAVKSTLERALGFYSAVQTSDGNWASDLGGP
MFLLPGLVIALHVTGVLMSVLSKHKRVEMCRYLYNKQNEDGGWGLHIEGTSTMFGSALNYVALR
LLGEDADGGDGGAMTKARAWILERGGATAITSWGKLWLSVLGVYEWSGNNPLPPEFWLLPYSLP
FHPGRMWCHCRMVYLPMSYLYGKRFVGPITPKVLSLRQELYTIPYHEIDWNKSRNTCAKEDLYY
PHPKMQDILWGSIYHVYEPLFTRWPGKRLREKALQAAMKHIHYEDENSRYICLGPVNKVLNMLC
CWVEDPYSDAFKLHLQRVHDYLWVAEDGMRMQGYNGSQLWDTAFSIQAIVATKLVDSYAPTLRK
AHDFVKDSQIQEDCPGDPNVWFRHIHKGAWPFSTRDHGWLISDCTAEGLKASLMLSKLPSTMVG
EPLEKNRLCDAVNVLLSLQNDNGGFASYELTRSYPWLELINPAETFGDIVIDYPYVECTAATME
ALTLFKKLHPGHRTKEIDTAIGKAANFLEKMQRADGSWYGCWGVCFTYAGWFGIKGLVAAGRTY
NSCLAIRKACEFLLSKELPGGGWGESYLSCQNKVYTNLEGNKPHLVNTAWVLMALIEAGQGERD
PAPLHRAARLLMNSQLENGDFVQQEIMGVFNKNCMITYAAYRNIFPIWALGEYCHRVLTE
Citrullus colocynthis (CcCDS1)
(SEQ ID NO: 43)
MWRLKVGAESVGEKEEKWLKSISNHLGRQVWEFCADQPTASPNHLQQIDNARKHFRNNRFHRKQ
SSDLFLAIQNEKEIANGTKGGGIKVKEEEDVRKETVKNTVERALSFYSAIQTNDGNWASDLGGP
MFLLPGLVIALYVTGVLNSVLSKHHRQEMCRYLYNHQNEDGGWGLHIEGTSTMFGSALNYVALR
LLGEDADGGEGGAMTKARGWILDRGGATAITSWGKLWLSVLGVYEWSGNNPLPPEFWLLPYCLP
FHPGRMWCHCRMVYLPMSYLYGKRFVGPITPIVLSLRKELYTIPYHEIDWNKSRNTCAKEDLYY
PHPKMQDILWGSIYHLYEPLETRWPGKRLREKALQMAMKHIHYEDENSRYICLGPVNKVLNMLC
CWVEDPYSDAFKFHLQRVPDYLWIAEDGMRMQGYNGSQLWDTAFSVQAIISTKLIDSFGTTLKK
AHDFVKDSQIQQDFPGDPNVWFRHIHKGAWPFSTRDHGWLISDCTAEGLKASLMLSKLPSKIVG
EPLEKSRLCDAVNVLLSLQNENGGFASYELTRSYPWLELINPAETFGDIVIDYPYVECTSATME
ALTLFKKLHPGHRTKEIDTAVAKAANFLENMQRTDGSWYGCWGVCFTYAGWFGIKGLVAAGRTY
STCVAIRKACDFLLSKELPGGGWGESYLSCQNKVYTNLEGNRPHLVNTAWVLMALIEAGQAERD
PAPLHRAARLLINSQLENGDFPQEEIMGVFNKNCMITYAAYRNIFPIWALGEYFHRVLTE
Citrullus colocynthis (CcCDS2)
(SEQ ID NO: 44)
MWRLKVGAESVGEKDEKWLKSISNHLGRQVWEFCAHQPTASPNHLQQIDNARNHFRNNRFHRKQ
SSDLFLAIQNEKEIANVTKGGGIKVKEEEDVRKETVKNTVERALSFYSAIQTNDGNWASDLGGP
MFLLPGLVIALYVTGVLNSVLSKHHRQEMCRYLYNHQNEDGGWGLHIEGTSTMFGSALNYVALR
LLGEDADGGEGGAMTKARSWILDRGGATAITSWGKLWLSVLGVYEWSGNNPLPPEFWLLPYCLP
FHPGRMWCHCRMVYLPMSYLYGKRFVGPITPIVLSLRKELYTIPYHEIDWNRSRNTCAKEDLYY
PHPKMQDILWGSIYHLYEPLFTRWPGKRLREKALQMAMKHIHYEDENSRYICLGPVNKVLNMLC
CWVEDPYSDAFKFHLQRVPDYLWVAEDGMRMQGYNGSQLWDTAFSVQAIISTKLIDSFGTTLKK
AHDFVKDSQIQQDCPGDPNVWFRHIHKGAWPFSTRDHGWLISDCTAEGLKASLMLSKLPSKIVG
EPLEKSRLCDAVNVLLSLQNENGGFASYELTRSYPWLELINPAETFGDIVIDYPYVECTSATME
ALTLFKKLHPGRRTKEIDIAVARAANFLENMQRTDGSWYGCWGVCFTYAGWFGIKGLVAAGRTY
NSCVAIRKACDFLLSKELPGGGWGESYLSCQNKVYTNLEGNRPHLVNTAWVLMALIEAGQAERD
PAPLHRAARLLINSQLENGDFPQEEIMGVFNKNCMITYAAYRNIFPIWALGEYFHRVLTE
Cucurbita moschata
(SEQ ID NO: 45)
MWRLKVGAESVGEKDEKWVKSVSNHLGRQVWEFCADAAAAATPRQLLQIQNARNHFHRNRFHRK
QSSDLFLAIQYEKEIAEGGKGGAVKVKEEEEVGKEAVKSTLERALSFYSAVQTSDGNWASDLGG
PMFLLPGLVIALYVTGVLNSVLSKHHRVEMCRYLYNHQNEDGGWGLHIEGTSTMFGSALNYVAL
RLLGEDADGGDDGAMTKARAWILERGGATAITSWGKLWLSVLGVYEWSGNNFLPPEFWLLPYSL
PFHPGRMWCHCRMVYLPMSYLYGKRFVGPITPKVLSLRQELYTVPYHEIDWNKSRNTCAKEDLY
YPHPKMQDILWGSIYHVYEPLFTRWPGKRLREKALQTAMKHIHYEDENSRYICLGPVNKVLNML
CCWVEDPYSDAFKLHLQRVHDYLWVAEDGMRMQGYNGSQLWDTAFSIQAIVATKLVDSFAPTLR
KAHDFVKDSQIQEDCPGDPNVWFRHIHKGAWPFSTRDHGWLISDCTAEGLKASLMLSKLPSTMV
GEPLEKNRLCDAVNVLLSLQNDNGGFASYELTRSYPWLELINPAETFGDIVIDYPYVECTAATM
EALTLFKKTHPGHRTKEIDTAVGKAANFLEKMQRADGSWYGCWGVCFTYAGWFGTKGTVAAGRT
YNSCLAIRKACEFLLSKELPGGGWGESYLSCQNKVYTNLEGNKPHLVNTAWVLMALIEAGQGER
DPAPLHRAARLLMNSQLENGDFVQQEIMGVFNKNCMITYAAYRNIFPIWALGEYCHRVLTE
Cucumis sativus
(SEQ ID NO: 46)
MWRLKVGKESVGEKEEKWIKSISNHLGRQVWEFCAENDDDDDDEAVIHVVANSSKHLLQQQRRQ
SSFENARKQFRNNRFHRKQSSDLFLTIQYEKEIARNGAKNGGNTKVKEGEDVKKEAVNNTLERA
LSFYSAIQTSDGNWASDLGGPMFLLPGLVIALYVTGVLNSVLSKHHRQEMCRYIYNHQNEDGGW
GLHIEGSSTMFGSALNYVALRLLGEDANGGECGAMTKARSWILERGGATAITSWGKLWLSVLGV
YEWSGNNPLPPEFWLLPYSLPFHPGRMWCHCRMVYLPMSYLYGKRFVGPITHMVLSLRKELYTI
PYHEIDWNRSRNTCAQEDLYYPHPKMQDILWGSIYHVYEPLFNGWPGRRLREKAMKIAMEHIHY
EDENSRYIYLGPVNKVLNMLCCWVEDPYSDAFKFHLQRIPDYLWLAEDGMRMQGYNGSQLWDTA
FSIQAILSTKLIDTFGSTLRKAHHFVKHSQIQEDCPGDPNVWFRHIHKGAWPFSTRDHGWLISD
CTAEGLKASLMLSKLPSKIVGEPLEKNRLCDAVNVLLSLQNENGGFASYELTRSYPWLELINPA
ETFGDIVIDYSYVECTSATMEALALFKKLHPGHRTKEIDAALAKAANFLENMQRTDGSWYGCWG
VCFTYAGWFGIKGLVAAGRTYNNCVAIRKACHFLLSKELPGGGWGESYLSCQNKVYTNLEGNRP
HLVNTAWVLMALIEAGQGERDPAPLHRAARLLINSQLENGDFPQQEIMGVFNKNCMITYAAYRN
IFPIWALGEYSHRVLTE
Cucumis melo
(SEQ ID NO: 47)
MWRLKVGKESVGEKEEKWIKSISNHLGRQVWEFCSGENENDDDEAIAVANNSASKFENARNHFR
NNRFHRKQSSDLFLAIQCEKEIIRNGAKNEGTTKVKEGEDVKKEAVKNTLERALSFYSAVQTSD
GNWASDLGGPMFLLPGLVIALYVTGVLNSVLSKHHRQEMCRYIYNHQNEDGGWGLHIEGSSTMF
GSALNYVALRLLGEAADGGEHGAMTKARSWILERGGATAITSWGKLWLSVLGVYEWSGNNPLPP
EFWLLPYSLPFHPGRMWCHCRMVYLPMSYLYGKRFVGPITPIVLSLRKELYTIPYHEIDWNRSR
NTCAKEDLYYPHPKMQDILWGSIYHVYEPLFSGWPGKRLREKAMKIAMEHIHYEDENSRYICLG
PVNKVLKMLCCWVEDPYSDAFKFHLQRIPDYLWLAEDGMRMQGYKGSQLWDTAFSIQAIISTKL
IDTFGPTLRKAHHFVKHSQIQEDCPGDPNVWFRHIHKGAWPFSTRDHGWLISDCTAEGLKASLM
LSKLPSKIVGEPLEKNRLCDAVNVLLSLQNENGGFASYELTRSYPWLELINPAETFGDIVIDYS
YVECTSATMEALALFKKLHPGHRTKEIDAAIAKAANFLENMQKTDGSWYGCWGVCFTYAGWFGI
KGLVAAGRTYNNCVAIRKACNFLLSKELPGGGWGESYLSCQNKVYTNLEGNKPHLVNTAWVMMA
LIEAGQGERDPAPLHRAARLLINSQLESGDFPQQEIMGVFNKNCMITYAAYRNIFPIWALGEYS
HRVLDM
Citrullus lanatus subsp. vulgaris
(SEQ ID NO: 48)
DGMWASDLGGPMFLLPGLVIALYVTGVLNSVLSKHHRQEMCRYLYNHQNEDGGWGLHIEGTSTM
FGSALNYVALRLLGEDADGGEGGAMTKARSWILDRGGATAITSWGKLWLSVLGVYEWSGKNPLP
PEFWLLPYCLPFHPGRMWCHCRMVYLPMSYLYGKRFVGPITPIVLSLRKELYTIPYHEIDWNRS
RNTCAKEDLYYPHPKMQDILWGSIYHLYEPLFTRWPGKRLREKALQMAMKHIHYEDENSRYICL
GPVNKVLNMLCCWVEDPYSDAFKFHLQRVPDYLWVAEDGMRMQGYNGSQLWDTAFSVQAIISTK
LIDSFGTTLKKAHDFVKDSQIQQDCPGDPNVWFRHIHKGAWPFSTRDHGWLISDCTAEGLKASL
MLSKLPSEIVGEPLEKSRLCDAVNVLLSLQNENGGFASYELTRSYPWLELINPAETFGDIVIDY
PYVECTSATMEALTLFKKLHPGRRTKEIDIAVARAANFLEMMQRTDGSWYGCWGVCFTYAGWFG
IKGLVAAGRTYNSCVAIRKACDFLLSKELPGGGWGESYLSCQNKVYTNLEGNRPHLVMTAWVLM
ALIEAGQAERDPAPLHRAARLLINSQLEKGDFPQEEIMGVFNKNCMITYAAYRNIFPIWALGEY
FHRVLTE
Theobroma cacao
(SEQ ID NO: 49)
MWRLKIGKESVGDNGAWLRSSNDHVGRQVWEFCPESGTPEELSKVEMARQSFSTDRLLKKHSSD
LLMRIQYAKENQFVTNFPQVKLKEFEDVKEEATLTTLRRALNFYSTIQADDGHWPGDYGGPMFL
LPGLVITLSVTGRLNAVLSKEHQYEMCRYLYNHQNRDGGWGLHIEGPSTMFGTVLNYVTLRLLG
EGFEGGQGAVEKACEWILEHGSATAITSWGKMWLSVLGAYEWSGNNPLPPEVWLCPYFLPIHPG
RMWCHCRMVYLPMSYLYGKRFVGPIITPILSLRKELYAVPYHEVDWNKARNTCAKEDLYYPHPL
VQDILWASLHYLYEPIFTRWPCKSLREKALRTVMQHIEYEGENTRYICIGPVNKVLNMLSCSWE
DPYSESFKLHLPRILDYLWIAEDGMKMQGYNGSQLWDTAFAVQAIISTGLADEYGPILRKAHDF
IKYSQVLEDCPGDLNFWYRHISKGAWPFSTVDHGWPISDCTSEGLKAVLLLSTLPSESVGEPLH
MMRLYDAVNVILSLQNVDGGFPTYELTRSYQWLELIMPAETFGDIVIDYPYVECTSAAIQALIS
FKKLFPEHRMEEIENCIGRAVEFIEKIQAADGSWYGSWGVCFTYAGWFGIKGLSAAGRTYNNSS
NIRKACDFLLSKELATGGWGESYLSCQNKVYTNLEGARPHIVNTSWALLALIEAGQAERDPTPL
HRAARILINSQMEDGDFPQEEIMGVFNKNCMISYSAYRNIFPIWALGEYTCRVLRAP
Ziziphus jujube
(SEQ ID NO: 50)
MWKLKIGAETVGEGGSDGWLRSVNSHLGRQVWEFHPELGTPEELRQIQDARDAFFNHRFHKQHS
SDLLMRIQFAKENPCVANPPQVKVKDTDEVTEESVTTTLRRAINFYSTIQAHDGHWAGDYGGPM
FLLPGLVITLSVTGALNAVLSKEHQCEMCRYIYNRQNEDGGWGLHIEGPSTMFGTVLNYVSLRL
LGEGAEDGLGTIENARKWILDHGGATAITSWGKMWLSVLGVYEWSGNNPLPPEVWLCPYTLPFK
PGRMWCHCRMVYLPMSYLYGKRFVGPITPTIRSLRKELYTAPYHEIDWNRARNECAKEDLYYPH
PLVQDVLWASLHYVYEPIFMRWPAKKLREKALSTVMQHIHYEDENTRYICIGPVNKVLMMLCCW
VEDPMSEAFKLHLPRISDYLWIAEDGMKMQGYNGSQLWDTAFAVQAIVSTDLAEEYGPTIRKAH
EYIKNSQVLEDCPGDLNFWYRHISKGAWPFSTADHGWPISDCTAEGLKAVLLLSQLSSETVGDS
LDVKRLFNAVNVILSLQNGDGGFATYELTRSYQWLELINPAETFGDIVIDYPYVECTSAALEAL
TLFKKSYPGHRREEVENCITNAAMFIENIQAKDGSWYGSWGVCFTYAGWFGIKGLVASGRTYEN
CPSIRKACDFLLSKELPSGGWGESYLSCQNKVYTNLKDNKPHIVNTAWAMLALIVARQAERDPM
PLHRAARILIKSQMHDGDFPQEEIMGVFNKNCMISYAAYRNIFPIWALGEYRLHVLRSL
Prunus avium
(SEQ ID NO: 51)
MWKLKIGAETVGEGGYQWLKSVNNHLGRQVWEFNPELGSPEELQRIEDARKAFWDNRFERRHSS
DLLMRIQFEKENQCVTNLPQLKVKYEEEVTEEVVKTTLRRAISFYSTIQAHDGHWPGDYGGPME
LLPGLVITLSITGALNDVLSKEHQHEMCRYLYNHQNKDGGWGLHIEGPSTMFGTALNYVTLRLF
GEGADDCEGAMELARKWILDHGGVTKITSWGKMWLSVLCTYEWSGNNPLPPEVWLCPYSLPFHP
GRMWCHCRMVYLPMSYLYGKRFVGPITPTIRSLRKELYGVPYHEVDWNQARNLCAKEDLYYPHP
MVQDILWASLHYVYEPVFTRWPAKKLRENALQTVMQHIHYEDENTRYICIGPVNKVLNMLCCWA
EDPNSDAFKLHLPRIPDYLWVAEDGMKMQGYNGSQSWDTSFAVQAIISTNLAEEFCPTLRKAHE
YIKDSQVLEDCPGDLNFWYRHISKGAWPFSTADHGWPISDCTAEGLKAVLLLSKLPTGTVGESL
DMKQLYDAVNVMLSLQNEDGGFATYELTRSYQWLELINPAETFGDIVIDYPYVECTSAAIQALT
MFRKLYPGHRREEIESCIARAAKFIEKIQATDGSWYGSWGVCFTYAGWFGIKGLAAAGRTYKDC
SSIRKACDFLLSKELPSGGWGESYLSCQNKVYTNLKDNRPHIVHTAWAMLALIGAGQAKRDPTP
LHRAARVLINSQMENGDFPQKEIMGVFNKNCMISYSAYRNIFPIWALGEYRCQVLEAL
Brassica napus
(SEQ ID NO: 52)
MWKLKIAEGGSPWLRTTNNH7GRQFWEFDPNLGTPEELAAVEEARKSFRENRFAKKHSSDLLMR
LQFSRESLSRPVLPQVNIKDSDDVTEKMVETTLKRGVDFYSTIQASDGHNAGDYGGPMFLLPGL
IITLSITGALNTVLSEQHKAEMRRYLHNHQNEDGGWGLHIEGPSTMFGSVLNYVTLRLLGEGPN
DGDGAMEKGRDWILRHGGATNITSWGKMWLSVLGAFEWSGMNPLPPEIWLLPYILPIHPGRMWC
HCEMVYLPMSYLYGKRFVGPITSTVLSLRKELFTVPYHEVDWNEARNLCAKEDLYYPHPLVQDI
LWASLHKIVEPVLTRWPGSNLREKALRTTLEHIHYEDENTRYICIGPVNKVLNMLCCWVEDPNS
EAFKLHLPRIHDYLWVAEDGIKMQGYNGSQLWDTSFAVQAVLATNFREEYGPVLKKAHSYVKNS
QVSEDCPGDLSYWYRHISKGAWPFSTADHGWPISDCTAEGLKAALLLSKVPKEIVGEPVDTKRL
YDAVNVIISLQNADGGFATYELTRSYPWLELINPAETFGDIVIDYPYVECTSAAIQALIAFRKL
YPGHRKKEVDECIEKAVKFIESIQESDGSWYGSWAVCFTYGTWEGVKGLEAAGKTLKNSPTVAK
ACEFLLSKQLPSGGWGESYLSCQDKVYSNLDGNRSHVVNTAWALLSLIGAGQVEVDQKPLHRAA
RYLINAQMESGDFPQQEIMGVFNRNCMITYAAYRNIFPIWALGEYRSKVLLQQGE
Spinacia oleracea
(SEQ ID NO: 53)
MWKLKIAEGGSPWLRTTNNHVGRQIWEFDPNLGTPEQIREVEEARENFWKNRFEQKHSSDLLMR
MQFAQENSSNVVLPQVKVKDEDEITEETVATTLRRALSYQSTIQAHDGHWPGDYGGPMFLMPGL
VIALSVTGALNAVLSKEHQKEMCRYLYNHQNKDGGWGLHIEGHSTMFGTVLTYVTLRLLGEGVD
DGDGAMERGRKWTLEHGSATATTSWGKMWLSVLGVFEWAGNNPMPPETWLLPYILPVHPGRMWC
HCRMVYLPMSYLYGKREVGPITPTVLSLRRELFDVPYHEIDWDRARNECAKEDLYYPHPLVQDI
LWASLHKAVEPILMRWPGKKLREKALSTVMEHIHYEDENTRYICIGPVNKVLNMLCCWVEDPNS
EAFKLHLPRIPDFLWVAEDGMKMQGYNGSQLWDTTEMVQAILATNLGEEYGGTLRKAHNFIKDS
QVREDCPGDLSYWYRHISKGAWPFSTADHGWPISDCTAEGLKAALLLSKVPSDIVGEPLEVKRL
YDSVNVLLSLQNGDGGFATYELTRSYPWLELINPAETFGDIVIDYPYVECTSAAIQALVSFKRL
YPGHRREEIENCIKKAAKFIEDIQAADGSWYGSWAVCFTYATWFGIKGLVAAGKNYDNCPAIRK
ACDFLLSKQLSNGGWGESYLSCQNKVYSNIEGNKAHVVNTGWAMLALIGAGQAKRDPMPLHRAA
KVLINSQMPNGDFPQQEIMGVFNRNCMITYAAYRNIFPTWALGEYRTQVLQK
Trigonella foenum-graecum
(SEQ ID NO: 54)
MWKLKVAEGGSPWLRTVNNYVGRQVWEFDPNSGSPQELDQIESVRQNFHNNRFSHKHSDDLLMR
IQLAKENPMGEVIPKVRVKDVEDVNEESVTTTLRRALNFYSTLQSRDGHWPGDYGGPMFLMPGL
VIALSITGALNAVLTDEHQKEMRRYLYNHQNKDGGWGLHIEGPSTMFGSVLCYVTLRLLGEGPN
DGEGEMEKARDWILEHGGATYITSWGKMWLSVLGVFEWSGNNPLPPEIWLLPYMLPIHPGRMWC
HCRMVYLPMSYLYGKRFVGPITPTVLSLRRELFDVPYHEIDWDRARNECAKEDLYYPHPLVQDI
LWASLHKFVEPIFMNWPGKKLREKAVETVMEHVHYEDENTRYICIGPVNKVLNMLCCWVEDPNS
EAFKLHLPRIPDFLWIAEDGMKMQGYNGSQLWDTTEMVQAILATNLGEEYGGTLRKAHNFIKDS
QVLEDCPGDLSKWYRHISKGAWPFSTADHGWPISDCTAEGLKAVLLLSKIGPEIVGEPLDAKGE
YDAVNVIISLQNEDGGLATYELTRSYKWLEIINPAETFGDIVIDYTYVECTSAAIQALSTFRKL
YPGHRREEIQHCIEKAAAFIEKIQASDGSWYGSWGVCFTYGTWFGVKGLIAAGKSFSNCLSIRK
ACDFLLSKQLPSGCWGESYLSCQNKVYSNLESNRSHVVNTGWAMLALIEAEQAKRDPTPLHHAA
VCLINSQMENGDFPQEEIMGVFNKNCMITYAAYRNIFPIWALGEYRRHVLQA
Ricinus communis
(SEQ ID NO: 55)
MWKLRIAEGSGNPWLRTTNDHIGRQVWEFDSSKIGSPEELSQIENARQNFTKNRFIHKHSSDLL
MRIQFSKENPICEVLPQVKVKESEQVTEEKVKITLRRALNYYSSIQADDGHWPGDYGGPMELMP
GLIIALSITGALNAILSEEHKREMCRYLYNHQNRDGGWGLHIEGPSTMFGSVLCYVSLRLLGEG
PNEGEGAVERGRNWILKHGGATAITSWGKMWLSVLGAYEWSGNNPLPPEMWLLPYILPVHPGRM
NCHCRMVYLPMSYLYGKRFVGPITPTVLSLRKELYTVPYHEIDWNQARNQCAKEDLYYPHPMLQ
DVLWATLHKFVEPILMHWPGKRLREKAIQTAIEHIHYEDENTRYICIGPVNKVLNMLCCWVEDP
NSEAFKLHLPRLYDYLWLAEDGMKMQGYNGSQLWDTAFAVQAIVSTNLIEEYGPTLKKAHSFTK
KMQVLENCPGDLNFWYRHISKGAWPFSTADHGWPISDCTAEGIKALMLLSKIPSEIVGEGLNAN
RLYDAVNVVLSLQNGDGGFPTYELSRSYSWLEFINPAETFGDIVIDYPYVECTSAAIQALTSFR
KSYPEHQREEIECCIKKAAKFMEKIQISDGSWYGSWGVCFTYGTWFGIKGLVAAGKSFGNCSSI
RKACDFLLSKQCPSGGWGESYLSCQKKVYSNLEGDRSHVVNTAWAMLSLIDAGQAERDPTPLHR
AARYLINAQMENGDFPQQEIMGVFNRNCMITYAAYRDIFPIWALGEYRCRVLKAS
Pisum sativum cycloartenol synthase (PsCAS_mut)
(SEQ ID NO: 191)
MAWKLKVAEGGTPWLRTLNNHVGRQVWEFDPHSGSPQDLDDIETARRNFHDNRFTHKHSDDLLM
RLQFAKENPMNEVLPKVKVKDVEDVTEEAVATTLRRGLNFYSTIQSHDGHWPGDLGGPMFLMPG
LVITLSVTGALNAVLTDEHRKEMRRYLYNHQNKDGGWGLHIEGPSTMFGSVLCYVTLRLLGEGP
NDGEGDMERGRDWILEHGGATYITSWGKMWLSVLGVFEWSGNNPMPPEIWLLPYALPVHPGRMW
CHCRMVYLPMSYLYGKRFVGPITPTVLSLRKELPTVPYHDIDWNQARNLCAKEDLYYPHPLVQD
ILWATLHKFVEPVFMNWPGKKLREKAIKTAIEHIHYEDENTRYICIGPVNKVLNMLCCWVEDPN
SEAFKLHLPRIYDYLWVAEDGMKMQGYNGSQLWDTAFAAQALISTNLIDEFGPTLKKAHAFIKN
SQVSEDCPGDLSKWYRHISKGAWPFSTADHGWPISDCTAEGLKAVLLLSKIAPEIVGEPLDSKR
LYDAVNVILSLQNENGGLATYELTRSYTWLEIINPAETFGDIVIDCPYVECTSAAIQALATFGK
LYPGHRREEIQCCIEKAVAFIEKIQASDGSWYGSWGVCFTYGTWFGIKGLIAAGKNFSNCLSIR
KACEFLLSKQLPSGGWAESYLSCQNKVYSNLEGNRSHVVNTGWAMLALIEAEQAKRDPTPLHRA
AVCLINSQLENGDFPQEEIMGVFNKNCMITYAAYRCIFPIWALGEYRRVLQAC
Cucurbita pepo subsp. pepo cycloartenol synthase (CpCAS_mut)
(SEQ ID NO: 192)
MAWQLKIGADTVPSDPSNAGGWLSTLNNHVGRQVWHFHPELGSPEDLQQIQQARQHFSDHRFEK
KHSADLLMRMQFAKENSSFVNLPQVKVKDKEDVTEEAVTRTLRRAINFYSTIQADDGHWPGDLG
GPMFLIPGLVITLSITGALNAVLSTEHQREICRYLYNHQNKDGGWGLHIEGPSTMFGSVLNYVT
LRLLGEEAEDGQGAVDKARKWILDHGGAAAITSWGKMWLSVLGVYEWAGNNPLPPELWLLPYLL
PCHPGRMWCHCRMVYLPMCYLYGKRFVGPITPIIRSLRKELYLVPYHEVDWNKARNQCAKEDLY
YPHPLVQDILWATLHHVYEPLFMHWPAKRLREKALQSVMQHIHYEDENTRYICIGPVNKVLNML
CCWAEDPHSEAFKLHIPRIYDYLWIAEDGMKMQGYNGSQLWDTAFAVQAIISTELAEEYETTLR
KAHKYIKDSQVLEDCPGDLQSWYRHISKGAWPFSTADHGWPISDCTAEGLKAVLLLSKLPSEIV
GKSIDEQQLYNAVNVILSLQNTDGGFATYELTRSYRWLELMNPAETFGDIVIDYPYVECSSAAI
QALAAFKKLYPGHRRDEIDNCIAEAADFIESIQATDGSWYGSWGVCFTYGGWFGIRGLVAAGRR
YNNCSSLRKACDFLLSKELAAGGWGESYLSCQNKVYTNIKDDRPHIVNTGWAMLSLIDAGQSER
DPTPLHRAARVLINSQMEDGDFPQEEIMGVFNKNCMISYSAYRNIFPIWALGEYRSRVLKPLK
Zostera marina cycloartenol synthase (ZmCAS_mut)
(SEQ ID NO: 193)
MAWKLKVAEGRDARLRTINGHVGRQIWEFDPDLGTDNERAEVEAVREKFRNNRFEKKHSSDLLM
RLQLAKENPVSSYLTQVKLEENEDITEEAVTMTLRRALNFHSSIQSFDGHWAGDLGGPMFLMPG
LVISLYITGVLNTVLSSEHQREMCRYLYNHQNEDGGWGLHIEGPSTVFGSTLTYITLRLLGENV
EDGDGAMEKGRKWILDHGGATYITSWGKMWLSVLGVFDWSGNNPLPPEMWLLPYFLPVHPGRMW
CHCRMVYLPMSYLYGKRFVGKITPLVLSLRNEIYTVSYNQIDWNKARNLCAKEDLYYPHPMVQD
LLWATLHKEVEPLLMHWPGTLLREKALNTTMQHLHYEDESTRYICIGPVNKVLNMLCCWVDDPD
SEAFKLHLPRISDYLWIAEDGMKCQGYNGSQLWDTAFAVQAYIATNLSDEFGPVLTKAHEYIKN
SQVPDDCSGDLSFWYRHISKGAWPFSTGDHGWPISDCTAEGLKASLLLSRISPEVVGKPLNAKR
FYDAVNVILSLMNSDGSFATYELTRSYTWLEMINPAETFGDIVIDYPYVECTSAAIQSLVAFTK
LYPGHRREEIDECITKAAKFIESIQKKDGSWYGSWAVCFTYGLWFGIKGLIAAGKTYKNSSAIR
KACEFLLSKQLASGGWGESYLSCQDKVYTNLEGNRAHAVNTGWAMLSLIDAGQAERDPSPLHRA
ARVLINSQMGNGDFPQEEIMGVFNRNCMISYSAYRNIFPIWALGEYRCKVLASKGHE
Artemisia annua (AaCASmut)
(SEQ ID NO: 219)
MAWKLKIAEGGDPWLRTTNDHIGRQIWEFDPTLGSVEELAEIEKLRKTFRDNRFEKKHSADLLM
RSQFAKENSVSVFPPKVNIKDVEDITEDKVTNVLRRAIGFHSTLQADDGHWPGDLGGPMFLLPG
LVITLSITGALNAVLSKEHKREMCRYLYNHQNIDGGWGLHIEGHSTMFGSALNYVTLRLLGEGA
NDGEGAMEKGRKWILDHGGATAITSWGKFWLSVLGVFEWPGNNPLPPEMWLLPYFLPVHPGRMW
CHCRMVYLPMSYLYGKRFVGPITSTVLALRKELFTVPYHDIDWNEARNLCAKEDLYYPHPLIQD
VLWATLDKFVEPVLMSWPGKKLREKALRTAMEHIHYEDENTRYICIGPVNKVLNMLCCWVEDPN
SEAFKLHLPRIQDYLWIAEDGMKMQGYNGSQLWDAAFTVQAIMSTNLIEEFGPTLKKGHIFIKK
SQVLDNCYGDLDYWYRHISKGAWPFSTADHGWPISDCTAEGLKAALLLSKLPSEIVDEPLDAKR
FYDAVNVILSLMNADGSFATYELTRSYSWLELINPAETFGDIVIDYPYVECTSAAIQALVAFKR
LYPGHRRDEVQGCIDKAAAFLEKIQEADGSWYGSWAVCFTYGTWFGVKGLVAAGKNYSNCSSIR
KACNFLLSKQLASGGWGESYLSCVDKVYTNLEGNRSHVVNTGWAMLALIDAEQAKRDPTPLHRA
ARVLINSQMENGEFPQQEIMGVFNRNCMITYAAYRNIFPIWALGEYRCRVLKVET
Citrullus colocynthis (CcCDS2)
(SEQ ID NO: 220)
MAWRLKVGAESVGEKEEKWLKSISNHLGRQVWEFCAHQPTASPNHLQQIDNARNHFRNNRFHRK
QSSDLELAIQNEKEIANVTKGGGIKVKEEEDVRKETVKNTVERALSFYSAIQTNDGNWASDLGG
PMFLLPGLVIALYVTGVLNSVLSKHHRQEMCRYLYNHQNEDGGWGLHIEGTSTMFGSALNYVAL
RLLGEDADGGEGGAMTKARSWILDRGGATAITSWGKLWLSVLGVYEWSGNNPLPPEFWLLPYCL
PFHPGRMWCHCRMVYLPMSYLYGKRFVGPITPIVLSLRKELYTIPYHEIDWNRSRNTCAKEDLY
YPHPKMQDILWGSIYHLYEPLETRWPGKRLREKALQMAMKHIHYEDENSRYICLGPVNKVLNML
CCWVEDPYSDAFKFHLQRVPDYLWVAEDGMRMQGYNGSQLWDTAFSVQAIISTKLIDSFGTTLK
KAHDFVKDSQIQQDCPGDPNVWFRHIHKGAWPFSTRDHGWLISDCTAEGLKASLMLSKLPSKIV
GEPLEKSRLCDAVNVLLSLQNENGGFASYELTRSYPWLELINPAETFGDIVIDYPYVECTSATM
EALTLFKKLHPGHRTKEIDIAVARAANFLENMQRTDGSWYGCWGVCFTYAGWFGIKGLVAAGRT
YNSCVAIRKACDFLLSKELPGGGWGESYLSCQNKVYTNLEGNRPHLVNTAWVLMALIEAGQAER
DPAPLHRAARLLINSQLENGDFPQEEIMGVFNKNCMITYAAYRNIFPIWALGEYFHRVLTE
Epoxide Hydrolase
Siraitia grosvenorii EPH1 (SgEPH1)
(SEQ ID NO: 56)
MEKIEHSTIATNGINMHVASAGSGPAVLFLHGFPELWYSWRHQLLYLSSLGYRAIAPDLRGFGD
TDAPPSPSSYTAHHIVGDLVGLLDQLGVDQVFLVGDWGAMMAWYFCLFRPDRVKALVNLSVHFT
PRNPAISPLDGFRLMLGDDFYVCKFQEPGVAEADFGSVDTATMFKKFLTMRDPRPPIIPNGFRS
LATPEALPSWLTEEDIDYFAAKFAKTGFTGGFNYYRAIDLTWELTAPWSGSEIKVPTKFIVGDL
DLVYHFPGVKEYIHGGGFKKDVPFLEEVVVMEGAAHFINQEKADEINSLIYDFIKQF
Siraitia grosvenorii EPH2 (SqEPH2)
(SEQ ID NO: 57)
MEKIEHTTISTNGINMHVASIGSGPAVLFLHGFPELWYSWRHQLLELSSMGYRAIAPDLRGFGD
TDAPPSPSSYTAHHIVGDLVGLLDQLGIDQVFLVGHDWGAMMAWYFCLFRPDRVKALVNLSVHF
LRRHPSIKFVDGFRALLGDDFYFCQFQEPGVAEADFGSVDVATMLKKFLTMRDPRPPMIPKEKG
FRALETPDPLPAWLTEEDIDYFAGKFRKTCFTGGFNYYRAFNLTWELTAPWSGSEIKVAAKFIV
GDLDLVYHFPGAKEYIHGGGFKKDVPLLEEVVVVDGAAHFINQERPAEISSLIYDFIKKF
Siraitia grosvenorii EPH3 (SgEPH3)
(SEQ ID NO: 58)
MDQIEHITINTNGIKMHIASVGTGPVVLLLHGFPELWYSWRHQLLYLSSVGYRAIAPDLRGYGD
TDSPASPTSYTALHIVGDLVGALDELGIEKVFLVGHDWGAIIAWYFCLFRPDRIKALVNLSVQE
IPRNPAIPFIEGFRTAFGDDFYMCRFQVPGEAEEDFASIDTAQLFKTSLCNRSSAPPCLPKEIG
FRAIPPPENLPSWLTEEDINYYAAKFKQTGFTGALNYYRAFDLTWELTAPWTGAQIQVPVKFIV
GDSDLTYHFPGAKEYIHNGGFKKDVPLLEEVVVVKDACHFINQERPQEINAHIHDFINKF
Momordica charantia
(SEQ ID NO: 59)
MEKIEHSTIAANGITIHVASVGSGPAVLLLHGFPELWYSWRHQLLFLASKGYRAIAPDLRGFGD
SDAPPSPSSYTPLHIVGDLVALLDHLGIDLVFLVGHDWGAMMAWHFCLLRPDRVKALVNLSVHF
MPRNPAMSPLDGMRLLLGDDFYVCRFQEPGAAEADFGSVDTATMMKKFLTMRDPRPPIIPNGFR
SLETPQALPPWLTEEDIDYFAAKFAKTGFTGGFNYYRAIGRTWELTAPWTGSKIKVPAKFIVGD
LDMVYHLPDAKEYIHGGGFKEDVPLLEEVVVIEGAAHFINQEKPDEISSLIYDFIKKF
Cucurbita moschata
(SEQ ID NO: 60)
MEKIEHSTIATNGINMHVASIGSGPPVLFLHGFPELWYSWRHQLLFLASKGFRAIAPDLRGFGD
SDVPPSPSSYTPFHIIGDLIGLLDHLGIEQVFLVGHDWGAMMAWYFCLFRPDRVKALVNLSVHY
NPRNPAISPLSRTRQFLGDDFYICKFQTPGVAEADFGSVDTATMMKKFLTIRDPSPPIIPNGFK
TLKTPETLPSWLTEEDIDYFASKFTKTGFTGGFNYYRAIEQTWELTGPWSGAKIKVPTKYVVGD
VDMVYHLPGAKQYIHGGGFKKDVPLLEEVVVMEGAAHFINQEKADEISAHIYDFIIKF
Cucurbita maxima
(SEQ ID NO: 61)
MENIEHTIVPTNGINMHIASIGSGPAVLFLHGFPELWYSWRHQLLFLASNGFRAIAPDLRGFGD
TDVPPSPSSYTAHHIVGDLIGLLDHLGIDRVFLVGHDWGAMMAWYFCLFRPDRVRALVNLSVHY
LHRHPSIKFVDGFRAFLGDDFYFCQFQEPGVAEADFGSVDTATMLKKFLTMRDPRPPMIPKEKG
FRALETPDPLPSWLTEEDVDYFASKFSKTGFTGGFNYYRAFDLSWELTAPWSGSQVKVPAKFIV
GDLDLVYHFPGAKEYIHGGRFKEDVPFLEEVVVIEGAAHFINQERADEISSLIYEFINKF
Prunus persica
(SEQ ID NO: 62)
MEKIEHTTVSTNGINMHIASIGTGPVVLFLHGFPELWYSWRHQLLSLSSLGYRCIAPDLRGFGD
TDAPPSPASYSALHIVGDLIGLLDHLGIDQVFLVGHDWGAVIAWWFCLFRPDRVKALVNMSVAF
SPRNPKRKPVDGFRALFGDDYYICRFQEPCEIEKEFAGYDTTSIMKKFLTGRSPKPPCLPKELC
LRAWKTPETLPPWLSEEDLNYFASKFSKTGFVGGLNYYRALNLTWELTGPWTGLQVKVPVKFIV
GDLDITYHIPGVKNYIHNGGFKRDVPFLQEVVVIEDGAHFINQERPDEISRHVYDFIQKF
Morus notabilis
(SEQ ID NO: 63)
MEKIEHSTVHTNGINMHVASVGTGPAILFLHGFPELWYSWRHQMISLSSLGYRCIAPDLRGYGD
TDAPPSPTSYTSLHIVGDLVGLIDHLVIEKLFLVGHDWGAMIAWYFCLFRPDRIKALVNLSVPF
FPRNPKINFVDGFRAELGDDFYICRFQEPGESEADFSSDTVAVFRRILANRDPKPPLIPKEIGF
RGVYEDPVALPSWLTEDDINHFANKFNETGFTGGLNYYRALNLTWELTAAWTGARVQVPTKFIM
GDLDLVYYFPGMKEYILNGGFKRDVPLLQELVIIEGAAHFINQEKPDEISSHIHHFIQKF
Ricinus communis
(SEQ ID NO: 64)
MEKIEHTTVATNGINMHVAAIGTGPEILFLHGFPELWYSWRHQLLSLSSRGYRCIAPDLRGYGD
TDAPESLTGYTALHIVGDLIGLLDSMGIEQVFLVGHDWGAMMAWYLCMFRPDRIKALVNTSVAY
MSRNPQLKSLELFRTVYGDDYYVCRFQEPGGAEEDFAQVDTAKLIRSVFTSRDPNPPIVPKEIG
FRSLPDPPSLPSWLSEEDVNYYADKFNKKCFTGGLNYYRNIDQNWELTAPWDGLQIKVPVKFVI
GDLDLTYHFPGIKDYIHNGGFKQVVPLLQEVVVMEGVAHFINQEKPEEISEHIYDFIKKF
Citrus unshiu
(SEQ ID NO: 65)
MEKIEHTTVGTNGINMHVASIGTGPVVLFIHGFPELWYSWRNQLLYLSSRGYRAIAPDLRGYGD
TDAPPSVTSYTALHLVGDLIGLLDKLGIHQVFLVGHDWGALIAWYFCLFRPDRVKALVNMSVPF
PPRNPAVRPLNNFRAVYGDDYYICRFQEPGEIEEEFAQIDTARLMKKFLCLRIAKPLCIPKDTG
LSTVPDPSALPSWLSEEDVNYYASKFNQKGFTGPVNYYRCSDLNWELMAPWTGVQLEVPVKFIV
GDQDLVYNNKGMKEYIHNGGFKKYVPYLQEVVVMEGVAHFINQEKAEEVGAHIYEFIKKF
Hevea brasiliensis
(SEQ ID NO: 66)
MEKIEHITVFTNGINMHIASIGTGPEILFLHGFPELWYSWRHQLLSLSSLGYRCIAPDLRGYGD
TDAPQSVNQYTVLHIVGDLVGLLDSLGIQQVFLVGHDWGAFIAWYFCIFRPDRIKALVNTSVAF
MPRNPQVKPLDGLRSMFGDDYYICQFQKPGKAEEDFAQVNTAKLIKLLFTSRDPRPPHFLKEVG
LKALQDPPSQQSWLTEEDVNFYAAKFNQKGFRGGLNYYQNINMNWELAAAWTGVQIKVPVKFII
GDLDLTYHFPGIKEYIHNGGFKKDVPLLQDVVVMEGVAHFLNQEKPEEVSKHIYDFIKKF
Handroanthus impetiginosus
(SEQ ID NO: 67)
MDKIQHKIIQTNGINIHVAEIGDGPAVLFLHGFPELWYSWRHQMLFLSSRGYRAIAPDLRGYGD
SDAPPCATSYTAFHLLGDLVGLLDAMGLDRVFLVGHDWGAVMAWYFCLLKPDRLKALVNLSVVF
QPRNPKRKPVESMRAKLGDDYYICRFQEPGEAEEEFARVDTARLIKKLLTTRNPAPPRLPKEVG
FGCLPHKPITMPSWLSEEDVQYYAAKFNQKGFTGGLNYYRAMDLSWELAAPWTGVQIKVPVKFI
VGDLDITYNTPGVKEYIHKGRFKQHVPFLQELVILEGVAHFLNQEKPDEINQHIYDFIHKF
Camelina sativa
(SEQ ID NO: 68)
MEKIEHTTVSTNGINMHVASIGSGPVILFLHGFPDLWYSWRHQLLSFAALGYRAIAPDLRGYGD
SDAPPSPESYTILHIVGDLVGLLDSLGVDRVFLVGHDWGAIVAWWLCMIRPDRVKALVNTSVVF
NPRNPSVKPVDKFRDLFGDDYYVCRFQETGEIEEDFAQVDTKKLITRFFVSRNPRPPCIPKSVG
FRGLPDPPSLPAWLTEQDVSFYGDKFSQKGFTGGLNYYRAMNLSWELTAPWAGLQIKVPVKFIV
GDLDITYNIPGTKEYIHGGGLKKHVPFLQEVVVMEGVGHFLQQEKPDEVTDHIYGFFEKFRTRE
TSSL
Coffea canephora
(SEQ ID NO: 69)
MDKIQHRQVPVNGINLHVAEIGDGPAILFLHGFPELWYSWRHQLLSLSAKGYRALAPDLRGYGD
SDAPPSPSNYTALHIVGDLVGLLDSLGLDRVFLVGHDWGAVMAWYFCLLRPDRIKALVNMSVVF
TPRNPKRKPLEAMRARFGDDYYICRFQEPGEAEEEFARVDTARIIKKFLTSRRPGPLCVPKEVG
FGGSPHNPIQLPSWLSEDDVNYFASKFSQKGFTGGLNYYRAMDLNWELTAPWTGLQIKVPVKFI
VGDLDVTFTTPGVKEYIQKGGFKRDVPFLQELVVMEGVAHFVNQEKPEEVSAHIYDFIQKF
Punica granatum
(SEQ ID NO: 70)
MEKIQHTTVRTNGINMHVATAGSGPDSILFVHGFPELWYTWRHQMVSLAALGYRTIAPDLRGYG
DTDAPPSHESYTAFHIVGDLVGLLDSMGIEKVFLVGHDWGAAIAWYFCLFRPDRIKALVNMSVV
FHPRNPNRKPVDGLRAILGDDYYICRFQAPGEIEEDFARADTANIIKFFLVSRNPRPPQIPKEG
FSCLANSRQMDLPSWLSEEDINYYASKFSEKGFTGGLNYYRVMNLNWELTAPFTCLQIKVPAKF
MVGDLDITYNTPGTKEFIHNGGLKKHVPFLQEVVVMEGVAHFINQEKPEEVTAHIYDFIKKF
Arabidopsis lyrata subsp. lyrata
(SEQ ID NO: 71)
MEKIEHTTVSTNGINMHVASIGSGPVILFLHGFPDLWYSWRHQLLSFAALGYRAIAPDLRGYGD
SDAPPSRESYTILHIVGDLVGLLNSLGVDRVFLVGHDWGAIVAWWLCMIRPDRVNALVNTSVVF
NPRNPSVKPVDAFRALFGDDYYICRFQEPGEIEEDFAQVDTKKLITRFFISRNPRPPCIPKSVG
FRGLPDPPSLPAWLTEEDVSFYGDKFSQKGFTGGLNYYRALNLSWELTAPWAGLQIKVPVKFIV
GDLDITYNIPGTKEYIHEGGLKKHVPFLQEVVVLEGVGHFLHQEKPDEITDHIYGFFKKFRTRE
TASL
Rhinolophus sinicus
(SEQ ID NO: 72)
MDKIEHTTVSTNGINMHVASIGSGPVILFLHGFPDLWYSWRHQLLSFAGLGYRAIAPDLRGYGD
SDSPPSHESYTILHIVGDLVGLLDSLGVDRVFLVGHDWGAVVAWWLCMIRPDRVNALVNTSVVF
NPRNPSVKPVDAFKALFGEDYYVCRFQEPGEIEEDFAQVDTKKLINRFFTSRNPRPPCIPKTLG
FRGLPDPPALPAWLTEQDVSFYADKFSQKGFTGGLNYYRAMNLSWELTAPWAGLQIKVPVKFIV
GDLDITYNIPGTKEYIHEGGLKKHVPFLQEVVVMEGVGHFLHQEKPDEVTDHIYGFFKKF
Gossypium raimondii (GrEPH)
(SEQ ID NO: 184)
MAEKIEHTTVTTNGIKMHVASIGSGPIILFLHGFPELWYTWRHQLLSLSSLGYRCVAPDLRGYG
DSDAPPSPESYTVFHIVGDLVGLLDALGVDKVFLVGHDWGAMIAWNFCLFRPDRIKALVNLSIP
YHPRNPKVKTVDGYRALFGDDFYICRFQVPGEAEAHFAQMDTAKVMKKFLTTRDPNPPCIPRET
GLKALPDPPALPSWLSEDEINYFATKFSQKGFTGGLNYYRAMNLNWELMAPWTGLQIQVPVKFI
VGDLDITYHIPGVKEYLQNGGFKKNVPFLQELVVMEGVAHFINQEKPQEISMHIYDFIKKF
Gossypium hirsutum (GhEPH)
(SEQ ID NO: 185)
MAEKIEHTTVTTNGIKMHVASIGSGPIILFLHGFPELWYTWRRQLLSLSSLGYRCVAPDLRGYG
DSDAPPSPESYTVFHVVGDLVGLLDALGVDKVFLVGHDWGAMIAWNFCLFRPDRIKALVNLSVP
YHPRNPKVKTVDGYRALFGDDFYICRFQVPGEAEAHFAQMDTAKVLKKFLTTRDPNPPCIPKET
GLKALPDPPALPSWLSEDEINYFATKFNQKGFTGGLNYYRAMNLNWELMAPWTGLQIQVPVKFI
VGDLDITYHIPGVKEYLQNGGFKKNVPFLQELVVMEGVAHFINQEKPQEISMHIYDFVKKF
Siraitia grosnevorii (SgEPH4)
(SEQ ID NO: 186)
MAENIEHTTVQTNGIKMHVAAIGTGPPVLLLHGFPELWYSWRHQLLYLSSAGYRAIAPDLRGYG
DTDAPPSPSSYTALHIVGDLVGLLDVLGIEKVFLIGHDWGAIIAWYFCLFRPDRIKALVNLSVQ
FFPRNPTTPFVKGFRAVLGDQFYMVRFQEPGKAEEEFASVDIREFFKNVLSNRDPQAPYLPNEV
KFEGVPPPALAPWLTPEDIDVYADKFAETGFTGGLNYYRAFDRTWELTAPWTGARIGVPVKFIV
GDLDLTYHFPGAQKYIHGEGFKKAVPGLEEVVVMEDTSHFINQERPHEINSHIHDFFSKFC
Cucumis melo (CmEPH1)
(SEQ ID NO: 187)
MADKIQHSTISTNGINIHFASIGSGPVVLFLHGFPELWYSWRHQLLFLASKGFRAIAPDLRGFG
DSDAPPSPSSYTPHHIVGDLTGLLDHLGIDQVFLVGHDWGAMMAWYFCLFRPDRVKALVNTSVH
YTPRNPAGSPLAVTRRYLGDDFYICKFQEPGVAEADFGSVDTATMMKKFLTMRDPRPAIIPNGF
KTLLETPEILPSWLTEEDIEYFASKFSKTGFTGGFNYYRALDITWELTGPWSRAQIKVPTKFIV
GDLDLVYNFPGAKEYIHGGGFKKDVPLLEDVVVIEGAAHFINQEKPDEISSLIYDFITKF
Cucumis melo (CmEPH2)
(SEQ ID NO: 188)
MAEKIEHTTIPTNGINMHVASIGSGPAVLFLHGFPQLWYSWRHQLLFLASKGFRALAPDLRGFG
DTDAPPSPSSYTFLHIIGDLIGLLDHLGLEKVFLVGHDWGAMIAWYFCLFRPDRVKALVNLSVY
YIKRHPSISFVDGFRAVAGDNFYICQFQEAGVAEADFGRVDTATMMKKFMGMRDPEAPLIFTKE
KGFSSMETPDPLPCWLTEEDIDFFATKFSKTGFTGGFNYYRALNLSWELTAAWNGSKIEVPVKF
IVGDLDLVYHFPGAKQYIHGGEFKKDVPFLEEVVVIKDAAHFIHQEKPHQINSLIYHFINKFST
STSPA
Trema orientale (ToEPH)
(SEQ ID NO: 189)
MAEKIEHTTINTNGVNLHVASIGTGPAVLFLHGFPELWYSWRHQMLALSSLGYRAIAPDLRGYG
DSDAPPSPESYSSLHIVGDLVGLIDQLGIDQIFLVGHDWGAVIAWQFCLFRFDRVKALVNMSVP
FRPRHPTRKPIETFRALFGDDYYVCRFQAPGEVEEDFASDDTANLLKKFYGGRNPRPPCVPKEI
GFKGLKAPELPSWLSEEDLNYFAEKFNQRGFTGGLNYYRALDLTWELTAAWTGVQVKVPTKFIV
GDLDITYHIPGAKEYINEGGLKKDVPYLQEVVVMEGVAHFVNQEKAEEVSAHIHDFIKKF
Arachis hypogaea (AhEPH)
(SEQ ID NO: 190)
MAEKIEHTWVNTNGIKMHVASIGSGPAVLFLHGFPELWYSWRHQLLSLSAQGYRCIAPDLRGYG
DTDAPPSPSSYSALHIVSDLVGLLDALRIDQVFLVGHDWGAAMAWYFCLFRPDRIKALVNMSVV
FRPRNPKWKPLQSLRAMLGDDYYICRFQKPGEAEEEFARAGTSRIIKTFLVSRDPRPPCVPKEI
GFGGSPNLQLALPSWLTEEDVNYYASKFDQKGFTGGLNYYRAIDLTWELTAPWTGVQIKVPVKF
IVGDLDVTYNTPGVKEYIHGGGFKKEVPFLQELVVMEGVAHFINQERPDEISAHIHDFIKKF
Mycobacterium tuberculosis (MtEPH)
(SEQ ID NO: 212)
MASQVHRILNCRGTRIHAVADSPPDQQGPLVVLLHGFPESWYSWRHQIPALAGAGYRVVAIDQR
GYGRSSKYRVQKAYRIKELVGDVVGVLDSYGAEQAFVVGHDWGAPVAWTFAWLHPDRCAGVVGI
SVPFAGRGVIGLPGSPFGERRPSDYHLELAGPGRVWYQDYFAVQDGIITEIEEDLRGWLLGLTY
TVSGEGMMAATKAAVDAGVDLESMDPIDVIRAGPLCMAEGARLKDAFVYPETMPAWFTEADLDF
YTGEFERSGFGGPLSFYHNIDNDWHDLADQQGKPLTPPALFIGGQYDVGTIWGAQAIERAHEVM
PNYRGTHMIADVGHWIQQEAPEETNRLLLDFLGGLRP
Cytochrome P450
Siraitia grosvenorii CYP87D18
(SEQ ID NO: 73)
MWTVVLGLATLFVAYYIHWINKWRDSKFNGVLPPGTMGLPLIGETIQLSRPSDSLDVHPFIQKK
VERYGPIFKTCLAGRPVVVSADAEFNNYIMLQEGRAVEMWYLDTLSKFFGLDTEWLKALGLIHK
YIRSITLNHFGAEALRERFLPFIEASSMEALHSWSTQPSVEVKNASALMVFRTSVNKMFGEDAK
KLSGNIPGKFTKLLGGFLSLPLNFPGTTYHKCLKDMKEIQKKLREVVDDRLANVGPDVEDFLGQ
AFKDKESEKFISEEFIIQLLFSISFASFESISTTLTLILKLLDEHPEVVKELEVEHEAIRKARA
DPDGPITWEEYKSMTFTLQVINETLRLGSVTPALLRKTVKDLQVKGKIIPEGWTIMLVTASRHR
DPKVYKDPHIFNPWRWKDLDSITIQKNFMPFGGGLRHCAGAEYSKVYLCTFLHILCTKYRWTKL
GGGTIARAHILSFEDGLHVKFTPKE
Cucumis melo
(SEQ ID NO: 74)
MWTILLGLATLAIAYYIHWVNKWKDSKENGVLPPGTMGLPLIGETIQLSRPSDSLDVHPFIQSK
VKRYGPIFKTCLAGRPVVVSTDAEFNHYIMLQEGRAVEMWYLDTLSKFFGLDTEWLKALGLIHK
YIRSITLNHFGAESLRERFLPRIEESARETLHYWSTQPSVEVKESAAAMVFRTSIVKMFSEDSS
KLLTAGLTKKFTGLLCGFLTLPLNVPGTTYHKCIKDMKEIQKKLKDILEERLAKCVSIDEDFLC
QAIKDKESQQFISEEFIIQLLFSISFASFESISTTLTLILNFLADHPDVAKELEAEHEAIRKAR
ADPDGPITWEEYKSMNFTLNVICETLRLGSVTPALLRKTTKEIQIKGYTIPEGWTVMLVTASRH
RDPEVYKDPDTFNPWRWKELDSITIQRNFMPFGGGLRHCAGAEYSKVYLCTFLHILFTKYRWRK
LKGGKIARAHILRFEDGLYVNFTPKE
Cucurbita maxima
(SEQ ID NO: 75)
MWTIVVGLATLAVAYYIHWINKWKDSKFNGVLPPGTMGLPLIGETLQLSRPSDSLDVHPFIKKK
VKRYGSIFKTCLAGRPVVVSTDAEFNNYIMLQEGRAVEMWYLDTLSKFFGLDTEWLKALGFIHK
YIRSITLNHFGAESLRERFLPRIEESAKETLCYWATQPSVEVKDSAAVMVFRTSMVKMVSKDSS
KLLTGGLTKKFTGLLGGFLTLPINVPGTTYNKCMKDMKEIQKKLREILEGRLASGAGSDEDFLG
QAVKDKGSQKFISDDFIIQLLFSISFASFESISTTLTLLLNYLADHPDVVKELEAEHEAIRNAR
ADPDGPITWEEYKSMTFTLHVIFETLRLGSVTPALLRKTTKELQINGYTIPEGWTVMLVTASRH
RDPAVYKDPHTFNPWRWKELDSITIQKNFMPFGGGLRHCAGAEYSKVYLCTFLHILFTKYRWTK
LKGGKVARAHILSFEDGLHMKFTPKE
Cucumis sativus
(SEQ ID NO: 76)
MWTILLGLATLAIAYYIHWVNKWKDSKFNGVLPPGTMGLPLIGETIQLSRPSDSLDVHPFIQRK
VKRYGPIFKTCLAGRPWVSTDAEFNHYIMLQEGRAVEMWYLDTLSKFFGLDTEWLKALGLIHK
YIRSITLNHFGAESLRERFLPRIEESARETLHYWSTQTSVEVKESAAAMVFRTSIVKMFSEDSS
KLLTEGLTKKFTGLLGGFLTLPLNLPGTTYHKCIKDMKQIQKKLKDILEERLAKGVKIDEDFLG
QAIKDKESQQFISEEFIIQLLFSISFASFESISTTLTLIINFLADHPDVVKELEAEHEAIRKAR
ADPDGPITWEEYKSMNFTLNVICETLRLGSVTPALLRKTTKEIQIKGYTIPEGWTVMLVTASRH
RDPEVYKDPDTFNPWRWKELDSITIQKNFMPFGGGLRHGAGAEYSKVYLCTFLHILFTKYRWRK
LKGGKIARAHILRFEDGLYVNFTPKE
Cucurbita moschata
(SEQ ID NO: 77)
MWAIVVGLATLAVAYYIHWINKWRDSRFNGVLPPGTMGLPLVGETLQLARPSDSLDVHPFIKKK
VKRYGSIFKTCLAGRPVVVSTDAEFNNYIMLQEGRAVEMWYLDTLSKFFGLDTEWLKALGFIHK
YIRSITLNHFGAESLRERFLPRIEESAKETLRYWATQPSVEVKDSAAVMVFRTSMVKMVSEDSS
KLLTGGLTKKFTGLLGGFLTLPINVPGTTYNKCMKDMKEIQKKLREILEGRLASGAGSDEDFLG
QAIKDKGSQQFISDDFIIQLLFSISFASFESISTTLTLVLNYLADHPDVVKELEAEHEAIRNAR
ADPDGPITWEEYKSMTFTLHVIFETLRLGSVTPALLRKTTKELQINGYTIPEGWTVMLVTASRH
RDPAVYKDPHTFNPWRWKELDSITIQKNFMPFGGGLRHCAGAEYSKVYLCTFLHILFTKYRWTK
LKGGRVARAHILSFEDGLHVRFTPKE
Prunus avium
(SEQ ID NO: 78)
MWTLVGLSLVALLVIYFTHWIIKWRNPKCNGVLPPGSMGLPLIGETLNLIIPSYSLDLHPFIKR
RLQRYGPIFRTSLAGRPVVVTADPEFNNYIFQQEGRMVELWYLDIFSKIFVHEGDSKTNAIGMV
HKYVRSIFLNEFGAERLKEKLLPQIEEFVNKSLCAWSSKASVEVKHAGSVMVFNFSAKQMISYD
AEKSSDDLSEKYTKIIDGLMSFPLNIPGTAYYNCSKHQKNVTTMLRDMLKERRISPETRRGDFL
DQLSIDMEKEKFLSEDFSVQLVFGGLFATFESISAVIALAFSLLADHPSVVEELTAEHEAILKN
RENPNSSITWDEYKSMTFTLQVINEILRLGNVAPGLLRRALKDIPVKGFTIPEGWTIMVVTSAL
QLSPNTFEDPLEFNPWRWKDLDSYAVSRNFMPFGGGMRQCAGAEYSRVFLATFLHVLVTRYRWT
TIKAARIARNPILGFGDGIHIKFEEKKT
Populus trichocarpa
(SEQ ID NO: 79)
MWAIGLVVVAIVVIYYTHMIFKWRSPKIEGVLPPGSMGWPLIGETLQFISPGKSLDLHPFVKKR
MEKYGPIFKTSLVGRPIIVSTDYEMNKYILQHEGTLVELWYLDSFAKFFALEGETRVNAIGTVH
KYLRSITLNHFGVESLKESLLPKIEDMLHTNLAKWASQGPVDVKQVISVMVFNFTANKIFGYDA
ENSKEKLSENYTKILNSFISLPLNIPGTSFHKCMQDREKMLKMLKDTLMERLNDPSKRRGDFLD
QAIDDMKTEKFLTEDFIPQLMFGILFASFESMSTTLTLTFKFLTENPRVVEELRAEHEAIVKKR
ENPNSRLTWEEYRSMTFTQMVVNETLRISNIPPGLFRKALKDFQVKGYTVPAGWTVMLVTPATQ
LNPDTFKDPVTFNPWRWQELDQVTISKNFMPFGGGTRQCAGAEYSKLVLSTFLHILVTNYSFTK
IRGGDVSRTPIISFGDGIHIKFTARA
Prunus persica
(SEQ ID NO: 80)
MWTLVGLSLVGLLVIYFTHWIIKWRNPKCNGVLPPGSMGLPFIGETLNLIIPSYSLDLHPFIKK
RLQRYGPIFRTSLAGRQVVVTADPEFNNYLFQQEGRMVELWYLDTFSKIFVHEGESKTNAVGMV
HKYVRSIFLNHFGAERLKEKLLPQIEEFVNKSLCAWSSKASVEVKHAGSVMVFNFSAKQMISYD
AEKSSDDLSEKYTKIIDGLMSFPLNIPGTAYYNCLKHQKNVTTMLRDMLKERQISPETRRGDFL
DQISIDMEKEKFLSEDFSVQLVFGGLFATFESISAVLALAFSLLAEHPSVVEELTAEHEAILKN
RENLNSSLTWDEYKSMTFTLQVINEILRLGNVAPGLLRRALKDIPVKGFTIPEGWTIMVVTSAL
QLSPNTFEDPLEFNPWRWKDLDSYAVSKNFMPFGGGMRQCAGAEYSRVFLATFLHVLVTKYRWT
TIKAARIARNPILGFGDGIHIKFEEKKT
Populus euphratica
(SEQ ID NO: 81)
MWTFVLCVVAVLVVYYTHWINKWRNPTCNGVLPPGSMGLPIIGETLELIIPSYSLDLHPFIKKR
IQRYGPIFRTNILGRPAVVSADPEINSYIFQNEGKLVEMWYMDTFSKLFAQSGESRTNAFGIIH
KYARSLTLTHFGSESLKERLLPQVENIVSKSLQMWSSDASVDVKPAVSIMVCDFTAKQLFGYDA
ENSSDKISEKFTKVIDAFMSLPLNIPGTTYHKCLKDKDSTLSILRNTLKERMNSPAESRGGDFL
DQIIADMDKEKFLTEDFTVNLIFGILFASFESISAALTLSLKLIGDHPSVLEELTVEHEAILKN
RENPDSPLTWAEYNSMTFSLQVINETLRLGNVAPGLLRRALQDMQVKGYTIPAGWVIMVVNSAL
HLNPATFKDPLEFNPWRWKDFDSYAVSKNLMPFGGGRRQCAGSEFTKLFMAIFLHKLVTKYRWN
IIKQGNIGRNPILGFGDGIHISFSPKDI
Juglans regia
(SEQ ID NO: 82)
MWKVGLCVVGVIVVWFTRWINKWRNPKCNGILPPGSMGPPLIGESLQLIIPSYSLDLHPFIKKR
VQRYGPIFRTSVVGQPMVVSTDVEFNHYLAKQEGRLVHFWYLDSFAEIFNLEDENAISAVGLIH
KYGRSIVLNHFGTDSLKKTLLSQIEEIVNKTLQTWSSLPSVEVKHAASVMAFDLTAKQCFGYDV
ENSAVKMSEKFLYTLDSLISFPFNIPGTVYHKCLKDKKEVLNMLRNIVKERMNSPEKYRGDFLD
QITADMNKESFLTQDFIVYLLYGLLFASFESISASLSLTLKTLAEHPAVLQQLTAEHEAILKNR
DNPNSSLTWDEYKSMTFTFQVINEALRLGNVAPGLLRRALKDIEFKGYTIPAGWTIMLANSAIQ
LNPNTYEDPLAFNPWRWQDLDPQIVSKNFMPFGGGIRQCAGAEYSKTFLATFLHVLVTKYRWTK
VKGGKMARNPILWFADGIHINFALKHN
Pyrus x bretschneideri
(SEQ ID NO: 83)
MWDVVGLSFVALLVIYLTYWITQWKNPKCNGVLPPGSMGLPLIGETLNLLIPSYSLDLHPFIRK
RLERYGPIFRTSLAGKPVLVSADPEFNNYVLKQEGRMVEFWYLDTFSKIFMQEGGNGTNQIGVI
HKYARSIFLNHFGAECIKEKLLTQIEGSINKHLRAWSNQESVEVKKAGSIMALNFCAEHMIGYD
AETATENLGEIYHRVFQGLISFPLNVPGTAYHNCLKIHKKATTMLRAMLRERRSSPEKRRGDFL
DQIIDDLDQEKFLSEDFCIHLIFGGLFAIFESISTVLTLFFSLLADHPAVLQELTAEHEALLKN
REDPNSALTWDEYKSMTFTLQVINETLRLVNTAPGLLRRALKDIPVKGYTIPAGWTILLVTPAL
HLTSNTFKDHLEFNPWRWKDLDSLVISKNFMPFGSGLRQCAGAEFSRAYLSTFLHVLVTKYRWT
TIKGARISRRPMLTFGDGAHIKFSEKKN
Morus notabilis
(SEQ ID NO: 84)
MWNTICLSVVGLVVIWISNWIRRWRNPKCNGVLPPGSMGFPLIGETLPLIIPTYSLDLHPFIKN
RLQRYGSIFRTSIVGRPVVISADPEFNNFLFQQEGSLVELYYLDTFSKIFVHEGVSRTNEFGVV
HKYIRSIFLNHFGAERLKEKLLPEIEQMVNKTLSAWSTQASVEVKHAASVLVLDFSAKQIISYD
AKKSSESLSETYTRIIQGFMSFPLNIPGTAYNQCVKDQKKIIAMLRDMLKERRASPETNRGDFL
DQISKDMDKEKFLSEDFVVQLIFGGLFATFESVSAVLALGFMLLSEHPSVLEEMIAEHETILKN
REHPNSLLAWGEYKSMTFTLQVINETLRLGNVAPGLLRKALKDIRVKGFTIPKGWAIMMVTSAL
QLSPSTFKNPLEFNPWRWKDLDSLVISKNFMPFGRGMRQCAGAEYSRAFMATFFHVLLTKYRWT
TIKVGNVSRNPILRFGNGIHIKFSKKN
Jatropha curcas (JcP450.1)
(SEQ ID NO: 85)
MWIIGLCFASLLVIYCTHFFYKWRNPKCKGVLPPGSMGLPIIGETLQLIIPSYSLDHHPFIQKR
IQRYGPIFRTNLVGRPVIVSADPEVNQYIFQQEGNSVEMWYLDAYAKIFQLDGESRLSAVGRVH
KYIRSITLNNFGIENLKENLLPQIQDLVNQSLQKWSNKASVDVKQAASVMVFNLTAKQMFSYGV
EKNSSEEMTEKFTGIFNSLMSLPLNIPGTTYHKCLKDREAMLKMLRDTLKQRLSSPDTHRGDFL
DQAIDDMDTEKFLTGDCIPQLIFGILLAGFETTATTLTLAFKFLAEHPLVLEELTAEHEKILSK
RENLESPLTWDEYKSMTFTHHVINETLRLANFLPGLLRKALKDIQVKNYTIPAGWTIMVVKSAM
QLNPEIYKDPLAFNPWRWKDLDSYTVSKNFMPFGGGSRQCAGADYSKLFMTIFLHVLVTKYRWR
KIKGGDIARNPILGFGDGLHIEVSAKN
Hevea brasiliensis
(SEQ ID NO: 86)
MLTVVLLLVGFFIIYYTYWISKWRNPNCNGVLPPGSMGFPLIGETLQLLIPSYSLDLHPFIKKR
IHRYGPIFRSNLAGRPVIVSADPEFNYYILSQEGRSVEIWYLDTFSKLFRQQGESRTNVAGYVH
KYLRGAFLSQIGSENLREKLLLHIQDMVNRTLCSWSNQESVEVKHSASLAVCDFTAKVLFGYDA
EKSPDNLSETFTRFVEGLISFPLNIPRTAYRQCLQDRQKALSILKNVLTDRRNSVENYRGDVLD
LLLNDMGKEKFLTEDFICLIMLGGLFASFESISTITTLLLKLFSAHPEVVQELEAEHEKILVSR
HGSDSLSITWDEYKSMTFTHQVINETLRLGNVAPGLLRRAIKDVQFKGYTIPSGWTIMMVTSAQ
QVNPEVYKDPLVFNPWRWKDFDSITVSKNFTPFGGGTRQCVGAEYSRLTLSLFIHLLVTKYRWT
KIKEGEIRRAPMLGFGDGIHFKFSEKE
Jatropha curcas (JcP450.2)
(SEQ ID NO: 87)
MKRAIYICLARITKQGLSLIEMLMTELLFGAFFIIFLTYWINRWRNPKCNGVLPPGSMGLPLLG
ETLQLLIPRYSLDLHPFIRKRIQRYGPIFRSNVAGRPIVFTADPELNHYIFIQERRLVELWYMD
TFSNLFVLDGESRPTGATGYIHKYMRGLFLTHFGAERLKDKLLHQIQELIHTTLQSWCKQPTIE
VKHAASAVICDFSAKFLFGYEAEKSPFNMSERFAKFAESLVSFPLNIPGTAYHQSLEDREKVMK
LLKNVLRERRNSTKKSEEDVLKQILDDMEKENFITDDFIIQILFGALFAISESIPMTIALLVKF
LSAQPSVVEELTAEHEEILKNKKEKGLDSSITWEDYKSMTFTLQVINETLRIANVAPGLLRRTL
RDIHYKGYTIPAGWTIMVLTSSRHMNPEIYKDPVEFNPWRWKDLDSQTISKNFTPFGGGTRQCA
GAEYSRAFISMFLHVLVTKYRWKNVKEGKICRGPILRIEDGIHIKLYEKH
Chenopodium quinoa
(SEQ ID NO: 88)
MWPTMGLYVATIVAICFILLELKRRNSREKQVVLPPGSKGFPLIGETLQLLVPSYSLDLPSFIR
TRIQRYGPIFKTRLVGRPVVMSADPGFNRYIVQQEGKSVEMWYLDTFSKLFAQDGEARTTAAGL
VHKYLRNLTLSHFGSESLRVNLLPHLESLVRNTLLGWSSKDTIDVKESALTMTIEFVAKQLFGY
DSDKSKEKIGEKFGNISQGLFSLPLNIPGTTYHSCLKSQREVMDMMRTALKDRLTTPESYRGDF
LDHALKDLSTEKFLSEEFILQIMFGLLFASSESTSMTLTLVLKLLSENPHVLKELEAEHERIIK
NKESPDSPLTWAEVKSMTFTLQVINESLRLGNVSLGILRRTLKDIEINGYTIPAGWTIMLVTSA
CQYNSDIYKDPLTFNPWRWKEMQPDVIAKNFMPFGGGTRQCAGAEFAKVLMTIFLHNLVTNYRW
EKIKGGEIVRTPILGFRNALRVKLTKKN
Spinacia oleracea
(SEQ ID NO: 89)
MVLLPGSKGFPFIGETLQLLLPSYSLDLPSFIRTRIQRYGPIFQTRLVGRPVVVSADPGFNRYI
VQQEGKMVEMWYLDTFSKIFAQQGEGRTNAAGLVHKYLRNITFTHFGSQTLRDKLLPHLEILVR
KTLHGWTSQESIDVKEAALTMTIEFVAKQLFGYDSDKSKERIGDKFANISQGLLSFPLNIPGTT
YHSCLKSQREVMDMMRKTLKERLASPDTCQGDFLDHALKDLNTDKFLTEDFILQIMFGLLFASS
ESTSITLTLILKFLSENPHVLEELEVEHERILKNRESPDSPLTWAEVKSMTFTLQVINESLRLG
NVSLGLLRRTLKDIEINGYTIPAGWTIMLVTSACQYNSDVYKDPLTFNPWRWKEMQPDVIAKNF
MPFGGGTRQCAGAEFAKVLMTIFLHVLVTTYRWEKIKGGEIIRTPILGFRNGLHVKLIKKARLS
Manihot esculenta
(SEQ ID NO: 90)
MEMWSVWLYIISLIIIIATHWTYRWRNPKCNGKLPPGSMGIPFIGETIQFLIPSKSLDVPNFIK
KRMNKYGPLFRTNLVGRPVIVSSDPDFNYYLLQREGKLVERWYMDSFSKLLHHDVTQIIIKHGS
IHKYLRNLVLGHFGPEPLKDKLLPQLESAISQRLQDWSKQPSIEAKSASSAMIFDFTAKILFSY
EPEKSGENIGEIFSNFLQGLMSIPLNIPGTAFHRCLKNQKRAIQMITEILKERRSNPEIHKGDF
LDQIVEDMKKDSFWTEEFAIYMMFGLLLASFETISSTLALAIIFLTDNPPVVQKLTEEHEAILK
ARENRDSGLSWKEYKSLSYTHQVVNESLRLASVAPGILRRAITDIQVDGYTIPKGWTIMVVPAA
VQLNPNTFEDPLVFNPSRWEDMGAVAMAKNFIAFGGGSRSCAGAEFSRVLMSVFVHVFVTNYRW
TKIKGGDMVRSPALGFGNGFHIRVSEKQL
Olea europaea var. sylvestris
(SEQ ID NO: 91)
MAALDLSTVGYLIVGLLTVYITHWIYKWRNPKCNGVLPPGSMGLPLIGETIQLVIPNASLDLPP
FIKKRMKRYGPIFRTNVAGRPVIITADPEFNHFLLRQDGKLVDTWSMDTFAEVFDQASQSSRKY
TRHLTLNHFGVEALREKLLPQMEDMVRTTLSNWSSQESVEVKSASVTMAIDYAARQIYSGNLEN
APLKISDLFRDLVDGLMSFPINIPGTAHHRCLQTHKKVREMMKDIVKTRLEEPERQYGDMLDHM
IEDMKKESFLDEDFIVQLMFGLFFVTSDSISTTLALAFKLLAEHPLVLEELTAEHEAILKKREK
SESHLTWNDYKSMTFTLQVINEVLRLGNIAPGFFRRALQDIPVNGYTIPSGWVIMIATAGLHLN
SNQFEDPLKFNPWRWKVCKVSSVIAKCFMPFGSGMKQCAGAEYSRVLLATFIHVLTTKYRWAIV
KGGKIVRSPIIRFPDGFHYKIIEKTN
Cucurbita pepo subsp. pepo
(SEQ ID NO: 171)
MWAIVVGLATLAVAYYIHWINKWKDSKFNGVLPPGTMGLPLVGETLQLARPSDSLDVHPFIKKK
VKRYGPIFKTCLAGRPVVVSTDAEFNNYIMLQEGRAVEMWYLDTLSKFFGLDTEWLKALGFIHK
YIRSITLNHFGAESLRERFLPRIEESAKETLRYWATQPSVEVKDSAAVMVFRTSMVKMVSEDSS
KLLTGGLTKKFTGLLGGFLTLPINVPGTTYNKCMKDMKEIQKKLREILEGRLASGGGSDEDFLG
QAIKDKGSQQFISDDFIIQLLFSISFASFESISTTLTLVLNYLADHPDVVKELEAEHEAIRNAR
ADPDGPITWEEYKSMTFTLHVIFETLRLGSVTPALLRKTTKELQINGYTIPEGWTVMLVTASRH
RDPAVYKDPHTFNPWRWKELDSITIQKNFMPFGGGLRHCAGAEYSKVYLCTFLHILFTKYRWTK
LKGGKVARAHILSFEDGLHVKFTPKE
Capsella rubella CYP705A38
(SEQ ID NO: 172)
MATLMTIDLQNCFIFIILSLLCYYLLFKKQKGSRAGCVLPPSPPSLPIIGHLHLLLSNLTHKSL
QNISTKFGSFLYLRVVNLPIVLVSSPSVAYEIYKTHDVNVSSRVATSLGDSLFLGSSGFITAPY
GDYWKFMKKMVATKLLRPQAIEQSRGGRAEELQMFYENLLDKAMKKESIEVSKEAMKLTNNIIC
RMSMGRSCSDENGEAERVRELLVKSTALTKKIFFANMFPRIPLFKKEIMGVSSEFDDLLERLLV
EHEERVEEHENKDMMDLLLEAYRDENAEYKISRKQIKSLFVEIFLGGTDTSAQTVQWILAELIN
KPNILERIREEIDSVVGKSRLMKETDLPNLPYLQATVKEGLRMHPPSPLLVRTFQESCEVKGFY
MPEKTMLVINVYALMRDPDTWEDPNEFKPERFLLSSRSRQEDEKEQGMMKYLPFGAGRRGCPGS
NLAYLFVGIAVGVMVQCFDWKIKEDKVNMEETTAGMNLAMAHPFKCTPVVRNDPLTLNLENPSS
Brassica rapa CYP705A37v2
(SEQ ID NO: 173)
MIVDFQNCSIFILLCFFTFLCYSVFFFFKKTNDLGPSPPSLPIIGHLHHFLSGLPHKAFQKIST
KYGPLLHLHIFSFPIVLVSSPTMAHEIFTTHDLNISSRNTPAIDESLLFGPSGFTVAPYGDYVK
FIKKLLATKLLRPRAIEKSRGVRAEELKQFYLKVQDKALKKESIEIGKETMKFTNNMICRMSIG
RSFSEENGEVETLRELIIKSFALSKQILFVNVLRRPLEMLGLMSLFKKDIMDVSRGFDELLERV
LAEHEEKREEDQDMDMMDLLLEACRDENAEYKITRNQIKSLFVEIFLGGTDTSAHTTQWTMAEL
VNNPNILGRLRDEIDLVVGKERLIQETDLPNLPYLQAVVKEGLRLHPPAPLLVRMFDKKCVIKD
FFKVPEKTTLVVNVYGVMRDPDSWEDPNEFKPERFLTSKQEEDKVLKYLPFAAGRRGCPATNVG
YIFVGISIGMMVQCFDWSIKEKVSMEEVYAGMSLSMAHPPTCTPVSRLSL
Siraitia qrosvenorii
(SEQ ID NO: 174)
MDFFSAFLLLLLTVLILLQIRTRRRNLPPSPPSLPIIGHLHLLKRPIHRNFHKIAAEYGPIFSL
RFGSRLAVIVSSLDIAEECFTKNDLIFANRPRLLISKHLGYNCTTMATSPYGDHWRNLRRLAAI
EIFSTARLNSSLSIRKDEIQRLLLKLHSGSSGEFTKVELKTMFSELAFNALMRIVAGKRYYGDE
VSDEEEAREFRGLMEEISLHGGASHWVDFMPILKWIGGGGFEKSLVRLTKRTDKFMQALIEERR
NKKVLERKNSLLDRLLELQASEPEYYTDQIIKGLVLVLLRAGTDTSAVTLNWAMAQLLNNPELL
AKAKAELDTKIGQDRPVDEPDLPNLSYLQAIVSETLRLHPAAPMLLSHYSSADCTVAGYDIPRG
TTLLVNAWAIHRDPKLWDDPTSFRPERFLGAANELQSKKLIAFGLGRRSCPGDTMALRFVGLTL
GLLIQCYQWKKCGDEKVDMGEGGGITIHKAKPLEAMCKARPAMYKLLLNALDKI
Camelina sativa
(SEQ ID NO: 175)
MATMMIFDFQNCFIFIILCFVSLLCYTILFKKQESSRTGCVLPPSPPSLPIIGHLHLLLSSLTH
KSLHNISSKFGPFLYLRVVNLPIVLVSSASVAYEIYKTQDVNVSSRVATSLGDSLFLGSSGFIT
APYGDYWKFMKKMVATKLLRPQAIEQSRGGRAEELQGLYENLLDKAMKKESIEISKEAMKFTNN
IICRMSMGRSCSDENGEAEIVRELLVKSTALTKKIFFANMFPRIPLFKKEIMGVSNQFDELLER
LLVEHEERVEEHENKDMMDLLLEAFRDEHAEYKISRKQIKSLFVEIFLGGTDTSAQTVQWIMAE
LINKPSIIEKIREEIDSVVGKTRLIKETDLPKLPYLQVVVKEGLRMHPPSPLVVRTFQESCEVK
GFYMPEKTMLVINVYALMRDPESWEDPNEFKPERFLPSSKSRQDEEKEQGLKYLPFGAGRRGCP
GSNLAYLFVGLAVGVMVQCFDWKIKEDKVNMEETTAGMNLAMAHPFKCTPVVRIDPLTFNLKSP
SP
Raphanus sativus
(SEQ ID NO: 176)
MAPMTIDFQTCFIFILLSFFSFFCYFFFFKKTNDLGPSPPSLPIIGHLHHFLSVLPHKAFQQIS
TKYGPLLHLRIFSFPIVLVSSATMAYEIFTTHDLNISSRNAPAIDESLVFGSSGFIVSPYGDYV
KFIKKLLATKLLRPRAIEKSRGVRAEELKQFYLKLHDKALKKESIEIGNETMKFTNNMICGMSM
GRSCSEENGETETVRGLINKSFALSRKILFVNVLRRPLEKLGLLSLFKKDILDVSNRFDELLER
ILLEHEEKPEEEQDMDMMDLLLEASRDENAEYKITRNQIKALFVEIFMGGTDTSAHTTQWTMAE
LVNNPNSLEKLRDEIDMVVGKSRLIQETDLPNLPYLQAVVKEGLRLHPPAPLLVRMFEKKCVIK
DFFNVPEKTTLVVNLYGVMRDPDSWEDPNEFKPERFLTSKQEEEKTLKYLPFAAGRRGCPATNV
AYIFVGISIGMMVQCFDWSIKDKVSMEEVYAGMSLSMAHPPKFTPVSRLSL
Cucumis sativus (CsCYP87D20)
(SEQ ID NO: 194)
MAWTILLGLATLAIAYYIHWVNKWKDSKFNGVLPPGTMGLPLIGETIQLSRPSDSLDVHPFIQR
KVKRYGPIFKTCLAGRPVVVSTDAEFNHYIMLQEGRAVEMWYLDTLSKFFGLDTEWLKALGLIH
KYIRSITLNHFGAESLRERFLPRIEESARETLHYWSTQTSVEVKESAAAMVFRTSIVKMFSEDS
SKLLTEGLTKKFTGLLGGFLTLPLNLPGTTYHKCIKDMRQIQKKLKDILEERLAKGVKIDEDFL
GQAIKDKESQQFISEEFIIQLLFSISFASFESISTTLTLLLNFLADHPDVVKELEAEHEAIRKA
RADPDGPITWEEYKSMNFTLNVICETLRLGSVTPALLRKTTKEIQIKGYTIPEGWTVMLVTASR
HRDPEVYKDPDTFNPWRWKELDSITIQKNFMPFGGGLRHCAGAEYSKVYLCTFLHILFTKYRWR
KLKGGKIARAHILRFEDGLYVNFTPKE
Cucumis sativus (sohB_CsCYP87D20)
(SEQ ID NO: 195)
MALLSEYGLFLAKIVTVVLAIAAIAAIIHWVNKWKDSKFNGVLPPGTMGLPLIGETIQLSRPSD
SLDVHPFIQRKVKRYGPIFKTCLAGRPVVVSTDAEFNHYIMLQEGRAVEMWYLDTLSKFFGLDT
EWLKALGLIHKYIRSITLNHFGAESLRERFLPRIEESARETLHYWSTQTSVEVKESAAAMVFRT
SIVKMFSEDSSKLLTEGLTKKFTGLLGGFLTLPLNLPGTTYHKCIKDMKQIQKKLKDILEERLA
KGVKIDEDFLGQAIKDKESQQFISEEFIIQLLFSISFASFESISTTLTLILNFLADHPDVVKEL
EAEHEAIRKARADPDGPITWEEYKSMNFTLNVICETLRLGSVTPALLRKTTKEIQIKGYTIPEG
WTVMLVTASRHRDPEVYKDPDTFNPWRWKELDSITIQKNFMPFGGGLRHCAGAEYSKVYLCTFL
HILFTKYRWRKLKGGKIARAHILRFEDGLYVNFTPKE
Cucumis sativus (zipA_CsCYP87D20)
(SEQ ID NO: 196)
MAQDLRLILIIVGAIAIIALLVHGFHWVNKWKDSKFNGVLPPGTMGLPLIGETIQLSRPSDSLD
VHPFIQRKVKRYGPIFKTCLAGRPVVVSTDAEFNHYIMLQEGRAVEMWYLDTLSKFFGLDTEWL
KALGLIHKYIRSITLNHFGAESLRERFLPRIEESARETLHYWSTQTSVEVKESAAAMVFRTSIV
KMFSEDSSKLLTEGLTKKFTGLLGGFLTLPLMLPGTTYHKCIKDMKQIQKKLKDILEERLAKGV
KIDEDFLGQAIKDKESQQFISEEFIIQLLFSISFASFESISTTLTLILNFLADHPDVVKELEAE
HEAIRKARADPDGPITWEEYKSMNFTLNVICETLRLGSVTPALLRKTTKEIQIKGYTIPEGWTV
MLVTASRHRDPEVYKDPDTFNPWRWKELDSITIQKNFMPFGGGLRHCAGAEYSKVYLCTFLHIL
FTKYRWRKLKGGKIARAHILRFEDGLYVNFTPKE
Cucumis sativus (CsCYP87D20_mut)
(SEQ ID NO: 197)
MAWTILLGLATLAIAYYIHWVNKWKDSKFNGVLPPGTMGLPLIGETIQFSRPSDSLDVHPFIQR
KVKRYGPIFKTCIAGRPVVVSTDAEFNHYIMLQEGRAVEMWYLDTFSKFLGLDTEWLKALGLIH
KYIRSITLNHFGAESLRERFLPRIEESARETLHYWSTQTSVEVKESAAAMVFRTSIVKMFSEDS
SKLLTEGLTKKFTGLLGGFLTLPLNLPGTTYHKCIKDMKQIQKKLKDILEERLAKGVKIDEDFL
GQAIKDKESQQFISEEFIIQLLFSISFASFASISTTLTLILNFLADHPDVVKELEAEHEAIRKA
RADPDGPITWEEYKSMNFTLNVICETLRLGSVTPALLRKTTKEIQIKGYTIPEGWTVMLVTASR
HRDPEVYKDPDTFNPWRWKELDSITIQKNFMPFGGGLRHCAGAEYSKVYLCTFLHILFTKYRWR
KLKGGKIARALILRFEDGLYVNFTPKE
Cucumis sativus (sohB_CsCYP87D20_mut)
(SEQ ID NO: 198)
MALLSEYGLFLAKIVTVVLAIAAIAAIIHWVNKWKDSKFNGVLPPCTMGLPLIGETIQFSRPSD
SLDVHPFIQRKVKRYGPIFKTCIAGRPVVVSTDAEFNHYIMLQEGRAVEMWYLDTFSKFLGLDT
EWLKALGLIHKYIRSITLNHFGAESLRERFLPRIEESARETLHYWSTQTSVEVKESAAAMVFRT
SIVKMFSEDSSKLLTEGLTKKFTGLLGGFLTLPLNLPGTTYHKCIKDMKQIQKKLKDILEERLA
KGVKIDEDFLGQAIKDKESQQFISEEFIIQLLFSISFASFASISTTLTLILNFLADHPDVVKEL
EAEHEAIRKARADPDGPITWEEYKSMNFTLNVICETLRLGSVTPALLRKTTKEIQIKGYTIPEG
WTVMLVTASRHRDPEVYKDPDTFNPWRWKELDSITIQKNFMPFGGGLRHCAGAEYSKVYLCTFL
HILFTKYRWRKLKGGKIARALILRFEDGLYVNFTPKE
Cucurbita pepo subsp. pepo (sohB_CppCYP)
(SEQ ID NO: 199)
MALLSEYGLFLAKIVTVVLAIAAIAAIIHWINKWKDSKFNGVLPPGTMGLPLVGETLQLARPSD
SLDVHPFIKKKVKRYGPIFKTCLAGRPVVVSTDAEFNNYIMLQEGRAVEMWYLDTLSKFFGLDT
EWLKALGFIHKYIRSITLNHFGAESLRERFLPRIEESAKETLRYWATQPSVEVKDSAAVMVFRT
SMVKMVSEDSSKLLTGGLTKKFTGLLGGFLTLPINVPGTTYNKCMKDMKEIQKKLREILEGRLA
SGGGSDEDFLGQAIKDKGSQQFISDDFIIQLLFSISFASFESISTTLTLVLNYLADHPDVVKEL
EAEHEAIRNARADPDGPITWEEYKSMTFTLHVIFETLRLGSVTPALLRKTTKELQINGYTIPEG
WTVMLVTASRHRDPAVYKDPHTFNPWRWKELDSITIQKNFMPFGGGLRHCAGAEYSKVYLCTFL
HILFTKYRWTKLKGGKVARAHILSFEDGLHVKFTPKE
Cucurbita pepo subsp. pepo (17alpha_CppCYP)
(SEQ ID NO: 200)
MALLLAVFHWINKWKDSKFNGVLPPGTMGLPLVGETLQLARPSDSLDVHPFIKKKVKRYGPIFK
TCLAGRPVVVSTDAEFNNYIMLQEGRAVEMWYLDTLSKFFGLDTEWLKALGFIHKYIRSITLNH
FGAESLRERFLPRIEESAKETLRYWATQPSVEVKDSAAVMVFRTSMVKMVSEDSSKLLTGGLTK
KFTGLLGGFLTLPINVPGTTYNKCMKDMKEIQKKLREILEGRLASGGGSDEDFLGQAIKDKGSQ
QFISDDFIIQLLFSISFASFESISTTLTLVLNYLADHPDVVKELEAEHEAIRNARADPDGPITW
EEYKSMTFTLHVIFETLRLGSVTPALLRKTTKELQINGYTIPEGWTVMLVTASRHRDPAVYKDP
HTFNPWRWKELDSITIQKNFMPFGGGLRHCAGAEYSKVYLCTFLHILFTKYRWTKLKGGKVARA
HILSFEDGLHVKFTPKE
Siraitia grosvenorii (CYP1798)
(SEQ ID NO: 221)
MEMSSSVAATISIWMVVVCIVGVGWRVVNWVWLRPKKLEKRLREQGLAGNSYRLLFGDLKERAA
MEEQANSKPINFSHDIGPRVFPSMYKTIQNYGKNSYMWLGPYPRVHIMDPQQLKTVFTLVYDIQ
KPNLNPLIKFLLDGIVTHEGEKWAKHRKIINPAFHLEKLKDMIPAFFHSCNEIVNEWERLISKE
GSCELDVMPYLQNLAADAISRTAFGSSYEEGKMIFQLLKELTDLVVKVAFGVYIPGWRFLPTKS
NNKMKEINRKIKSLLLGIINKRQKAMEEGEAGQSDLLGILMESNSNEIQGEGNNKEDGMSIEDV
IEECKVFYIGGQETTARLLIWTMILLSSHTEWQERARTEVLKVFGNKKPDFDGLSRLKVVTMIL
NEVLRLYPPASMLTRIIQKETRVGKLTLPAGVILIMPIILIHRDHDLWGEDANEFKPERFSKGV
SKAAKVQPAFFPFGWGPRICMGQNFAMIEAKMALSLILQRFSFELSSSYVHAPTVVFTTQPQHG
AHIVLRKL
Cytochrome P450 Reductase
Stevia rebaudiana (SrCPR1)
(SEQ ID NO: 92)
MAQSDSVKVSPFDLVSAAMNGKAMEKLNASESEDPTTLPALKMLVENRELLTLFTTSFAVLIGC
LVFLMWRRSSSKKLVQDPVPQVIVVKKKEKESEVDDGKKKVSIFYGTQTGTAEGFAKALVEEAK
VRYEKTSFKVIDLDDYAADDDEYEEKLKKESLAFFFLATYGDGEPTDNAANFYKWFTEGDDKGE
NLKKLQYGVFGLGNRQYEHFNKIAIVVDDKLTEMGAKRLVPVGLGDDDQCIEDDFTAWKELVWP
ELDQLLRDEDDTSVTTPYTAAVLEYRVVYHDKPADSYAEDQTHTNGHVVHDAQHPSRSNVAFKK
ELHTSQSDRSCTHLEFDISHTGLSYETGDHVGVYSENLSEVVDEALKLLGLSPDTYFSVHADKE
DGTPIGGASLPPPFPPCTLRDALTRYADVLSSPKKVALLALAAHASDPSEADRLKFLASPAGKD
EYAQWIVANQRSLLEVMQSFPSAKPPLGVFFAAVAPRLQPRYYSISSSPKMSPNRIHVTCALVY
ETTPAGRIHRGLCSTWMKNAVPLTESPDCSQASIFVRTSNFRLPVDPKVPVIMIGPGTGLAPFR
GFLQERLALKESGTELGSSIFFFGCRNRKVDFIYEDELNNFVETGALSELIVAFSREGTAKEYV
QHKMSQKASDIWKLLSEGAYLYVCGDAKGMAKDVHRTLHTIVQEQGSLDSSKAELYVKNLQMSG
RYLRDVW
Arabidopsis thaliana CPR1 (AtCPR1)
(SEQ ID NO: 93)
MATSALYASDLFKQLKSIMGTDSLSDDVVLVIATTSLALVAGFVVLLWKKTTADRSGELKPLMI
PKSLMAKDEDDDLDLGSGKTRVSIFFGTQTGTAEGFAKALSEEIKARYEKAAVKVIDLDDYAAD
DDQYEEKLKKETLAFFCVATYGDGEPTDNAARFYKWFTEENERDIKLQQLAYGVFALGNRQYEH
FNKIGIVLDEELCKKGAKRLIEVGLGDDDQSIEDDFNAWKESLWSELDKLLKDEDDKSVATPYT
AVIPEYRVVTHDPRFTTQKSMESNVANGNTTIDIHHPCRVDVAVQKELHTHESDRSCIHLEFDI
SRTGITYETGDHVGVYAENHVEIVEEAGKLLGHSLDLVFSIHADKEDGSPLESAVPPPFPGPCT
LGTGLARYADLLNPPRKSALVALAAYATEPSEAEKLKHLTSPDGKDEYSQWIVASQRSLLEVMA
AFPSAKPPLGVFFAAIAPRLQPRYYSISSSPRLAPSRVHVTSALVYGPTPTGRIHKGVCSTWMK
NAVPAEKSHECSGAPIFIRASNFKLPSNPSTPIVMVGPGTGLAPFRGFLQERMALKEDGEELGS
SLLffGCRNRQMDELYEDELNNFVDQGVISELIMAFSREGAQKEYVQHKMMEKAAQVWDLIKEE
GYLYVCGDAKGMARDVHRTLHTIVQEQEGVSSSEAEAIVKKLQTEGRYLRDVW
Arabidopsis thaliana CPR2 (AtCPR2)
(SEQ ID NO: 94)
MASSSSSSSTSMIDLMAAIIKGEPVIVSDPANASAYESVAAELSSMLIENRQFAMIVTTSIAVL
IGCIVMLVWRRSGSGNSKRVEPLKPLVIKPREEEIDDGRKKVTIFFGTQTGTAEGFAKALGEEA
KARYEKTRFKIVDLDDYAADDDEYEEKLKKEDVAFFFLATYGDGEPTDNAARFYKWFTEGNDRG
EWLKNLKYGVFGLGNRQYEHFNKVAKVVDDILVEQGAQRLVQVGLGDDDQCIEDDFTAWREALW
PELDTILREEGDTAVATPYTAAVLEYRVSIHDSEDAKFNDINMANGNGYTVFDAQHPYKANVAV
KRELHTPESDRSCIHLEFDIAGSGLTYETGDHVGVLCDNLSETVDEALRLLDMSPDTYFSLHAE
KEDGTPISSSLPPPFPPCNLRTALTRYACLLSSPKKSALVALAAHASDPTEAERLKHLASPAGK
DEYSKWVVESQRSLLEVMAEFPSAKPPLGVFFAGVAPRLQPRFYSISSSPKIAETRIHVTCALV
YEKMPTGRIHKGVCSTWMKNAVPYEKSENCSSAPIEVRQSNFKLPSDSKVPIIMIGPGTGLAPF
RGFLQERLALVESGVELGPSVLFFGCRNRRMDFIYEEELQRFVESGALAELSVAFSREGPTKEY
VQHKMMDKASDIWNMISQGAYLYVCGDAKGMARDVHRSLHTIAQEQGSMDSTKAEGFVKNLQTS
GRYLRDVW
Arabidopsis thaliana (AtCPR3)
(SEQ ID NO: 95)
MASSSSSSSTSMIDLMAAIIKGEPVIVSDPANASAYESVAAELSSMLIENRQFAMIVTTSIAVL
IGCIVMLVWRRSGSGNSKRVEPLKPLVIKPREEEIDDGRKKVTIFFGTQTGTAEGFAKALGEEA
KARYEKTRFKIVDLDDYAADDDEYEEKLKKEDVAFFFLATYGDGEPTDNAARFYKWFTEGNDRG
EWLKNLKYGVFGLGNRQYEHFNKVAKVVDDILVEQGAQRLVQVGLGDDDQCIEDDFTAWREALW
PELDTILREEGDTAVATPYTAAVLEYRVSIHDSEDAKFNDITLANGNGYTVFDAQHPYKANVAV
KRELHTPESDRSCIHLEFDIAGSGLTMKLGDHVGVLCDNLSETVDEALRLLDMSPDTYFSLHAE
KEDGTPISSSLPPPFPPCNLRTALTRYACLLSSPKKSALVALAAHASDPTEAERLKHLASPAGK
DEYSKWVVESQRSLLEVMAEFPSAKPPLGVFEAGVAPRLQPRPYSISSSPKIAETRIHVTCALV
YEKMPTGRIHKGVCSTWMKNAVPYEKSEKLFLGRPIFVRQSNFKLPSDSKVPIIMIGPGTGLAP
FRGFLQERLALVESGVELGPSVIFFGCRNRRMDFIYEEELQRFVESGALAELSVAFSREGPTKE
YVQHKMMDKASDIWNMISQGAYLYVCGDAKGMARDVHRSLHTIAQEQGSMDSTKAEGFVKNLQT
SGRYLRDVW
Stevia rebaudiana CPR2 (SrCPR2)
(SEQ ID NO: 96)
MAQSESVEASTIDLMTAVLKDTVIDTANASDNGDSKMPPALAMMFEIRDLLLILTTSVAVLVGC
FVVLVWKRSSGKKSGKELEPPKIVVPKRRLEQEVDDGKKKVTIFFGTQTGTAEGFAKALFEEAK
ARYEKAAFKVIDLDDYAADLDEYAEKLKKETYAFFFLATYGDGEPTDNAAKFYKWFTEGDEKGV
WLQKLQYGVFGLGNRQYEHFNKIGIVVDDGLTEQGAKRIVPVGLGDDDQSIEDDFSAWKELVWP
ELDLLLRDEDDKAAATPYTAAIPEYRVVFHDKPDAFSDDHTQTNGHAVHDAQHPCRSNVAVKKE
LHTPESDRSCTHLEFDISHTGLSYETGDHVGVYCENLIEVVEEAGKLLGLSTDTYFSLHIDNED
GSPLGGPSLQPPFPPCTLRKALTNYADLLSSPKKSTLLALAAHASDPTEADRLRFLASREGKDE
YAEWVVANQRSLLEVMEAFPSARPPLGVFFAAVAPRLQPRYYSISSSPKMEFNRIHVTCALVYE
KTPAGRIHKGICSTWMKNAVPLTESQDCSWAPIFVRTSNFRLPIDPKVPVIMIGPGTGLAPFRG
FLQERLALKESGTELGSSILFFGCRNRKVDYIYENELNNFVENGALSELDVAFSRDGPTKEYVQ
HKMTQKASEIWNMLSEGAYLYVCGDAKGMAKDVHRTLHTIVQEQGSLDSSKAELYVKNLQMSGR
YLRDVW
Stevia rebaudiana CPR3 (SrCPR3)
(SEQ ID NO: 97)
MAQSNSVKISPLDLVTALFSGKVLDTSNASESGESAMLPTIAMIMENRELLMILTTSVAVLIGC
VVVLVWRRSSTKKSALEPPVIVVPKRVQEEEVDDGKKKVTVFFGTQTGTAEGFAKALVEEAKAR
YEKAVFKVIDLDDYAADDDEYEEKLKKESLAFFFLATYGDGEPTDNAARFYKWFTEGDAKGEWL
NKLQYGVFGLGNRQYEHFNKIAKVVDDGLVEQGAKRLVPVGLGDDDQCIEDDFTAWKELVWPEL
DQLLRDEDDTTVATPYTAAVAEYRVVEHEKPDALSEDYSYTNGHAVHDAQHPCRSNVAVKKELH
SPESDRSCTHLEFDISNTGLSYETGDHVGVYCENLSEVVNDAERLVGLPPDTYFSIHTDSEDGS
PLGGASLPPPFPPCTLRKALTCYADVLSSPKKSALLALAAHATDPSEADRLKFLASPAGKDEYS
QWIVASQRSLLEVMEAFPSAKPSLGVFFASVAPRLQPRYYSISSSPKMAPDRIHVTCALVYEKT
PAGRIHKGVCSTWMKNAVPMTESQDCSWAPIYVRTSNFRLPSDPKVPVIMIGPGTGLAPFRGFL
QERLALKEAGTDLGLSILFFGCRNRKVDFIYENELNNFVETGALSELIVAFSREGPTKEYVQHK
MSEKASDIWNLLSEGAYLYVCGDAKGMAKDVHRTLHTIVQEQGSLDSSKAELYVKNLQMSGRYL
RDVW
Artemisia annua CPR (AaCPR)
(SEQ ID NO: 98)
MAQSTTSVKLSPFDLMTALLNGKVSFDTSNTSDTNIPLAVFMENRELLMILTTSVAVLIGCVVV
LVWRRSSSAAKKAAESPVIVVPKKVTEDEVDDGRKKVTVFFGTQTGTAEGFAKALVEEAKARYE
KAVFKVIDLDDYAAEDDEYEEKLKKESLAFFFLATYGDGEPTDNAARFYKWFTEGEEKGEWLDK
LQYAVFGLGNRQYEHFNKIAKVVDEKLVEQGAKRLVPVGMGDDDQCIEDDFTAWKELVWPELDQ
LLRDEDDTSVATPYTAAVAEYRVVFHDKPETYDQDQLTNGHAVHDAQHPCRSNVAVKKELHSPL
SDRSCTHLEFDISNTGLSYETGDHVGVYVENLSEVVDEAEKLIGLPPHTYFSVHADNEDGTPLG
GASLPPPFPPCTLRKALASYADVLSSPKKSALLALAAHATDSTEADRLKFLASPAGKDEYAQWI
VASHRSLLEVMEAFPSAKPPLGVFFASVAPRLQPRYYSISSSPRFAPNRIHVTCALVYEQTPSG
RVHKGVCSTWMKNAVPMTESQDCSWAPIYVRTSNFRLPSDPKVPVIMIGPGTGLAPFRGFLQER
LAQKEAGTELGTAILFFGCPURKVDFIYEDELNNFVETGALSELVTAFSREGATKEYVQHKMTQ
KASDIWNLLSEGAYLYVCGDAKGMAKDVHRTLHTIVQEQGSLDSSKAELYVKNLQMAGRYLRDV
W
CPR (PgCPR)
(SEQ ID NO: 99)
MAQSSSGSMSPFDFMTAIIKGKMEPSNASLGAAGEVTAMILDNRELVMILTTSIAVLIGCVVVF
IWRRSSSQTPTAVQPLKPLLAKETESEVDDGKQKVTIFFGTQTGTAEGFAKALADEAKARYDKV
TFKVVDLDDYAADDEEYEEKLKKETLAFFFLATYGDGEPTDNAARFYKWFLEGKERGEWLQNLK
FGVFGLGNRQYEHFNKIAIVVDEILAEQGGKRLISVGLGDDDQCIEDDFTAWRESLWPELDQLL
RDEDDTTVSTPYTAAVLEYRVVFHDPADAPTLEKSYSNANGHSVVDAQHPLRANVAVRRELHTP
ASDRSCTHLEFDISGTGIAYETGDHVGVYCENLAETVEEALELLGLSPDTYFSVHADKEDGTPL
SGSSLPPPFPPCTLRTALTLHADLLSSPKKSALLALAAHASDPTEADRLRHLASPAGKDEYAQW
IVASQRSLLEVMAEFPSAKPPLGVFFASVAPRLQPRYYSISSSPRIAPSRIHVTCALVYEKTPT
GRVHKGVCSTWMKNSVPSEKSDECSWAPIFVRQSNFKLPADAKVPIIMIGPGTGLAPFRGFLQE
RLALKEAGTELGPSILFFGCRNSKMDYIYEDELDNFVQNGALSELVLAFSREGPTKEYVQHKMM
EKASDIWNLISQGAYLYVCGDAKGMARDVHRTLHTIAQEQGSLDSSKAESMVKNLQMSGRYLRD
VW
Camptotheca acuminate CaCPR
(SEQ ID NO: 201)
MAQSSSVKVSTFDLMSAILRGRSMDQTNVSFESGESPALAMLIENRELVMILTTSVAVLIGCFV
VLLWRRSSGKSGKVTEPPKPLMVKTEPEPEVDDGKKKVSIFYGTQTGTAEGFAKALAEEAKVRY
EKASFKVIDLDDYAADDEEYEEKLKKETLTFFFLATYGDGEPTDNAARFYKWFMEGKERGDWLK
NLHYGVFGLGNRQYEHFNRIAKVVDDTIAEQGGKRLIPVGLGDDDQCIEDDFAAWRELLWPELD
QLLQDEDGTTVATPYTAAVLEYRVVFHDSPDASLLDKSFSKSNGHAVHDAQHPCRANVAVRREL
HTPASDRSCTHLEFDISGTGLVYETGDHVGVYCENLIEVVEEAEMLLGLSPDTFFSIHTDKEDG
TPLSGSSLPPPFPPCTLRRALTQYADLLSSPKKSSLLALAAHCSDPSEADRLRHLASPSGKDEY
AQWVVASQRSLLEVMAEFPSAKPPIGAFFAGVAPRLQPRYYSISSSPRMAPSRIHVTCALVFEK
TPVGRIHKGVCSTWMKNAVPLDESRDCSWAPIFVRQSNFKLPADTKVPVLMIGPGTGLAPFRGF
LQERLALKEAGAELGPAILFFGCRNRQMDYIYEDELNNFVETGALSELIVAFSREGPKKEYVQH
KMMEKASDIWNMISQEGYIYVCGDAKGMARDVHRTLHTIVQEQGSLDSSKTESMVKNLQMNGRY
LRDVW
Non-heme iron oxidase
Acetobacter pasteurianus subsp. ascendens (ApGA2ox)
(SEQ ID NO: 100)
MSVSKTTETFTSIPVIDISKLYSSDLAERKAVAEKLGDAARNIGFLYISGHNVSADLIEGVRKA
ARDFFAEPFEKKMEYYIGTSATHKGFVPEGEEVYSAGRPDHKEAFDIGYEVPANHPLVQAGTPL
LGPNNWPDIPGFRSAAEAYYRTVFDLGRTLFRGFALALGLNESYFQTVANFPPSKLRMIHYPYD
ADAQDAPGIGAHTDYECFTILLADKPGLEVMNGNGDWIDAPPIPGAFVVNIGDMLEVMTAGEFV
ATAHRVRKVSEERYSFPLFYACDYHTQIRPLPAFAKKIDASYETITIGEHMWAQALQTYQYLVK
KVEKGELKLPKGARKTATFGHFKRNSAA
Cucurbita maxima (CmGA2ox)
(SEQ ID NO: 101)
MAAASSFSAAFYSGIPLIDLSAPDAKQLIVKACEELGFFKVVKHGVPMELISSLESESTKFFSL
PLSEKQRAGPPSPFGYGNKQIGRNGDVGWVEYLLLNTHLESNSDGFLSMFGQDPQKLRSAVNDY
ISAVRNMAGEILELMAEGLKIQQRNVFSKLVMDEQSDSVFRVNHYPPCPDLQALKGTNMIGFGE
HTDPQIISVLRSNNTSGFQISLADGNWISVPPDHSSFFINVGDSLQVMTNGRFKSVKHRVLTNS
SKSRVSMIYFGGPPLSEKIAPLASLMQGEERSLYKEFTWFEYKRSAYNSRLADNRLVPFERIAA
S
Dendrobium catenatum (DcGA3ox)
(SEQ ID NO: 102)
MPSLSKEHFDLYSAFHVPETHAWSSSHLHDHPIAGDGATIPVIDISDPDAASMVGGACRSWGVF
YATSHGIPADLLHQVESHARRLFSLPLHRKLQTAPRDGSLSGYGRPPISAFFPKLMWSEGFTLA
GHDDHLAVTSQLSPFDSLSFCEVMEAYRKEMKKLAGRLFRLLILSLGLEEEEMGQVGPLKELSQ
AADAIQLNSYPTCPEPERAIGMAAHTDSAFLTVLHQTDGAGGLQVLRDQDESGSARWVDVLPRP
DCLVVNVGDLLHILSNGRFKSVRHRAVVNRADHRISAAYFIGPPAHMKVGSITKLVDMRTGPMY
RPVTWPEYLGIRTRLFDKALDSVKFQEKELEKD
Cucurbita maxima (CmGA3ox)
(SEQ ID NO: 103)
MATTIADVFKSFPVHIPAHKNLDFDSLHELPDSYAWIQPDSFPSPTHKHHNSILDSDSDSVPLI
DLSLPNAAALIGNAFRSWGAFQVINHGVPISLLQSIESSADTLFSLPPSHKLKAARTPDGISGY
GLVRISSFFPKRMWSEGFTIVGSPLDHFRQLWPHDYHKHCEIVEEYDREMRSLCGRLMWLGLGE
LGITRDDMKWAGPDGDFKTSPAATQFNSYPVCPDPDRAMGLGPHTDTSLLTIVYQSNTRGLQVL
REGKRWVTVEPVAGGLVVQVGDLLHILTNGLYPSALHQAVVNRTRKRLSVAYVFGPPESAEISP
LKKLLGPTQPPLYRPVTWTEYLGKKAEHFNNALSTVRLCAPITGLLDVNDHSRVKVG
Cucurbita maxima (CmGA20ox)
(SEQ ID NO: 104)
MHVVTSTPEARHDGAPLVFDASVLRHQHNIPKQFIWPDEEKPAATCPELEVPLIDLSGFLSGEK
DAAAEAVRLVGEACEKHGFFLVVNHGVDRKLIGEAHKYMDEFFELPLSQKQSAQRKAGEHCGYA
SSFTGRFSSKLPWKETLSFRFAADESLNNLVLHYLNDKLGDQFAKFGRVYQDYCEAMSGLSLGI
MELLGKSLGVEEQCFKNFFKDNDSIMRLNFYPPCQKPHLTLGTGPHCDPTSLTILHQDQVGGLQ
VFVDNQWRLITPNFDAFVVNIGDTFMALSNGRYKSCLHRAVVNSERTRKSLAFFLCPRNDKVVR
PPRELVDTQNPRRYPDFTWSMLLRFTQTHYRADMKTLEAFSAWLQQEQQEQQEQQFNI
Agapanthus praecox subsp. orientalis (ApoGA20ox)
(SEQ ID NO: 105)
MVLQPFVFDAALLRDEHNIPTQFIWPEEDKPSPDASEELILPFIDLKAFLSGDPDSPFQVSKQV
GEACESLGAFQVTNHGIDFDLLEEAHSCIQKFFSMPLCERQRALRKAGESYGYASSFTGRFCSK
LPWKETLSFRYSSSSSDIVQNYFVRTLGEEFRHFGEVYQKYCESMSKLSLMIMEVLGLSLGVGR
MHFREFFEGNDSTMRLNYYPPCKKPDLTLGTGPHCDPTSLTILHQDDVSGLQVFTGGKWLTVRP
KTDAFVVNIGDTFTALSNGRYKSCLHRAVVNSKTARKSLAFFLCPAMNKIVRPPRELVDIDHPR
AYPDFTWSALLEFTQKHYRADMQTLNEFSKYILQAQGTLHK
Arabidopsis thaliana (AtF3H)
(SEQ ID NO: 106)
MAPGTLTELAGESKLNSKFVRDEDERPKVAYNVFSDEIPVISLAGIDDVDGKRGEICRQIVEAC
ENWGIFQVVDHGVDTNLVADMTRLARDFFALPPEDKLRFDMSGGKKGGFIVSSHLQGEAVQDWR
EIVTYFSYPVRNRDYSRWPDKPEGWVKVTEEYSERLMSLACKLLEVLSEAMGLEKESLTNACVD
MDQKIVVNYYPKCPQPDLTLGLKRHTDPGTTTLLLQDQVGGLQATRDNGKTWITVQPVEGAFVV
NLGDHGHFLSNGRFKNADHQAVVNSNSSRLSIATFQNPAPDATVYPLKVREGEKAILEEPITFA
EMYKRKMGRDLELARLKKLAKEERDHKEVDKPVDQIFA
Chrysosplenium americanum (CaF6H)
(SEQ ID NO: 107)
QEKTLNSRFVARDEDSLERPKVSAIYNGSFDEIPVLISLAGIDMTGAGTDAAARRSEICRKIVE
ACEDWGIFGEIDDDHGKRAEICDKIVKACEDWGVFQPDEKLESVMSAAKKGDFVVDHGVDAEVI
SQWTTFAKPTSHTQFETETTRDFPNKPEGWKATTEQYSRTLMGLACKLLGVISEAMGLEKEALT
KACVDMDQKVVVNYYPKCPQPDLTLGLKRHTDPGTITLLLQDQVGGLQATRDGGKTWITVQPVK
DNGWILLHIGDSNGHRHGHFLSNGRFKSHQAYRYRRPTRGSPTFGTKVSNYPPCPEQSLVRPPA
GRPYGRALNALDAKKLASAKQQLESAAILLISELAVAYIILAILPSSEIIAEEGYL
Datura stramonium (DsH6H)
(SEQ ID NO: 108)
MATFVSNWSTNNVSESFIAPLEKRAEKDVALGNDVPIIDLQQDHLLIVQQITKACQDFGLFQVI
NHGVPEKLMVEAMEVYKEFFALPAEEKEKFQPKGEPAKFELPLEQKAKLYVEGERRCNEEFLYW
KDTLAHGCYPLHEELLNSWPEKPPTYRDVIAKYSVEVRKLTMRILDYICEGLGLKLGYFDNELT
QIQMLLANYYPSCPDPSSTIGSGGHYDGNLITLLQQDLVGLQQLIVKDDRWIAVEPIPTAFVVN
LGLTLKVMSNEKFEGSIHRVVTHPTRNRISIGTLIGPDYSCTIEPIKELLSQENPPLYKPYPYA
KFAEIYLSDKSDYDAGVKPYKINQFPN
Arabidopsis thaliana (AtH6DH)
(SEQ ID NO: 109)
MENHTTMKVSSLNCIDLANDDLNHSVVSLKQACLDCGFFYVINHGISEEFMDDVFEQSKKLFAL
PLEEKMKVLRNEKHRGYTPVLDELLDPKNQINGDHKEGYYIGIEVPKDDPHWDKPFYGPNPWPD
ADVLPGWRETMEKYHQEALRVSMAIARLLALALDLDVGYFDRTEMLGKPIATMRLLRYQGISDP
SKGIYACGAHSDFGMMTLLATDGVMGLQICKDKNAMPQKWEYVPPIKGAFIVNLGDMLERWSNG
FFKSTLHRVLGNGQERYSIPFFVEPNHDCLVECLPTCKSESELPKYPPIKCSTYLTQRYEETHA
NLSIYHQQT
Solanum lycopersicum (SlF35H)
(SEQ ID NO: 110)
MALRINELFVAAIIYIIVHIIISKLITTVRERGRRLPLPPGPTGWPVIGALPLLGSMPHVALAK
MAKKYGPIMYLKVGTCGMVVASTPNAAKAFLKTLDINFSNRPPNAGATHLAYNAQDMVFAPYGP
RWKLLRKLSNLHMLGGKALENWANVRANELGHMLKSMFDASQDGECVVIADVLTFAMANMIGQV
MLSKRVFVEKGVEVNEFKNMVVELMTVAGYFNIGDFIPKLAWMDIQGIEKGMKNLHKKFDDLLT
KMFDEHFATSNERKENPDFLDVVMANRDNSEGERLSTTNIKALLLNLFTAGTDTSSSVIEWALA
EMMKNPKIFEKAQQEMDQVIGKNRRLIESDIPNLPYLRAICKETFRKHPSTPLNLPRVSSEPCT
VDGYYIPKNTRLSVNIWAIGRDPDVWENPLEFTPERFLSGKNAKIEPRGNDFELIPFGAGRRIC
AGTRMGIVMVEYILGTLVHSFDWKLPNNVIDINMEESFGLALQKAVPLEAMVTPRLSLDVYRC
D4H
(SEQ ID NO: 111)
MPKSWPIVISSHSFCFLPNSEQERKMKDLNFHAATLSEEESLRELKAFDETKAGVKGIVDTGIT
KIPRIFIDQPKNLDRISVCRGKSDIKIPVINLNGLSSNSEIRREIVEKIGEASEKYGFFQIVNH
GIPQDVMDEMVDGVRKFHEQDDQIKRQYYSRDRFNKNFLYSSNYVLIFGIACNWRDTMECIMNS
NQFDPQEFPDVCRDILMKYSNYVRNLGLILFELLSEALGLKPNHLEEMDCAEGLILLGHYYPAC
PQPELTFGTSKKSDSGFLTILKQDQIGGLQILLENQWIDVPFIPGALVINIADLLQLITNDKFK
SVEHRVLANKVGPRISVAVAFGIKTQTQEGVSPRLYGPIKELISEENPPIYKEVTVKDFITIRF
AKRFDDSSSLSPFRLNN
Catharanthus roseus (crD4Hlike)
(SEQ ID NO: 112)
MKELNNSEEELKAFDDTKAGVKALVDSGITEIPRIFLDHPTNLDQISSKDREPKFKKNIPVIDL
DGISTNSEIRREIVEKIREASEKWGFHQIVNHGIPQEVMDDMIVGIRRFHEQDNEIKKQFYTRD
RTKSFRYTSNFVLKPKIACNWRDTFECTMAPHQPNPQDLPDICRDIMMKYISYTRNLGLTLFEL
LSEALGLKSNRLKDMHCDEGVELVGHYYPACPQPELTLGTSKHTDTGFLTMLQQDQIGGLQVLY
ENHQWVDVPFIPGALIINIGDFLQIISNDKFKSAPHRVLANKNGPRISTASVFMPNFLESAEVR
LYGPIKELLSEENPPIYEQITAKDYVTVQFSRGLDGDSFLSPFMLNKDNMEK
Zea mays (ZmBX6)
(SEQ ID NO: 113)
MAPTTATKDDSGYGDERRRELQAFDDTKLGVKGLVDSGVKSIPSIFHHPPEALSDIISPAPLPS
SPPSGAAIPVVDLSVTRREDLVEQVRHAAGTVGFFWLVNHGVAEELMGGMLRGVRGFNEGPVEA
KQALYSRDLARNLRFASNFDLFKAAAADWRDTLFCEVAPNPPPREELPEPLRNVMLEYGAAVTK
LARFVFELLSESLGMPSDHLYEMECMQNLNVVCQYYPPCPEPHRTVGVKRHTDPGFFTILLQDG
MGGLQVRLGNNGQSGGCWVDIAPRPGALMVNIGDLLQLVTNDRFRSVEHRVPANKSSDTARVSV
ASFFNTDVRRSERMYGPIPDPSKPPLYRSVRARDFIAKFNTIGLDGRALDHFRL
Hordeum vulgare subsp. vulgare (HvIDS2)
(SEQ ID NO: 114)
MAKVMNLTPVHASSIPDSFLLPADRLHPATTDVSLPIIDMSRGRDEVRQAILDSGKEYGFIQVV
NHGISEPMLHEMYAVCHEFFDMPAEDKAEFFSEDRSERNKLFCGSAFETLGEKYWIDVLELLYP
LPSGDTKDWPHKPQMLREVVGNYTSLARGVAMEILRLLCEGLGLRPDFFVGDISGGRVVVDINY
YPPSPNPSRTLGLPPHCDRDLMTVLLPGAVPGLEIAYKGGWIKVQPVPNSLVINFGLQLEVVTN
GYLKAVEHRAATNFAEPRLSVASFIVPADDCVVGPAEEFVSEDNPPRYRTLTVGEFKRKHNVVN
LDSSINQIININNNQKGI
Hordeum vulgare subsp. vulgare (HvIDS3)
(SEQ ID NO: 115)
MENILHATPAPVSLPESFVFASDKVPPATKAVVSLPIIDLSCGRDEVRRSILEAGKELGFFQVV
NHGVSKQVMRDMEGMCEQFFHLPAADKASLYSEERHKPNRLFSGATYDTGGEKYWRDCLRLACP
FPVDDSINEWPDTPKGLRDVIEKFTSQTRDVGKELLRLLCEGMGIRADYFEGDLSGGNVILNIN
HYPSCPNPDKALGQPPHCDRNLITLLLPGAVNGLEVSYKGDWIKVDPAPNAFVVNFGQQLEVVT
NGLLKSIEHRAMTNSALARTSVATFIMPTQECLIGPAKEFLSKENPPCYRTTMFRDFMRIYNVV
KLGSSLNLTTNLKNVQKEI
Uridine diphosphate dependent glycosyltransferase (UGT)
Siraitia grosvenorii UGT720-269-1
(SEQ ID NO: 116)
MEDRNAMDMSRIKYEPQPLRPASMVQPRVLLFPFPALGHVKPFLSLAELLSDAGIDVVFLSTEY
NHRRISNTEALASRFPTLHFETIPDGLPPNESRALADGPLYFSMREGTKPRFRQLIQSLNDGRW
PITCIITDIMLSSPIEVAEEFGIPVIAFCPCSARYLSIHFFIPKLVEEGQIPYADDDPIGEIQG
VPLFEGLLRRNHLPGSWSDKSADISFSHGLINQTLAAGRASALILNTFDELEAPFLTHLSSIFN
KIYTIGPLHALSKSRLGDSSSSASALSGFWKEDRACMSWLDCQPPRSVVFVSFGSTMKMKADEL
REFWYGLVSSGKPFLCVLRSDVVSGGEAAELIEQMAEEEGAGGKLGMVVEWAAQEKVLSHPAVG
GFLTHCGWNSTVESIAAGVPMMCWPILGDQPSNATWIDRVWKIGVERNNREWDRLTVEKMVRAL
MEGQKRVEIQRSMEKLSKLANEKVVRGINLHPTISLKKDTPTTSEHPRHEFENMRGMNYEMLVG
NAIKSPTLTKK
Siraitia grosvenorii UGT94-289-3
(SEQ ID NO: 117)
MTIFFSVEILVLGLAEFAAIAMDAAQQGDTTTILMLPWLGYGHLSAFLELAKSLSRRNFHIYFC
STSVNLDAIKPKLPSSFSDSIQFVELHLPSSPEFPPHLHTTNGLPPTLMPALHQAFSMAAQHFE
SILQTLAPHLLIYDSLQPWAPRVASSLKIPAINFNTTGVFVISQGLHPIHYPHSKFPFSEFVLH
NHWKAMYSTADGASTERTRKRGEAFLYCLHASCSVILINSFRELEGKYMDYLSVLLNKKVVPVG
PLVYEPNQDGEDEGYSSIKNWLDKKEPSSTVFVSFGSEYFPSKEEMEEIAHGLEASEVNFIWVV
RFPQGDNTSGIEDALPKGFLERAGERGMVVKGWAPQAKILKHWSTGGFVSHCGWNSVMESMMFG
VPIIGVPMHVDQPFNAGLVEEAGVGVEAKRDPDGKIQRDEVAKLIKEVVVEKTREDVRKKAREM
SEILRSKGEEKFDEMVAEISLLLKI
Siraitia grosvenorii UGT74-345-2
(SEQ ID NO: 118)
MDETTVNGGRRASDVVVFAFPRHGHMSPMLQFSKRLVSKGLRVTFLITTSATESLRLNLPPSSS
LDLQVISDVPESNDIATLEGYLRSFKATVSKTLADFIDGIGNPPKFIVYDSVMPWVQEVARGRG
LDAAPFFTQSSAVNHILNHVYGGSLSIPAPENTAVSLPSMPVLQAEDLPAFPDDPEVVMNFMTS
QFSNFQDAKWIFFNTFDQLECKKQSQVVNWMADRWPIKTVGPTIPSAYLDDGRLEDDRAFGLNL
LKPEDGKNTRQWQWLDSKDTASVLYISFGSLAILQEEQVKELAYFLKDTNLSFLWVLRDSELQK
LPHNFVQETSHRGLVVNWCSQLQVLSHRAVSCFVTHCGWNSTLEALSLGVPMVAIPQWVDQTTN
AKFVADVWRVGVRVKKKDERIVTKEELEASIRQVVQGEGRNEFKHNAIKWKKLAKEAVDEGGSS
DKNIEEFVKTIA
Siraitia grosvenorii UGT75-281-2
(SEQ ID NO: 119)
MGDNGDGGEKKELKENVKKGKELGRQAIGEGYINPSLQLARRLISLGVNVTFATTVLAGRRMKN
KTHQTATTPGLSFATFSDGFDDETLKPNGDLTHYFSELRRCGSESLTHLITSAANEGRPITFVI
YSLLLSWAADIASTYDIPSALFFAQPATVLALYFYYFHGYGDTICSKLQDPSSYIELPGLPLLT
SQDMPSFFSPSGPHAFILPPMREQAEFLGRQSQPKVLVNTFDALEADALRAIDKLKMLAIGPLI
PSALLGGNDSSDASFCGDLFQVSSEDYIEWLNSKPDSSVVYISVGSICVLSDEQEDELVHALLN
SGHTFLWVKRSKENNEGVKQETDEEKLKKLEEQGKMVSWCRQVEVLKHPALGCFLTHCGWNSTI
ESLVSGLPVVAFPQQIDQATNAKLIEDVWKTGVRVKANTEGIVEREEIRRCLDLVMGSRDGQKE
EIERNAKKWKELARQAIGEGGSSDSNLKTFLWEIDLEI
Siraitia grosvenorii UGT720-269-4
(SEQ ID NO: 120)
MAEQAHDLLHVLLFPFPAEGHIKPFLCLAELLCNAGFHVTFLNTDYNHRRLHNLHLLAARFPSL
HFESISDGLPPDQPRDILDPKFFISICQVTKPLFRELLLSYKRISSVQTGRPPITCVITDVIFR
FPIDVAEELDIPVFSFCTFSARFMFLYFWIPKLIEDGQLPYPNGNINQKLYGVAPEAEGLLRCK
DLPGHWAFADELKDDQLNFVDQTTASSRSSGLILNTFDDLEAPFLGRLSTIFKKIYAVGPIHSL
LNSHHCCLWKEDHSCLAWLDSRAAKSVVFVSFGSLVKITSRQLMEFWHGLLNSGKSFLFVLRSD
VVEGDDEKQVVKEIYETKAEGKWLVVGWAPQEKVLAHEAVGGFLTHSGWNSILESIAAGVPMIS
CPKIGDQSSNCTWISKVWKIGLEMEDRYDRVSVETMVRSIMEQEGEKMQKTIAELAKQAKYKVS
KDGTSYQNLECLIQDIKKLNQIEGFINNPNFSDLLRV
Siraitia grosvenorii UGT94-289-2
(SEQ ID NO: 121)
MDAQQGHTTTILMLPWVGYGHLLPFLELAKSLSRRKLFHIYFCSTSVSLDAIKPKLPPSISSDD
SIQLVELRLPSSPELPPHLHTTNGLPSHLMPALHQAFVMAAQHFQVILQTLAPHLLIYDILQPW
APQVASSLNIPAINFSTTGASMLSRTLHPTHYPSSKFPISEEVLHNHWRAMYTTADGALTEEGH
KIEETLANCLHTSCGVVLVNSFRELETKYIDYLSVLLNKKVVPVGPLVYEPNQEGEDEGYSSIK
NWLDKKEPSSTVFVSFGTEYFPSKEEMEEIAYGLELSEVNFIWVLRFPQGDSTSTIEDALPKGF
LERAGERAMVVKGWAPQAKILKHWSTGGLVSHCGWNSMMEGMMFGVPIIAVPMHLDQPFNAGLV
EEAGVGVEAKRDSDGKIQREEVAKSIKEVVIEKTREDVRKKAREMDTKHGPTYFSRSKVSSFGR
LYKINRPTTLTVGRFWSKQIKMKRE
Siraitia grosvenorii UGT94-289-1
(SEQ ID NO: 122)
MDAQRGHTTTILMFPWLGYGHLSAFLELAKSLSRRNFHIYFCSTSVNLDAIKPKLPSSSSSDSI
QLVELCLPSSPDQLPPHLHTTNALPPHLMPTLHQAFSMAAQHFAAILHTLAPHLLIYDSFQPWA
PQLASSLNIPAINFNTTGASVLTRMLHATHYPSSKFPISEFVLHDYWKAMYSAAGGAVTKKDHK
IGETLANCLHASCSVILINSFRELEEKYMDYLSVLLNKKVVPVGPLVYEPNQDGEDEGYSSIKN
WLDKKEPSSTVFVSFGSEYFPSKEEMEEIAHGLEASEVHFIWVVRFPQGDNTSAIEDALPKGFL
ERVGERGMVVKGWAPQAKILKHWSTGGFVSHCGWNSVMESMMFGVPIIGVPMHLDQPFNAGLAE
EAGVGVEAKRDPDGKIQRDEVAKLIKEVVVEKTREDVRKKAREMSEILRSKGEEKMDEMVAAIS
LFLKI
Momordica charantia 1 (McUGT1)
(SEQ ID NO: 123)
MAQPQTQARVLVFPYPTVGHIKPFLSLAELLADGGLDVVFLSTEYNHRRIPNLEALASRFPTLH
FDTIPDGLPIDKPRVIIGGELYTSMRDGVKQRLRQVLQSYNDGSSPITCVICDVMLSGPIEAAE
ELGIPVVTFCPYSARYLCAHFVMPKLIEEGQIPFTDGNLAGEIQGVPLFGGLLRRDHLPGFWFV
KSLSDEVWSHAFLNQTLAVGRTSALIINTLDELEAPFLAHLSSTFDKIYPIGPLDALSKSRLGD
SSSSSTVLTAFWKEDQACMSWLDSQPPKSVIFVSFGSTMRMTADKLVEFNHGLVNSGTRFLCVL
RSDIVEGGGAADLIKQVGETGNGIVVEWAAQEKVLAHRAVGGFLTHCGWNSTMESIAAGVPMMC
WQIYGDQMINATWIGKVWKIGIERDDKWDRSTVEKMIKELMEGEKGAEIQRSMEKFSKLANDKV
VKGGTSFENLELIVEYLKKLKPSN
Momordica charantia 2 (McUGT2)
(SEQ ID NO: 124)
MAQPRVLLFPFPAMGHVKPFLSLAELLSDAGVEVVFLSTEYNHRRIPDIGALAARFPTLHFETI
PDGLPPDQPRVLADGHLYFSMLDGTKPRFRQLIQSLNGNPRPITCIINDVMLSSPIEVAEEFGI
PVIAFCPCSARFLSVHFFMPNFIEEAQIPYTDENPMGKIEEATVFEGLLRRKDLPGLWCAKSSN
ISFSHRFINQTIAAGRASALILNTFDELESPFLNHLSSIFPKIYCIGPLNALSRSRLGKSSSSS
SALAGFWKEDQAYMSWLESQPPRSVIFVSFGSTMKMEAWKLAEFWYGLVNSGSPFLFVFRPDCV
INSGDAAEVMEGRGRGMVVEWASQEKVLAHPAVGGFLTHCGWNSTVESIVAGVPMMCCPIVADQ
LSNATWIHKVWKIGIEGDEKWDRSTVEMMIKELMESQKGTEIRTSIEMLSKLANEKVVKGGTSL
NNFELLVEDIKTLRRPYT
Momordica charantia 3 (McUGT3)
(SEQ ID NO: 125)
MEQSDSNSDDHQHHVLLFPFPAKGHIKPFLCLAQLLCGAGLQVTFLNTDHNHRRIDDRHRRLLA
TQFPMLHFKSISDGLPPDHPRDLLDGKLIASMRRVTESLFRQLLLSYNGYGNGTNNVSNSGRRP
PISCVITDVIFSFPVEVAEELGIPVFSFATFSARFLFLYFWIPKLIQEGQLPFPDGKTNQELYG
VPGAEGIIRCKDLPGSWSVEAVAKNDPMNFVKQTLASSRSSGLILNTFEDLEAPFVTHLSNTFD
KIYTIGPIHSLLGTSHCGLWKEDYACLAWLDARPRKSVVFVSFGSLVKTTSRELMELWHGLVSS
GKSFLLVLRSDVVEGEDEEQVVKEILESNGEGKWLVVGWAPQEEVLAHEAIGGFLTHSGWNSTM
ESIAAGVPMVCWPKIGDQPSNCTWVSRVWKVGLEMEERYDRSTVARMARSMMEQEGKEMERRIA
ELAKRVKYRVGKDGESYRNLESLIRDIKITKSSN
Momordica charantia 4 (McUGT4)
(SEQ ID NO: 126)
MDAHQQAEHTTTILMLPWVGYGHLTAYLELAKALSRRNFHIYYCSTPVNIESIKPKLTIPCSSI
QFVELHLPSSDDLPPNLHTTNGLPSHLMPTLHQAFSAAAPLFEEILQTLCPHLLIYDSLQPWAP
KIASSLKIPALNFNTSGVSVIAQALHAIHHPDSKFPLSDFILHNYWKSTYTTADGGASEKTRRA
REAFLYCLNSSGNAILINTFRELEGEYIDYLSLLLNKKVIPIGPLVYEPNQDEDQDEEYRSIKN
WLDKKEPCSTVFVSFGSEYFPSNEEMEEIAPGLEESGANFIWVVRFPKLENRNGIIEEGLLERA
GERGMVIKEWAPQARILRHGSIGGFVSHCGWNSVMESIICGVPVIGVPMRVDQPYNAGLVEEAG
VGVEAKRDPDGKIQRHEVSKLIKQVVVEKTRDDVRKKVAQMSEILRRKGDEKIDEMVALISLLP
KG
Momordica charantia 5 (McUGT5)
(SEQ ID NO: 127)
MDARQQAEHTTTILMLPWVGYGHLSAYLELAKALSRRNFHIYYCSTPVNIESIKPKLTIPCSSI
QFVELHLPFSDDLPPNLHTTNGLPSHLMPALHQAFSAAAPLFEAILQTLCPHLLIYDSLQPWAP
QIASSLKIPALNFNTTGVSVIARALHTIHHPDSKFPLSEIVLHNYWKATHATADGANPEKFRRD
LEALLCCLHSSCNAILINTFRELEGEYIDYLSLLLNKKVTPIGPLVYEPNQDEEQDEEYRSIKN
WLDKKEPYSTIFVSFGSEYFPSNEEMEEIARGLEESGANFIWVVRFHKLENGNGITEEGLLERA
GERGMVIQGWAPQARILRHGSIGGFVSHCGWNSVMESIICGVPVIGVPMGLDQPYNAGLVEEAG
VGVEAKRDPDGKIQRHEVSKLIKQVVVEKTRDDVRKKVAQMSEILRRKGDEKIDEMVALISLLL
KG
Cucumis sativus
(SEQ ID NO: 128)
MGLSPTDHVLLFPFPAKGHIKPFFCLAHLLCNAGLRVTFLSTEHHHQKLHNLTHLAAQIPSLHF
QSISDGLSLDHPRNLLDGQLFKSMPQVTKPLFRQLLLSYKDGTSPITCVITDLILRFPMDVAQE
LDIPVFCFSTFSARFLFLYFSIPKLLEDGQIPYPEGNSNQVLHGIPGAEGLLRCKDLPGYWSVE
AVANYNPMNFVNQTIATSKSHGLILNTFDELEVPFITNLSKIYKKVYTIGPIHSLLKKSVQTQY
EFWKEDHSCLAWLDSQPPRSVMFVSFGSIVKLKSSQLKEFWNGLVDSGKAFLLVLRSDALVEET
GEEDEKQKELVIKEIMETKEEGRWVIVNWAPQEKVLEHKAIGGFLTHSGWNSTLESVAVGVPMV
SWPQIGDQPSNATWLSKVWKIGVEMEDSYDRSTVESKVRSIMEHEDKKMENAIVELAKRVDDRV
SKEGTSYQNLQRLIEDIEGFKLN
Cucurbita maxima 1 (CmaUGT1)
(SEQ ID NO: 129)
MELSHTHHVLLFPFPAKGHIKPFFSLAQLLCNAGLRVTFLNTDHHHRRIHDLNRLAAQLPTLHF
DSVSDGLPPDEPRNVFDGKLYESIRQVTSSLFRELLVSYNNGTSSGRPPITCVITDVMFRFPID
IAEELGIPVFTFSTFSARFLFLIFWIPKLLEDGQLRYPEQELHGVPGAEGLIRWKDLPGFWSVE
DVADWDPMNFVNQTLATSRSSGLILNTFDELEAPFLTSLSKIYKKIYSLGPINSLLKNFQSQPQ
YNLWKEDHSCMAWLDSQPRKSVVFVSFGSVVKLTSRQLMEFWNGLVNSGMPFLLVLRSDVIEAG
EEVVREIMERKAEGRWVIVSWAPQEEVLAHDAVGGFLTHSGWNSTLESLAAGVPMISWPQIGDQ
TSNSTWISKVWRIGLQLEDGFDSSTIETMVRSIMDQTMEKTVAELAERAKNRASKNGTSYRNFQ
TLIQDITNIIETHI
Cucurbita maxima 2 (CmaUGT2)
(SEQ ID NO: 130)
MDAQKAVDTPPTTVLMLPWIGYGHLSAYLELAKALSRRNFHVYFCSTPVNLDSIKPNLIPPPSS
IQFVDLHLPSSPELPPHLHTTNGLPSHLKPTLHQAFSAAAQHFEAILQTLSPHLLIYDSLQPWA
PRIASSLNIPAINFNTTAVSIIAHALHSVHYPDSKFPFSDFVLHDYWKAKYTTADGATSEKIRR
GAEAFLYCLNASCDVVLVNSFRELEGEYMDYLSVLLKKKVVSVGPLVYEPSEGEEDEEYWRIKK
WLDEKEALSTVLVSFGSEYFPSKEEMEEIAHGLEESEANFIWVVRFPKGEESCRGIEEALPKGF
VERAGERAMVVKKWAPQGKILKHGSIGGFVSHCGWNSVLESIRFGVPVIGVPMHLDQPYNAGLL
EEAGIGVEAKRDADGKIQRDQVASLIKRVVVEKTREDIWKTVREMREVLRRRDDDMIDEMVAEI
SVVLKI
Cucurbita maxima 3 (CmaUGT3)
(SEQ ID NO: 131)
MSSNLFLKISIPFGRLRDSALNCSVFHCKLHLAIAIAMDAQQAANKSPTATTIFMLPWAGYGHL
SAYLELAKALSTRNFHIYFCSTPVSLASIKPRLIPSCSSIQFVELHLPSSDEFPPHLHTTNGLP
SRLVPTFHQAFSEAAQTFEAFLQTLRPHLLIYDSLQPWAPRIASSLNIPAINFFTAGAFAVSHV
LRAFHYPDSQFPSSDFVLHSRWKIKNTTAESPTQAKLPKIGEAIGYCLNASRGVILTNSFRELE
GKYIDYLSVILKKRVFPIGPLVYQPNQDEEDEDYSRIKNWLDRKEASSTVLVSFGSEFFLSKEE
TEAIAHGLEQSEANFIWGIRFPKGAKKNAIEEALPEGFLERAGGRAMVVEEWVPQGKILKHGSI
GGFVSHCGWNSAMESIVCGVPIIGIPMQVDQPFNAGILEEAGVGVEAKRDSDGKIQRDEVAKLI
KEVVVERTREDIRNKLEKINEILRSRREEKLDELATEISLLSRN
Cucurbita moschata 1 (CmoUGT1)
(SEQ ID NO: 132)
MELSPTHHLLLFPFPAKGHIKPFFSLAQLLCNAGARVTFLNTDHHHRRIHDLDRLAAQLPTLHF
DSVSDGLPPDESRNVFDGKLYESIRQVTSSLFRELLVSYNNGTSSGRPPITCVITDCMFRFPID
IAEELGIPVFTFSTFSARFLFLFFWIPKLLEDGQLRYPEQELHGVPGAEGLIRCKDLPGFLSDE
DVAHWKPINFVNQILATSRSSGLILNTFDELEAPFLTSLSKIYKKIYSLGPINSLLKNFQSQPQ
YNLWKEDHSCMAWLDSQPPKSVVFVSFGSVVKLTNRQLVEFWNGLVNSGKPFLLVLRSDVIEAG
EEVVRENMERKAEGRWMIVSWAPQEEVLAHDAVGGFLTHSGWNSTLESLAAGVPMISWTQIGDQ
TSNSTWVSKVWRIGLQLEDGFDSFTIETMVRSVMDQTMEKTVAELAERAKNRASKNGTSYRNFQ
TLIQDITNIIETHI
Cucurbita moschata 2 (CmoUGT2)
(SEQ ID NO: 133)
MDAQKAVDTPPTTVLMLPWIGYGHLSAYLELAKALSRRNFHVYFCSTPVNLDSIKPNLIPPPPS
IQFVDLHLPSSPELPPHLHTTNGLPSHLKPTLHQAFSAAAQHFEAILQTLSPHLLIYDSLQPWA
PRIASSLNIPAINFNTTAVSIIAHALHSVHYPDSKFPFSDFVLHDYWKAKYTTADGATSEKTRR
GVEAFLYCLNASCDVVLVNSFRELEGEYMDYLSVLLKKKVVSVGPLVYEPSEGEEDEEYWRIKK
WLDEKEALSTVLVSFGSEYFPPKEEMEEIAHGLEESEANFIWVVRFPKGEESSSRGIEEALPKG
FVERAGERAMVVKKWAPQGKILKHGSIGGFVSHCGWNSVLESIRFGVPVIGAPMHLDQPYNAGL
LEEAGIGVEAKRDADGKIQRDQVASLIKQVVVEKTREDTWKKVREMREVLRRRDDDDMMIDEMV
AVISVVLKI
Cucurbita moschata 3 (CmoUGT3)
(SEQ ID NO: 134)
MDAQQAANKSPTASTIFMLPWVGYGHLSAYLELAKALSTRNFHVYFCSTPVSLASIKPRLIPSC
SSIQFVELHLPSSDEFPPHLHTTNGLPAHLVPTIHQAFAAAAQTFEAFLQTLRPHLLIYDSLQP
WAPRIASSLNIPAINFFTAGAFAVSHVLRAFHYPDSQFPSSDFVLHSRWKIKNTTAESPTQVKI
PKIGEAIGYCLNASRGVILTNSFRELEGKYIDYLSVILKKRVLPIGPLVYQPNQDEEDEDYSRI
KNWLDRKEASSTVLVSFGSEFFLSKEETEAIAHGLEQSEANFIWGIRFPKGAKKNAIEEALPEG
FLERVGGRAMVVEEWVPQGKILKHGNIGGFVSHCGWNSAMESIMCGVPVIGIPMQVDQPFNAGI
LEEAGVGVEAKRDSDGKIQRDEVAKLIKEVVVERTREDIRNKLEEINEILRTRREEKLDELATE
ISLLCKN
Prunus persica
(SEQ ID NO: 135)
MAMKQPHVIIFPFPLQGHMKPLLCLAELLCHAGLHVTYVNTHHNHQRLANRQALSTHFPTLHFE
SISDGLPEDDPRTLNSQLLIALKTSIRPHFRELLKTISLKAESNDTLVPPPSCIMTDGLVTFAF
DVAEELGLPILSFNVPCPRYLWTCLCLPKLIENGQLPFQDDDMNVEITGVPGMEGLLHRQDLPG
FCRVKQADHPSLQFAINETQTLKRASALILDTVYELDAPCISHMALMFPKIYTLGPLHALLNSQ
IGDMSRGLASHGSLWKSDLNCMTWLDSQPSKSIIYVSFGTLVHLTRAQVIEFWYGLVNSGHPFL
WVMRSDITSGDHQIPAELENGTKERGCIVDWVSQEEVLAHKSVGGFLTHSGWNSTLESIVAGLP
MICWPKLGDHYIISSTVCRQWKIGLQLNENCDRSNIESMVQTLMGSKREEIQSSMDAISKLSRD
SVAEGGSSHNNLEQLIEYIRNLQHQN
Theobroma cacao
(SEQ ID NO: 136)
MRQPHVLVLPFPAQGHIKPMLCLAELLCQAGLRVTFLNTHHSHRRLNNLQDLSTRFPTLHFESV
SDGLPEDHPRNLVHFMHLVHSIKNVTKPLLRDLLTSLSLKTDIPPVSCIIADGILSFAIDVAEE
LQIKVIIFRTISSCCLWSYLCVPKLIQQGELQFSDSDMGQKVSSVPEMKGSLRLHDRPYSFGLK
QLEDPNFQFFVSETQAMTRASAVIFNTFDSLEAPVLSQMIPLLPKVYTIGPLHALRKARLGDLS
QHSSFNGNLREADHNCITWLDSQPLRSVVYVSFGSHVVLTSEELLEFWHGLVNSGKRFLWVLRP
DIIAGEKDHNQIIAREPDLGTKEKGLLVDWAPQEEVLAHPSVGGFLTHCGWNSTLESMVAGVPM
LCWPKLPDQLVNSSCVSEVWKIGLDLKDMCDRSTVEKMVRALMEDRREEVMRSVDGISKLARES
VSHGGSSSSNLEMLIQELET
Corchorus capsularis
(SEQ ID NO: 137)
MDSKQKKMSVLMFPWLAYGHISPFLELAKKLSKRNFHTFFFSTPINLNSIKSKLSPKYAQSIQF
VELHLPSLPDLPPHYHTTNGLPPHLMNTLKKAFDMSSLQFSKILKTLNPDLLVYDFIQPWAPLL
ALSNKIPAVHFLCTSAAMSSFSVHAFKKPCEDFPFPNIYVHGNFMNAKFNNMENCSSDDSISDQ
DRVLQCFERSTKIILVKTFEELEGKFMDYLSVLLNKKIVPTGPLTQDPNEDEGDDDERTKLLLE
WLNKKSKSSTVFVSFGSEYFLSKEEREEIAYGLELSKVNFIWVIRFPLGENKTNLEEALPQGFL
QRVSERGLVVENWAPQAKILQHSSIGGFVSHCGWSSVMESLKFGVPIIAIPMHLDQPLNARLVV
DVGVGLEVIRNHGSLEREEIAKLIKEVVLGNGNDGEIVRRKAREMSNHIKKKGEKDMDELVEEL
MLICKMKPNSCHLS
Ziziphus jujube
(SEQ ID NO: 138)
MMERQRSIKVLMFPWLAHGHISPFLELAKRLTDRNFQIYFCSTPVNLTSVKPKLSQKYSSSIKL
VELHLPSLPDLPPHYHTTNGLALNLIPTLKKAFDMSSSSFSTILSTIKPDLLIYDFLQPWAPQL
ASCMNIPAVNFLSAGASMVSFVLHSIKYNGDDHDDEFLTTELHLSDSMEAKFAEMTESSPDEHI
DRAVTCLERSNSLILIKSFRELEGKYLDYLSLSFAKKVVPIGPLVAQDTNPEDDSMDIINWLDK
KEKSSTVFVSFGSEYYLTNEEMEEIAYGLELSKVNFIWVVRFPLGQKMAVEEALPKGFLERVGE
KGMVVEDWAPQMKILGHSSIGGFVSHCGWSSLMESLKLGVPIIAMPMQLDQPINAKLVERSGVG
LEVKRDKNGRIEREYLAKVIREIVVEKARQDIEKKAREMSNIITEKGEEEIDNVVEELAKLCGM
Vitis vinifera
(SEQ ID NO: 139)
MDARQSDGISVLMFPWLAHGHISPFLQLAKKLSKRNFSIYFCSTPVNLDPIKGKLSESYSLSIQ
LVKLHLPSLPELPPQYHTTNGLPPHLMPTLKMAFDMASPNFSNILKTLHPDLLIYDFLQPWAPA
AASSLNIPAVQFLSTGATLQSFLAHRHRKPGIEFPFQEIHLPDYEIGRLNRFLEPSAGRISDRD
RANQCLERSSRFSLIKTFREIEAKYLDYVSDLTKKKMVTVGPLLQDPEDEDEATDIVEWLNKKC
EASAVFVSFGSEYFVSKEEMEEIAHGLELSNVDFIWVVRFPMGEKIRLEDALPPGFLHRLGDRG
MVVEGWAPQRKILGHSSIGGFVSHCGWSSVMEGMKFGVPIIAMPMHLDQPINAKLVEAVGVGRE
VKRDENRKLEREEIAKVIKEVVGEKNGENVRRKARELSETLRKKGDEEIDVVVEELKQLCSY
Juglans regia
(SEQ ID NO: 140)
MDTARKRIRVVMLPWLAHGHISPFLELSKKLAKRNFHIYFCSTPVNLSSIKPKLSGKYSRSIQL
VELHLPSLPELPPQYHTTKGLPPHLNATLKRAFDMAGPHFSNILKTLSPDLLIYDFLQPWAPAI
AASQNTPAINFLSTGAAMTSFVLHAMKKPGDEFPFPEIHLDECMKTRFVDLPEDHSPSDDHNHI
SDKDRALKCFERSSGFVMMKTFEELEGKYINFLSHLMQKKIVPVGPLVQNPVRGDHEKAKTLEW
LDKRKQSSAVFVSFGTEYFLSKEEMEEIAYGLELSNVNFIWVVRFPEGEKVKLEEALPEGFLQR
VGEKGMVVEGWAPQAKILMHPSIGGFVSHCGWSSVMESIDFGVPIVAIPMQLDQPVNAKVVEQA
GVGVEVKRDRDGKLEREEVATVIREVVMGNIGESVRKKEREMRDNIRKKGEEKMDGVAQELVQL
YGNGIKNV
Hevea brasiliensis
(SEQ ID NO: 141)
METLQRRKISVLMFPWLAHGHLSPFLELSKKLNKRNFHVYFCSTPVNLDSIKPKLSAEYSFSIQ
LVELHLPSSPELPLHYHTTNGLPPHLMKNLKNAFDMASSSFFNILKTLKPDLLIYDFIQPWAPA
LASSLNIPAVNFLCTSMAMSCFGLHLNNQEAKFPFPGIYPRDYMRMKVFGALESSSNDIKDGER
AGRCMDQSFHLILAKTFRELEGKYIDYLSVKLMKKIVPVGPLVQDPIFEDDEKIMDHHQVIKWL
EKKERLSTVFVSFGTEYFLSTEEMEEIAYGLELSKAHFIWVVRFPTGEKINLEESLPKRYLERV
QERGKIVEGWAPQQKILRHSSIGGFVSHCGWSSIMESMKFGVPIIAMPMNLDQPVNSRIVEDAG
VGIEVRRNKSGELEREEIAKTIRKVVVEKDGKNVSRKAREMSDTIRKKGEEEIDGVVDELLQLC
DVKTNYLQ
Manihot esculenta
(SEQ ID NO: 142)
MATAQTRKISVLMFPWLAHGHLSPFLELSKKLANRNFHVYFCSTPVNLDSIKPKLSPEYHFSIQ
FVELHLPSSPELPSHYHTTNGLPPHLMKTLKKAFDMASSSFFNILKTLNPDLLIYDFLQPWAPA
LASSLNIPAVNFLCSSMAMSCFGLNLNKNKEIKFLFPEIYPRDYMEMKLFRVFESSSNQIKDGE
RAGRCIDQSFHVILAKTFRELEGKYIDYVSVKCNKKIVPVGPLVEDTIHEDDEKTMDHHHHHHD
EVIKWLEKKERSTTVFVSFGSEYFLSKEEMEEIAHGLELSKVNFIWVVRFPKGEKINLEESLPE
GYLERIQERGKIVEGWAPQRKILGHSSIGGFVSHCGWSSIMESMKLGVPIIAMPMNLDQPINSR
IVEAAGVGIEVSRNQSGELEREEMAKTIRKVVVEREGVYVRRKAREMSDVLRKKGEEEIDGVVD
ELVQLCDMKTNYL
Cephalotus follicularis
(SEQ ID NO: 143)
MDLKRRSIRVLMLPWLAHGHISPFLELAKKLTNRNFLIYFCSTPINLNSIKPKLSSKYSFSIQL
VELHLPSLPELPPHYHTTNGLPLHLMNTLKTAFDMASPSFLNILKTLKPDLLICDHLQPWAPSL
ASSLNIPAIIFPTNSAIMMAFSLHHAKNPGEEFPFPSININDDMVKSINFLHSASNGLTDMDRV
LQCLERSSNTMLLKTFRQLEAKYVDYSSALLKKKIVLAGPLVQVPDNEDEKIEIIKWLDSRGQS
STVFVSFGSEYFLSKEEREDIAHGLELSKVNFIWVVRFPVGEKVKLEEALPNGFAERIGERGLV
VEGWAPQAMILSHSSIGGFVSHCGWSSMMESMKFGVPIIAMPMHIDQPLNARLVEDVGVGLEIK
RNKDGRFEREELARVIKEVLVYKNGDAVRSKAREMSEHIKKNGDQEIDGVADALVKLCEMKTNS
LNQD
Stevia rebaudiana UGT74G1
(SEQ ID NO: 144)
MAEQQKIKKSPHVLLIPFPLQGHINPFIQFGKRLISKGVKTTLVTTIHTLNSTLNHSNTTTTSI
EIQAISDGCDEGGFMSAGESYLETFKQVGSKSLADLIKKLQSEGTTIDAIIYDSMTEWVLDVAI
EFGIDGGSFFTQACVVNSLYYHVHKGLISLPLGETVSVPGFPVLQRWETPLILQNHEQIQSPWS
QMLFGQFANIDQARWVFTNSFYKLEEEVIEWTRKIWNLKVIGPTLPSMYLDKRLDDDKDNGFNL
YKANHHECMNWLDDKPKESVVYVAFGSLVKHGPEQVEEITRALIDSDVNFLWVIKHKEEGKLPE
NLSEVIKTGKGLIVAWCKQLDVLAHESVGCFVTHCGFNSTLEAISLGVPVVAMPQFSDQTTNAK
LLDEILGVGVRVKADENGIVRRGNLASCIKMIMEEERGVIIRKNAVKWKDLAKVAVHEGGSSDN
DIVEFVSELIKA
Stevia rebaudiana UGT76G1
(SEQ ID NO: 145)
MENKTETTVRRRRRIILFPVPFQGHINPILQLANVLYSKGFSITIFHTNFNKPKTSNYPHFTFR
FILDNDPQDERISNLPTHGPLAGMRIPIINEHGADELRRELELLMLASEEDEEVSCLITDALWY
FAQSVADSLNLRRLVLMTSSLFNFHAHVSLPQFDELGYLDPDDKTRLEEQASGFPMLKVKDIKS
AYSNWQILKEILGKMIKQTKASSGVIWNSFKELEESELETVIREIPAPSFLIPLPKHLTASSSS
LLDHDRTVFQWLDQQPPSSVLYVSFGSTSEVDEKDFLEIARGLVDSKQSFLWVVRPGFVKGSTW
VEPLPDGFLGERGRIVKWVPQQEVLAHGAIGAFWTHSGWNSTLESVCEGVPMIFSDFGLDQPLN
ARYMSDVLKVGVYLENGWERGEIANAIRRVMVDEEGEYIRQNARVLKQKADVSLMKGGSSYESL
ESLVSYISSL
Stevia rebaudiana UGT85C2
(SEQ ID NO: 146)
MDAMATTEKKPHVIFIPFPAQSHIKAMLKLAQLLHHKGLQITFVNTDFIHNQFLESSGPHCLDG
APGFRFETIPDGVSHSPEASIPIRESLLRSIETNFLDRFIDLVTKLPDPPTCIISDGFLSVFTI
DAAKKLGIPVMMYWTLAACGFMGFYHIHSLIEKGFAPLKDASYLTNGYLDTVIDWVPGMEGIRL
KDFPLDWSTDLNDKVLMFTTEAPQRSHKVSHHIFHTFDELEPSIIKTLSLRYNHIYTIGPLQLL
LDQIPEEKKQTGITSLHGYSLVKEEPECFQWLQSKEPNSVVYVNFGSTTVMSLEDMTEFGWGLA
NSNHYFLWIIRSNLVIGENAVLPPELEEHIKKRGFIASWCSQEKVLKHPSVGGFLTHCGWGSTI
ESLSAGVPMICWPYSWDQLTNCRYICKEWEVGLEMGTKVKRDEVKRLVQELMGEGGHKMRNKAK
DWKEKARIAIAPNGSSSLNIDKMVKEITVLARN
Stevia rebaudiana UGT91D1
(SEQ ID NO: 147)
MYNVTYHQNSKAMATSDSIVDDRKQLHVATFPWLAFGHILPFLQLSKLIAEKGHKVSFLSTTRN
IQRLSSHISPLINVVQLTLPRVQELPEDAEATTDVHPEDIQYLKKAVDGLQPEVTRFLEQHSPD
WIIYDFTHYWLPSIAASLGISRAYFCVITPWTIAYLAPSSDAMINDSDGRTTVEDLTTPPKWFP
FPTKVCWRKHDLARMEPYEAPGISDGYRMGMVFKGSDCLLFKCYHEFGTQWLPLLETLHQVPVV
PVGLLPPEIPGDEKDETWVSIKKWLDGKQKGSVVYVALGSEALVSQTEVVELALGLELSGLPFV
WAYRKPKGPAKSDSVELPDGFVERTRDRGLVWTSWAPQLRILSHESVCGFLTHCGSGSIVEGLM
FGHPLIMLPIFCDQPLNARLLEDKQVGIEIPRNEEDGCLTKESVARSLRSVVVENEGEIYKANA
RALSKIYNDTKVEKEYVSQFVDYLEKNARAVAIDHES
Stevia rebaudiana UGT91D2
(SEQ ID NO: 148)
MATSDSIVDDRKQLHVATFPWLAFGHILPYLQLSKLIAEKGHKVSFLSTTRNIQRLSSHISPLI
NVVQLTLPRVQELPEDAEATTDVHPEDIPYLKKASDGLQPEVTRFLEQHSPDWIIYDYTHYWLP
SIAASLGISRAHFSVTTPWAIAYMGPSADAMINGSDGRTTVEDLTTPPKWFPFPTKVCWRKHDL
ARLVPYKAPGISDGYRMGLVLKGSDCLLSKCYHEFGTQWLPLLETLHQVPVVPVGLLPPEVPGD
EKDETWVSIKKWLDGKQKGSVVYVALGSEVLVSQTEVVELALGLELSGLPFVWAYRKPKGPAKS
DSVELPDGFVERTRDRGLVWTSWAPQLRILSHESVCGFLTHCGSGSIVEGLMFGHPLIMLPIFG
DQPLNARLLEDKQVGIEIPRNEEDGCLTKESVARSLRSVVVEKEGEIYKANARELSKIYNDTKV
EKEYVSQFVDYLEKNTRAVAIDHES
Stevia rebaudiana UGT91D2e
(SEQ ID NO: 149)
MATSDSIVDDRKQLHVATFPWLAFGHILPYLQLSKLIAEKGHKVSFLSTTRNIQRLSSHISPLI
NVVQLTLPRVQELPEDAEATTDVHPEDIPYLKKASDGLQPEVTRFLEQHSPDWIIYDYTHYWLP
SIAASLGISRAHFSVTTPWAIAYMGPSADAMINGSDGRTTVEDLTTPPKWFPFPTKVCWRKHDL
ARLVPYKAPGISDGYRMGLVLKGSDCLLSKCYHEFGTQWLPLLETLHQVPVVPVGLLPPEIPGD
EKDETWVSIKKWLDGKQKGSVVYVALGSEVLVSQTEVVELALGLELSGLPFVWAYRKPKGPAKS
DSVELPDGFVERTRDRGLVWTSWAPQLRILSHESVCGFLTHCGSGSIVEGLMFGHPLIMLPIFG
DQPLNARLLEDKQVGIEIPRNEEDGCLTKESVARSLRSVVVEKEGEIYKANARELSKIYNDTKV
EKEYVSQFVDYLEKNARAVAIDHES
OsUGT1-2
(SEQ ID NO: 150)
MDSGYSSSYAAAAGMHVVICPWLAFGHLLPCLDLAQRLASRGHRVSFVSTPRNISRLPPVRPAL
APLVAFVALPLPRVEGLPDGAESTNDVPHDRPDMVELHRRAFDGLAAPFSEFLGTACADWVIVD
VFHHWAAAAALEHKVPCAMMLLGSAHMIASIADRRLERAETESPAAAGQGRPAAAPTFEVARMK
LIRTKGSSGMSLAERFSLTLSRSSLVVGRSCVEFEPETVPLLSTLRGKPITFLGLMPPLHEGRR
EDGEDATVRWLDAQPAKSVVYVALGSEVPLGVEKVHELALGLELAGTRFLWALRKPTGVSDADL
LPAGFEERTRGRGVVATRWVPQMSILAHAAVGAFLTHCGWNSTIEGLMFGHPLIMLPIFGDQGP
NARLIEAKNAGLQVARNDGDGSFDREGVAAAIRAVAVEEESSKVFQAKAKKLQEIVADMACHER
YIDGFIQQLRSYKD
Arabidopsis thaliana AAN72025.1
(SEQ ID NO: 151)
MGSISEMVFETCPSPNPIHVMLVSFQGQGHVNPLLRLGKLIASKGLLVTFVTTELWGKKMRQAN
KIVDGELKPVGSGSIRFEFFDEEWAEDDDRRADFSLYIAHLESVGIREVSKLVRRYEEANEPVS
CLINNPFIPWVCHVAEEFNIPCAVLWVQSCACFSAYYHYQDGSVSFPTETEPELDVKLPCVPVL
KNDEIPSFLHPSSRFTGFRQAILGQFKNLSKSFCVLIDSFDSLEREVIDYMSSLCPVKTVGPLF
KVARTVTSDVSGDICKSTDKCLEWLDSRPKSSVVYISFGTVAYLKQEQIEEIAHGVLKSGLSFL
WVIRPPPHDLKVETHVLPQELKESSAKGKGMIVDWCPQEQVLSHPSVACFVTHCGWNSTMESLS
SGVPVVCCPQWGDQVTDAVYLIDVFKTGVRLGRGATEERVVPREEVAEKLLEATVGEKAEELRK
NALKWKAEAEAAVAPGGSSDKNFREFVEKLGAGVTKTKDNGY
Arabidopsis thaliana AAF87256.1
(SEQ ID NO: 152)
MGSHVAQKQHVVCVPYPAQGHINPMMKVAKLLYAKGFHITFVNTVYNHNRLLRSRGPNAVDGLP
SFRFESIPDGLPETDVDVTQDIPTLCESTMKHCLAPFKELLRQINARDDVPPVSCIVSDGCMSF
TLDAAEELGVPEVLFWTTSACGFLAYLYYYRFIEKGLSPIKDESYLTKEHLDTKIDWIPSMKNL
RLKDIPSFIRTTNPDDIMLNFIIREADRAKRASAIILNTFDDLEHDVIQSMKSIVPPVYSIGPL
HLLEKQESGEYSEIGRTGSNLWREETECLDWLNTKARNSVVYVNFGSITVLSAKQLVEFAWGLA
ATGKEFLWVIRPDLVAGDEAMVPPEFLTATADRRMLASWCPQEKVLSHPAIGGFLTHCGWNSTL
ESLCGGVPMVCWPFFAEQQTNCKFSRDEWEVGIEIGGDVKREEVEAVVRELMDEEKGKNMREKA
EEWRRLANEATEHKHGSSKLNFEMLVNKVLLGE
Columba livia ClUGT1
(SEQ ID NO: 153)
MIHCGKKHICAFVTCILISASILMYSWKDPQLQNNITRKIFQATSALPASQLCRGKPAQNVITA
LEDNRTFIISPYFDDRESKVTRVIGIVHHEDVKQLYCWFCCQPDGKIYVARAKIDVHSDRFGFP
YGAADIVCLEPENCNPTHVSIHQSPHANIDQLPSFKIKNRKSETFSVDFTVCISAMFGNYNNVL
QFIQSVEMYKILGVQKVVIYKNNCSQLMEKVLKFYMEEGTVEIIPWPINSHLKVSTKWHFSMDA
KDIGYYGQITALNDCIYRNMQRSKFVVLNDADEIILPLKHLDWKAMMSSLQEQNPGAGIFLFEN
HIFPKTVSTPVFNISSWNRVPGVNILQHVHREPDRKEVFNPKKMIIDPRQVVQTSVHSVLRAYG
NSVNVPADVALVYHCRVPLQEELPRESLIRDTALWRYNSSLITNVNKVLHQTVL
Haemophilus ducreyi LgtF Q9L875
(SEQ ID NO: 154)
MPTLTVAMIVKNEAQDLAECLKTVDGWVDEIVIVDSGSTDDTLKIATQFNAKVYVNSDWQGFGP
QRQFAQQYVTSDYVLWLDADERVTPELKASILQAVQHNQKNTVYKVSRLSEIFGKEIRYSGWYP
DYVVRLYPTYLAKYGDELVHEKVHYPADSRVEKLQGDLLHFTYKNIHHYLVKSASYAKAWAMQR
AKAGKKASLLDGVTHAIACFLKMYLFKAGFLDGKQGFLLAVLSAHSTFVKYADLWDRTRS
Neisseria gonorrhoeae Q5F735
(SEQ ID NO: 155)
MKKVSVLIVAKNEANHIRECIESCRFDKEVIVIDDHSADNTAEIAEGLGAKVFRRHLNGDFGAQ
KTFAIEQAGGEWVFLIDADERCTPELSDEISKIVRTGDYAAYFVERRNLFPNHPATHGAMRPDS
VCRLMPKKGGSVQGKVHETVQTPYPERRLKHFMYHYTYDNWEQYFNKFNKYTSISAEKYREQGK
PVSFVRDIILRPIWGFFKIYILNKGFLDGKMGWIMSVNHSYYTMIKYVKLYYLYKSGGKF
Rhizobium meliloti (strain 1021) ExoM P33695
(SEQ ID NO: 156)
MPNETLHIDIGVCTYRRPELAETLRSLAAMNVPERARLRVIVADNDAEPSARALVEGLRPEMPF
DILYVHCPHSNISIARNCCLDNSTGDFLAFLDDDETVSGDWLTRLLETARTTGAAAVLGPVRAH
YGPTAPRWMRSGDFHSTLPVWAKGEIRTGYTCNALLRRDAASLLGRRFKLSLGKSGGEDTDFFT
GMHCAGGTIAFSPEAWVHEPVPENRASLAWLAKRRFRSGQTHGRLLAEKAHGLRQAWNIALAGA
KSGFCATAAVLCFPSAARRNRFALRAVLHAGVISGLLGLKEIEQYGAREVTSA
Rhizobium radiobacter Q44418
(SEQ ID NO: 157)
MCRCGRAVRSRPVCRPGQLVVRRSPRPRSRNHSRCRPLRLSVFPRPHRRVRHHCQRDLRWEPGR
WIAVRWKAARSHRRFRRCPFPRQLVWPVRERHRDAGDRRNQRERRRRDAYHEISEPKFRTRKRT
ESFWMNKAITVIVWLLVSLCVLAIITMPVSLQTHLVATAISLILLATIKSFNGQGAWRLVALGF
GTAIVLRYVYWRTTSTLPPVNQLENFIPGFLLYLAEMYSVVMLGLSLVIVSMPLPSRKTRPGSP
DYRPTVDVFVPSYNEDAELLANTLAAAKNMDYPADRFTVWLLDDGGSVQKRNAANIVEAQAAQR
RHEELKKLCEDLDVRYLTRERNVHAKAGNLNNGLAHSTGELVTVFDADHAPARDFLLETVGYFD
EDPRLFLVQTPHFFVNPDPIERNLRTFETMPSENEMFYGIIQRGLDKWNGAFFCGSAAVLRREA
LQDSDGFSGVSITEDCETALALHSRGWNSVYVDKPLIAGLQPATFASFIGQRSRWAQGMMQILI
FRQPLFKRGLSFTQRLCYMSSTLFWLFPFPRTIFLFAPLFYLFFDLQIFVASGGEFLAYTAAYM
LVNLMMQNYLYGSFRWPWISELYEYVQTVHLLPAVVSVIFNPGKPTFKVTAKDESIAEARLSEI
SRPFFVIFALLLVAMAFAVWRIYSEPYKADVTLVVGGWNLLNLIFAGCALGVVSERGDKSASRR
ITVKRRCEVQLGGSDTWVPASIDNVSVHGLLINIFDSATNIEKGATAIVKVKPHSEGVPETMPL
NVVRTVRGEGFVSIGCTFSPQRAVDHRLIADLIFANSEQWSEFQRVRRKKPGLIRGTAIFLAIA
LFQTQRGLYYLVRARRPAPKSAKPVGAVK
Streptococcus agalactiae cpsI O87183
(SEQ ID NO: 158)
MIKKIEKDLISVIVPIYNVEDYLVECIESLIVQTYRNIEILLINDGSTDNCATIAKEFSERDCR
VIYIEKSNGGLSEARNYGIYHSKGKYLTFVDSDDKVSSDYIANLYNAIQKHDSSIAIGGYLEFY
ERHNSIRNYEYLDKVIPVEEALLNMYDIKTYGSIFITAWGKLFHKSIFNDLEFALNKYHEDEFF
NYKAYLKANSITYIDKPLYHYRIRVGSIMNNSDNVIIARKKLDVLSALDERIKLITSLRKYSVF
LQKTEIFYVNQYFRTKKFLKQQSVMFKEDNYIDAYRMYGRLLRKVKLVDKLKLIKNRFF
Streptococcus pneumoniae cps33 Q54611
(SEQ ID NO: 159)
MYTFILMLLDFFQNHDFHFFMLFFVFILIRWAVIYFHAVRYKSYSCSVSDEKLFSSVIIPVVDE
PLNLFESVLNRISRHKPSEIIVVINGPKNSRLVKLCHDFNEKLENNMTPIQCYYTPVPGKRNAI
RVGLEHVDSQSDITVLVDSDTVWTPRTLSELLKPFVCDKKIGGVTTRQKILDPERNLVTMFANL
LEEIRAEGTMKAMSVTGKVGCLPGRTIAFRNIVERVYTKFIEETFMGFHKEVSDDRSLTNLTLK
KGYKTVMQDTSVVYTDAPTSWKKFIRQQLRWAEGSQYNNLKMTPWMIRNAPLMFFIYFTDMILP
MLLISFGVNIFLLKILNITTIVYTASWWEEILYVLLGMIFSFGGRNFKAMSRMKWYYVFLIPVF
IIVLSIIMCPIRLLGLMRCSDDLGWGTRNLTE
MbUGTc13
(SEQ ID NO: 160)
MADAMATTEKKPHVIFIPFPAQSHIKAMLKLAQLLHHKGLQITFVNTDFIHNQFLESSGPHCLD
GAPGFRFETIPDGVSHSPEASIPIRESLLRSIETNFLDRFIDLVTKLPDPPTCIISDGFLSVFT
IDAAKKLGIPVMMYWTLAACGFMGFYHIHSLIEKGFAPLKDASYLTNGYLDTVIDWVPGMEGIR
LKDFPLDWSTDLNDKVLMFTTEATQRSHKVSHHIFHTFDELEPSIIKTLSLRYNHIYTIGPLQL
LLDQIPEEKKQTGITSLHGYSLVKEEPECFQWLQSKEPNSVVYVNFGSTTVMSLEDMTEFGWGL
ANSNHYFLWIIRSNLVIGENAVLPPELEEHIKKRGFIASWCSQEKVLKHPSVGGFLTHCGWGST
IESLSAGVPMICWPYSWDQLTNCRYICKEWEVGLEMGTKVKRDEVKRLVQELMGEGGHKMRNKA
KDWKEKARIAIAPNGSSSLNIDKMVKEITVLARN
MbUGTc19
(SEQ ID NO: 161)
MANHHECMNWLDDKPKESVVYVAFGSLVKHGPEQVEEITRALIDSDVNFLWVIKHKEEGKLPEN
LSEVIKTGKGLIVAWCKQLDVLAHESVGCFVTHCGFNSTLEAISLGVPVVAMPQFSDQTTNAKL
LDEILGVGVRVKADENGIVRRGNLASCIKMIMEEERGVIIRKNAVKWKDLAKVAVHEGGSSDND
IVEFVSELIKAGSGEQQKIKKSPHVLLIPFPLQGHINPFIQFGKRLISKGVKTTLVTTIHTLNS
TLNHSNTTTTSIEIQAISDGCDEGGFMSAGESYLETFKQVGSKSLADLIKKLQSEGTTIDAIIY
DSMTEWVLDVAIEFGIDGGSFFTQACVVNSLYYHVHKGLISLPLGETVSVPGFPVLQRWETPLI
LQNHEQIQSPWSQMLFGQFANIDQARWVFTNSFYKLEEEVIEWTRKIWNLKVIGPTLPSMYLDK
RLDDDKDNGFNLYKA
MbUGT1-3
(SEQ ID NO: 162)
MENKTETTVRRRRRIILFPVPFQGHINPILQLANVLYSKGFSITIFHTNFNKPKTSNYPHFTFR
FILDNDPQDERISNLPTHGPLAGMRIPIINEHGADELRRELELLMLASEEDEEVSCLITDALWY
FAQSVADSLNLRRLVLMTSSLFNFHAHVSLPQFDELGYLDPDDKTRLEEQASGFPMLKVKDIKS
AYSNWQILKEILGKMIKQTKASSGVIWNSFKELEESELETVIREIPAPSFLIPLPKHLTASSSS
LLDHDRTVFQWLDQQPPSSVLYVSFGSTSEVDEKDFLEIARGLVDSKQSFLWVVRPGFVKGSTW
VEPLPDGFLGERGRIVKWVPQQEVLAHGAIGAFWTHSGWNSTLESVCEGVPMIFSDFGLDQPLN
ARYMSDVLKVGVYLENGWERGEIANAIRRVMVDEEGEYIRQNARVLKQKADVSLMKGGSSYESL
ESLVSYISSL
MbUGT1-2
(SEQ ID NO: 163)
MATKGSSGMSLAERFWLTLSRSSLVVGRSCVEFEPETVPLLSTLRGKPITFLGLMPPLHEGRRE
DGEDATVRWLDAQPAKSVVYVALGSEVPLGVEKVHELALGLELAGTRFLWALRKPTGVSDADLL
PAGFEERTRGRGVVATRWVPQMSILAHAAVGAFLTHCGWNSTIEGLMFGHPLIMLPIFGDQGPN
ARLIEAKNAGLQVARNDGDGSFDREGVAAAIRAVAVEEESSKVFQAKAKKLQEIVADMACHERY
IDGFIQQLRSYKDDSGYSSSYAAAAGMHVVICPWLAFGHLLPCLDLAQRLASRGHRVSFVSTPR
NISRLPPVRPALAPLVAFVALPLPRVEGLPDGAESTNDVPHDRPDMVELHRRAFDGLAAPFSEF
LGTACADWVIVDVFHHWAAAAALEHKVPCAMMLLGSAEMIASIADERLEHAETESPAAAGQGRP
AAAPTFEVARMKLIR
Coffea arabica (CaUGT_1, 6)
(SEQ ID NO: 164)
MAENHATFNVLMLPWLAHGHVSPYLELAKKLTARNFNVYLCSSPATLSSVRSKLTEKFSQSIHL
VELHLPKLPELPAEYHTTNGLPPHLMPTLKDAFDMAKPNFCNVLKSLKPDLLIYDLLQPWAPEA
ASAFNIPAVVFISSSATMTSFGLHFFKNPGTKYPYGNAIFYRDYESVFVENLTRRDRDTYRVIN
CMERSSKIILIKGFNEIEGKYFDYFSCLTGKKVVPVGPLVQDPVLDDEDCRIMQWLNKKEKGST
VFVSFGSEYFLSKKDMEEIAHGLEVSNVDFIWVVRFPKGENIVIEETLPKGFFERVGERGLVVN
GWAPQAKILTHPNVGGFVSHCGWNSVMESMKFGLPIIAMPMHLDQPINARLIEEVGAGVEVLRD
SKGKLHRERMAETINKVMKEASGESVRKKARELQEKLELKGDEEIDDVVKELVQLCATKNKRNG
LHYY
Stevia rebaudiana UGT85C1
(SEQ ID NO: 165)
MADQMAKIDEKKPHVVFIPFPAQSHIKCMLKLARILHQKGLYITFINTDTNHERLVASGGTQWL
ENAPGFWFKTVPDGFGSAKDDGVKPTDALRELMDYLKTNFFDLFLDLVLKLEVPATCIICDGCM
TFANTIRAAEKLNIPVILFWTMAACGFMAFYQAKVLKEKEIVPVKDETYLTNGYLDMEIDWIPG
MKRIRLRDLPEFILATKQNYFAFEFLFETAQLADKVSHMIIHTFEELEASLVSEIKSIFPNVYT
IGPLQLLLNKITQKETNNDSYSLWKEEPECVEWLNSKEPNSVVYVNFGSLAVMSLQDLVEFGWG
LVNSNHYFLWIIRANLIDGKPAVMPQELKEAMNEKGFVGSWCSQEEVLNHPAVGGFLTHCGWGS
IIESLSAGVPMLGWPSIGDQRANCRQMCKEWEVGMEIGKNVKRDEVEKLVRMLMEGLEGERMRK
KALEWKKSATLATCCNGSSSLDVEKLANEIKKLSRN
Arabidopsis thaliana AtUGT73C3
(SEQ ID NO: 202)
MATEKTHQFHPSLHFVLFPFMAQGHMIPMIDIARLLAQRGVTITIVTTPHNAARFKNVLNRAIE
SGLAINILHVKFPYQEFGLPEGKENIDSLDSTELMVPFFKAVNLLEDPVMKLMEEMKPRPSCLI
SDWCLPYTSIIAKNFNPKIVFHGMGCFNLLCMHVLRRNLEILENVKSDEEYFLVPSFPDRVEFT
KLQLPVKANASGDWKEIMDEMVKAEYTSYGVIVNTFQELEPPYVKDYKEAMDGKVWSIGPVSLC
NKAGADKAERGSKAAIDQDECLQWLDSKEEGSVLYVCLGSICNLPLSQLKELGLGLEESRRSFI
WVIRGSEKYKELFEWMLESGFEERIKERGLLIKGWAPQVLILSHPSVGGFLTHCGWNSTLEGIT
SGIPLITWPLFGDQFCNQKLVVQVLKAGVSAGVEEVMKWGEEDKIGVLVDKEGVKKAVEELMGD
SDDAKERRRRVKELGELAHKAVEKGGSSHSNITLLLQDIMQLAQFKN
Hordeum vulgare subsp. Vulgare HvUGT_B1
(SEQ ID NO: 204)
MAQAESERMRVVMFPWLAHGHINPYLELAKRLIASASGDHHLDVVVHLVSTPANLAPLAHHQTD
RLRLVELHLPSLPDLPPALHTTKGLPARLMPVLKRACDLAAPRFGALLDELCPDILVYDFIQPW
APLEAEARGVPAFHFATCGAAATAFFIHCLKTDRPPSAFPFESISLGGVDEDAKYTALVTVRED
STALVAERDRLPLSLERSSGFVAVKSSADIERKYMEYLSQLLGKEIIPTGPLLVDSGGSEEQRD
GGRIMRWLDGEEPGSVVFVSFGSEYFMSEHQMAQMARGLELSGVPFLWVVRFPNAEDDARGAAR
SMPPGFEPELGLVVEGWAPQRRILSHPSCGAFLTHCGWSSVLESMAAGVPMVALPLHIDQPLNA
NLAVELGAAAARVKQERFGEFTAEEVARAVRAAVKGKEGEAARRRARELQEVVARNNGNDGQIA
TLLQRMARLCGKDQAVPN
Hordeum vulgare subsp. Vulgare HvUGT_B3
(SEQ ID NO: 205)
MAEANDGGKMHVVMLPWLAFGHVLPFTEFAKRVARQGHRVTLLSAPRNTRRLIDIPPGLAGLIR
VVHVPLPRVDGLPEHAEATIDLPSDHLRPCLRRAFDAAFERELSRLLQEEAKPDWVLVDYASYW
APTAAARHGVPCAFLSLFGAAALSFFGTPETLLGIGRHAKTEPAHLTVVPEYVPFPTTVAYRGY
EARELFEPGMVPDDSGVSEGYRFAKTIEGCQLVGIRSSSEFEPEWLRLLGELYRKPVIPVGLFP
PAPQDDVAGHEATLRWLDGQAPSSVVYAAFGSEVKLTGAQLQRIALGLEASGLPFIWAFRAPTS
TETGAASGGLPEGFEERLAGRGVVCRGWVPQVKFLAHASVGGFLTHAGWNSIAEGLAHGVRLVL
LPLVFEQGLNARNIVDKNIGVEVARDEQDGSFAAGDIAAALRRVMVEDEGEGFGAKVKELAKVF
GDDEVNDQCVREFLMHLSDHSKKNQGQD
MbUGT1,2.2
(SEQ ID NO: 206)
MATKGSSGMSLAERFWLTLSRSSLVVGRSCVEFEPETVPLLSTLRGKPITFLGLMPPLHEGRRE
DGEDATVRWLDAQPAKSVVYVALGSEVPLGVEKVHELALGLELAGTRFLWALRKPTGVSDADLL
PAGFEERTRGRGVVATRWVPQMSILAHAAVGAFLTHCGWNSTIEGLMFGHPLIMLPIFGDQGPN
ARLIEAKNAGLQVARNDGDGSFDREGVAAAIRAVAVEEESSKVFQAKAKKLQEIVADMACHERY
IDGFIQQLRSYKDDSGYSSSYAAAAGMHVVICPWLAFGHLLPCLDLAQRLASRGHRVSFVSTPR
NISRLPPVRPALAPLVAFVALPLPRVEGLPDGAESTNDVPHDRPDMVELHRRAFDGLAAPFSEF
LGTACADWVIVDVFHHWAAAAALEHKVPCAMMLLGSAEMIASIADERLEHAETESPAAAGQGRP
AAAPTFEVARMKLIR
Coffea canephora (CcUGT_1,6) (207)
MAENHATFNVLMLPWLARGHVSPYLELAMKLTARNFNVYLCSSPATLSSVRSKLTEKFSQSIHL
VELHLPKLPELPAEYHTTNGLPPHLMPTLKDAFDMAKPNFCNVLKSLKPDLLIYDLLQPWAPEA
ASAFNTPAVVFISSSATMTSFGLHFFKNPGTKYPYGNTIFYRDYESVFVENLKKRDRDTYRVVN
CMERSSKIILIKGFKEIEGKYFDYFSCLTGKKVVPVGPLVQDPVLDDEDCRIMQWLNKKEKGST
VFVSFGSEYFLSKEDMEEIAHGLELSNVDFIWVVRFPKGENIVIEETLPKGFFERVGERGLVVN
GWAPQAKILTHPNVGGFVSHCGWNSVMESMKFGLPIVAMPMHLDQPINARLIEEVGAGVEVLRD
SKGKLHRERMAETINKVTKEASGEPARKKARELQEKLELKGDEEIDDVVKELVQLCATKNKRNG
LHCYN
Coffea eugenioides (CeUGT_1,6) (208)
MAENHATFNVLMLPWLAHGHVSPYLELAKKLTARNFNVYLCSSPATLSSVRSKLTEKFSQSIHL
VELHLPKLPELPAEYHTTNGLPPHLMPTLKDAFDMAEPNFCNVLKSLKPDLLIYDLLQPWAPEA
ASAFNIPAVVFISSSATMTSFGLHFFKNPGTKYPYGNTIFYRDYESVFVENLKRRDRDTYRVVN
CMERSSKIILIKGFKEIEGKYFDYFSCLTGKKVVPVGPLVQDPVLDDEDCRIMQWLNKKEKGST
VFVSFGSEYFLSKEDMEEIAHGLELSNVDFIWVVRFPKGENIVIEETLPKGFFERVGERGLVVN
GWAPQAKILTHPNVGGFVSHCGWNSVMESMKFGLPIIAMPMHLDQPINARLIEEVGAGVEVLRD
SKGKLHRERMAETINKVTKEASGESVRKKARELQEKLELKGDEEIDDVVKELVQLCATKNKRNG
LHYN
Coffea eugenioides (CeUGT_1,6.2) (209)
MAENHATFNVLMLPWLAHGHVSPYLELAKKLTARNFNVYLCSSPATLSSVRSKLTEKFSQSIHL
VELHLPKLPELPAEYHTTNGLPPHLMPTLKDAFDMAKPNFCNVLKSLKPDLLIYDLLQPWAPEA
ASAFNIPAVVFISSSATMTSFGLHFFKNPGTKYPYGNAIFYRDYESVFVENLTRRDRDTYRVIN
CMERSSKIILIKGFNEIEGKYFDYFSCLTGKKVVPVGPLVQDPVLDDEDCEIMQWLNKKEKVST
VFVSFGSEYFLSKKDMEEIAHGLELSNVDFIWVVRFPKGENIVIEETLPKGFFERVGERGLVVN
GWAPQAKILTHPNVGGFVSHCGWNSVMESMKFGLPIIAMPMHLDQPINARLIEEVGAGVEVLRD
SKGKLHRERMAETINKVMKEASGESVRKKARELQEKMDLKGDEEIDDVVKELVQLCATKNKRNG
LHYY
Siraitia grosvenorii (SgUGT94-289-3.2) (210)
MADAAQQGDTTTILMLPWLGYGHLSAFLELAKSLSRRNFHIYFCSTSVNLDAIKPKLPSSFSDS
IQFVELHLPSSPEFPPHLHTTNGLPPTLMPALHQAFSMAAQHFESILQTLAPHLLIYDSLQPWA
PRVASSLKIPAINFNTTGVFVISQGLHPIHYPHSKFPFSEFVLHNHWKAMYSTADGASTERTRK
RGEAFLYCLHASCSVILINSFRELEGKYMDYLSVLLNKKVVPVGPLVYEPNQDGEDEGYSSIKN
WLDKKEPSSTVFVSFGSEYFPSKEEMEEIAHGLEASEVNFIWVVRFPQGDNTSGIEDALPKGFL
ERAGERGMVVKGWAPQAKILKHWSTGGFVSHCGWNSVMESMMFGVPIIGVPMHVDQPFNAGLVE
EAGVGVEAKRDPDGKIQRDEVAKLIKEVVVEKTREDVRKKAREMSEILRSKGEEKFDEMVAEIS
LLLKI
Oryza sativa (OsJUGT_1,6)
(SEQ ID NO: 211)
MAQAERERLRVLMFPWLAHGHINPYLELATRLTTTSSSQIDVVVHLVSTPVNLAAVAHRRTDRI
SLVELHLPELPGLPPALHTTKHLPPRLMPALKRACDLAAPAFGALLDELSPDVVLYDFIQPWAP
LEAAARGVPAVHFSTCSAAATAFFLHFLDGGGGGGGRGAFPFEAISLGGAEEDARYTMLTCRDD
CTALLPKGERLPLSFARSSEFVAVKTCVEIESKYMDYLSKLVGKEIIPCGPLLVDSGDVSAGSE
ADGVMRWLDGQEPGSVVLVSFGSEYFMTEKQLAEMARGLELSGAAFVWVVRFPQQSPDGDEDDH
GAAAARAMPPGFAPARGLVVEGWAPQRRVLSHRSCGAFLTHCGWSSVMESMSAGVPMVALPLHI
DQPVGANLAAELGVAARVRQERFGEFEAEEVARAVRAVMRGGEALRRRATELREVVARRDAECD
EQIGALLHRMARLCGKGTGRAAQLGH
Panax ginseng (PsUGT94_B1)
(SEQ ID NO: 213)
MADNQNGRISIALLPFLAHGHISPFFELAKQLAKRNCNVFLCSTPINLSSIKDKDSSASIKLVE
LHLPSSPDLPPHYHTTNGLPSHLMLPLRNAFETAGPTFSEILKTLNPDLLIYDFNPSWAPEIAS
SHNIPAVYFLTTAAASSSIGLHAFKNPGEKYPFPDFYDNSNITPEPPSADNMKLLHDFIACFER
SCDIILIKSFRELEGKYIDLLSTLSDKTLVPVGPLVQDPMGHNEDPKTEQIINWLDKRAESTVV
FVCFGSEYFLSNEELEEVAIGLEISTVNFIWAVRLIEGEKKGILPEGFVQRVGDRGLVVEGWAP
QARILGHSSTGGFVSHCGWSSIAESMKFGVPVIAMARHLDQPLNGKLAAEVGVGMEVVRDENGK
YKREGIAEVIRKVVVEKSGEVIRRKARELSEKMKEKGEQEIDRALEELVQICKKKKDEQ
Stevia rebaudiana (SrUGT73E1, with optional His tag)
(SEQ ID NO: 214)
MAHHHHHHVGTGSNDDDDKSPDPNWASTSELVFIPSPGAGHLPPTVELAKLLLHRDQRLSVTII
VMNLWLGPKHNTEARPCVPSLRFVDIPCDESTMALISPNTFISAFVEHHKPRVRDIVRGIIESD
SVRLAGFVLDMFCMPMSDVANEFGVPSYNYETSGAATLGLMFHLQWKRDHEGYDATELKNSDTE
LSVPSYVNPVPAKVLPEVVLDKEGGSKMFLDLAERIRESKGIIVNSCQAIERHALEYLSSNNNG
IPPVFPVGPILNLENKKDDAKTDEIMRWLNEQPESSVVFLCFGSMGSFNEKQVKEIAVAIERSG
HRFLWSLRRPTPKEKIEFPKEYENLEEVLPEGFLKRTSSIGKVIGWAPQMAVLSHPSVGGFVSH
CGWNSTLESMWCGVPMAAWPLYAEQTLNAFLLVVELGLAAEIRMDYRTDTKAGYDGGMEVTVEE
IEDGIRKLMSDGEIRNKVKDVKEKSRAAVVEGGSSYASIGKFIEHVSNVTI
Oryza sativa (OsUGT1-2)
(SEQ ID NO: 215)
MADSGYSSSYAAAAGMHVVICPWLAFGHLLPCLDLAQRLASRGHRVSFVSTPRNISRLPPVRPA
LAPLVAFVALPLPRVEGLPDGAESTNDVPHDRPDMVELHRRAFDGLAAPFSEFLGTACADWVIV
DVFHHWAAAAALEHKVPCAMMLLGSAHMIASIADRRLERAETESPAAAGQGRPAAAPTFEVARM
KLIRTKGSSGMSLAERFSLTLSRSSLVVGRSCVEFEPETVPLLSTLRGKPITFLGLMPPLHEGR
REDGEDATVRWLDAQPAKSVVYVALGSEVPLGVEKVHELALGLELAGTRFLWALRKPTGVSDAD
LLPAGFEERTRGRGVVATRWVPQMSILAHAAVGAFLTHCGWNSTIEGLMFGHPLIMLPIFGDQG
PNARLIEAKNAGLQVARNDGDGSFDREGVAAAIRAVAVEEESSKVFQAKAKKLQEIVADMACHE
RYIDGFIQQLRSYKD
Camelina sativa (XP_010516905.1)
(SEQ ID NO: 216)
MASEKTLQVHPPLHFVLFPFMAQGHMIPMVDIARLLAQRGATVTIVTTRYNAGRFENVLSRAVE
SGLPINIVHVKFPYEEVGLPKGKENIDSLDSMELMVPFFKAVNMLQDPVVKLMEEMESRPSCII
SDLLLPYTSKIAKKFNIPKIVFHGISCFCLLCVHVLRRNLEILTNLKSDKEYFLVPSFPDRVEF
TKPQVTVETNASGDWKEFLDEMVEAEDTSYGVIINTFEELEPAYVKDYKDARAGNVWSIGPVSL
CNKAGVDKAERGNKATIDQDECLKWLDSKEEGSVLYVCLGSICNLPLVQLKELGLGLEESQRPF
IWVIRGWEKYNELSEWMVESGFEERIRERGLLIRGWAPQVLILSHPSVGGFLTHCGWNSTVEGI
TSGVPLITWPLFGDQFCNQTLVVQVLKAGVSVGVEEVMKWGEEEKIGVLVDKEGVKKAVEDLMG
ESDDAKERTKRVKELGGLAHKAVEEGGSSHSNITLFLQDIRQVQSV
Glycyrrhiza uralensis (UGT73F24)
(SEQ ID NO: 217)
MADVAEEQPLKIYFIPYLAAGHMIPLCDIATLFASRGHHVTIITTPSNAQTLRESHHFRVQTIQ
FPSQEVGLPAGVQNLTAVTNLDDSYKIYHATMLLRKHIEDFVERDPPDCIVADFLFPWVDDVAT
KLHIPRLVFNGFTLFTICAMESHKAHPLPVDAASGSFVIPDFPHHVTINSTPPKRTKEFVDPLL
TEAFKSHGFLINSFVELDGEECVEHYERITGGHKAWHLGPAFLVHRTAQDRGEKSVVSTQECLS
WLDSKRDNSVLYICFGTICYFPDKQLYEIASAIEASGHEFIWVVPEKRGNADESEEEKEKWLPK
GFEERNNGKKGMIIRGWAPQVAILGHPAVGGFLTHCGWNSTVEAVSAGVPMITWPVHSDQYFNE
KLITQVRGIGVEVGAEEWIVTAFRETEKLVGRDRIERAVRRVMDGGDEAVQIRRRARELGEMAR
QAVQEGGSSHTNLTALINDLKRWRDSKQLN
Glycyrrhiza uralensis (UGT73C33)
(SEQ ID NO: 218)
MAVFQANQPHFVLFPLMAQGHIIPMIDIARLLAQRGAIVTIFTTPKNASRFTSVLSRAVSSGLQ
IRLVHLHFPSKEAGLPEGCENLDMVASHDMICNIFQAIRMLQKQAEELFETLTPKPSCIISDFC
IPWTTQVAEKHHIPRISFHGFSCFCLHCMLKIHTSKVLEGITSESEYFTVPGIPDQIQVTKQQV
PGPMIDEMKEFGEQMRDAEIRSYGVIINTFEELEKAYVNDYKKERNGKVWCIGPVSLCNKDGLD
KAQRGNKASISEHHCLEWLDLQQPNSVIYVCLGSLCNLTPPQLMELALGLEATKRPFIWVIREG
NKFEELEKWISEEGFEERIKGRGLIIRGWAPQVLILSHPSIGGFLTHCGWNSTLEGVTAGVPMV
TWPLFADQFLNEKLVTQVLRIGVSLGVDVPLKWGEEEKVGVQVKKEGIEKAICMVMDEGEESKE
RRERAKELSEMAKRAVEKDGSSHLNMTMLIQDIMQQSSSKVET

1-171. (canceled)

172. A method for making a mogrol glycoside, comprising:
contacting mogrol or mogrol glycoside substrate having a C24 hydroxyl with a recombinant uridine diphosphate (UDP)-dependent glycosyltransferase (UGT) polypeptide comprising an amino acid sequence having at least 80% sequence identity with SEQ ID NO: 217, and in the presence of UDP-glucose co-factor; and
recovering a mogrol glycoside having a glycosylation of the C24 hydroxyl.

173. The method of claim 172, wherein the recombinant polypeptide is expressed in a microbial cell.

174. The method of claim 173, wherein the mogrol or mogrol glycoside substrate is fed to the microbial cell or a cell lysate thereof, or is contacted with the recombinant UGT enzyme in purified or partially purified form.

175. The method of claim 173, wherein the mogrol or mogrol glycoside substrate is produced by the microbial cell.

176. The method of claim 172, wherein the recombinant polypeptide comprises an amino acid sequence having at least 85% sequence identity to SEQ ID NO: 217.

177. The method of claim 172, wherein the recombinant polypeptide comprises an amino acid sequence having at least 90% sequence identity to SEQ ID NO: 217.

178. The method of claim 172, wherein the recombinant polypeptide comprises an amino acid sequence having at least 95% sequence identity to SEQ ID NO: 217.

179. The method of claim 172, wherein the recombinant polypeptide comprises one or more amino acid substitutions selected from A74E, I91F, H101P, Q241E, and I436L, with respect to SEQ ID NO: 217.

180. The method of claim 172, wherein the recombinant polypeptide comprises one or more amino acid substitutions selected from A74E, I91F, and H101P, with respect to SEQ ID NO: 217.

181. The method of claim 175, wherein the microbial cell expresses a heterologous enzyme pathway catalyzing the conversion of isopentenyl pyrophosphate (IPP) and dimethylallyl pyrophosphate (DMAPP) to mogrol.

182. The method of claim 181, wherein the heterologous enzyme pathway comprises a farnesyl diphosphate synthase (FPPS), squalene synthase (SQS), a squalene epoxidase (SQE), a triterpene cyclase (TTC), an epoxide hydrolase (EPH), and a cytochrome P450 enzyme capable of hydroxylating C11 of C24,25 dihydroxycucurbitadienol to thereby produce mogrol.

183. The method of claim 172, wherein the mogrol glycoside having a C24 glycosylation comprises one or more selected from Mog.II-E, Mog.III, Mog.III-A1, Mog.III-A2, Mog.IV, Mog.IV-A, siamenoside, Mog. V, and Mog. VI.

184. The method of claim 183, wherein the mogrol glycoside having a C24 glycosylation comprises Mog. V or siamenoside.

185. The method of claim 182, wherein the microbial cell further expresses at least three UGT enzymes: the UGT enzyme catalyzing glycosylation at the C24 hydroxyl of mogrol, a second UGT enzyme catalyzing primary glycosylation at a C3 hydroxyl of mogrol, and a third UGT enzyme catalyzing one or more branching glycosylation reactions.

186. The method of claim 185, wherein the second UGT enzyme comprises an amino acid sequence having at least 90% sequence identity with SEQ ID NO: 165, 202, 129, or 116.

187. The method of claim 185, wherein the third UGT enzyme comprises an amino acid sequence having at least 90% sequence identity with SEQ ID NO: 122.

188. The method of 173, wherein the microbial cell is prokaryotic.

189. The method of claim 188, wherein the microbial cell is selected from Escherichia coli, Bacillus subtilis, Corynebacterium glutamicum, Rhodobacter capsulatus, Rhodobacter sphaeroides, Zymomonas mobilis, Vibrio natriegens, and Pseudomonas putida.

190. The method of claim 188, wherein the microbial cell is Escherichia coli.

191. The method of claim 189, wherein the microbial cell has one or more genetic modifications that increase production or availability of UDP-glucose co-factor.

192. The method of 173, wherein the microbial cell is a yeast cell.

193. The method of claim 192, wherein the microbial cell is Saccharomyces cerevisiae, Pichia pastoris, or Yarrowia lipolytica.

194. The method of claim 175, wherein the mogrol glycoside products are recovered from the extracellular media.

195. A method for making a composition comprising a mogrol glycoside, comprising: producing a mogrol glycoside according to the method of claim 172, and incorporating the mogrol glycoside into the composition; wherein the composition is a sweetener composition, a flavoring composition, a food, a beverage, chewing gum, texturant, pharmaceutical composition, tobacco product, nutraceutical composition, or oral hygiene composition.


SEQUENCE LISTING (221 sequences)

SEQ ID NO: 1 - protein, 352 aa, synthetic construct
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAINDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 2 - protein, 417 aa, synthetic construct
MGSLGAILRHPDDFYPLLKLKMAARHAEKQIPPEPHWGFCYTMLHKVSRSFALVIQQLAPELRNAICIFYLVLRALDTVEDDTSIQTDIKVPILKAFHCHIYNRDWHFSCGTKDYKVLMDQFHHVSTAFLELGKGYQEAIEDITKRMGAGMAKFICKEVETVDDYDEYCHYVAGLVGLGLSKLFHASDLEDLAPDSLSNSMGLLLQKTNIIRDYLEDINEIPKSRMFWPREIWGKYADKLEDFKYEENSVKAVQCLNDLVTNALNHVEDCLKYMSNLRDLSIFRFCAIPQIMAIGTLALCYNNVEVFRGVVKMRRGLTAKVIDRTQTMADVYGAFFDFSVMLKAKVNSSDPNATKTLSRIEAIQKTCEQSGLLNKRKLYAVKSEPMFNPTLIVILFSLLCIILAYLSAKRLPANQPV

SEQ ID NO: 3 - protein, 417 aa, synthetic construct
MGSLGAILRHPDDFYPLLKLKMAARHAEKQIPPEPHWGFCYTMLHKVSRSFALVIQQLAPELRNAICIFYLVLRALDTVEDDTSIQTDIKVPILKAFHCHIYNRDWHFSCGTKDYKVLMDQFHHVSTAFLELGKGYQEAIEDITKRMGAGMAKFICKEVETVDDYDEYCHYVAGLVGLGLSKLFHASDLEDLAPDSLSNSMGLLLQKTNIIRDYLEDINEIPKSRMFWPREIWGKYADKLEDFKYEENSVKAVQCLNDLVTNALNHVEDCLKYMSNLRDLSIFRFCAIPQIMAIGTLALCYNNVEVFRGVVKMRRGLTAKVIDRTQTMADVYGAFFDFSVMLKAKVNNSDPNATKTLSRIEAIQKTCEQSGLLNKRKLYAVKSEPMFNPTLIVILFSLLCIILAYLSAKRLPANQPV

SEQ ID NO: 4 - protein, 417 aa, synthetic construct
MGSLGAILKHPDDFYPLLKLKIAARHAEKQIPPEPHWGFCYTMLHKVSRSFALVIQQLKPELRNAVCIFYLVLRALDTVEDDTSIQTDIKVPILKAFHCHIYNRDWHFSCGTKDYKVLMDEFHHVSTAFLELGKGYQEAIEDITKRMGAGMAKFICKEVETVDDYDEYCHYVAGLVGLGLSKLFHAAELEDLAPDSLSNSMGLFLQKTNIIRDYLEDINEIPKSRMFWPREIWGKYADKLEDFKYEENSVKAVQCLNDLVTNALNHVEDCLKYMSNLRDLSIFRFCAIPQIMAIGTLALCYNNVEVFRGVVKMRRGLTAKVIDRTKTMADVYGAFFDFSVMLKAKVNSNDPNASKTLSRIEAIQKTCKQSGILNRRKLYVVRSEPMFNPAVIVILFSLLCIILAYLSAKRLPANQSV

SEQ ID NO: 5 - protein, 417 aa, synthetic construct
MGSLGAILKHPDDFYPLLKLKMAARHAEKQIPPESHWGFCYTMLHKVSRSFALVIQQLKPELRNAVCIFYLVLRALDTVEDDTSIQTDIKVPILKAFHCHIYNRDWHFSCGTKDYKVLMDEFHHVSTAFLELGKGYQEAIEDITKRMGAGMAKFICKEVETVDDYDEYCHYVAGLVGLGLSKLFHAAELEDLAPDSLSNSMGLFLQKTNIIRDYLEDINEIPKSRMFWPREIWGKYADKLEDFKYEENSVKAVQCLNDLVTNALNHVEDCLKYMSNLRDLSIFRFCAIPQIMAIGTLALCYNNVEVFRGVVKMRRGLTAKVIDRTKTMADVYGAFFDFSVMLKAKVNSNDPNASKTLSRIEAIQQTCQQSGLMNKRKLYVVRSEPMYNPAVIVILFSLLCIILAYLSAKRLPANQSV

SEQ ID NO: 6 - protein, 417 aa, synthetic construct
MGSLGAILKHPDDFYPLLKLKMAARHAEKQIPPESHWGFCYTMLHKVSRSFALVIQQLKPELRNAVCIFYLVLRALDTVEDDTSIQTDIKVPILKAFHCHIYNRDWHFSCGTKDYKVLMDEFHHVSTAFLELGKGYQEAIEDITKRMGAGMAKFICKEVETVDDYDEYCHYVAGLVGLGLSKLFHAAELEDLAPDSLSNSMGLFLQKTNIIRDYLEDINEIPKSRMFWPREIWGKYADKLEDFKYEENSVKAVQCLNDLVTNALNHVEDCPKYMSNLRDLSIFRFCAIPQIMAIGTLALCYNNVEVFRGVVKMRRGLTAKVIDRTKTMADVYGAFFDFSVMLKAKVNSNDPNASKTLSRIEAIQQTCQQSGLMNKRKLYVVRSEPMYNPAVIVILFSLLCIILAYLSAKRLPANQSV

SEQ ID NO: 7 - protein, 417 aa, synthetic construct
MGSLGAILRHPDDIYPLLKLKMAARHAEKQIPPESHWGFCYTMLHKVSRSFALVIQQLKPELRNAVCIFYLVLRALDTVEDDTSIQTDIKVPILKAFHCHIYNRDWHFSCGTKDYKVLMDEFHHVSTAFLELGRGYQEAIEDITKRMGAGMAKFICKEVETVEDYDEYCHYVAGLVGLGLSKLFHASKSENLAPDSLSNSMGLFLQKTNIIRDYLEDINEIPKSRMFWPREIWSKYADKLEDFKYEKNSVKAVQCLNDLVTNALTHVEDCLEYMSNLKDLSIFRFCAIPQIMAIGTLALCYNNVDVFRGVVKMRRGLTAKVIYRTKTMADVYGAFFDFSVMLKAKVNSSDPNASKTLTRIEAIQKTCKQSGLLNKRELYAVRSEPMCNPAAIVVLFSLLCIILAYLSAKLLPANQPV

SEQ ID NO: 8 - protein, 417 aa, synthetic construct
MGSLGAILSHPDDLYPLLKLKMAAKHAEKQIPPDPHWGFCFSMLHKVSRSFALVIQQLKPELRNAVCIFYLVLRALDTVEDDTGIHPDIKVPILQAFHCHIYNRDWHFSCGTKHYKVLMDEFHHVSTAFLELGKGYQEAIEDVTERMGAGMAKFICKEVETVDDYDEYCHYVAGLVGLGLSKLFHAAELEDLAPDSLSNSMGLFLQKTNIIRDYLEDINEIPKSRMFWPREIWNKYADKLEDFKYEENSVKAVQCLNDLVTNALNHVEDCLKYMSNLKDLSTFRFCAIPQIMAIGTLALCYDNVEVFRGVVKMRRGLTAKIIDRTKKIADVYGAFFDFSVMLKAKVNSSDPNAAKTLSRIEAIEKTCKESGLLNKRKLYVIRSEPLFNPAVLVILFSLICILLAYLSAKRLPANQPV

SEQ ID NO: 9 - protein, 415 aa, synthetic construct
MGSLGAILKHPDDFYPLLKLKFAARHAEKQIPPEPHWAFCYSMLHKVSRSFGLVIQQLGPQLRDAVCIFYLVLRALDTVEDDTSIPTEVKVPILMAFHRHIYDKDWHFSCGTKEYKVLMDEFHHVSNAFLELGSGYQEAIEDITMRMGAGMAKFICKEVETIDDYDEYCHYVAGLVGLGLSKLFHASGAEDLATDSLSNSMGLFLQKTNIIRDYLEDINEIPKSRMFWPRQIWSKYVDKLEDLKYEENSAKAVQCLNDMVTDALVHAEDCLKYMSDLRDPAIFRFCAIPQIMAIGTLALCFNNTQVFRGVVKMRRGLTAKVIDRTKTMSDVYGAFFDFSCLLKSKVDNNDPNATKTLSRLEAIQKTCKESGTLSKRKSYIIESESGHNSALIAIIFIILAILYAYLSSNLLLNKQ

SEQ ID NO: 10 - protein, 412 aa, synthetic construct
MGALSTMLKHPDDIYPLLKLKIASRQIEKQIPAEPHWAFCYTMLQKVSRSFALVIQQLGTELRNAVCLFYLVLRALDTVEDDTSVATDVKVPILLAFHRHIYDPDWHFACGTNNYKVLMDEFHHVSTAFLELGTGYQEAIEDITKRMGAGMAKFILKEVETIDDYDEYCHYVAGLVGLGLSKLFHAAGKEDLASDSLSNSMGLFLQKTNIIRDYLEDINEIPKSRMFWPRQIWSKYVNKLEDLKYEENSEKAVQCLNDMVTNALIHMEDCLKYMAALRDPAIFKFCAIPQIMAIGTLALCYNNIEVFRGVVKMRRGLTAKVIDRTKSMDDVYGAFFDFSSILKSKVDKNDPNATKTLSRVEAVQKLCRDSGALSKRKSYIANREQSYNSTLIVALFIILAIIYAYLSASPRI

SEQ ID NO: 11 - protein, 418 aa, synthetic construct
MSSLKAVLKHPDDFYPLLKLKMAAKKAEKQIPSQPHWAFSYSMLHKVSRSFALVIQQLNPQLRDAVCIFYLVLRALDTVEDDTSIAADIKVPILIAFHKHIYNRDWHFACGTKEYKVLMDQFHHVSTAFLELKRGYQEAIEDITMRMGAGMAKFICKEVETVDDYDEYCHYVAGLVGIGLSKLFHSSGTEILFSDSISNSMGLFLQKTNIIRDYLEDINEIPKSRMFWPREIWSKYVNKLEDLKYEENSEKAVQCLNDMVTNALIHIEDCLKYMSQLKDPAIFRFCAIPQIMAIGTLALCYNNIEVFRGVVKLRRGLTAKVIDRTKTMADVYQAFSDFSDMLKSKVDMHDPNAQTTITRLEAAQKICKDSGTLSNRKSYIVKRESSYSAALLALLFTILAILYAYLSANRPNKIKFTL

SEQ ID NO: 12 - protein, 408 aa, synthetic construct
MDQRSEDEFYPLLKLKIVARNAEKQIPPEPHWAFCYTMLHKVSRSFALVIQQLGIELRNAVCIFYLVLRALDTVEDDTSIETDVKVPILIAFHRHIYDRDWHFSCGTKEYKVLMGQFHHVSTAFLELGKNYQEAIEDITKRMGAGMAKFICKEVETIDDYDEYCHYVAGLVGLGLSKLFHASGSEDLAPDDLSNSMGLFLQKTNIIRDYLEDINEIPKSRMFWPRQIWSEYVNKLEDLKYEENSVKAVQCLNDMVTNALMHAEDCLTYMAALRDPPIFRFCAIPQIMAIGTLALCYNNIEVFRGVVKMRRGLTAKVIDRTKTMADVYGAFFDFASMLEPKVDKNDPNATKTLSRLEAIQKTCRESGLLSKRKSYIVNDESGYGSTMIVILVIMVSIIFAYLSANHHNS

SEQ ID NO: 13 - protein, 415 aa, synthetic construct
MGSLAAMLRHPDDVYPLVKLKMAARHAEKQIPPEPHWAFCYTMLHKVSRSFGLVIQQLGTELRNAVCIFYLVLRALDTVEDDTSIATEVKVPILLAFHHHIYDRDWHFSCGTREYKVLMDEFHHVSTAFLELGKGYQEAIEDITMRMGAGMAKFICKEVETIDDYDEYCHYVAGLVGLGLSKLFHASGLEDLAPDSLSNSMGLFLQKTNIIRDYLEDINEIPKSRMFWPRQIWSKYVNKLEDLKYEKNSVKSVQCLNDMVTNALIHVDDCLKYMSALRDPAIFRFCAIPQIMAIGTLALCYNNIEVFRGVVKMRRGLTAKVIDQTKTISDVYGAFFDFSCMLKSKVEKNDPNSTKTLSRIEAIQKTCRESGTLSKRKSYILRSKRTHNSTLIFVLFIILAILFAYLSANRPPINM

SEQ ID NO: 14 - protein, 410 aa, synthetic construct
MGSLGAILKHPDDFYPLLKLKMAAKHAEKQIPAQPHWGFCYSMLHKVSRSFSLVIQQLGTELRDAVCIFYLVLRALDTVEDDTSIPTDVKVPILIAFHKHIYDPEWHFSCGTKEYKVLMDQIHHLSTAFLELGKSYQEAIEDITKKMGAGMAKFICKEVETVDDYDEYCHYVAGLVGLGLSKLFDASGFEDLAPDDLSNSMGLFLQKTNIIRDYLEDINEIPKSRMFWPRQIWSKYVNKLEDLKYEENSVKAVQCLNDMVTNALIHMDDCLKYMSALRDPAIFRFCAIPQIMAIGTLALCYNNVEVFRGVVKMRRGLTAKVIDRTRTMADVYRAFFDFSCMMKSKVDRNDPNAEKTLNRLEAVQKTCKESGLLNKRRSYINESKPYNSTMVILLMIVLAIILAYLSKRAN

SEQ ID NO: 15 - protein, 413 aa, synthetic construct
MGSLGAILKHPDDFYPLMKLKMAARRAEKNIPPEPHWGFCYSMLHKVSRSFALVIQQLDTELRNAVCIFYLVLRALDTVEDDTSIATEVKVPILMAFHRHIYDRDWHFSCGTKEYKVLMDEFHHVSTAFSELGRGYQEAIEDITMRMGAGMAKFICKEVETIDDYDEYCHYVAGLVGLGLSKLFHASGSEDLASDSLSNSMGLFLQVFLLTCIKTNIIRDYLEDINEIPKSRMFWPRQIWSKYVNKLEDLKDKENSVKAVECLNDMVTNALIHVEDCLTYMSALRDPSIFRFCAIPQIMAIGTLALCYNNIEVFRGVVKMRRGLTAKVIDRTKTMSDVYGGFFDFSCMLKSKVNKSDPNAMKALSRLEAIQKICRESGTLNKRKSYIIKSEPRYNSTLVFVLFIILAILFAYL

SEQ ID NO: 16 - protein, 414 aa, synthetic construct
MGSLGAILKHPDDFYPLLKLKFAARHAEKQIPPEPHWAFCYSMLHKVSRSFGLVIQQLDAQLRDAVCIFYLVLRALDTVEDDTSIPTEVKVPILMAFHRHIYDKDWHFSCGTKEYKVLMDEFHHVSNAFLELGSGFQEAIEDITMRMGAGMAKFICKEVETIDDYDEYCHYVAGLVGLGLSKLFHASGAEDLATDSLSNSMGLFLQKTNIIRDYLEDINEIPKSRMFWPRQIWSKYVDKLENLKYEENSAKAVQCLNDMVTNALLHAEDCLKYMSNLRDPAIFRFCAIPQIMAIGTLALCFNNIQVFRGVVKMRRGLTAKVIDRTKTMSDVYGAFFDFSCLLKSKVDNNDPNATKTLSRLEAIQKTCKESGTLSKRKSYIIESKSAHNSALIAIIFIILAILYAYLSSNLPNNQ

SEQ ID NO: 17 - protein, 528 aa, synthetic construct
MVDQCALGWILASALGLVIALCFFVAPRRNHRGVDSKERDECVQSAATTKGECRFNDRDVDVIVVGAGVAGSALAHTLGKDGRRVHVIERDLTEPDRIVGELLQPGGYLKLIELGLQDCVEEIDAQRVYGYALFKDGKNTRLSYPLENFHSDVSGRSFHNGRFIQRMREKAASLPNVRLEQGTVTSLLEEKGTIKGVQYKSKNGEEKTAYAPLTIVCDGCFSNLRRSLCNPMVDVPSYFVGLVLENCELPFANHGHVILGDPSPILFYQISRTEIRCLVDVPGQKVPSIANGEMEKYLKTVVAPQVPPQIYDSFIAAIDKGNIRTMPNRSMPAAPHPTPGALLMGDAFNMRHPLTGGGMTVALSDIVVLRNLLKPLKDLSDASTLCKYLESFYTLRKPVASTINTLAGALYKVFCASPDQARKEMRQACFDYLSLGGIFSNGPVSLLSGLNPRPLSLVLHFFAVAIYGVGRLLLPFPSVKGIWIGARLIYSASGIIFPIIRAEGVRQMFFPATVPAYYRSPPVFKPIV

SEQ ID NO: 18 - protein, 524 aa, synthetic construct
MVDQCALGWILASVLGAAALYFLFGRKNGGVSNERRHESIKNIATTNGEYKSSNSDGDIIIVGAGVAGSALAYTLGKDGRRVHVIERDLTEPDRIVGELLQPGGYLKLTELGLEDCVDDIDAQRVYGYALFKDGKDTRLSYPLEKFHSDVAGRSFHNGRFIQRMREKAASLPKVSLEQGTVTSLLEENGIIKGVQYKTKTGQEMTAYAPLTIVCDGCFSNLRRSLCNPKVDVPSCFVGLVLENCDLPYANHGHVILADPSPILFYRISSTEIRCLVDVPGQKVPSISNGEMANYLKNVVAPQIPSQLYDSFVAAIDKGNIRTMPNRSMPADPYPTPGALLMGDAFNMRHPLTGGGMTVALSDVVVLRDLLKPLRDLNDAPTLSKYLEAFYTLRKPVASTINTLAGALYKVFCASPDQARKEMRQACFDYLSLGGIFSNGPVSLLSGLNPRPISLVLHFFAVAIYGVGRLLIPFPSPKRVWIGARIISGASAIIFPIIKAEGVRQMFFPATVAAYYRAPRVVKGR

SEQ ID NO: 19 - protein, 526 aa, synthetic construct
MVDECALGWILAAALGAVIALCLFVAPKTNNQDGGVDSKATPECVQTTNGECRSDGDSDVIIVGAGVAGSALAHTLGKDGRRVHVIERDLTEPDRIVGELLQPGGYLKLIELGLADCVEEIDAQRVYGYALFKDGKNTRLSYPLEKFHSDVSGRSFHNGRFIQRMREKADSLPNVRLEQGTVTSLLEEKGTIKGVQYKSKDGKEKTAYAPLTIVCDGCFSNLRRSLCNPMVDVPSCFVGLVLENCQLPFANHGHVVLGDPSPILFYPISSTEIRCLVDVPGQKVPSISNGEMEKYLKTVVAPQVPPQIYDAFIAAIDKGNIRTMPNRSMPAAPHPTPGALLMGDAFNMRHPLTGGGMTVALSDIVVLRNLLKPLKDLHDAPTLCKYLESFYTLRKPVASTINTLAGALYKVFCASPDQARKEMRQACFDYLSLGGMFSNGPVSLLSGLNPRPLSLVLHFFAVAIYGVGRLLFPFPSPKGIWIGARLIYSASGIIFPIIKAEGVRQMFFPATVPAYYRSPPALKPVA

SEQ ID NO: 20 - protein, 529 aa, synthetic construct
MVDYCAFGWILAAVLGLAIALSFFVSPRRNRRGGADSTPRSEGVRSSSTTNGECRSVDGDADVIIVGAGVAGSALAHTLGKDGRLVHVIERDLTEPDRIVGELLQPGGYLKLIELGLQDCVEEIDAQKVYGYALFKDGKNTQLSYPLEKFQSDVSGRSFHNGRFIQRMREKAASLPNVRLEQGTVTSLLEEKGTIKGVQYKSKNGEEKTAYAPLTIVCDGCFSNLRRSLCKPMVDVPSCFVGLVLENCQLPFANHGHVVLGDPSPILFYPISSTEIRCLVDVPGQKIPSISNGEMEKYLKTIVAPQVPPQIHDAFIAAIDKGNIRTMPNRSMPAAPQPTPGALLMGDAFNMRHPLTGGGMTVALSDIVVLRNLLKPLKDLNDAPTLCKYLESFYTLRKPVASTINTLAGALYKVFCASPDQARKEMRQACFDYLSLGGIFSNGPVSLLSGLNPRPLSLVLHFFAVAIYGVGRLLLPFPSPKGIWIGARLVYSASGIIFPIIKAEGVRQMFFPATVPAYYRSPPVHKSIA

SEQ ID NO: 21 - protein, 529 aa, synthetic construct
MVDYCAFGWILAAVLGLAIALSFFVSPRRNRRGGADSTPRSEGVRSSSTTNGECRSVDCDADVIIVGAGVAGSALAHTLGKDGRLVHVIERDLTEPDRIVGELLQPGGYLKLIELGLQDCVEEIDAQKVYGYALFKDGKNTQLSYPLEKFQSDVSGRSFHNGRFIQRMREKAASLPNVRLEQGTVTSLLEEKGTIKGVQYKSKNGEEKTAHAPLTIVCDGCFSNLRRSLCKPMVDVPSCFVGLVLENCQLPFANHGHVVLGDPSPILFYPISSTEIRCLVDVPGQKVPSISNGEMEKYLKTIVAPQVPPQIHDAFIAAIDKGNIRTMPNRSMPAAPQPTPGALLMGDAFNMRHPLTGGGMTVALSDIVVLRNLLKPLKDLNDAPTLCKYLESFYTLRKPVASTINTLAGALYKVFCASPDQARKEMRQACFDYLSLGGIFSNGPVSLLSGLNPRPLSLVLHFFAVAIYGVGRLLLPFPSPKGIWIGARLVYSASGIIFPIIKAEGVRQMFFPATVPAYYRSPPVLKTIA

SEQ ID NO: 22 - protein, 531 aa, synthetic construct
MMVDHCAFAWILDVVLGLVVAVTFFVAAPRRNRRGGTDSTASKDCVISTAIANGECKPDDADAEVIIVGAGVAGSALAYTLGKDGRRVHVIERDLTEPDRIVGEFLQPGGYLKLIELGLGDCVEEIDAQKLYGYALFKDGKNTRVSYPLGNFHSDVSGRSFHNGRFIQRMREKAASLPNVRLEQGTVTSLLETKGTIKGVQYKSKNGEEKTAYAPLTIVCDGCFSNLRRSLCKPMVDVPSCFVGLVLENCQLPFANHGHVVLGDPSPILFYPISSTEIRCLVDVPGQKVPSISNGDMEKYLKTVVAPQVPPQIHDAFIAAIEKGNVRTMPNRSMPAAPHPTPGALLMGDAFNMRHPLTGGGMTVALSDIVVLRNLLKPLKDLNDASTLCKYLESFYTLRKPVASTINTLAGALYKVFCASPDQARKEMRQACFDYLSLGGVFSNGPISLLSGLNPRPSSLVLHFFAVAIYGVGRLLLPFPSLKGIWIGARLIYSASGIILPIIKAEGVRQMFFPATVPAYYRSPPVHKPIT

SEQ ID NO: 23 - protein, 528 aa, synthetic construct
MVDHCTFGWIFSAFLAFVIAFSFFLSPRKNRRGRGTNSTPRRDCLSSSATTNGECRSVDGDADVIIVGAGVAGSALAHTLGKDGRRVHVIERDLTEPDRIVGELLQPGGYLKLIELGLQDCVEEIDAQKVYGYALFKDGKSTRLSYPLENFQSDVSGRSFHNGRFIQRMREKAAFLPNVRLEQGTVTSLLEEKGTITGVQYKSKNGEQKTAYAPLTIVCDGCFSNLRRSLCNPMVDVPSCFVGLVLENCQLPYANLGHVVLGDPSPILFYPISSTEIRCLVDVPGQKVPSISNGEMEKYLKTVVAPQVPPQIHDAFIAAIEKGNIRTMPNRSMPAAPQPTPGALLMGDAFNMRHPLTGGGMTVALSDIVVLRNLLKPLKDLNDAPTLCKYLESFYTLRKPVASTINTLAGALYKVFCASSDQARKEMRQACFDYLSLGGIFSNGPVSLLSGLNPRPLSLVLHFFAVAIYGVGRLLLPFPSPKGIWIGARLVYSASGIIFPIIKAEGVRQMFFPATVPAYYRTPPVFNS

SEQ ID NO: 24 - protein, 528 aa, synthetic construct
MVDHCAFGWIFSALLAFPIALSLFLSPWRNRRVRGTDSTPRSASVSSSATTNGECRSVDGDADVVIVGAGVAGSALAHTLGKDGRRVHVIERDLTEPDRIVGELLQPGGYLKLIELGLQDCVEEIDAQKVYGYALFKDGKNTRLSYPLENFHSDVSGRSFHNGRFIQRMREKAASLPNVRLEQGTVTSLLEEKGTITGVQYKSKNGEQKTAYAPLTIVCDGCFSNLRRSLCTPMVDVPSYFVGLVLENCQLPYANLGHVVLGDPSPILFYPISSTEIRCLVDVPGQKVPSISNGEMEKYLKTVVAPQVPPQIHDAFIAAIEKGNIRTMPNRSMPAAPQPTPGALLMGDAFNMRHPLTGGGMTVALSDIVVLRNLLKPLKDLNDAPTLCKYLESFYTLRKPVASTINTLAGALYKVFCASPDQARKEMRQACFDYLSLGGIFSNGPVSLLSGLNPRPLSLVLHFFAVAIYGVGRLLLPFPSLKGIWIGARLVYSASGIIFPIIKAEGVRQMFFPATVPAYYRTPPVLNS

SEQ ID NO: 25 - protein, 533 aa, synthetic construct
MMVEHCAYGWILAAVLGLVVAVTFFVAVPRRNRRGGTDSTASKDCVISPAIANGECEPEDADADADVIIVGAGVAGSALAHTLGKDGRRVHVIERDLTEPDRIVGEFLQPGGHLKLIELGLGDCVEEIDAQKLYGYALFKDGKNTRVSYPLGNFHSDVSGRSFHNGRFIQRMREKAASLPNVRLEQGTVTSLLEKKGTIKGVQYKSKNGEEKTAYAPLTIVCDGCFSNLRRSLCKPMVDVPSCFVGLVLENCRLPFANHGHVVLGDPSPILFYPISSTEIRCLVDVPGQKVPSIPNGDMEKYLKTVVAPQVPPQIHDAFIAAIEKGNIRTMPNRSMPAAPHPTPGALLMGDAFNMRHPLTGGGMTVALSDIVVLRNLLKPLKDLNDAPTLCKYLESYYTLRKPVASTINTLAGALYKVFCASPDQARKEMRQACFDYLSLGGVFSNGPISLLSGLNPRPSCLVLHFFAVAIYGVGRLLLPFPSLKGIWIGARLIYSASGIILPIIKAEGVRQMFFPATVPAYYRSPPVHKPIT

SEQ ID NO: 26 - protein, 522 aa, synthetic construct
MLDQCPLGWILASVLGLFVLCNLIVKNRNSKASLEKRSECVKSIATTNGECRSKSDDVDVIIVGAGVAGSALAHTLGKDGRRLHVIERDLTEPDRIVGELLQPGGYLKLIELGLQDCVEEIDAQRVFGYALFKDGKDTRLSYPLEKFHSDVSGRSFHNGRFIQRMREKSASLPNVRLEQGTVTSLLEEKGTIKGVQYKTKTGQELTAFAPLTIVCDGCFSNLRRSLCNPKVDVPSCFVGLVLENCELPYANHGHVILADPSPILFYPISSTEVRCLVDVPGQKVPSISNGEMAKYLKSVVAPQIPPQIYDAFIAAVDKGNIRTMPNRSMPASPFPTPGALLMGDAFNMRHPLTGGGMTVALSDIVVLRDLLKPLGDLNDAATLCKYLESFYTLRKPVASTINTLAGALYKVFCASPDQARKEMRQACFDYLSLGGIFSTGPVSLLSGLNPRPLSLVLHFFAVAIYGVGRLLLPFPSPKRIWIGARLISGASGIIFPIIKAEGVRQMFFPATVPAYYRAAPVE

SEQ ID NO: 27 - protein, 524 aa, synthetic construct
MADPYTMGWILASLLGLFALYYLFVNNKNHREASLQESGSECVKSVAPVKGECRSKNGDADVIIVGAGVAGSALAHTLGKDGRRVHVIERDLAEPDRIVGELLQPGGYLKLIELGLQDCVEEIDSQRVYGYALFKDGKDTRLSYPLEKFHSDVSGRSFHNGRFIQRMREKAASLPNVQLEQGTVTSLLEENGTIKGVQYKTKTGQELTAYAPLTIVCDGCFSNLRRSLCIPKVDVPSCFVGLVLENCNLPYANHGHVVLADPSPILFYPISSTEVRCLVDVPGQKVPSISNGEMAKYLKTVVASQIPPQIYDSFVAAVDKGNIRTMPNRSMPAAPHPTPGALLMGDAFNMRHPLTGGGMTVALSDIVVLRDLLKPLRDLNDSVTLCKYLESFYTLRKPVASTINTLAGALYKVFCASPDQARKEMREACFDYLSLGGVFSEGPVSLLSGLNPRPLSLVCHFFAVAIYGVGRLLLPFPSPKRLWIGARLISGASGIIFPIIRAEGVRQMFFPATIPAYYRAPRPN

SEQ ID NO: 28 - protein, 527 aa, synthetic construct
MVDPYALGWSFASVLMGLVALYILVDKKNRSRVSSEARSEGVESVTTTTSGECRLTDGDADVIIVGAGVAGSALAHTLGKDGRRVHVIERDLTEPDRIVGELLQPGGYLKLIELGLEDCVEDIDAQRVFGYALFKDGKNTRLSYPLEKFHSDVSGRSFHNGRFIQRMREKAASLLNVRLEQGTVTSLLEENGTVKGVQYKTKDGNELTAHAPLTIVCDGCFSNLRRSLCNPQVDVPSSFVGLVLENCELPYANHGHVILADPSPILFYPISSTEVRCLVDVPGKKVPSIANGEMEKYLKNMVAPQLPPEIYDSFVAAVDRGNIRTMPNRSMPAAPHPTPGALLMGDAFNMRHPLTGGGMTVALSDIVVLRDLLKPLRDLNDAPTLCKYLESFYTLRKPVASTINTLAGALYKVFCASPDRARKEMRQACFDYLSLGGVFSMGPVSLLSGLNPRPLSLVLHFFAVAVYGVGRLLVPFPSPSRIWIGARLISGASAIIFPIIKAEGVRQMFFPATVPAYYRAPPVKRDH

SEQ ID NO: 29 - protein, 524 aa, synthetic construct
MVDQCALGWILASVLGASALYLLFGKKNCGVLNERRRESLKNIATTNGECKSSNSDGDIIIVGAGVAGSALAYTLAKDGRQVHVIERDLSEPDRIVGELLQPGGYLKLTELGLEDCVDDIDAQRVYGYALFKDGKDTRLSYPLEKFHSDVSGRSFHNGRFIQRMREKAASLPNVRLEQGTVTSLLEENGTIKGVQYKNKSGQEMTAYAPLTIVCDGCFSNLRRSLCNPKVDVPSCFVGLILENCDLPYANHGHVILADPSPILFYPISSTEIRCLVDVPGQKVPSISNGEMANYLKNVVAPQIPPQLYNSFIAAIDKGNIRTMPNRSMPADPYPTPGALLMGDAFNMRHPLTGGGMTVALSDIVVLRDLLKPLRDLNDAPTLCKYLEAFYTLRKPVASTINTLAGALYKVFCASPDQARKEMRQACFDYLSLGGIFSNGPVSLLSGLNPRPLSLVLHFFAVAIYGVGRLLIPFPSPKRVWIGARLISGASAIIFPIIKAEGVRQMFFPKTVAAYYRAPPVVRER

SEQ ID NO: 30 - protein, 524 aa, synthetic construct
MVDQCALGWILASVLGASALYLLFGKKNCGVSNERRRESLKNIATTNGECKSSNSDGDIIIVGAGVAGSALAYTLAKDGRQVHVIERDLSEPDRIVGELLQPGGYLKLTELGLEDCVDEIDAQRVYGYALFKDGKDTRLSYPLEKFHSDVSGRSFHNGRFIQRMREKAASLPNVRLEQGTVTSLLEENGTIRGVQYKNKSGQEMTAYAPLTIVCDGCFSNLRRSLCNPKVDVPSCFVGLILENCDLPHANHGHVILADPSPILFYPISSTEIRCLVDVPGQKVPSISNGEMANYLKNVVAPQIPPQLYNSFIAAIDKGNIRTMPNRSMPADPYPTPGALLMGDAFNMRHPLTGGGMTVALSDIVVLRDLLKPLRDLNDAPTLCKYLEAFYTLRKPVASTINTLAGALYKVFCASPDQARKEMRQACFDYLSLGGIFSNGPVSLLSGLNPRPLSLVLHFFAVAIYGVGRLLIPFPSPKRVWIGARLISGASAIIFPIIKAEGVRQMFFPKTVAAYYRAPPIVRER

SEQ ID NO: 31 - protein, 537 aa, synthetic construct
MVDQYALGLILASVLGFVVLYNLMAKKNRIRVSSEARTEGVQTVITTTNGECRSIEGDVDVIIVGAGVAGSALAHTLGKDGRKVHVIERDLSEPDRIVGELLQPGGYLKLVELGLQDSVEDIDAQRVFGYALFKDGKNTRLSYPLEKFHSDVSGRSFHNGRFIQRMREKAASLPNIRLEQGTVTSLLEENGTIKGVQYKTKDGKELAAHAPLTIVCDGCFSNLRRSLCNPQVDVPSSFVGLVLENCELPYANHGHVVLADPSPILFYPISSTEVRCLVDVPGQKVPSISNGEMAKYLKTMVAPQVPPEIYDSFVAAVDRGNIRTMPNRSMPAAPQPTPGALLMGDAFNMRHPLTGGGMTVALSDIVVLRDLLRPLRDLNDAPTLCKYLESFYTLRKPVASTINTLAGALYKVFCASPDRARNEMRQACFDYLSLGGVFSTGPVSLLSGLNPRPLSLVLHFFAVAVYGVGRLLVPFPSPSRMWIGARLISGASAIIFPIIKAEGVRQMFFPATVPAYYRAPPVNCQARSLKPDALKGL

SEQ ID NO: 32 - protein, 523 aa, synthetic construct
MADSYVWGWILGSVMTLVALCGVVLKRRKGSGISATRTESVKCVSSINGKCRSADGSDADVIIVGAGVAGSALAHTLGKDGRRVHVIERDLTEPDRIVGELLQPGGYLKLIELGLEDCVEEIDAQQVFGYALFKDGKHTRLSYPLEKFHSDVSGRSFHNGRFIQRMREKSASLPNVRLEQGTVTSLLEEKGTIRGVQYKTKDGRELTAFAPLTIVCDGCFSNLRRSLCNPKVDVPSCFVGLVLENCNLPYSNHGHVILADPSPILFYPISSTEVRCLVDVPGQKVPSIANGEMANYLKTIVAPQVPPEIYNSFVAAVDKGNIRTMPNRSMPAAPYPTPGALLMGDAFNMRHPLTGGGMTVALSDIVVLRDLLRPLRDLNDAPTLCKYLESFYTLRKPIASTINTLAGALYKVFCASPDQARKEMRQACFDYLSLGGVFSTGPISLLSGLNPRPVSLVLHFFAVAIYGVGRLLLPFPSPKRIWIGARLISGASGIIFPIIKAEGVRQMFFPATVPAYYRAPPVE

SEQ ID NO: 33 - protein, 531 aa, synthetic construct; identical to SEQ ID NO: 22

SEQ ID NO: 34 - protein, 525 aa, synthetic construct
MLDTYVFGWIICAALSVFVIRNFVFAGKKCCASSETDASMCAENITTAAGECRSSMRDGEFDVLIVGAGVAGSALAYTLGKDGRQVLVIERDLSEPDRIVGELLQPGGYLKLIELGLEDCVDKIDAQQVFGYALFKDGKHIRLSYPLEKFHSDVAGRSFHNGRFIQRMREKAASLPNVRLEQGTVTSLLEEKGVIKGVQYKTKDSQELSVCAPFTIVCDGCFSNLRRSLCDPKVDVPSCFVGLVLENCELPCANHGHVILGEPSPVLFYPISSTEIRCLVDVPGQKVPSISNGEMAKYLKTVIAPQVPHELHNAFIAAVDKGSIRTMPNRSMPAAPYPTPGALLMGDAFNMRHPLTGGGMTVALSDIVVLRNLLRPLRDLNDAPSLCKYLESFYTLRKPVASTINTLAGALYKVFCASSDPARKEMRQACFDYLSLGGQFSEGPISLLSGLNPRPLTLVLHFFAVATYGVGRLLLPFPSPKRMWIGLRLISSASGIIMPIIKAEGVRQMFFPATVPAYYRNPPAA

SEQ ID NO: 35 - protein, 533 aa, synthetic construct
MKMADHYLLGWILASVMGLFAFYYIVYLLVKPEEDNNRRSLPQPRSDFVKTMTATNGECRSDDDSDVDVIIVGAGVAGAALAHTLGKDGRRVHVIERDLTEPDRIVGELLQPGGYLKLIELGLEDCVEEIDAQRVFGYALFKDGKHTQLAYPLEKFHSEVAGRSFHNGRFIQRMREKAASLPSVKLEQGTVTSLLEEKGTIKGVLYKTKTGEELTAFAPLTIVCDGCFSNLRRSLCNPKVDVPSCFVGLVLENCRLPYANNGHVILADPSPILFYPISSTEVRSLVDVPGQKVPSVSSGEMANYLKNVVAPQVPPEIYDSFVAAVDKGNIRTMPNRSMPASPYPTPGALLMGDAFNMRHPLTGGGMTVALSDIVVLRDLLKPLRDLHDAPTLCRYLESFYTLRKPVASTINTLAGALYKVFCASPDEARKEMRQACFDYLSLGGVFSTGPVSLLSGLNPRPLSLVLHFFAVAIYGVGRLLLPFPSPHRIWVGARLISGASGIIFPIIKAEGVRQMFFPATVPAYYRAPPIKCN

SEQ ID NO: 36 - protein, 529 aa, synthetic construct
MAAAAAAASGVGFQLIGAAAATLLAAVLVAAVLGRRRRRARPQAPLVEAKPAPEGGCAVGDGRTDVIIVGAGVAGSALAYTLGKDGRRVHVIERDLTEPDRIVGELLQPGGYLKLIELGLEDCVEEIDAQRVLGYALFKDGRNTKLAYPLEKFHSDVAGRSFHNGRFIQRMRQKAASLPNVQLEQGTVTSLLEENGTVKGVQYKTKSGEELKAYAPLTIVCDGCFSNLRRALCSPKVDVPSCFVGLVLENCQLPHPNHGHVILANPSPILFYPISSTEVRCLVDVPGQKVPSIASGEMANYLKTVVAPQIPPEIYDSFIAAIDKGSIRTMPNRSMPAAPHPTPGALLMGDAFNMRHPLTGGGMTVALSDIVVLRNLLKPLHNLHDASSLCKYLESFYTLRKPVASTINTLAGALYKVFSASPDQARNEMRQACFDYLSLGGVFSNGPIALLSGLNPRPLSLVAHFFAVAIYGVGRLMLPLPSPKRMWIGARLISGACGIILPIIKAEGVRQMFFPATVPAYYRAAPMGE

SEQ ID NO: 37 - protein, 484 aa, synthetic construct
MRKNLEEAGCAVSDGGTDVIIVGAGVAGSALAYTLGKDGRRVHVIERDLTEPDRIVGELLQPGGYLKLIELGLQDCVEEIDAQRVLGYALFKDGRNTKLAYPLEKFHSDVAGRSFHNGRFIQRMRQKAASLPNVQLEQGTVTSLLEENGTVKGVQYKTKSGEELKAYAPLTIVCDGCFSNLRRALCSPKVDVPSCFVGLVLENCQLPHPNHGHVILANPSPILFYPISSTEVRCLVDVPGQKVPSIATGEMANYLKTVVAPQIPPEIYDSFIAAIDKGSIRTMPNRSMPAAPHPTPGALLMGDAFNMRHPLTGGGMTVALSDIVVLRNLLKPLRNLHDASSLCKYLESFYTLRKPVASTINTLAGALYKVFSASPDQARNEMRQACFDYLSLGGVFSNGPIALLSGLNPRPLSLVAHFFAVAIYGVGRLMLPLPSPKRMWIGARLISGACGIILPIIKAEGVRQMFFPATVPAYYRAAPTGEKA

SEQ ID NO: 38 - protein, 524 aa, synthetic construct
MDLYNIGWILSSVLSLFALYNLIFSGKRNYHDVNDKVKDSVTSTDAGDIQSEKLNGDADVIIVGAGIAGAALAHTLGKDGRRVHIIERDLSEPDRIVGELLQPGGYLKLVELGLQDCVDNIDAQRVFGYALFKDGKHTRLSYPLEKFHSDVSGRSFHNGRFIQRMREKAASLPNVNMEQGTVISLLEEKGTIKGVQYKNKDGQALTAYAPLTIVCDGCFSNLRRSLCNPKVDNPSCFVGLILENCELPCANHGHVILGDPSPILFYPISSTEIRCLVDVPGTKVPSISNGDMTKYLKTTVAPQVPPELYDAFIAAVDKGNIRTMPNRSMPADPRPTPGAVLMGDAFNMRHPLTGGGMTVALSDIVVLRNLLKPMRDLNDAPTLCKYLESFYTLRKPVASTINTLAGALYKVFSASPDEARKEMRQACFDYLSLGGLFSEGPISLLSGLNPRPLSLVLHFFAVAVFGVGRLLLPFPSPKRVWIGARLLSGASGIILPIIKAEGIRQMFFPATVPAYYRAPPVNAF

SEQ ID NO: 39 - protein, 437 aa, synthetic construct
MKEEFDICIIGAGMAGATISAYLAPKGIKIALIDHCYKEKKRIVGELLQPGAVLSLEQMGLSHLLDGFEAQTVKGYALLQGNEKTTIPYPSQHEGIGLHNGRFLQQIRASALENSSVTQIHGKALQLLENERNEIIGVSYRESITSQIKSIYAPLTITSDGFFSNFRAHLSNNQKTVTSYFIGLILKDCEMPFPKHGHVFLSGPTPFICYPISDNEVRLLIDFPGEQLPRKNLLQEHLDTNVTPYIPECMRSSYAQAIQEGGFKVMPNHYMAAKPIVRKGAVMLGDALNMRHPLTGGGLTAVFSDIQILSAHLLAMPDFKNTDLIHEKIEAYYRDRKRANANLNILANALYAVMSNDLLKTAVFKYLQCGGANAQESIAVLAGLNRKHFSLIKQFCFLAVFGACNLLQQSISNIPKALKLLKDAFVIIKPLIKNELS

SEQ ID NO: 40 - protein, 759 aa, synthetic construct
MWRLKVGAESVGENDEKWLKSISNHLGRQVWEFCPDAGTQQQLLQVHKARKAFHDDRFHRKQSSDLFITIQYGKEVENGGKTAGVKLKEGEEVRKEAVESSLERALSFYSSIQTSDGNWASDLGGPMFLLPGLVIALYVTGVLNSVLSKHHRQEMCRYVYNHQNEDGGWGLHIEGPSTMFGSALNYVALRLLGEDANAGAMPKARAWILDHGGATGITSWGKLWLSVLGVYEWSGNNPLPPEFWLFPYFLPFHPGRMWCHCRMVYLPMSYLYGKRFVGPITPIVLSLRKELYAVPYHEIDWNKSRNTCAKEDLYYPHPKMQDILWGSLHHVYEPLFTRWPAKRLREKALQTAMQHIHYEDENTRYICLGPVNKVLNLLCCWVEDPYSDAFKLHLQRVHDYLWVAEDGMKMQGYNGSQLWDTAFSIQAIVSTKLVDNYGPTLRKAHDFVKSSQIQQDCPGDPNVWYRHIHKGAWPFSTRDHGWLISDCTAEGLKAALMLSKLPSETVGESLERNRLCDAVNVLLSLQNDNGGFASYELTRSYPWLELINPAETFGDIVIDYPYVECTSATMEALTLFKKLHPGHRTKEIDTAIVRAANFLENMQRTDGSWYGCWGVCFTYAGWFGIKGLVAAGRTYNNCLAIRKACDFLLSKELPGGGWGESYLSCQNKVYTNLEGNRPHLVNTAWVLMALIEAGQAERDPTPLHRAARLLINSQLENGDFPQQEIMGVFNKNCMITYAAYRNIFPIWALGEYCHRVLTE

SEQ ID NO: 41 - protein, 762 aa, synthetic construct
MWRLKVGAESVGENDEKWVKSISNHLGRQVWEFCPDAGTPQQLLQIEKARKAFQDNRFHRKQTSDLLVSIQCEKGTTNGARVPGTKLKEGEEVRKEAVKSTLERALSFYSSIQTSDGNWASDLGGPMFLLPGLVIALCVTGALNSVLSKHHRQEMCRYLYNHQNEDGGWGLHIESPSTMFGSALNYVALRLLGEDADGGEGRAMTKARAWILGHGGATAITSWGKLWLSVLGVYEWSGNNPLPPEFWLLPYFLPFHPGRMWCHCRMVYLPMSYLYGKRFVGPITPVVLSLRKELYTVPYHEIDWNKSRNTCAKEDLYYPHSKMQDILWGSIHHMYEPLFTHWPAKRLREKALKTAMQHIHYEDENTRYICLGPVNKVLNMLCCWVEDPYSEAFKLHLQRVHDYLWVAEDGMKMQGYNGSQLWDTAFSVQAIISTKLVDNYGPTLRKAHDYVKNSQIQQDCPGEPNVWFRHIHKGAWPFSTRDHGWLISDCTAEGLKASLMLSKLPSETVGEPLERNRLCDAVNVLLSLQNDNGGFASYELTRSYPWLELINPAETFGDIVIDYPYVECTSATMEALALFKKLHPGHRTKEIDTAIARAADFLENMQRTDGSWYGCWGVCFTYAGWFGIKGLVAAGRAYSNCLAIRKACDFLLSKELPGGGWGESYLSCQNKVYTNLEGNRPHLVNTAWVLMALIEAGQGERDPAPLHRAARLLINSQLENGDFPQEEIMGVFNKNCMITYAAYRNIFPIWALGEYCHRVLTE

SEQ ID NO: 42 - protein, 764 aa, synthetic construct
MWRLKVGAESVGEKDEKWVKSVSNHLGRQVWEFCADAAADTPHQLLQIQNARNHFHHNRFHRKQSSDLFLAIQYEKEIAKGAKGGAVKVKEGEEVGKEAVKSTLERALGFYSAVQTSDGNWASDLGGPMFLLPGLVIALHVTGVLNSVLSKHHRVEMCRYLYNHQNEDGGWGLHIEGTSTMFGSALNYVALRLLGEDADGGDGGAMTKARAWILERGGATAITSWGKLWLSVLGVYEWSGNNPLPPEFWLLPYSLPFHPGRMWCHCRMVYLPMSYLYGKRFVGPITPKVLSLRQELYTIPYHEIDWNKSRNTCAKEDLYYPHPKMQDILWGSIYHVYEPLFTRWPGKRLREKALQAAMKHIHYEDENSRYICLGPVNKVLNMLCCWVEDPYSDAFKLHLQRVHDYLWVAEDGMRMQGYNGSQLWDTAFSIQAIVATKLVDSYAPTLRKAHDFVKDSQIQEDCPGDPNVWFRHIHKGAWPFSTRDHGWLISDCTAEGLKASLMLSKLPSTMVGEPLEKNRLCDAVNVLLSLQNDNGGFASYELTRSYPWLELINPAETFGDIVIDYPYVECTAATMEALTLFKKLHPGHRTKEIDTAIGKAANFLEKMQRADGSWYGCWGVCFTYAGWFGIKGLVAAGRTYNSCLAIRKACEFLLSKELPGGGWGESYLSCQNKVYTNLEGNKPHLVNTAWVLMALIEAGQGERDPAPLHRAARLLMNSQLENGDFVQQEIMGVFNKNCMITYAAYRNIFPIWALGEYCHRVLTE

SEQ ID NO: 43 - protein, 764 aa, synthetic construct
MWRLKVGAESVGEKEEKWLKSISNHLGRQVWEFCADQPTASPNHLQQIDNARKHFRNNRFHRKQSSDLFLAIQNEKEIANGTKGGGIKVKEEEDVRKETVKNTVERALSFYSAIQTNDGNWASDLGGPMFLLPGLVIALYVTGVLNSVLSKHHRQEMCRYLYNHQNEDGGWGLHIEGTSTMFGSALNYVALRLLGEDADGGEGGAMTKARGWILDRGGATAITSWGKLWLSVLGVYEWSGNNPLPPEFWLLPYCLPFHPGRMWCHCRMVYLPMSYLYGKRFVGPITPIVLSLRKELYTIPYHEIDWNKSRNTCAKEDLYYPHPKMQDILWGSIYHLYEPLFTRWPGKRLREKALQMAMKHIHYEDENSRYICLGPVNKVLNMLCCWVEDPYSDAFKFHLQRVPDYLWIAEDGMRMQGYNGSQLWDTAFSVQAIISTKLIDSFGTTLKKAHDFVKDSQIQQDFPGDPNVWFRHIHKGAWPFSTRDHGWLISDCTAEGLKASLMLSKLPSKIVGEPLEKSRLCDAVNVLLSLQNENGGFASYELTRSYPWLELINPAETFGDIVIDYPYVECTSATMEALTLFKKLHPGHRTKEIDTAVAKAANFLENMQRTDGSWYGCWGVCFTYAGWFGIKGLVAAGRTYSTCVAIRKACDFLLSKELPGGGWGESYLSCQNKVYTNLEGNRPHLVNTAWVLMALIEAGQAERDPAPLHRAARLLINSQLENGDFPQEEIMGVFNKNCMITYAAYRNIFPIWALGEYFHRVLTE

SEQ ID NO: 44 - protein, 764 aa, synthetic construct
MWRLKVGAESVGEKEEKWLKSISNHLGRQVWEFCAHQPTASPNHLQQIDNARNHFRNNRFHRKQSSDLFLAIQNEKEIANVTKGGGIKVKEEEDVRKETVKNTVERALSFYSAIQTNDGNWASDLGGPMFLLPGLVIALYVTGVLNSVLSKHHRQEMCRYLYNHQNEDGGWGLHIEGTSTMFGSALNYVALRLLGEDADGGEGGAMTKARSWILDRGGATAITSWGKLWLSVLGVYEWSGNNPLPPEFWLLPYCLPFHPGRMWCHCRMVYLPMSYLYGKRFVGPITPIVLSLRKELYTIPYHEIDWNRSRNTCAKEDLYYPHPKMQDILWGSIYHLYEPLFTRWPGKRLREKALQMAMKHIHYEDENSRYICLGPVNKVLNMLCCWVEDPYSDAFKFHLQRVPDYLWVAEDGMRMQGYNGSQLWDTAFSVQAIISTKLIDSFGTTLKKAHDFVKDSQIQQDCPGDPNVWFRHIHKGAWPFSTRDHGWLISDCTAEGLKASLMLSKLPSKIVGEPLEKSRLCDAVNVLLSLQNENGGFASYELTRSYPWLELINPAETFGDIVIDYPYVECTSATMEALTLFKKLHPGHRTKEIDIAVARAANFLENMQRTDGSWYGCWGVCFTYAGWFGIKGLVAAGRTYNSCVAIRKACDFLLSKELPGGGWGESYLSCQNKVYTNLEGNRPHLVNTAWVLMALIEAGQAERDPAPLHRAARLLINSQLENGDFPQEEIMGVFNKNCMITYAAYRNIFPIWALGEYFHRVLTE

SEQ ID NO: 45 - protein, 765 aa, synthetic construct
MWRLKVGAESVGEKDEKWVKSVSNHLGRQVWEFCADAAAAATPRQLLQIQNARNHFHRNRFHRKQSSDLFLAIQYEKEIAEGGKGGAVKVKEEEEVGKEAVKSTLERALSFYSAVQTSDGNWASDLGGPMFLLPGLVIALYVTGVLNSVLSKHHRVEMCRYLYNHQNEDGGWGLHIEGTSTMFGSALNYVALRLLGEDADGGDDGAMTKARAWILERGGATAITSWGKLWLSVLGVYEWSGNNPLPPEFWLLPYSLPFHPGRMWCHCRMVYLPMSYLYGKRFVGPITPKVLSLRQELYTVPYHEIDWNKSRNTCAKEDLYYPHPKMQDILWGSIYHVYEPLFTRWPGKRLREKALQTAMKHIHYEDENSRYICLGPVNKVLNMLCCWVEDPYSDAFKLHLQRVHDYLWVAEDGMRMQGYNGSQLWDTAFSIQAIVATKLVDSFAPTLRKAHDFVKDSQIQEDCPGDPNVWFRHIHKGAWPFSTRDHGWLISDCTAEGLKASLMLSKLPSTMVGEPLEKNRLCDAVNVLLSLQNDNGGFASYELTRSYPWLELINPAETFGDIVIDYPYVECTAATMEALTLFKKLHPGHRTKEIDTAVGKAANFLEKMQRADGSWYGCWGVCFTYAGWFGIKGLVAAGRTYNSCLAIRKACEFLLSKELPGGGWGESYLSCQNKVYTNLEGNKPHLVNTAWVLMALIEAGQGERDPAPLHRAARLLMNSQLENGDFVQQEIMGVFNKNCMITYAAYRNIFPIWALGEYCHRVLTE

SEQ ID NO: 46 - protein, 785 aa, synthetic construct
MWRLKVGKESVGEKEEKWIKSISNHLGRQVWEFCAENDDDDDDEAVIHVVANSSKHLLQQQRRQSSFENARKQFRNNRFHRKQSSDLFLTIQYEKEIARNGAKNGGNTKVKEGEDVKKEAVNNTLERALSFYSAIQTSDGNWASDLGGPMFLLPGLVIALYVTGVLNSVLSKHHRQEMCRYIYNHQNEDGGWGLHIEGSSTMFGSALNYVALRLLGEDANGGECGAMTKARSWILERGGATAITSWGKLWLSVLGVYEWSGNNPLPPEFWLLPYSLPFHPGRMWCHCRMVYLPMSYLYGKRFVGPITHMVLSLRKELYTIPYHEIDWNRSRNTCAQEDLYYPHPKMQDILWGSIYHVYEPLFNGWPGRRLREKAMKIAMEHIHYEDENSRYIYLGPVNKVLNMLCCWVEDPYSDAFKFHLQRIPDYLWLAEDGMRMQGYNGSQLWDTAFSIQAILSTKLIDTFGSTLRKAHHFVKHSQIQEDCPGDPNVWFRHIHKGAWPFSTRDHGWLISDCTAEGLKASLMLSKLPSKIVGEPLEKNRLCDAVNVLLSLQNENGGFASYELTRSYPWLELINPAETFGDIVIDYSYVECTSATMEALALFKKLHPGHRTKEIDAALAKAANFLENMQRTDGSWYGCWGVCFTYAGWFGIKGLVAAGRTYNNCVAIRKACHFLLSKELPGGGWGESYLSCQNKVYTNLEGNRPHLVNTAWVLMALIEAGQGERDPAPLHRAARLLINSQLENGDFPQQEIMGVFNKNCMITYAAYRNIFPIWALGEYSHRVLTE

SEQ ID NO: 47 - protein, 774 aa, synthetic construct
MWRLKVGKESVGEKEEKWIKSISNHLGRQVWEFCSGENENDDDEAIAVANNSASKFENARNHFRNNRFHRKQSSDLFLAIQCEKEIIRNGAKNEGTTKVKEGEDVKKEAVKNTLERALSFYSAVQTSDGNWASDLGGPMFLLPGLVIALYVTGVLNSVLSKHHRQEMCRYIYNHQNEDGGWGLHIEGSSTMFGSALNYVALRLLGEAADGGEHGAMTKARSWILERGGATAITSWGKLWLSVLGVYEWSGNNPLPPEFWLLPYSLPFHPGRMWCHCRMVYLPMSYLYGKRFVGPITPIVLSLRKELYTIPYHEIDWNRSRNTCAKEDLYYPHPKMQDILWGSIYHVYEPLFSGWPGKRLREKAMKIAMEHIHYEDENSRYICLGPVNKVLNMLCCWVEDPYSDAFKFHLQRIPDYLWLAEDGMRMQGYNGSQLWDTAFSIQAIISTKLIDTFGPTLRKAHHFVKHSQIQEDCPGDPNVWFRHIHKGAWPFSTRDHGWLISDCTAEGLKASLMLSKLPSKIVGEPLEKNRLCDAVNVLLSLQNENGGFASYELTRSYPWLELINPAETFGDIVIDYSYVECTSATMEALALFKKLHPGHRTKEIDAAIAKAANFLENMQKTDGSWYGCWGVCFTYAGWFGIKGLVAAGRTYNNCVAIRKACNFLLSKELPGGGWGESYLSCQNKVYTNLEGNKPHLVNTAWVMMALIEAGQGERDPAPLHRAARLLINSQLESGDFPQQEIMGVFNKNCMITYAAYRNIFPIWALGEYSHRVLDM

SEQ ID NO: 48 - protein, 647 aa, synthetic construct
DGNWASDLGGPMFLLPGLVIALYVTGVLNSVLSKHHRQEMCRYLYNHQNEDGGWGLHIEGTSTMFGSALNYVALRLLGEDADGGEGGAMTKARSWILDRGGATAITSWGKLWLSVLGVYEWSGNNPLPPEFWLLPYCLPFHPGRMWCHCRMVYLPMSYLYGKRFVGPITPIVLSLRKELYTIPYHEIDWNRSRNTCAKEDLYYPHPKMQDILWGSIYHLYEPLFTRWPGKRLREKALQMAMKHIHYEDENSRYICLGPVNKVLNMLCCWVEDPYSDAFKFHLQRVPDYLWVAEDGMRMQGYNGSQLWDTAFSVQAIISTKLIDSFGTTLKKAHDFVKDSQIQQDCPGDPNVWFRHIHKGAWPFSTRDHGWLISDCTAEGLKASLMLSKLPSEIVGEPLEKSRLCDAVNVLLSLQNENGGFASYELTRSYPWLELINPAETFGDIVIDYPYVECTSATMEALTLFKKLHPGRRTKEIDIAVARAANFLENMQRTDGSWYGCWGVCFTYAGWFGIKGLVAAGRTYNSCVAIRKACDFLLSKELPGGGWGESYLSCQNKVYTNLEGNRPHLVNTAWVLMALIEAGQAERDPAPLHRAARLLINSQLENGDFPQEEIMGVFNKNCMITYAAYRNIFPIWALGEYFHRVLTE

SEQ ID NO: 49 - protein, 761 aa, synthetic construct
MWRLKIGKESVGDNGAWLRSSNDHVGRQVWEFCPESGTPEELSKVEMARQSFSTDRLLKKHSSDLLMRIQYAKENQFVTNFPQVKLKEFEDVKEEATLTTLRRALNFYSTIQADDGHWPGDYGGPMFLLPGLVITLSVTGALNAVLSKEHQYEMCRYLYNHQNRDGGWGLHIEGPSTMFGTVLNYVTLRLLGEGPEGGQGAVEKACEWILEHGSATAITSWGKMWLSVLGAYEWSGNNPLPPEVWLCPYFLPIHPGRMWCHCRMVYLPMSYLYGKRFVGPITPIILSLRKELYAVPYHEVDWNKARNTCAKEDLYYPHPLVQDILWASLHYLYEPIFTRWPCKSLREKALRTVMQHIHYEDENTRYICIGPVNKVLNMLSCWVEDPYSESFKLHLPRILDYLWIAEDGMKMQGYNGSQLWDTAFAVQAIISTGLADEYGPILRKAHDFIKYSQVLEDCPGDLNFWYRHISKGAWPFSTVDHGWPISDCTSEGLKAVLLLSTLPSESVGEPLHMMRLYDAVNVILSLQNVDGGFPTYELTRSYQWLELINPAETFGDIVIDYPYVECTSAAIQALISFKKLFPEHRMEEIENCIGRAVEFIEKIQAADGSWYGSWGVCFTYAGWFGIKGLSAAGRTYNNSSNIRKACDFLLSKELATGGWGESYLSCQNKVYTNLEGARPHIVNTSWALLALIEAGQAERDPTPLHRAARILINSQMEDGDFPQEEIMGVFNKNCMISYSAYRNIFPIWALGEYTCRVLRAP

SEQ ID NO: 50 - protein, 763 aa, synthetic construct
MWKLKIGAETVGEGGSDGWLRSVNSHLGRQVWEFHPELGTPEELRQIQDARDAFFNHRFHKQHSSDLLMRIQFAKENPCVANPPQVKVKDTDEVTEESVTTTLRRAINFYSTIQAHDGHWAGDYGGPMFLLPGLVITLSVTGALNAVLSKEHQCEMCRYIYNHQNEDGGWGLHIEGPSTMFGTVLNYVSLRLLGEGAEDGLGTIENARKWILDHGGATAITSWGKMWLSVLGVYEWSGNNPLPPEVWLCPYTLPFHPGRMWCHCRMVYLPMSYLYGKRFVGPITPTIRSLRKELYTAPYHEIDWNRARNECAKEDLYYPHPLVQDVLWASLHYVYEPIFMRWPAKKLREKALSTVMQHIHYEDENTRYICIGPVNKVLNMLCCWVEDPNSEAFKLHLPRISDYLWIAEDGMKMQGYNGSQLWDTAFAVQAIVSTDLAEEYGPTIRKAHEYIKNSQVLEDCPGDLNFWYRHISKGAWPFSTADHGWPISDCTAEGLKAVLLLSQLSSETVGDSLDVKRLFNAVNVILSLQNGDGGFATYELTRSYQWLELINPAETFGDIVIDYPYVECTSAALEALTLFKKSYPGHRREEVENCITNAAMFIENIQAKDGSWYGSWGVCFTYAGWFGIKGLVASGRTYENCPSIRKACDFLLSKELPSGGWGESYLSCQNKVYTNLKDNKPHIVNTAWAMLALIVARQAERDPMPLHRAARILIKSQMHDGDFPQEEIMGVFNKNCMISYAAYRNIFPIWALGEYRLHVLRSL

SEQ ID NO: 51 - protein, 762 aa, synthetic construct
MWKLKIGAETVGEGGYQWLKSVNNHLGRQVWEFNPELGSPEELQRIEDARKAFWDNRFERRHSSDLLMRIQFEKENQCVTNLPQLKVKYEEEVTEEVVKTTLRRAISFYSTIQAHDGHWPGDYGGPMFLLPGLVITLSITGALNDVLSKEHQHEMCRYLYNHQNKDGGWGLHIEGPSTMFGTALNYVTLRLFGEGADDGEGAMELARKWILDHGGVTKITSWGKMWLSVLGTYEWSGNNPLPPEVWLCPYSLPFHPGRMWCHCRMVYLPMSYLYGKRFVGPITPTIRSLRKELYGVPYHEVDWNQARNLCAKEDLYYPHPMVQDILWASLHYVYEPVFTRWPAKKLRENALQTVMQHIHYEDENTRYICIGPVNKVLNMLCCWAEDPNSDAFKLHLPRIPDYLWVAEDGMKMQGYNGSQSWDTSFAVQAIISTNLAEEFGPTLRKAHEYIKDSQVLEDCPGDLNFWYRHISKGAWPFSTADHGWPISDCTAEGLKAVLLLSKLPTGTVGESLDMKQLYDAVNVMLSLQNEDGGFATYELTRSYQWLELINPAETFGDIVIDYPYVECTSAAIQALTMFRKLYPGHRREEIESCIARAAKFIEKIQATDGSWYGSWGVCFTYAGWFGIKGLAAAGRTYKDCSSIRKACDFLLSKELPSGGWGESYLSCQNKVYTNLKDNRPHIVHTAWAMLALIGAGQAKRDPTPLHRAARVLINSQMENGDFPQKEIMGVFNKNCMISYSAYRNIFPIWALGEYRCQVLEAL

SEQ ID NO: 52 - protein, 759 aa, synthetic construct
MWKLKIAEGGSPWLRTTNNHVGRQFWEFDPNLGTPEELAAVEEARKSFRENRFAKKHSSDLLMRLQFSRESLSRPVLPQVNIKDGDDVTEKMVETTLKRGVDFYSTIQASDGHWAGDYGGPMFLLPGLIITLSITGALNTVLSEQHKAEMRRYLHNHQNEDGGWGLHIEGPSTMFGSVLNYVTLRLLGEGPNDGDGAMEKGRDWILNHGGATNITSWGKMWLSVLGAFEWSGNNPLPPEIWLLPYILPIHPGRMWCHCRMVYLPMSYLYGKRFVGPITSTVLSLRKELFTVPYHEVDWNEARNLCAKEDLYYPHPLVQDILWASLHKIVEPVLTRWPGSNLREKALRTTLEHIHYEDENTRYICIGPVNKVLNMLCCWVEDPNSEAFKLHLPRIHDYLWVAEDGMKMQGYNGSQLWDTSFAVQAVLATNFVEEYGPVLKKAHSYVKNSQVSEDCPGDLSYWYRHISKGAWPFSTADHGWPISDCTAEGLKAALLLSKVPKEIVGEPVDTKRLYDAVNVIISLQNADGGFATYELTRSYPWLELINPAETFGDIVIDYPYVECTSAAIQALIAFRKLYPGHRKKEVDECIEKAVKFIESIQESDGSWYGSWAVCFTYGTWFGVKGLEAAGKTLKNSPTVAKACEFLLSKQLPSGGWGESYLSCQDKVYSNLDGNRSHVVNTAWALLSLIGAGQVEVDQKPLHRAARYLINAQMESGDFPQQEIMGVFNRNCMITYAAYRNIFPIWALGEYRSKVLLQQGE

SEQ ID NO: 53 - protein, 756 aa, synthetic construct
MWKLKIAEGGSPWLRTTNNHVGRQIWEFDPNLGTPEQIREVEEARENFWKNRFEQKHSSDLLMRMQFAQENSSNVVLPQVKVKDEDEITEETVATTLRRALSYQSTIQAHDGHWPGDYGGPMFLMPGLVIALSVTGALNAVLSKEHQKEMCRYLYNHQNKDGGWGLHIEGHSTMFGTVLTYVTLRLLGEGVDDGDGAMERGRKWTLEHGSATAITSWGKMWLSVLGVFEWAGNNPMPPETWLLPYILPVHPGRMWCHCRMVYLPMSYLYGKRFVGPITPTVLSLRRELFDVPYHEIDWDRARNECAKEDLYYPHPLVQDILWASLHKAVEPILMRWPGKKLREKALSTVMEHIHYEDENTRYICIGPVNKVLNMLCCWVEDPNSEAFKLHLPRIPDFLWIAEDGMKMQGYNGSQLWDTTFMVQAILATNLGEEYGGTLRKAHNFIKDSQVREDCPGDLSYWYRHISKGAWPFSTADHGWPISDCTAEGLKAALLLSKVPSDIVGEPLEVKRLYDSVNVLLSLQNGDGGFATYELTRSYPWLELINPAETFGDIVIDYPYVECTSAAIQALVSFKRLYPGHRREEIENCIKKAAKFIEDIQAADGSWYGSWAVCFTYATWFGIKGLVAAGKNYDNCPAIRKACDFLLSKQLSNGGWGESYLSCQNKVYSNIEGNKAHVVNTGWAMLALIGAGQAKRDPMPLHRAAKVLINSQMPNGDFPQQEIMGVFNRNCMITYAAYRNIFPTWALGEYRTQVLQK

SEQ ID NO: 54 - protein, 756 aa, synthetic construct
MWKLKVAEGGSPWLRTVNNYVGRQVWEFDPNSGSPQELDQIESVRQNFHNNRFSHKHSDDLLMRIQLAKENPMGEVIPKVRVKDVEDVNEESVTTTLRRALNFYSTLQSRDGHWPGDYGGPMFLMPGLVIALSITGALNAVLTDEHQKEMRRYLYNHQNKDGGWGLHIEGPSTMFGSVLCYVTLRLLGEGPNDGEGEMEKARDWILEHGGATYITSWGKMWLSVLGVFEWSGNNPLPPEIWLLPYMLPIHPGRMWCHCRMVYLPMSYLYGKRFVGPITPTVLSLRKELFTVPYHDIDWNQARNLCAKEDLYYPHPLVQDILWASLHKFVEPIFMNWPGKKLREKAVETVMEHVHYEDENTRYICIGPVNKVLNMLCCWVEDPNSEAFKLHLPRIHDFLWIAEDGMKMQGYNGSQLWDTAFAVQAXISTNLIDEFAPTLRKAHTFIKNSQVLEDCPGDLSKWYRHISKGAWPFSTADHGWPISDCTAEGLKAVLLLSKIGPEIVGEPLDAKGFYDAVNVIISLQNEDGGLATYELTRSYKWLEIINPAETFGDIVIDYTYVECTSAAIQALSTFRKLYPGHRREEIQHCIEKAAAFIEKIQASDGSWYGSWGVCFTYGTWFGVKGLIAAGKSFSNCLSIRKACDFLLSKQLPSGGWGESYLSCQNKVYSNLESNRSHVVNTGWAMLALIEAEQAKRDPTPLHHAAVCLINSQMENGDFPQEEIMGVFNKNCMITYAAYRNIFPIWALGEYRRHVLQA

SEQ ID NO: 55 - protein, 759 aa, synthetic construct
MWKLRIAEGSGNPWLRTTNDHIGRQVWEFDSSKIGSPEELSQIENARQNFTKNRFIHKHSSDLLMRIQFSKENPICEVLPQVKVKESEQVTEEKVKITLRRALNYYSSIQADDGHWPGDYGGPMFLMPGLIIALSITGALNAILSEEHKREMCRYLYNHQNRDGGWGLHIEGPSTMFGSVLCYVSLRLLGEGPNEGEGAVERGRNWILKHGGATAITSWGKMWLSVLGAYEWSGNNPLPPEMWLLPYILPVHPGRMWCHCRMVYLPMSYLYGKRFVGPITPTVLSLRKELYTVPYHEIDWNQARNQCAKEDLYYPHPMLQDVLWATLHKFVEPILMHWPGKRLREKAIQTAIEHIHYEDENTRYICIGPVNKVLNMLCCWVEDPNSEAFKLHLPRLYDYLWLAEDGMKMQGYNGSQLWDTAFAVQAIVSTNLIEEYGPTLKKAHSFIKKMQVLENCPGDLNFWYRHISKGAWPFSTADHGWPISDCTAEGIKALMLLSKIPSEIVGEGLNANRLYDAVNVVLSLQNGDGGFPTYELSRSYSWLEFINPAETFGDIVIDYPYVECTSAAIQALTSFRKSYPEHQREEIECCIKKAAKFMEKIQISDGSWYGSWGVCFTYGTWFGIKGLVAAGKSFGNCSSIRKACDFLLSKQCPSGGWGESYLSCQKKVYSNLEGDRSHVVNTAWAMLSLIDAGQAERDPTPLHRAARYLINAQMENGDFPQQEIMGVFNRNCMITYAAYRDIFPIWALGEYRCRVLKAS

SEQ ID NO: 56 - protein, 313 aa, synthetic construct
MEKIEHSTIATNGINMHVASAGSGPAVLFLHGFPELWYSWRHQLLYLSSLGYRAIAPDLRGFGDTDAPPSPSSYTAHHIVGDLVGLLDQLGVDQVFLVGDWGAMMAWYFCLFRPDRVKALVNLSVHFTPRNPAISPLDGFRLMLGDDFYVCKFQEPGVAEADFGSVDTATMFKKFLTMRDPRPPIIPNGFRSLATPEALPSWLTEEDIDYFAAKFAKTGFTGGFNYYRAIDLTWELTAPWSGSEIKVPTKFIVGDLDLVYHFPGVKEYIHGGGFKKDVPFLEEVVVMEGAAHFINQEKADEINSLIYDFIKQF

SEQ ID NO: 57 - protein, 316 aa, synthetic construct
MEKIEHTTISTNGINMHVASIGSGPAVLFLHGFPELWYSWRHQLLFLSSMGYRAIAPDLRGFGDTDAPPSPSSYTAHHIVGDLVGLLDQLGIDQVFLVGHDWGAMMAWYFCLFRPDRVKALVNLSVHFLRRHPSIKFVDGFRALLGDDFYFCQFQEPGVAEADFGSVDVATMLKKFLTMRDPRPPMIPKEKGFRALETPDPLPAWLTEEDIDYFAGKFRKTGFTGGFNYYRAFNLTWELTAPWSGSEIKVAAKFIVGDLDLVYHFPGAKEYIHGGGFKKDVPLLEEVVVVDGAAHFINQERPAEISSLIYDFIKKF

SEQ ID NO: 58 - protein, 316 aa, synthetic construct
MDQIEHITINTNGIKMHIASVGTGPVVLLLHGFPELWYSWRHQLLYLSSVGYRAIAPDLRGYGDTDSPASPTSYTALHIVGDLVGALDELGIEKVFLVGHDWGAIIAWYFCLFRPDRIKALVNLSVQFIPRNPAIPFIEGFRTAFGDDFYMCRFQVPGEAEEDFASIDTAQLFKTSLCNRSSAPPCLPKEIGFRAIPPPENLPSWLTEEDINYYAAKFKQTGFTGALNYYRAFDLTWELTAPWTGAQIQVPVKFIVGDSDLTYHFPGAKEYIHNGGFKKDVPLLEEVVVVKDACHFINQERPQEINAHIHDFINKF

SEQ ID NO: 59 - protein, 314 aa, synthetic construct
MEKIEHSTIAANGITIHVASVGSGPAVLLLHGFPELWYSWRHQLLFLASKGYRAIAPDLRGFGDSDAPPSPSSYTPLHIVGDLVALLDHLGIDLVFLVGHDWGAMMAWHFCLLRPDRVKALVNLSVHFMPRNPAMSPLDGMRLLLGDDFYVCRFQEPGAAEADFGSVDTATMMKKFLTMRDPRPPIIPNGFRSLETPQALPPWLTEEDIDYFAAKFAKTGFTGGFNYYRAIGRTWELTAPWTGSKIKVPAKFIVGDLDMVYHLPDAKEYIHGGGFKEDVPLLEEVVVIEGAAHFINQEKPDEISSLIYDFIKKF

SEQ ID NO: 60 - protein, 314 aa, synthetic construct
MEKIEHSTIATNGINMHVASIGSGPPVLFLHGFPELWYSWRHQLLFLASKGFRAIAPDLRGFGDSDVPPSPSSYTPFHIIGDLIGLLDHLGIEQVFLVGHDWGAMMAWYFCLFRPDRVKALVNLSVHYNPRNPAISPLSRTRQFLGDDFYICKFQTPGVAEADFGSVDTATMMKKFLTIRDPSPPIIPNGFKTLKTPETLPSWLTEEDIDYFASKFTKTGFTGGFNYYRAIEQTWELTGPWSGAKIKVPTKYVVGDVDMVYHLPGAKQYIHGGGFKKDVPLLEEVVVMEGAAHFINQEKADEISAHIYDFIIKF

SEQ ID NO: 61 - protein, 316 aa, synthetic construct
MENIEHTIVPTNGINMHIASIGSGPAVLFLHGFPELWYSWRHQLLFLASNGFRAIAPDLRGFGDTDVPPSPSSYTAHHIVGDLIGLLDHLGIDRVFLVGHDWGAMMAWYFCLFRPDRVRALVNLSVHYLHRHPSIKFVDGFRAFLGDDFYFCQFQEPGVAEADFGSVDTATMLKKFLTMRDPRPPMIPKEKGFRALETPDPLPSWLTEEDVDYFASKFSKTGFTGGFNYYRAFDLSWELTAPWSGSQVKVPAKFIVGDLDLVYHFPGAKEYIHGGRFKEDVPFLEEVVVIEGAAHFINQERADEISSLIYEFINKF

SEQ ID NO: 62 - protein, 316 aa, synthetic construct
MEKIEHTTVSTNGINMHIASIGTGPVVLFLHGFPELWYSWRHQLLSLSSLGYRCIAPDLRGFGDTDAPPSPASYSALHIVGDLIGLLDHLGIDQVFLVGHDWGAVIAWWFCLFRPDRVKALVNMSVAFSPRNPKRKPVDGFRALFGDDYYICRFQEPGEIEKEFAGYDTTSIMKKFLTGRSPKPPCLPKELGLRAWKTPETLPPWLSEEDLNYFASKFSKTGFVGGLNYYRALNLTWELTGPWTGLQVKVPVKFIVGDLDITYHIPGVKNYIHNGGFKRDVPFLQEVVVIEDGAHFINQERPDEISRHVYDFIQKF

SEQ ID NO: 63 - protein, 316 aa, synthetic construct
MEKIEHSTVHTNGINMHVASVGTGPAILFLHGFPELWYSWRHQMISLSSLGYRCIAPDLRGYGDTDAPPSPTSYTSLHIVGDLVGLIDHLVIEKLFLVGHDWGAMIAWYFCLFRPDRIKALVNLSVPFFPRNPKINFVDGFRAELGDDFYICRFQEPGESEADFSSDTVAVFRRILANRDPKPPLIPKEIGFRGVYEDPVALPSWLTEDDINHFANKFNETGFTGGLNYYRALNLTWELTAAWTGARVQVPTKFIMGDLDLVYYFPGMKEYILNGGFKRDVPLLQELVIIEGAAHFINQEKPDEISSHIHHFIQKF

SEQ ID NO: 64 - protein, 316 aa, synthetic construct
MEKIEHTTVATNGINMHVAAIGTGPEILFLHGFPELWYSWRHQLLSLSSRGYRCIAPDLRGYGDTDAPESLTGYTALHIVGDLIGLLDSMGIEQVFLVGHDWGAMMAWYLCMFRPDRIKALVNTSVAYMSRNPQLKSLELFRTVYGDDYYVCRFQEPGGAEEDFAQVDTAKLIRSVFTSRDPNPPIVPKEIGFRSLPDPPSLPSWLSEEDVNYYADKFNKKGFTGGLNYYRNIDQNWELTAPWDGLQIKVPVKFVIGDLDLTYHFPGIKDYIHNGGFKQVVPLLQEVVVMEGVAHFINQEKPEEISEHIYDFIKKF

SEQ ID NO: 65 - protein, 316 aa, synthetic construct
MEKIEHTTVGTNGINMHVASIGTGPVVLFIHGFPELWYSWRNQLLYLSSRGYRAIAPDLRGYGDTDAPPSVTSYTALHLVGDLIGLLDKLGIHQVFLVGHDWGALIAWYFCLFRPDRVKALVNMSVPFPPRNPAVRPLNNFRAVYGDDYYICRFQEPGEIEEEFAQIDTARLMKKFLCLRIAKPLCIPKDTGLSTVPDPSALPSWLSEEDVNYYASKFNQKGFTGPVNYYRCSDLNWELMAPWTGVQLEVPVKFIVGDQDLVYNNKGMKEYIHNGGFKKYVPYLQEVVVMEGVAHFINQEKAEEVGAHIYEFIKKF

SEQ ID NO: 66 - protein, 316 aa, synthetic construct
MEKIEHITVFTNGINMHIASIGTGPEILFLHGFPELWYSWRHQLLSLSSLGYRCIAPDLRGYGDTDAPQSVNQYTVLHIVGDLVGLLDSLGIQQVFLVGHDWGAFIAWYFCIFRPDRIKALVNTSVAFMPRNPQVKPLDGLRSMFGDDYYICQFQKPGKAEEDFAQVNTAKLIKLLFTSRDPRPPHFLKEVGLKALQDPPSQQSWLTEEDVNFYAAKFNQKGFRGGLNYYQNINMNWELAAAWTGVQIKVPVKFIIGDLDLTYHFPGIKEYIHNGGFKKDVPLLQDVVVMEGVAHFLNQEKPEEVSKHIYDFIKKF

SEQ ID NO: 67 - protein, 317 aa, synthetic construct
MDKIQHKIIQTNGINIHVAEIGDGPAVLFLHGFPELWYSWRHQMLFLSSRGYRAIAPDLRGYGDSDAPPCATSYTAFHIIGDLVGLLDAMGLDRVFLVGHDWGAVMAWYFCLLRPDRIKALVNLSVVFQPRNPKRKPVESMRAKLGDDYYICRFQEPGEAEEEFARVDTARLIKKLLTTRNPAPPRLPKEVGFGCLPHKPITMPSWLSEEDVQYYAAKFNQKGFTGGLNYYRAMDLSWELAAPWTGVQIKVPVKFIVGDLDITYNTPGVKEYIHKGRFKQHVPFLQELVILEGVAHFLNQEKPDEINQHIYDFIHKF

SEQ ID NO: 68 - protein, 324 aa, synthetic construct
MEKIEHTTVSTNGINMHVASIGSGPVILFLHGFPDLWYSWRHQLLSFAALGYRAIAPDLRGYGDSDAPPSPESYTILHIVGDLVGLLDSLGVDRVFLVGHDWGAIVAWWLCMIRPDRVKALVNTSVVFNPRNPSVKPVDKFRDLFGDDYYVCRFQETGEIEEDFAQVDTKKLITRFFVSRNPRPPCIPKSVGFRGLPDPPSLPAWLTEQDVSFYGDKFSQKGFTGGLNYYRAMNLSWELTAPWAGLQIKVPVKFIVGDLDITYNIPGTKEYIHGGGLKKHVPFLQEVVVMEGVGHFLQQEKPDEVTDHIYGFFEKFRTRETSSL

SEQ ID NO: 69 - protein, 317 aa, synthetic construct
MDKIQHRQVPVNGINLHVAEIGDGPAILFLHGFPELWYSWRHQLLSLSAKGYRALAPDLRGYGDSDAPPSPSNYTALHIVGDLVGLLDSLGLDRVFLVGHDWGAVMAWYFCLLRPDRIKALVNMSVVFTPRNPKRKPLEAMRARFGDDYYICRFQEPGEAEEEFARVDTARIIKKFLTSRRPGPLCVPKEVGFGGSPHNPIQLPSWLSEDDVNYFASKFSQKGFTGGLNYYRAMDLNWELTAPWTGLQIKVPVKFIVGDLDVTFTTPGVKEYIQKGGFKRDVPFLQELVVMEGVAHFVNQEKPEEVSAHIYDFIQKF

SEQ ID NO: 70 - protein, 318 aa, synthetic construct
MEKIQHTTVRTNGINMHVATAGSGPDSILFVHGFPELWYTWRHQMVSLAALGYRTIAPDLRGYGDTDAPPSHESYTAFHIVGDLVGLLDSMGIEKVFLVGHDWGAAIAWYFCLFRPDRIKALVNMSVVFHPRNPNRKPVDGLRAILGDDYYICRFQAPGEIEEDFARADTANIIKFFLVSRNPRPPQIPKEGFSCLANSRQMDLPSWLSEEDINYYASKFSEKGFTGGLNYYRVMNLNWELTAPFTGLQIKVPAKFMVGDLDITYNTPGTKEFIHNGGLKKHVPFLQEVVVMEGVAHFINQEKPEEVTAHIYDFIKKF

SEQ ID NO: 71 - protein, 324 aa, synthetic construct
MEKIEHTTVSTNGINMHVASIGSGPVILFLHGFPDLWYSWRHQLLSFAALGYRAIAPDLRGYGDSDAPPSRESYTILHIVGDLVGLLNSLGVDRVFLVGHDWGAIVAWWLCMIRPDRVNALVNTSVVFNPRNPSVKPVDAFRALFGDDYYICRFQEPGEIEEDFAQVDTKKLITRFFISRNPRPPCIPKSVGFRGLPDPPSLPAWLTEEDVSFYGDKFSQKGFTGGLNYYRALNLSWELTAPWAGLQIKVPVKFIVGDLDITYNIPGTKEYIHEGGLKKHVPFLQEVVVLEGVGHFLHQEKPDEITDHIYGFFKKFRTRETASL

SEQ ID NO: 72 - protein, 316 aa, synthetic construct
MDKIEHTTVSTNGINMHVASIGSGPVILFLHGFPDLWYSWRHQLLSFAGLGYRAIAPDLRGYGDSDSPPSHESYTILHIVGDLVGLLDSLGVDRVFLVGHDWGAVVAWWLCMIRPDRVNALVNTSVVFNPRNPSVKPVDAFKALFGEDYYVCRFQEPGEIEEDFAQVDTKKLINRFFTSRNPRPPCIPKTLGFRGLPDPPALPAWLTEQDVSFYADKFSQKGFTGGLNYYRAMNLSWELTAPWAGLQIKVPVKFIVGDLDITYNIPGTKEYIHEGGLKKHVPFLQEVVVMEGVGHFLHQEKPDEVTDHIYGFFKKF

SEQ ID NO: 73 - protein, 473 aa, synthetic construct
MWTVVLGLATLFVAYYIHWINKWRDSKFNGVLPPGTMGLPLIGETIQLSRPSDSLDVHPFIQKKVERYGPIFKTCLAGRPVVVSADAEFNNYIMLQEGRAVEMWYLDTLSKFFGLDTEWLKALGLIHKYIRSITLNHFGAEALRERFLPFIEASSMEALHSWSTQPSVEVKNASALMVFRTSVNKMFGEDAKKLSGNIPGKFTKLLGGFLSLPLNFPGTTYHKCLKDMKEIQKKLREVVDDRLANVGPDVEDFLGQAFKDKESEKFISEEFIIQLLFSISFASFESISTTLTLILKLLDEHPEVVKELEVEHEAIRKARADPDGPITWEEYKSMTFTLQVINETLRLGSVTPALLRKTVKDLQVKGKIIPEGWTIMLVTASRHRDPKVYKDPHIFNPWRWKDLDSITIQKNFMPFGGGLRHCAGAEYSKVYLCTFLHILCTKYRWTKLGGGTIARAHILSFEDGLHVKFTPKE

SEQ ID NO: 74 - protein, 474 aa, synthetic construct
MWTILLGLATLAIAYYIHWVNKWKDSKFNGVLPPGTMGLPLIGETIQLSRPSDSLDVHPFIQSKVKRYGPIFKTCLAGRPVVVSTDAEFNHYIMLQEGRAVEMWYLDTLSKFFGLDTEWLKALGLIHKYIRSITLNHFGAESLRERFLPRIEESARETLHYWSTQPSVEVKESAAAMVFRTSIVKMFSEDSSKLLTAGLTKKFTGLLGGFLTLPLNVPGTTYHKCIKDMKEIQKKLKDILEERLAKGVSIDEDFLGQAIKDKESQQFISEEFIIQLLFSISFASFESISTTLTLILNFLADHPDVAKELEAEHEAIRKARADPDGPITWEEYKSMNFTLNVICETLRLGSVTPALLRKTTKEIQIKGYTIPEGWTVMLVTASRHRDPEVYKDPDTFNPWRWKELDSITIQRNFMPFGGGLRHCAGAEYSKVYLCTFLHILFTKYRWRKLKGGKIARAHILRFEDGLYVNFTPKE

SEQ ID NO: 75 - protein, 474 aa, synthetic construct
MWTIVVGLATLAVAYYIHWINKWKDSKFNGVLPPGTMGLPLIGETLQLSRPSDSLDVHPFIKKKVKRYGSIFKTCLAGRPVVVSTDAEFNNYIMLQEGRAVEMWYLDTLSKFFGLDTEWLKALGFIHKYIRSITLNHFGAESLRERFLPRIEESAKETLCYWATQPSVEVKDSAAVMVFRTSMVKMVSKDSSKLLTGGLTKKFTGLLGGFLTLPINVPGTTYNKCMKDMKEIQKKLREILEGRLASGAGSDEDFLGQAVKDKGSQKFISDDFIIQLLFSISFASFESISTTLTLILNYLADHPDVVKELEAEHEAIRNARADPDGPITWEEYKSMTFTLHVIFETLRLGSVTPALLRKTTKELQINGYTIPEGWTVMLVTASRHRDPAVYKDPHTFNPWRWKELDSITIQKNFMPFGGGLRHCAGAEYSKVYLCTFLHILFTKYRWTKLKGGKVARAHILSFEDGLHMKFTPKE

SEQ ID NO: 76 - protein, 474 aa, synthetic construct
MWTILLGLATLAIAYYIHWVNKWKDSKFNGVLPPGTMGLPLIGETIQLSRPSDSLDVHPFIQRKVKRYGPIFKTCLAGRPVVVSTDAEFNHYIMLQEGRAVEMWYLDTLSKFFGLDTEWLKALGLIHKYIRSITLNHFGAESLRERFLPRIEESARETLHYWSTQTSVEVKESAAAMVFRTSIVKMFSEDSSKLLTEGLTKKFTGLLGGFLTLPLNLPGTTYHKCIKDMKQIQKKLKDILEERLAKGVKIDEDFLGQAIKDKESQQFISEEFIIQLLFSISFASFESISTTLTLILNFLADHPDVVKELEAEHEAIRKARADPDGPITWEEYKSMNFTLNVICETLRLGSVTPALLRKTTKEIQIKGYTIPEGWTVMLVTASRHRDPEVYKDPDTFNPWRWKELDSITIQKNFMPFGGGLRHCAGAEYSKVYLCTFLHILFTKYRWRKLKGGKIARAHILRFEDGLYVNFTPKE

SEQ ID NO: 77 - protein, 474 aa, synthetic construct
MWAIVVGLATLAVAYYIHWINKWKDSKFNGVLPPGTMGLPLVGETLQLARPSDSLDVHPFIKKKVKRYGSIFKTCLAGRPVVVSTDAEFNNYIMLQEGRAVEMWYLDTLSKFFGLDTEWLKALGFIHKYIRSITLNHFGAESLRERFLPRIEESAKETLRYWATQPSVEVKDSAAVMVFRTSMVKMVSEDSSKLLTGGLTKKFTGLLGGFLTLPINVPGTTYNKCMKDMKEIQKKLREILEGRLASGAGSDEDFLGQAIKDKGSQQFISDDFIIQLLFSISFASFESISTTLTLVLNYLADHPDVVKELEAEHEAIRNARADPDGPITWEEYKSMTFTLHVIFETLRLGSVTPALLRKTTKELQINGYTIPEGWTVMLVTASRHRDPAVYKDPHTFNPWRWKELDSITIQKNFMPFGGGLRHCAGAEYSKVYLCTFLHILFTKYRWTKLKGGKVARAHILSFEDGLHVKFTPKE

SEQ ID NO: 78 - protein, 476 aa, synthetic construct
MWTLVGLSLVALLVIYFTHWIIKWRNPKCNGVLPPGSMGLPLIGETLNLIIPSYSLDLHPFIKKRLQRYGPIFRTSLAGRPVVVTADPEFNNYIFQQEGRMVELWYLDTFSKIFVHEGDSKTNAIGMVHKYVRSIFLNHFGAERLKEKLLPQIEEFVNKSLCAWSSKASVEVKHAGSVMVFNFSAKQMISYDAEKSSDDLSEKYTKIIDGLMSFPLNIPGTAYYNCSKHQKNVTTMLRDMLKERRISPETRRGDFLDQLSIDMEKEKFLSEDFSVQLVFGGLFATFESISAVIALAFSLLADHPSVVEELTAEHEAILKNRENPNSSITWDEYKSMTFTLQVINEILRLGNVAPGLLRRALKDIPVKGFTIPEGWTIMVVTSALQLSPNTFEDPLEFNPWRWKDLDSYAVSKNFMPFGGGMRQCAGAEYSRVFLATFLHVLVTKYRWTTIKAARIARNPILGFGDGIHIKFEEKKT

SEQ ID NO: 79 - protein, 474 aa, synthetic construct
MWAIGLVVVALVVIYYTHMIFKWRSPKIEGVLPPGSMGWPLIGETLQFISPGKSLDLHPFVKKRMEKYGPIFKTSLVGRPIIVSTDYEMNKYILQHEGTLVELWYLDSFAKFFALEGETRVNAIGTVHKYLRSITLNHFGVESLKESLLPKIEDMLHTNLAKWASQGPVDVKQVISVMVFNFTANKIFGYDAENSKEKLSENYTKILNSFISLPLNIPGTSFHKCMQDREKMLKMLKDTLMERLNDPSKRRGDFLDQAIDDMKTEKFLTEDFIPQLMFGILFASFESMSTTLTLTFKFLTENPRVVEELRAEHEAIVKKRENPNSRLTWEEYRSMTFTQMVVNETLRISNIPPGLFRKALKDFQVKGYTVPAGWTVMLVTPATQLNPDTFKDPVTFNPWRWQELDQVTISKNFMPFGGGTRQCAGAEYSKLVLSTFLHILVTNYSFTKIRGGDVSRTPIISFGDGIHIKFTARA

SEQ ID NO: 80 - protein, 476 aa, synthetic construct
MWTLVGLSLVGLLVIYFTHWIIKWRNPKCNGVLPPGSMGLPFIGETLNLIIPSYSLDLHPFIKKRLQRYGPIFRTSLAGRQVVVTADPEFNNYLFQQEGRMVELWYLDTFSKIFVHEGESKTNAVGMVHKYVRSIFLNHFGAERLKEKLLPQIEEFVNKSLCAWSSKASVEVKHAGSVMVFNFSAKQMISYDAEKSSDDLSEKYTKIIDGLMSFPLNIPGTAYYNCLKHQKNVTTMLRDMLKERQISPETRRGDFLDQISIDMEKEKFLSEDFSVQLVFGGLFATFESISAVLALAFSLLAEHPSVVEELTAEHEAILKNRENLNSSLTWDEYKSMTFTLQVINEILRLGNVAPGLLRRALKDIPVKGFTIPEGWTIMVVTSALQLSPNTFEDPLEFNPWRWKDLDSYAVSKNFMPFGGGMRQCAGAEYSRVFLATFLHVLVTKYRWTTIKAARIARNPILGFGDGIHIKFEEKKT

SEQ ID NO: 81 - protein, 476 aa, synthetic construct
MWTFVLCVVAVLVVYYTHWINKWRNPTCNGVLPPGSMGLPIIGETLELIIPSYSLDLHPFIKKRIQRYGPIFRTNILGRPAVVSADPEINSYIFQNEGKLVEMWYMDTFSKLFAQSGESRTNAFGIIHKYARSLTLTHFGSESLKERLLPQVENIVSKSLQMWSSDASVDVKPAVSIMVCDFTAKQLFGYDAENSSDKISEKFTKVIDAFMSLPLNIPGTTYHKCLKDKDSTLSILRNTLKERMNSPAESRGGDFLDQIIADMDKEKFLTEDFTVNLIFGILFASFESISAALTLSLKLIGDHPSVLEELTVEHEAILKNRENPDSPLTWAEYNSMTFSLQVINETLRLGNVAPGLLRRALQDMQVKGYTIPAGWVIMVVNSALHLNPATFKDPLEFNPWRWKDFDSYAVSKNLMPFGGGRRQCAGSEFTKLFMAIFLHKLVTKYRWNIIKQGNIGRNPILGFGDGIHISFSPKDI

SEQ ID NO: 82 - protein, 475 aa, synthetic construct
MWKVGLCVVGVIVVWFTRWINKWRNPKCNGILPPGSMGPPLIGESLQLIIPSYSLDLHPFIKKRVQRYGPIFRTSVVGQPMVVSTDVEFNHYLAKQEGRLVHFWYLDSFAEIFNLEDENAISAVGLIHKYGRSIVLNHFGTDSLKKTLLSQIEEIVNKTLQTWSSLPSVEVKHAASVMAFDLTAKQCFGYDVENSAVKMSEKFLYTLDSLISFPFNIPGTVYHKCLKDKKEVLNMLRNIVKERMNSPEKYRGDFLDQITADMNKESFLTQDFIVYLLYGLLFASFESISASLSLTLKLLAEHPAVLQQLTAEHEAILKNRDNPNSSLTWDEYKSMTFTFQVINEALRLGNVAPGLLRRALKDIEFKGYTIPAGWTIMLANSAIQLNPNTYEDPLAFNPWRWQDLDPQIVSKNFMPFGGGIRQCAGAEYSKTFLATFLHVLVTKYRWTKVKGGKMARNPILWFADGIHINFALKHN

SEQ ID NO: 83 - protein, 476 aa, synthetic construct
MWDVVGLSFVALLVIYLTYWITQWKNPKCNGVLPPGSMGLPLIGETLNLLIPSYSLDLHPFIRKRLERYGPIFRTSLAGKPVLVSADPEFNNYVLKQEGRMVEFWYLDTFSKIFMQEGGNGTNQIGVIHKYARSIFLNHFGAECIKEKLLTQIEGSINKHLRAWSNQESVEVKKAGSIMALNFCAEHMIGYDAETATENLGEIYHRVFQGLISFPLNVPGTAYHNCLKIHKKATTMLRAMLRERRSSPEKRRGDFLDQIIDDLDQEKFLSEDFCIHLIFGGLFAIFESISTVLTLFFSLLADHPAVLQELTAEHEALLKNREDPNSALTWDEYKSMTFTLQVINETLRLVNTAPGLLRRALKDIPVKGYTIPAGWTILLVTPALHLTSNTFKDHLEFNPWRWKDLDSLVISKNFMPFGSGLRQCAGAEFSRAYLSTFLHVLVTKYRWTTIKGARISRRPMLTFGDGAHIKFSEKKN

SEQ ID NO: 84 - protein, 475 aa, synthetic construct
MWNTICLSVVGLVVIWISNWIRRWRNPKCNGVLPPGSMGFPLIGETLPLIIPTYSLDLHPFIKNRLQRYGSIFRTSIVGRPVVISADPEFNNFLFQQEGSLVELYYLDTFSKIFVHEGVSRTNEFGVVHKYIRSIFLNHFGAERLKEKLLPEIEQMVNKTLSAWSTQASVEVKHAASVLVLDFSAKQIISYDAKKSSESLSETYTRIIQGFMSFPLNIPGTAYNQCVKDQKKIIAMLRDMLKERRASPETNRGDFLDQISKDMDKEKFLSEDFVVQLIFGGLFATFESVSAVLALGFMLLSEHPSVLEEMIAEHETILKNREHPNSLLAWGEYKSMTFTLQVINETLRLGNVAPGLLRKALKDIRVKGFTIPKGWAIMMVTSALQLSPSTFKNPLEFNPWRWKDLDSLVISKNFMPFGRGMRQCAGAEYSRAFMATFFHVLLTKYRWTTIKVGNVSRNPILRFGNGIHIKFSKKN

SEQ ID NO: 85 - protein, 475 aa, synthetic construct
MWIIGLCFASLLVIYCTHFFYKWRNPKCKGVLPPGSMGLPIIGETLQLIIPSYSLDHHPFIQKRIQRYGPIFRTNLVGRPVIVSADPEVNQYIFQQEGNSVEMWYLDAYAKIFQLDGESRLSAVGRVHKYIRSITLNNFGIENLKENLLPQIQDLVNQSLQKWSNKASVDVKQAASVMVFNLTAKQMFSYGVEKNSSEEMTEKFTGIFNSLMSLPLNIPGTTYHKCLKDREAMLKMLRDTLKQRLSSPDTHRGDFLDQAIDDMDTEKFLTGDCIPQLIFGILLAGFETTATTLTLAFKFLAEHPLVLEELTAEHEKILSKRENLESPLTWDEYKSMTFTHHVINETLRLANFLPGLLRKALKDIQVKNYTIPAGWTIMVVKSAMQLNPEIYKDPLAFNPWRWKDLDSYTVSKNFMPFGGGSRQCAGADYSKLFMTIFLHVLVTKYRWRKIKGGDIARNPILGFGDGLHIEVSAKN

SEQ ID NO: 86 - protein, 475 aa, synthetic construct
MLTVVLLLVGFFIIYYTYWISKWRNPNCNGVLPPGSMGFPLIGETLQLLIPSYSLDLHPFIKKRIHRYGPIFRSNLAGRPVIVSADPEFNYYILSQEGRSVEIWYLDTFSKLFRQQGESRTNVAGYVHKYLRGAFLSQIGSENLREKLLLHIQDMVNRTLCSWSNQESVEVKHSASLAVCDFTAKVLFGYDAEKSPDNLSETFTRFVEGLISFPLNIPRTAYRQCLQDRQKALSILKNVLTDRRNSVENYRGDVLDLLLNDMGKEKFLTEDFICLIMLGGLFASFESISTITTLLLKLFSAHPEVVQELEAEHEKILVSRHGSDSLSITWDEYKSMTFTHQVINETLRLGNVAPGLLRRAIKDVQFKGYTIPSGWTIMMVTSAQQVNPEVYKDPLVFNPWRWKDFDSITVSKNFTPFGGGTRQCVGAEYSRLTLSLFIHLLVTKYRWTKIKEGEIRRAPMLGFGDGIHFKFSEKE

SEQ ID NO: 87 - protein, 498 aa, synthetic construct
MKRAIYICLARITKQGLSLIEMLMTELLFGAFFIIFLTYWINRWRNPKCNGVLPPGSMGLPLLGETLQLLIPRYSLDLHPFIRKRIQRYGPIFRSNVAGRPIVFTADPELNHYIFIQERRLVELWYMDTFSNLFVLDGESRPTGATGYIHKYMRGLFLTHFGAERLKDKLLHQIQELIHTTLQSWCKQPTIEVKHAASAVICDFSAKFLFGYEAEKSPFNMSERFAKFAESLVSFPLNIPGTAYHQSLEDREKVMKLLKNVLRERRNSTKKSEEDVLKQILDDMEKENFITDDFIIQILFGALFAISESIPMTIALLVKFLSAQPSVVEELTAEHEEILKNKKEKGLDSSITWEDYKSMTFTLQVINETLRIANVAPGLLRRTLRDIHYKGYTIPAGWTIMVLTSSRHMNPEIYKDPVEFNPWRWKDLDSQTISKNFTPFGGGTRQCAGAEYSRAFISMFLHVLVTKYRWKNVKEGKICRGPILRIEDGIHIKLYEKH

SEQ ID NO: 88 - protein, 476 aa, synthetic construct
MWPTMGLYVATIVAICFILLELKRRNSREKQVVLPPGSKGFPLIGETLQLLVPSYSLDLPSFIRTRIQRYGPIFKTRLVGRPVVMSADPGFNRYIVQQEGKSVEMWYLDTFSKLFAQDGEARTTAAGLVHKYLRNLTLSHFGSESLRVNLLPHLESLVRNTLLGWSSKDTIDVKESALTMTIEFVAKQLFGYDSDKSKEKIGEKFGNISQGLFSLPLNIPGTTYHSCLKSQREVMDMMRTALKDRLTTPESYRGDFLDHALKDLSTEKFLSEEFILQIMFGLLFASSESTSMTLTLVLKLLSENPHVLKELEAEHERIIKNKESPDSPLTWAEVKSMTFTLQVINESLRLGNVSLGILRRTLKDIEINGYTIPAGWTIMLVTSACQYNSDIYKDPLTFNPWRWKEMQPDVIAKNFMPFGGGTRQCAGAEFAKVLMTIFLHNLVTNYRWEKIKGGEIVRTPILGFRNALRVKLTKKN

SEQ ID NO: 89 - protein, 448 aa, synthetic construct
MVLLPGSKGFPFIGETLQLLLPSYSLDLPSFIRTRIQRYGPIFQTRLVGRPVVVSADPGFNRYIVQQEGKMVEMWYLDTFSKIFAQQGEGRTNAAGLVHKYLRNITFTHFGSQTLRDKLLPHLEILVRKTLHGWTSQESIDVKEAALTMTIEFVAKQLFGYDSDKSKERIGDKFANISQGLLSFPLNIPGTTYHSCLKSQREVMDMMRKTLKERLASPDTCQGDFLDHALKDLNTDKFLTEDFILQIMFGLLFASSESTSITLTLILKFLSENPHVLEELEVEHERILKNRESPDSPLTWAEVKSMTFTLQVINESLRLGNVSLGLLRRTLKDIEINGYTIPAGWTIMLVTSACQYNSDVYKDPLTFNPWRWKEMQPDVIAKNFMPFGGGTRQCAGAEFAKVLMTIFLHVLVTTYRWEKIKGGEIIRTPILGFRNGLHVKLIKKARLS

SEQ ID NO: 90 - protein, 477 aa, synthetic construct
MEMWSVWLYIISLIIIIATHWIYRWRNPKCNGKLPPGSMGIPFIGETIQFLIPSKSLDVPNFIKKRMNKYGPLFRTNLVGRPVIVSSDPDFNYYLLQREGKLVERWYMDSFSKLLHHDVTQIIIKHGSIHKYLRNLVLGHFGPEPLKDKLLPQLESAISQRLQDWSKQPSIEAKSASSAMIFDFTAKILFSYEPEKSGENIGEIFSNFLQGLMSIPLNIPGTAFHRCLKNQKRAIQMITEILKERRSNPEIHKGDFLDQIVEDMKKDSFWTEEFAIYMMFGLLLASFETISSTLALAIIFLTDNPPVVQKLTEEHEAILKARENRDSGLSWKEYKSLSYTHQVVNESLRLASVAPGILRRAITDIQVDGYTIPKGWTIMVVPAAVQLNPNTFEDPLVFNPSRWEDMGAVAMAKNFIAFGGGSRSCAGAEFSRVLMSVFVHVFVTNYRWTKIKGGDMVRSPALGFGNGFHIRVSEKQL

SEQ ID NO: 91 - protein, 474 aa, synthetic construct
MAALDLSTVGYLIVGLLTVYITHWIYKWRNPKCNGVLPPGSMGLPLIGETIQLVIPNASLDLPPFIKKRMKRYGPIFRTNVAGRPVIITADPEFNHFLLRQDGKLVDTWSMDTFAEVFDQASQSSRKYTRHLTLNHFGVEALREKLLPQMEDMVRTTLSNWSSQESVEVKSASVTMAIDYAARQIYSGNLENAPLKISDLFRDLVDGLMSFPINIPGTAHHRCLQTHKKVREMMKDIVKTRLEEPERQYGDMLDHMIEDMKKESFLDEDFIVQLMFGLFFVTSDSISTTLALAFKLLAEHPLVLEELTAEHEAILKKREKSESHLTWNDYKSMTFTLQVINEVLRLGNIAPGFFRRALQDIPVNGYTIPSGWVIMIATAGLHLNSNQFEDPLKFNPWRWKVCKVSSVIAKCFMPFGSGMKQCAGAEYSRVLLATFIHVLTTKYRWAIVKGGKIVRSPIIRFPDGFHYKIIEKTN

SEQ ID NO: 92 - protein, 711 aa, synthetic construct
MAQSDSVKVSPFDLVSAAMNGKAMEKLNASESEDPTTLPALKMLVENRELLTLFTTSFAVLIGCLVFLMWRRSSSKKLVQDPVPQVIVVKKKEKESEVDDGKKKVSIFYGTQTGTAEGFAKALVEEAKVRYEKTSFKVIDLDDYAADDDEYEEKLKKESLAFFFLATYGDGEPTDNAANFYKWFTEGDDKGEWLKKLQYGVFGLGNRQYEHFNKIAIVVDDKLTEMGAKRLVPVGLGDDDQCIEDDFTAWKELVWPELDQLLRDEDDTSVTTPYTAAVLEYRVVYHDKPADSYAEDQTHTNGHVVHDAQHPSRSNVAFKKELHTSQSDRSCTHLEFDISHTGLSYETGDHVGVYSENLSEVVDEALKLLGLSPDTYFSVHADKEDGTPIGGASLPPPFPPCTLRDALTRYADVLSSPKKVALLALAAHASDPSEADRLKFLASPAGKDEYAQWIVANQRSLLEVMQSFPSAKPPLGVFFAAVAPRLQPRYYSISSSPKMSPNRIHVTCALVYETTPAGRIHRGLCSTWMKNAVPLTESPDCSQASIFVRTSNFRLPVDPKVPVIMIGPGTGLAPFRGFLQERLALKESGTELGSSIFFFGCRNRKVDFIYEDELNNFVETGALSELIVAFSREGTAKEYVQHKMSQKASDIWKLLSEGAYLYVCGDAKGMAKDVHRTLHTIVQEQGSLDSSKAELYVKNLQMSGRYLRDVW

SEQ ID NO: 93 - protein, 693 aa, synthetic construct
MATSALYASDLFKQLKSIMGTDSLSDDVVLVIATTSLALVAGFVVLLWKKTTADRSGELKPLMIPKSLMAKDEDDDLDLGSGKTRVSIFFGTQTGTAEGFAKALSEEIKARYEKAAVKVIDLDDYAADDDQYEEKLKKETLAFFCVATYGDGEPTDNAARFYKWFTEENERDIKLQQLAYGVFALGNRQYEHFNKIGIVLDEELCKKGAKRLIEVGLGDDDQSIEDDFNAWKESLWSELDKLLKDEDDKSVATPYTAVIPEYRVVTHDPRFTTQKSMESNVANGNTTIDIHHPCRVDVAVQKELHTHESDRSCIHLEFDISRTGITYETGDHVGVYAENHVEIVEEAGKLLGHSLDLVFSIHADKEDGSPLESAVPPPFPGPCTLGTGLARYADLLNPPRKSALVALAAYATEPSEAEKLKHLTSPDGKDEYSQWIVASQRSLLEVMAAFPSAKPPLGVFFAAIAPRLQPRYYSISSSPRLAPSRVHVTSALVYGPTPTGRIHKGVCSTWMKNAVPAEKSHECSGAPIFIRASNFKLPSNPSTPIVMVGPGTGLAPFRGFLQERMALKEDGEELGSSLLFFGCRNRQMDFIYEDELNNFVDQGVISELIMAFSREGAQKEYVQHKMMEKAAQVWDLIKEEGYLYVCGDAKGMARDVHRTLHTIVQEQEGVSSSEAEAIVKKLQTEGRYLRDVW

SEQ ID NO: 94 - protein, 712 aa, synthetic construct
MASSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 95 - protein, 713 aa, synthetic construct
MASSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDITLANGNGYTVFDAQHPYKANVAVKRELHTPESDRSCIHLEFDIAGSGLTMKLGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSEKLFLGRPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 96 - protein, 710 aa, synthetic construct
MAQSESVEASTIDLMTAVLKDTVIDTANASDNGDSKMPPALAMMFEIRDLLLILTTSVAVLVGCFVVLVWKRSSGKKSGKELEPPKIVVPKRRLEQEVDDGKKKVTIFFGTQTGTAEGFAKALFEEAKARYEKAAFKVIDLDDYAADLDEYAEKLKKETYAFFFLATYGDGEPTDNAAKFYKWFTEGDEKGVWLQKLQYGVFGLGNRQYEHFNKIGIVVDDGLTEQGAKRIVPVGLGDDDQSIEDDFSAWKELVWPELDLLLRDEDDKAAATPYTAAIPEYRVVFHDKPDAFSDDHTQTNGHAVHDAQHPCRSNVAVKKELHTPESDRSCTHLEFDISHTGLSYETGDHVGVYCENLIEVVEEAGKLLGLSTDTYFSLHIDNEDGSPLGGPSLQPPFPPCTLRKALTNYADLLSSPKKSTLLALAAHASDPTEADRLRFLASREGKDEYAEWVVANQRSLLEVMEAFPSARPPLGVFFAAVAPRLQPRYYSISSSPKMEPNRIHVTCALVYEKTPAGRIHKGICSTWMKNAVPLTESQDCSWAPIFVRTSNFRLPIDPKVPVIMIGPGTGLAPFRGFLQERLALKESGTELGSSILFFGCRNRKVDYIYENELNNFVENGALSELDVAFSRDGPTKEYVQHKMTQKASEIWNMLSEGAYLYVCGDAKGMAKDVHRTLHTIVQEQGSLDSSKAELYVKNLQMSGRYLRDVW

SEQ ID NO: 97 - protein, 708 aa, synthetic construct
MAQSNSVKISPLDLVTALFSGKVLDTSNASESGESAMLPTIAMIMENRELLMILTTSVAVLIGCVVVLVWRRSSTKKSALEPPVIVVPKRVQEEEVDDGKKKVTVFFGTQTGTAEGFAKALVEEAKARYEKAVFKVIDLDDYAADDDEYEEKLKKESLAFFFLATYGDGEPTDNAARFYKWFTEGDAKGEWLNKLQYGVFGLGNRQYEHFNKIAKVVDDGLVEQGAKRLVPVGLGDDDQCIEDDFTAWKELVWPELDQLLRDEDDTTVATPYTAAVAEYRVVFHEKPDALSEDYSYTNGHAVHDAQHPCRSNVAVKKELHSPESDRSCTHLEFDISNTGLSYETGDHVGVYCENLSEVVNDAERLVGLPPDTYFSIHTDSEDGSPLGGASLPPPFPPCTLRKALTCYADVLSSPKKSALLALAAHATDPSEADRLKFLASPAGKDEYSQWIVASQRSLLEVMEAFPSAKPSLGVFFASVAPRLQPRYYSISSSPKMAPDRIHVTCALVYEKTPAGRIHKGVCSTWMKNAVPMTESQDCSWAPIYVRTSNFRLPSDPKVPVIMIGPGTGLAPFRGFLQERLALKEAGTDLGLSILFFGCRNRKVDFIYENELNNFVETGALSELIVAFSREGPTKEYVQHKMSEKASDIWNLLSEGAYLYVCGDAKGMAKDVHRTLHTIVQEQGSLDSSKAELYVKNLQMSGRYLRDVW

SEQ ID NO: 98 - protein, 705 aa, synthetic construct
MAQSTTSVKLSPFDLMTALLNGKVSFDTSNTSDTNIPLAVFMENRELLMILTTSVAVLIGCVVVLVWRRSSSAAKKAAESPVIVVPKKVTEDEVDDGRKKVTVFFGTQTGTAEGFAKALVEEAKARYEKAVFKVIDLDDYAAEDDEYEEKLKKESLAFFFLATYGDGEPTDNAARFYKWFTEGEEKGEWLDKLQYAVFGLGNRQYEHFNKIAKVVDEKLVEQGAKRLVPVGMGDDDQCIEDDFTAWKELVWPELDQLLRDEDDTSVATPYTAAVAEYRVVFHDKPETYDQDQLTNGHAVHDAQHPCRSNVAVKKELHSPLSDRSCTHLEFDISNTGLSYETGDHVGVYVENLSEVVDEAEKLIGLPPHTYFSVHADNEDGTPLGGASLPPPFPPCTLRKALASYADVLSSPKKSALLALAAHATDSTEADRLKFLASPAGKDEYAQWIVASHRSLLEVMEAFPSAKPPLGVFFASVAPRLQPRYYSISSSPRFAPNRIHVTCALVYEQTPSGRVHKGVCSTWMKNAVPMTESQDCSWAPIYVRTSNFRLPSDPKVPVIMIGPGTGLAPFRGFLQERLAQKEAGTELGTAILFFGCRNRKVDFIYEDELNNFVETGALSELVTAFSREGATKEYVQHKMTQKASDIWNLLSEGAYLYVCGDAKGMAKDVHRTLHTIVQEQGSLDSSKAELYVKNLQMAGRYLRDVW

SEQ ID NO: 99 - protein, 706 aa, synthetic construct
MAQSSSGSMSPFDFMTAIIKGKMEPSNASLGAAGEVTAMILDNRELVMILTTSIAVLIGCVVVFIWRRSSSQTPTAVQPLKPLLAKETESEVDDGKQKVTIFFGTQTGTAEGFAKALADEAKARYDKVTFKVVDLDDYAADDEEYEEKLKKETLAFFFLATYGDGEPTDNAARFYKWFLEGKERGEWLQNLKFGVFGLGNRQYEHFNKIAIVVDEILAEQGGKRLISVGLGDDDQCIEDDFTAWRESLWPELDQLLRDEDDTTVSTPYTAAVLEYRVVFHDPADAPTLEKSYSNANGHSVVDAQHPLRANVAVRRELHTPASDRSCTHLEFDISGTGIAYETGDHVGVYCENLAETVEEALELLGLSPDTYFSVHADKEDGTPLSGSSLPPPFPPCTLRTALTLHADLLSSPKKSALLALAAHASDPTEADRLRHLASPAGKDEYAQWIVASQRSLLEVMAEFPSAKPPLGVFFASVAPRLQPRYYSISSSPRIAPSRIHVTCALVYEKTPTGRVHKGVCSTWMKNSVPSEKSDECSWAPIFVRQSNFKLPADAKVPIIMIGPGTGLAPFRGFLQERLALKEAGTELGPSILFFGCRNSKMDYIYEDELDNFVQNGALSELVLAFSREGPTKEYVQHKMMEKASDIWNLISQGAYLYVCGDAKGMARDVHRTLHTIAQEQGSLDSSKAESMVKNLQMSGRYLRDVW

SEQ ID NO: 100 - protein, 348 aa, synthetic construct
MSVSKTTETFTSIPVIDISKLYSSDLAERKAVAEKLGDAARNIGFLYISGHNVSADLIEGVRKAARDFFAEPFEKKMEYYIGTSATHKGFVPEGEEVYSAGRPDHKEAFDIGYEVPANHPLVQAGTPLLGPNNWPDIPGFRSAAEAYYRTVFDLGRTLFRGFALALGLNESYFDTVANFPPSKLRMIHYPYDADAQDAPGIGAHTDYECFTILLADKPGLEVMNGNGDWIDAPPIPGAFVVNIGDMLEVMTAGEFVATAHRVRKVSEERYSFPLFYACDYHTQIRPLPAFAKKIDASYETITIGEHMWAQALQTYQYLVKKVEKGELKLPKGARKTATFGHFKRNSAA

SEQ ID NO: 101 - protein, 321 aa, synthetic construct
MAAASSFSAAFYSGIPLIDLSAPDAKQLIVKACEELGFFKVVKHGVPMELISSLESESTKFFSLPLSEKQRAGPPSPFGYGNKQIGRNGDVGWVEYLLLNTHLESNSDGFLSMFGQDPQKLRSAVNDYISAVRNMAGEILELMAEGLKIQQRNVFSKLVMDEQSDSVFRVNHYPPCPDLQALKGTNMIGFGEHTDPQIISVLRSNNTSGFQISLADGNWISVPPDHSSFFINVGDSLQVMTNGRFKSVKHRVLTNSSKSRVSMIYFGGPPLSEKIAPLASLMQGEERSLYKEFTWFEYKRSAYNSRLADNRLVPFERIAAS

SEQ ID NO: 102 - protein, 353 aa, synthetic construct
MPSLSKEHFDLYSAFHVPETHAWSSSHLHDHPIAGDGATIPVIDISDPDAASMVGGACRSWGVFYATSHGIPADLLHQVESHARRLFSLPLHRKLQTAPRDGSLSGYGRPPISAFFPKLMWSEGFTLAGHDDHLAVTSQLSPFDSLSFCEVMEAYRKEMKKLAGRLFRLLILSLGLEEEEMGQVGPLKELSQAADAIQLNSYPTCPEPERAIGMAAHTDSAFLTVLHQTDGAGGLQVLRDQDESGSARWVDVLPRPDCLVVNVGDLLHILSNGRFKSVRHRAVVNRADHRISAAYFIGPPAHMKVGSITKLVDMRTGPMYRPVTWPEYLGIRTRLFDKALDSVKFQEKELEKD

SEQ ID NO: 103 - protein, 377 aa, synthetic construct
MATTIADVFKSFPVHIPAHKNLDFDSLHELPDSYAWIQPDSFPSPTHKHHNSILDSDSDSVPLIDLSLPNAAALIGNAFRSWGAFQVINHGVPISLLQSIESSADTLFSLPPSHKLKAARTPDGISGYGLVRISSFFPKRMWSEGFTIVGSPLDHFRQLWPHDYHKHCEIVEEYDREMRSLCGRLMWLGLGELGITRDDMKWAGPDGDFKTSPAATQFNSYPVCPDPDRAMGLGPHTDTSLLTIVYQSNTRGLQVLREGKRWVTVEPVAGGLVVQVGDLLHILTNGLYPSALHQAVVNRTRKRLSVAYVFGPPESAEISPLKKLLGPTQPPLYRPVTWTEYLGKKAEHFNNALSTVRLCAPITGLLDVNDHSRVKVG

SEQ ID NO: 104 - protein, 378 aa, synthetic construct
MHVVTSTPEARHDGAPLVFDASVLRHQHNIPKQFIWPDEEKPAATCPELEVPLIDLSGFLSGEKDAAAEAVRLVGEACEKHGFFLVVNHGVDRKLIGEAHKYMDEFFELPLSQKQSAQRKAGEHCGYASSFTGRFSSKLPWKETLSFRFAADESLNNLVLHYLNDKLGDQFAKFGRVYQDYCEAMSGLSLGIMELLGKSLGVEEQCFKNFFKDNDSIMRLNFYPPCQKPHLTLGTGPHCDPTSLTILHQDQVGGLQVFVDNQWRLITPNFDAFVVNIGDTFMALSNGRYKSCLHRAVVNSERTRKSLAFFLCPRNDKVVRPPRELVDTQNPRRYPDFTWSMLLRFTQTHYRADMKTLEAFSAWLQQEQQEQQEQQFNI

SEQ ID NO: 105 - protein, 361 aa, synthetic construct
MVLQPFVFDAALLRDEHNIPTQFIWPEEDKPSPDASEELILPFIDLKAFLSGDPDSPFQVSKQVGEACESLGAFQVTNHGIDFDLLEEAHSCIQKFFSMPLCEKQRALRKAGESYGYASSFTGRFCSKLPWKETLSFRYSSSSSDIVQNYFVRTLGEEFRHFGEVYQKYCESMSKLSLMIMEVLGLSLGVGRMHFREFFEGNDSTMRLNYYPPCKKPDLTLGTGPHCDPTSLTILHQDDVSGLQVFTGGKWLTVRPKTDAFVVNIGDTFTALSNGRYKSCLHRAVVNSKTARKSLAFFLCPAMNKIVRPPRELVDIDHPRAYPDFTWSALLEFTQKHYRADMQTLNEFSKYILQAQGTLHK

SEQ ID NO: 106 - protein, 358 aa, synthetic construct
MAPGTLTELAGESKLNSKFVRDEDERPKVAYNVFSDEIPVISLAGIDDVDGKRGEICRQIVEACENWGIFQVVDHGVDTNLVADMTRLARDFFALPPEDKLRFDMSGGKKGGFIVSSHLQGEAVQDWREIVTYFSYPVRNRDYSRWPDKPEGWVKVTEEYSERLMSLACKLLEVLSEAMGLEKESLTNACVDMDQKIVVNYYPKCPQPDLTLGLKRHTDPGTITLLLQDQVGGLQATRDNGKTWITVQPVEGAFVVNLGDHGHFLSNGRFKNADHQAVVNSNSSRLSIATFQNPAPDATVYPLKVREGEKAILEEPITFAEMYKRKMGRDLELARLKKLAKEERDHKEVDKPVDQIFA

SEQ ID NO: 107 - protein, 376 aa, synthetic construct
QEKTLNSRFVARDEDSLERPKVSAIYNGSFDEIPVLISLAGIDMTGAGTDAAARRSEICRKIVEACEDWGIFGEIDDDHGKRAEICDKIVKACEDWGVFQPDEKLESVMSAAKKGDFVVDHGVDAEVISQWTTFAKPTSHTQFETETTRDFPNKPEGWKATTEQYSRTLMGLACKLLGVISEAMGLEKEALTKACVDMDQKVVVNYYPKCPQPDLTLGLKRHTDPGTITLLLQDQVGGLQATRDGGKTWITVQPVKDNGWILLHIGDSNGHRHGHFLSNGRFKSHQAYRYRRPTRGSPTFGTKVSNYPPCPEQSLVRPPAGRPYGRALNALDAKKLASAKQQLESAAILLISELAVAYIILAILPSSEIIAEEGYL

SEQ ID NO: 108 - protein, 347 aa, synthetic construct
MATFVSNWSTNNVSESFIAPLEKRAEKDVALGNDVPIIDLQQDHLLIVQQITKACQDFGLFQVINHGVPEKLMVEAMEVYKEFFALPAEEKEKFQPKGEPAKFELPLEQKAKLYVEGERRCNEEFLYWKDTLAHGCYPLHEELLNSWPEKPPTYRDVIAKYSVEVRKLTMRILDYICEGLGLKLGYFDNELTQIQMLLANYYPSCPDPSSTIGSGGHYDGNLITLLQQDLVGLQQLIVKDDKWIAVEPIPTAFVVNLGLTLKVMSNEKFEGSIHRVVTHPTRNRISIGTLIGPDYSCTIEPIKELLSQENPPLYKPYPYAKFAEIYLSDKSDYDAGVKPYKINQFPN

SEQ ID NO: 109 - protein, 329 aa, synthetic construct
MENHTTMKVSSLNCIDLANDDLNHSVVSLKQACLDCGFFYVINHGISEEFMDDVFEQSKKLFALPLEEKMKVLRNEKHRGYTPVLDELLDPKNQINGDHKEGYYIGIEVPKDDPHWDKPFYGPNPWPDADVLPGWRETMEKYHQEALRVSMAIARLLALALDLDVGYFDRTEMLGKPIATMRLLRYQGISDPSKGIYACGAHSDFGMMTLLATDGVMGLQICKDKNAMPQKWEYVPPIKGAFIVNLGDMLERWSNGFFKSTLHRVLGNGQERYSIPFFVEPNHDCLVECLPTCKSESELPKYPPIKCSTYLTQRYEETHANLSIYHQQT

SEQ ID NO: 110 - protein, 511 aa, synthetic construct
MALRINELFVAAIIYIIVHIIISKLITTVRERGRRLPLPPGPTGWPVIGALPLLGSMPHVALAKMAKKYGPIMYLKVGTCGMVVASTPNAAKAFLKTLDINFSNRPPNAGATHLAYNAQDMVFAPYGPRWKLLRKLSNLHMLGGKALENWANVRANELGHMLKSMFDASQDGECVVIADVLTFAMANMIGQVMLSKRVFVEKGVEVNEFKNMVVELMTVAGYFNIGDFIPKLAWMDIQGIEKGMKNLHKKFDDLLTKMFDEHEATSNERKENPDFLDVVMANRDNSEGERLSTTNIKALLLNLFTAGTDTSSSVIEWALAEMMKNPKIFEKAQQEMDQVIGKNRRLIESDIPNLPYLRAICKETFRKHPSTPLNLPRVSSEPCTVDGYYIPKNTRLSVNIWAIGRDPDVWENPLEFTPERFLSGKNAKIEPRGNDFELIPFGAGRRICAGTRMGIVMVEYILGTLVHSFDWKLPNNVIDINMEESFGLALQKAVPLEAMVTPRLSLDVYRC

SEQ ID NO: 111 - protein, 401 aa, synthetic construct
MPKSWPIVISSHSFCFLPNSEQERKMKDLNFHAATLSEEESLRELKAFDETKAGVKGIVDTGITKIPRIFIDQPKNLDRISVCRGKSDIKIPVINLNGLSSNSEIRREIVEKIGEASEKYGFFQIVNHGIPQDVMDKMVDGVRKFHEQDDQIKRQYYSRDRFNKNFLYSSNYVLIPGIACNWRDTMECIMNSNQPDPQEFPDVCRDILMKYSNYVRNLGLILFELLSEALGLKPNHLEEMDCAEGLILLGHYYPACPQPELTFGTSKHSDSGFLTILMQDQIGGLQILLENQWIDVPFIPGALVINIADLLQLITNDKFKSVEHRVLANKVGPRISVAVAFGIKTQTQEGVSPRLYGPIKELISEENPPIYKEVTVKDFITIRFAKRFDDSSSLSPFRLNN

SEQ ID NO: 112 - protein, 372 aa, synthetic construct
MKELNNSEEELKAFDDTKAGVKALVDSGITEIPRIFLDHPTNLDQISSKDREPKFKKNIPVIDLDGISTNSEIRREIVEKIREASEKWGFFQIVNHGIPQEVMDDMIVGIRRFHEQDNEIKKQFYTRDRTKSFRYTSNFVLNPKIACNWRDTFECTMAPHQPNPQDLPDICRDIMMKYISYTRNLGLTLFELLSEALGLKSNRLKDMHCDEGVELVGHYYPACPQPELTLGTSKHTDTGFLTMLQQDQIGGLQVLYENHQWVDVPFIPGALIINIGDFLQIISNDKFKSAPHRVLANKNGPRISTASVFMPNFLESAEVRLYGPIKELLSEENPPIYEQITAKDYVTVQFSRGLDGDSFLSPFMLNKDNMEK

SEQ ID NO: 113 - protein, 374 aa, synthetic construct
MAPTTATKDDSGYGDERRRELQAFDDTKLGVKGLVDSGVKSIPSIFHHPPEALSDIISPAPLPSSPPSGAAIPVVDLSVTRREDLVEQVRHAAGTVGFFWLVNHGVAEELMGGMLRGVRQFNEGPVEAKQALYSRDLARNLRFASNFDLFKAAAADWRDTLFCEVAPNPPPREELPEPLRNVMLEYGAAVTKLARFVFELLSESLGMPSDHLYEMECMQNLNVVCQYYPPCPEPHRTVGVKRHTDPGFFTILLQDGMGGLQVRLGNNGQSGGCWVDIAPRPGALMVNIGDLLQLVTNDRFRSVEHRVPANKSSDTARVSVASFFNTDVRRSERMYGPIPDPSKPPLYRSVRARDFIAKFNTIGLDGRALDHFRL

SEQ ID NO: 114 - protein, 338 aa, synthetic construct
MAKVMNLTPVHASSIPDSFLLPADRLHPATTDVSLPIIDMSRGRDEVRQAILDSGKEYGFIQVVNHGISEPMLHEMYAVCHEFFDMPAEDKAEFFSEDRSERNKLFCGSAFETLGEKYWIDVLELLYPLPSGDTKDWPHKPQMLREVVGNYTSLARGVAMEILRLLCEGLGLRPDFFVGDISGGRVVVDINYYPPSPNPSRTLGLPPHCDRDLMTVLLPGAVPGLEIAYKGGWIKVQPVPNSLVINFGLQLEVVTNGYLKAVEHRAATNFAEPRLSVASFIVPADDCVVGPAEEFVSEDNPPRYRTLTVGEFKRKHNVVNLDSSINQIININNNQKGI

SEQ ID NO: 115 - protein, 339 aa, synthetic construct
MENILHATPAPVSLPESFVFASDKVPPATKAVVSLPIIDLSCGRDEVRRSILEAGKELGFFQVVNHGVSKQVMRDMEGMCEQFFHLPAADKASLYSEERHKPNRLFSGATYDTGGEKYWRDCLRLACPFPVDDSINEWPDTPKGLRDVIEKFTSQTRDVGKELLRLLCEGMGIRADYFEGDLSGGNVILNINHYPSCPNPDKALGQPPHCDRNLITLLLPGAVNGLEVSYKGDWIKVDPAPNAFVVNFGQQLEVVTNGLLKSIEHRAMTNSALARTSVATFIMPTQECLIGPAKEFLSKENPPCYRTTMFRDFMRIYNVVKLGSSLNLTTNLKNVQKEI

SEQ ID NO: 116 - protein, 523 aa, synthetic construct
MEDRNAMDMSRIKYRPQPLRPASMVQPRVLLFPFPALGHVKPFLSLAELLSDAGIDVVFLSTEYNHRRISNTEALASRFPTLHFETIPDGLPPNESRALADGPLYFSMREGTKPRFRQLIQSLNDGRWPITCIITDIMLSSPIEVAEEFGIPVIAFCPCSARYLSIHFFIPKLVEEGQIPYADDDPIGEIQGVPLFEGLLRRNHLPGSWSDKSADISFSHGLINQTLAAGRASALILNTFDELEAPFLTHLSSIFNKIYTIGPLHALSKSRLGDSSSSASALSGFWKEDRACMSWLDCQPPRSVVFVSFGSTMKMKADELREFWYGLVSSGKPFLCVLRSDVVSGGEAAELIEQMAEEEGAGGKLGMVVEWAAQEKVLSHPAVGGFLTHCGWNSTVESIAAGVPMMCWPILGDQPSNATWIDRVWKIGVERNNREWDRLTVEKMVRALMEGQKRVEIQRSMEKLSKLANEKVVRGINLHPTISLKKDTPTTSEHPRHEFENMRGMNYEMLVGNAIKSPTLTKK

SEQ ID NO: 117 - protein, 473 aa, synthetic construct
MTIFFSVEILVLGIAEFAAIAMDAAQQGDTTTILMLPWLGYGHLSAFLELAKSLSRRNFHIYFCSTSVNLDAIKPKLPSSFSDSIQFVELHLPSSPEFPPHLHTTNGLPPTLMPALHQAFSMAAQHFESILQTLAPHLLIYDSLQPWAPRVASSLKIPAINFNTTGVFVISQGLHPIHYPHSKFPFSEFVLHNHWKAMYSTADGASTERTRKRGEAFLYCLHASCSVILINSFRELEGKYMDYLSVLLNKKVVPVGPLVYEPNQDGEDEGYSSIKNWLDKKEPSSTVFVSFGSEYFPSKEEMEEIAHGLEASEVNFIWVVRFPQGDNTSGIEDALPKGFLERAGERGMVVKGWAPQAKILKHWSTGGFVSHCGWNSVMESMMFGVPIIGVPMHVDQPFNAGLVEEAGVGVEAKRDPDGKIQRDEVAKLIKEVVVEKTREDVRKKAREMSEILRSKGEEKFDEMVAEISLLLKI

SEQ ID NO: 118 - protein, 460 aa, synthetic construct
MDETTVNGGRRASDVVVFAFPRHGHMSPMLQFSKRLVSKGLRVTFLITTSATESLRLNLPPSSSLDLQVISDVPESNDIATLEGYLRSFKATVSKTLADFIDGIGNPPKFIVYDSVMPWVQEVARGRGLDAAPFFTQSSAVNHILNHVYGGSLSIPAPENTAVSLPSMPVLQAEDLPAFPDDPEVVMNFMTSQFSNFQDAKWIFFNTFDQLECKKQSQVVNWMADRWPIKTVGPTIPSAYLDDGRLEDDRAFGLNLLKPEDGKNTRQWQWLDSKDTASVLYISFGSLAILQEEQVKELAYFLKDTNLSFLWVLRDSELQKLPHNFVQETSHRGLVVNWCSQLQVLSHRAVSCFVTHCGWNSTLEALSLGVPMVAIPQWVDQTTNAKFVADVWRVGVRVKKKDERIVTKEELEASIRQVVQGEGRNEFKHNAIKWKKLAKEAVDEGGSSDKNIEEFVKTIA

SEQ ID NO: 119 - protein, 486 aa, synthetic construct
MGDNGDGGEKKELKENVKKGKELGRQAIGEGYINPSLQLARRLISLGVNVTFATTVLAGRRMKNKTHQTATTPGLSFATFSDGFDDETLKPNGDLTHYFSELRRCGSESLTHLITSAANEGRPITFVIYSLLLSWAADIASTYDIPSALFFAQPATVLALYFYYFHGYGDTICSKLQDPSSYIELPGLPLLTSQDMPSFFSPSGPHAFILPPMREQAEFLGRQSQPKVLVNTFDALEADALRAIDKLKMLAIGPLIPSALLGGNDSSDASFCGDLFQVSSEDYIEWLNSKPDSSVVYISVGSICVLSDEQEDELVHALLNSGHTFLWVKRSKENNEGVKQETDEEKLKKLEEQGKMVSWCRQVEVLKHPALGCFLTHCGWNSTIESLVSGLPVVAFPQQIDQATNAKLIEDVWKTGVRVKANTEGIVEREEIRRCLDLVMGSRDGQKEEIERNAKKWKELARQAIGEGGSSDSNLKTFLWEIDLEI

SEQ ID NO: 120 - protein, 485 aa, synthetic construct
MAEQAHDLLHVLLFPFPAEGHIKPFLCLAELLCNAGFHVTFLNTDYNHRRLHNLHLLAARFPSLHFESISDGLPPDQPRDILDPKFFISICQVTKPLFRELLLSYKRISSVQTGRPPITCVITDVIFRFPIDVAEELDIPVFSFCTFSARFMFLYFWIPKLIEDGQLPYPNGNINQKLYGVAPEAEGLLRCKDLPGHWAFADELKDDQLNFVDQTTASSRSSGLILNTFDDLEAPFLGRLSTIFKKIYAVGPIHSLLNSHHCGLWKEDHSCLAWLDSRAAKSVVFVSFGSLVKITSRQLMEFWHGLLNSGKSFLFVLRSDVVEGDDEKQVVKEIYETKAEGKWLVVGWAPQEKVLAHEAVGGFLTHSGWNSILESIAAGVPMISCPKIGDQSSNCTWISKVWKIGLEMEDRYDRVSVETMVRSIMEQEGEKMQKTIAELAKQAKYKVSKDGTSYQNLECLIQDIKKLNQIEGFINNPNFSDLLRV

SEQ ID NO: 121 - protein, 473 aa, synthetic construct
MDAQQGHTTTILMLPWVGYGHLLPFLELAKSLSRRKLFHIYFCSTSVSLDAIKPKLPPSISSDDSIQLVELRLPSSPELPPHLHTTNGLPSHLMPALHQAFVMAAQHFQVILQTLAPHLLIYDILQPWAPQVASSLNIPAINFSTTGASMLSRTLHPTHYPSSKFPISEFVLHNHWRAMYTTADGALTEEGHKIEETLANCLHTSCGVVLVNSFRELETKYIDYLSVLLNKKVVPVGPLVYEPNQEGEDEGYSSIKNWLDKKEPSSTVFVSFGTEYFPSKEEMEEIAYGLELSEVNFIWVLRFPQGDSTSTIEDALPKGFLERAGERAMVVKGWAPQAKILKHWSTGGLVSHCGWNSMMEGMMFGVPIIAVPMHLDQPFNAGLVEEAGVGVEAKRDSDGKIQREEVAKSIKEVVIEKTREDVRKKAREMDTKHGPTYFSRSKVSSFGRLYKINRPTTLTVGRFWSKQIKMKRE

SEQ ID NO: 122 - protein, 453 aa, synthetic construct
MDAQRGHTTTILMFPWLGYGHLSAFLELAKSLSRRNFHIYFCSTSVNLDAIKPKLPSSSSSDSIQLVELCLPSSPDQLPPHLHTTNALPPHLMPTLHQAFSMAAQHFAAILHTLAPHLLIYDSFQPWAPQLASSLNIPAINFNTTGASVLTRMLHATHYPSSKFPISEFVLHDYWKAMYSAAGGAVTKKDHKIGETLANCLHASCSVILINSFRELEEKYMDYLSVLLNKKVVPVGPLVYEPNQDGEDEGYSSIKNWLDKKEPSSTVFVSFGSEYFPSKEEMEEIAHGLEASEVHFIWVVRFPQGDNTSAIEDALPKGFLERVGERGMVVKGWAPQAKILKHWSTGGFVSHCGWNSVMESMMFGVPIIGVPMHLDQPFNAGLAEEAGVGVEAKRDPDGKIQRDEVAKLIKEVVVEKTREDVRKKAREMSEILRSKGEEKMDEMVAAISLFLKI

SEQ ID NO: 123 - protein, 472 aa, synthetic construct
MAQPQTQARVLVFPYPTVGHIKPFLSLAELLADGGLDVVFLSTEYNHRRIPNLEALASRFPTLHFDTIPDGLPIDKPRVIIGGELYTSMRDGVKQRLRQVLQSYNDGSSPITCVICDVMLSGPIEAAEELGIPVVTFCPYSARYLCAHFVMPKLIEEGQIPFTDGNLAGEIQGVPLFGGLLRRDHLPGFWFVKSLSDEVWSHAFLNQTLAVGRTSALIINTLDELEAPFLAHLSSTFDKIYPIGPLDALSKSRLGDSSSSSTVLTAFWKEDQACMSWLDSQPPKSVIFVSFGSTMRMTADKLVEFWHGLVNSGTRFLCVLRSDIVEGGGAADLIKQVGETGNGIVVEWAAQEKVLAHRAVGGFLTHCGWNSTMESIAAGVPMMCWQIYGDQMINATWIGKVWKIGIERDDKWDRSTVEKMIKELMEGEKGAEIQRSMEKFSKLANDKVVKGGTSFENLELIVEYLKKLKPSN

SEQ ID NO: 124 - protein, 466 aa, synthetic construct
MAQPRVLLFPFPAMGHVKPFLSLAELLSDAGVEVVFLSTEYNHRRIPDIGALAARFPTLHFETIPDGLPPDQPRVLADGHLYFSMLDGTKPRFRQLIQSLNGNPRPITCIINDVMLSSPIEVAEEFGIPVIAFCPCSARFLSVHFFMPNFIEEAQIPYTDENPMGKIEEATVFEGLLRRKDLPGLWCAKSSNISFSHRFINQTIAAGRASALILNTFDELESPFLNHLSSIFPKIYCIGPLNALSRSRLGKSSSSSSALAGFWKEDQAYMSWLESQPPRSVIFVSFGSTMKMEAWKLAEFWYGLVNSGSPFLFVFRPDCVINSGDAAEVMEGRGRGMVVEWASQEKVLAHPAVGGFLTHCGWNSTVESIVAGVPMMCCPIVADQLSNATWIHKVWKIGIEGDEKWDRSTVEMMIKELMESQKGTEIRTSIEMLSKLANEKVVKGGTSLNNFELLVEDIKTLRRPYT

SEQ ID NO: 125 - protein, 482 aa, synthetic construct
MEQSDSNSDDHQHHVLLFPFPAKGHIKPFLCLAQLLCGAGLQVTFLNTDHNHRRIDDRHRRLLATQFPMLHFKSISDGLPPDHPRDLLDGKLIASMRRVTESLFRQLLLSYNGYGNGTNNVSNSGRRPPISCVITDVIFSFPVEVAEELGIPVFSFATFSARFLFLYFWIPKLIQEGQLPFPDGKTNQELYGVPGAEGIIRCKDLPGSWSVEAVAKNDPMNFVKQTLASSRSSGLILNTFEDLEAPFVTHLSNTFDKIYTIGPIHSLLGTSHCGLWKEDYACLAWLDARPRKSVVFVSFGSLVKTTSRELMELWHGLVSSGKSFLLVLRSDVVEGEDEEQVVKEILESNGEGKWLVVGWAPQEEVLAHEAIGGFLTHSGWNSTMESIAAGVPMVCWPKIGDQPSNCTWVSRVWKVGLEMEERYDRSTVARMARSMMEQEGKEMERRIAELAKRVKYRVGKDGESYRNLESLIRDIKITKSSN

SEQ ID NO: 126 - protein, 450 aa, synthetic construct
MDAHQQAEHTTTILMLPWVGYGHLTAYLELAKALSRRNFHIYYCSTPVNIESIKPKLTIPCSSIQFVELHLPSSDDLPPNLHTTNGLPSHLMPTLHQAFSAAAPLFEEILQTLCPHLLIYDSLQPWAPKIASSLKIPALNFNTSGVSVIAQALHAIHHPDSKFPLSDFILHNYWKSTYTTADGGASEKTRRAREAFLYCLNSSGNAILINTFRELEGEYIDYLSLLLNKKVIPIGPLVYEPNQDEDQDEEYRSIKNWLDKKEPCSTVFVSFGSEYFPSNEEMEEIAPGLEESGANFIWVVRFPKLENRNGIIEEGLLERAGERGMVIKEWAPQARILRHGSIGGFVSHCGWNSVMESIICGVPVIGVPMRVDQPYNAGLVEEAGVGVEAKRDPDGKIQRHEVSKLIKQVVVEKTRDDVRKKVAQMSEILRRKGDEKIDEMVALISLLPKG

SEQ ID NO: 127 - protein, 450 aa, synthetic construct
MDARQQAEHTTTILMLPWVGYGHLSAYLELAKALSRRNFHIYYCSTPVNIESIKPKLTIPCSSIQFVELHLPFSDDLPPNLHTTNGLPSHLMPALHQAFSAAAPLFEAILQTLCPHLLIYDSLQPWAPQIASSLKIPALNFNTTGVSVIARALHTIHHPDSKFPLSEIVLHNYWKATHATADGANPEKFRRDLEALLCCLHSSCNAILINTFRELEGEYIDYLSLLLNKKVTPIGPLVYEPNQDEEQDEEYRSIKNWLDKKEPYSTIFVSFGSEYFPSNEEMEEIARGLEESGANFIWVVRFHKLENGNGITEEGLLERAGERGMVIQGWAPQARILRHGSIGGFVSHCGWNSVMESIICGVPVIGVPMGLDQPYNAGLVEEAGVGVEAKRDPDGKIQRHEVSKLIKQVVVEKTRDDVRKKVAQMSEILRRKGDEKIDEMVALISLLLKG

SEQ ID NO: 128 - protein, 471 aa, synthetic construct
MGLSPTDHVLLFPFPAKGHIKPFFCLAHLLCNAGLRVTFLSTEHHHQKLHNLTHLAAQIPSLHFQSISDGLSLDHPRNLLDGQLFKSMPQVTKPLFRQLLLSYKDGTSPITCVITDLILRFPMDVAQELDIPVFCFSTFSARFLFLYFSIPKLLEDGQIPYPEGNSNQVLHGIPGAEGLLRCKDLPGYWSVEAVANYNPMNFVNQTIATSKSHGLILNTFDELEVPFITNLSKIYKKVYTIGPIHSLLKKSVQTQYEFWKEDHSCLAWLDSQPPRSVMFVSFGSIVKLKSSQLKEFWNGLVDSGKAFLLVLRSDALVEETGEEDEKQKELVIKEIMETKEEGRWVIVNWAPQEKVLEHKAIGGFLTHSGWNSTLESVAVGVPMVSWPQIGDQPSNATWLSKVWKIGVEMEDSYDRSTVESKVRSIMEHEDKKMENAIVELAKRVDDRVSKEGTSYQNLQRLIEDIEGFKLN

SEQ ID NO: 129 - protein, 462 aa, synthetic construct
MELSHTHHVLLFPFPAKGHIKPFFSLAQLLCNAGLRVTFLNTDHHHRRIHDLNRLAAQLPTLHFDSVSDGLPPDEPRNVFDGKLYESIRQVTSSLFRELLVSYNNGTSSGRPPITCVITDVMFRFPIDIAEELGIPVFTFSTFSARFLFLIFWIPKLLEDGQLRYPEQELHGVPGAEGLIRWKDLPGFWSVEDVADWDPMNFVNQTLATSRSSGLILNTFDELEAPFLTSLSKIYKKIYSLGPINSLLKNFQSQPQYNLWKEDHSCMAWLDSQPRKSVVFVSFGSVVKLTSRQLMEFWNGLVNSGMPFLLVLRSDVIEAGEEVVREIMERKAEGRWVIVSWAPQEEVLAHDAVGGFLTHSGWNSTLESLAAGVPMISWPQIGDQTSNSTWISKVWRIGLQLEDGFDSSTIETMVRSIMDQTMEKTVAELAERAKNRASKNGTSYRNFQTLIQDITNIIETHI

SEQ ID NO: 130 - protein, 454 aa, synthetic construct
MDAQKAVDTPPTTVLMLPWIGYGHLSAYLELAKALSRRNFHVYFCSTPVNLDSIKPNLIPPPSSIQFVDLHLPSSPELPPHLHTTNGLPSHLKPTLHQAFSAAAQHFEAILQTLSPHLLIYDSLQPWAPRIASSLNIPAINFNTTAVSIIAHALHSVHYPDSKFPFSDFVLHDYWKAKYTTADGATSEKIRRGAEAFLYCLNASCDVVLVNSFRELEGEYMDYLSVLLKKKVVSVGPLVYEPSEGEEDEEYWRIKKWLDEKEALSTVLVSFGSEYFPSKEEMEEIAHGLEESEANFIWVVRFPKGEESCRGIEEALPKGFVERAGERAMVVKKWAPQGKILKHGSIGGFVSHCGWNSVLESIRFGVPVIGVPMHLDQPYNAGLLEEAGIGVEAKRDADGKIQRDQVASLIKRVVVEKTREDIWKTVREMREVLRRRDDDMIDEMVAEISVVLKI

SEQ ID NO: 131 - protein, 492 aa, synthetic construct
MSSNLFLKISIPFGRLRDSALNCSVFHCKLHLAIAIAMDAQQAANKSPTATTIFMLPWAGYGHLSAYLELAKALSTRNFHIYFCSTPVSLASIKPRLIPSCSSIQFVELHLPSSDEFPPHLHTTNGLPSRLVPTFHQAFSEAAQTFEAFLQTLRPHLLIYDSLQPWAPRIASSLNIPAINFFTAGAFAVSHVLRAFHYPDSQFPSSDFVLHSRWKIKNTTAESPTQAKLPKIGEAIGYCLNASRGVILTNSFRELEGKYIDYLSVILKKRVFPIGPLVYQPNQDEEDEDYSRIKNWLDRKEASSTVLVSFGSEFFLSKEETEAIAHGLEQSEANFIWGIRFPKGAKKNAIEEALPEGFLERAGGRAMVVEEWVPQGKILKHGSIGGFVSHCGWNSAMESIVCGVPIIGIPMQVDQPFNAGILEEAGVGVEAKRDSDGKIQRDEVAKLIKEVVVERTREDIRNKLEKINEILRSRREEKLDELATEISLLSRN

SEQ ID NO: 132 - protein, 462 aa, synthetic construct
MELSPTHHLLLFPFPAKGHIKPFFSLAQLLCNAGARVTFLNTDHHHRRIHDLDRLAAQLPTLHFDSVSDGLPPDESRNVFDGKLYESIRQVTSSLFRELLVSYNNGTSSGRPPITCVITDCMFRFPIDIAEELGIPVFTFSTFSARFLFLFFWIPKLLEDGQLRYPEQELHGVPGAEGLIRCKDLPGFLSDEDVAHWKPINFVNQILATSRSSGLILNTFDELEAPFLTSLSKIYKKIYSLGPINSLLKNFQSQPQYNLWKEDHSCMAWLDSQPPKSVVFVSFGSVVKLTNRQLVEFWNGLVNSGKPFLLVLRSDVIEAGEEVVRENMERKAEGRWMIVSWAPQEEVLAHDAVGGFLTHSGWNSTLESLAAGVPMISWTQIGDQTSNSTWVSKVWRIGLQLEDGFDSFTIETMVRSVMDQTMEKTVAELAERAKNRASKNGTSYRNFQTLIQDITNIIETHI

SEQ ID NO: 133 - protein, 457 aa, synthetic construct
MDAQKAVDTPPTTVLMLPWIGYGHLSAYLELAKALSRRNFHVYFCSTPVNLDSIKPNLIPPPPSIQFVDLHLPSSPELPPHLHTTNGLPSHLKPTLHQAFSAAAQHFEAILQTLSPHLLIYDSLQPWAPRIASSLNIPAINFNTTAVSIIAHALHSVHYPDSKFPFSDFVLHDYWKAKYTTADGATSEKTRRGVEAFLYCLNASCDVVLVNSFRELEGEYMDYLSVLLKKKVVSVGPLVYEPSEGEEDEEYWRIKKWLDEKEALSTVLVSFGSEYFPPKEEMEEIAHGLEESEANFIWVVRFPKGEESSSRGIEEALPKGFVERAGERAMVVKKWAPQGKILKHGSIGGFVSHCGWNSVLESIRFGVPVIGAPMHLDQPYNAGLLEEAGIGVEAKRDADGKIQRDQVASLIKQVVVEKTREDIWKKVREMREVLRRRDDDDMMIDEMVAVISVVLKI

SEQ ID NO: 134 - protein, 455 aa, synthetic construct
MDAQQAANKSPTASTIFMLPWVGYGHLSAYLELAKALSTRNFHVYFCSTPVSLASIKPRLIPSCSSIQFVELHLPSSDEFPPHLHTTNGLPAHLVPTIHQAFAAAAQTFEAFLQTLRPHLLIYDSLQPWAPRIASSLNIPAINFFTAGAFAVSHVLRAFHYPDSQFPSSDFVLHSRWKIKNTTAESPTQVKIPKIGEAIGYCLNASRGVILTNSFRELEGKYIDYLSVILKKRVLPIGPLVYQPNQDEEDEDYSRIKNWLDRKEASSTVLVSFGSEFFLSKEETEAIAHGLEQSEANFIWGIRFPKGAKKNAIEEALPEGFLERVGGRAMVVEEWVPQGKILKHGNIGGFVSHCGWNSAMESIMCGVPVIGIPMQVDQPFNAGILEEAGVGVEAKRDSDGKIQRDEVAKLIKEVVVERTREDIRNKLEEINEILRTRREEKLDELATEISLLCKN

SEQ ID NO: 135 - protein, 474 aa, synthetic construct
MAMKQPHVIIFPFPLQGHMKPLLCLAELLCHAGLHVTYVNTHHNHQRLANRQALSTHFPTLHFESISDGLPEDDPRTLNSQLLIALKTSIRPHFRELLKTISLKAESNDTLVPPPSCIMTDGLVTFAFDVAEELGLPILSFNVPCPRYLWTCLCLPKLIENGQLPFQDDDMNVEITGVPGMEGLLHRQDLPGFCRVKQADHPSLQFAINETQTLKRASALILDTVYELDAPCISHMALMFPKIYTLGPLHALLNSQIGDMSRGLASHGSLWKSDLNCMTWLDSQPSKSIIYVSFGTLVHLTRAQVIEFWYGLVNSGHPFLWVMRSDITSGDHQIPAELENGTKERGCIVDWVSQEEVLAHKSVGGFLTHSGWNSTLESIVAGLPMICWPKLGDHYIISSTVCRQWKIGLQLNENCDRSNIESMVQTLMGSKREEIQSSMDAISKLSRDSVAEGGSSHNNLEQLIEYIRNLQHQN

SEQ ID NO: 136 - protein, 468 aa, synthetic construct
MRQPHVLVLPFPAQGHIKPMLCLAELLCQAGLRVTFLNTHHSHRRLNNLQDLSTRFPTLHFESVSDGLPEDHPRNLVHFMHLVHSIKNVTKPLLRDLLTSLSLKTDIPPVSCIIADGILSFAIDVAEELQIKVIIFRTISSCCLWSYLCVPKLIQQGELQFSDSDMGQKVSSVPEMKGSLRLHDRPYSFGLKQLEDPNFQFFVSETQAMTRASAVIFNTFDSLEAPVLSQMIPLLPKVYTIGPLHALRKARLGDLSQHSSFNGNLREADHNCITWLDSQPLRSVVYVSFGSHVVLTSEELLEFWHGLVNSGKRFLWVLRPDIIAGEKDHNQIIAREPDLGTKEKGLLVDWAPQEEVLAHPSVGGFLTHCGWNSTLESMVAGVPMLCWPKLPDQLVNSSCVSEVWKIGLDLKDMCDRSTVEKMVRALMEDRREEVMRSVDGISKLARESVSHGGSSSSNLEMLIQELET

SEQ ID NO: 137 - protein, 462 aa, synthetic construct
MDSKQKKMSVLMFPWLAYGHISPFLELAKKLSKRNFHTFFFSTPINLNSIKSKLSPKYAQSIQFVELHLPSLPDLPPHYHTTNGLPPHLMNTLKKAFDMSSLQFSKILKTLNPDLLVYDFIQPWAPLLALSNKIPAVHFLCTSAAMSSFSVHAFKKPCEDFPFPNIYVHGNFMNAKFNNMENCSSDDSISDQDRVLQCFERSTKIILVKTFEELEGKFMDYLSVLLNKKIVPTGPLTQDPNEDEGDDDERTKLLLEWLNKKSKSSTVFVSFGSEYFLSKEEREEIAYGLELSKVNFIWVIRFPLGENKTNLEEALPQGFLQRVSERGLVVENWAPQAKILQHSSIGGFVSHCGWSSVMESLKFGVPIIAIPMHLDQPLNARLVVDVGVGLEVIRNHGSLEREEIAKLIKEVVLGNGNDGEIVRRKAREMSNHIKKKGEKDMDELVEELMLICKMKPNSCHLS

SEQ ID NO: 138 - protein, 448 aa, synthetic construct
MMERQRSIKVLMFPWLAHGHISPFLELAKRLTDRNFQIYFCSTPVNLTSVKPKLSQKYSSSIKLVELHLPSLPDLPPHYHTTNGLALNLIPTLKKAFDMSSSSFSTILSTIKPDLLIYDFLQPWAPQLASCMNIPAVNFLSAGASMVSFVLHSIKYNGDDHDDEFLTTELHLSDSMEAKFAEMTESSPDEHIDRAVTCLERSNSLILIKSFRELEGKYLDYLSLSFAKKVVPIGPLVAQDTNPEDDSMDIINWLDKKEKSSTVFVSFGSEYYLTNEEMEEIAYGLELSKVNFIWVVRFPLGQKMAVEEALPKGFLERVGEKGMVVEDWAPQMKILGHSSIGGFVSHCGWSSLMESLKLGVPIIAMPMQLDQPINAKLVERSGVGLEVKRDKNGRIEREYLAKVIREIVVEKARQDIEKKAREMSNIITEKGEEEIDNVVEELAKLCGM

SEQ ID NO: 139 - protein, 446 aa, synthetic construct
MDARQSDGISVLMFPWLAHGHISPFLQLAKKLSKRNFSIYFCSTPVNLDPIKGKLSESYSLSIQLVKLHLPSLPELPPQYHTTNGLPPHLMPTLKMAFDMASPNFSNILKTLHPDLLIYDFLQPWAPAAASSLNIPAVQFLSTGATLQSFLAHRHRKPGIEFPFQEIHLPDYEIGRLNRFLEPSAGRISDRDRANQCLERSSRFSLIKTFREIEAKYLDYVSDLTKKKMVTVGPLLQDPEDEDEATDIVEWLNKKCEASAVFVSFGSEYFVSKEEMEEIAHGLELSNVDFIWVVRFPMGEKIRLEDALPPGFLHRLGDRGMVVEGWAPQRKILGHSSIGGFVSHCGWSSVMEGMKFGVPIIAMPMHLDQPINAKLVEAVGVGREVKRDENRKLEREEIAKVIKEVVGEKNGENVRRKARELSETLRKKGDEEIDVVVEELKQLCSY

SEQ ID NO: 140 - protein, 456 aa, synthetic construct
MDTARKRIRVVMLPWLAHGHISPFLELSKKLAKRNFHIYFCSTPVNLSSIKPKLSGKYSRSIQLVELHLPSLPELPPQYHTTKGLPPHLNATLKRAFDMAGPHFSNILKTLSPDLLIYDFLQPWAPAIAASQNIPAINFLSTGAAMTSFVLHAMKKPGDEFPFPEIHLDECMKTRFVDLPEDHSPSDDHNHISDKDRALKCFERSSGFVMMKTFEELEGKYINFLSHLMQKKIVPVGPLVQNPVRGDHEKAKTLEWLDKRKQSSAVFVSFGTEYFLSKEEMEEIAYGLELSNVNFIWVVRFPEGEKVKLEEALPEGFLQRVGEKGMVVEGWAPQAKILMHPSIGGFVSHCGWSSVMESIDFGVPIVAIPMQLDQPVNAKVVEQAGVGVEVKRDRDGKLEREEVATVIREVVMGNIGESVRKKEREMRDNIRKKGEEKMDGVAQELVQLYGNGIKNV

SEQ ID NO: 141 - protein, 456 aa, synthetic construct
METLQRRKISVLMFPWLAHGHLSPFLELSKKLNKRNFHVYFCSTPVNLDSIKPKLSAEYSFSIQLVELHLPSSPELPLHYHTTNGLPPHLMKNLKNAFDMASSSFFNILKTLKPDLLIYDFIQPWAPALASSLNIPAVNFLCTSMAMSCFGLHLNNQEAKFPFPGIYPRDYMRMKVFGALESSSNDIKDGERAGRCMDQSFHLILAKTFRELEGKYIDYLSVKLMKKIVPVGPLVQDPIFEDDEKIMDHHQVIKWLEKKERLSTVFVSFGTEYFLSTEEMEEIAYGLELSKAHFIWVVRFPTGEKINLEESLPKRYLERVQERGKIVEGWAPQQKILRHSSIGGFVSHCGWSSIMESMKFGVPIIAMPMNLDQPVNSRIVEDAGVGIEVRRNKSGELEREEIAKTIRKVVVEKDGKNVSRKAREMSDTIRKKGEEEIDGVVDELLQLCDVKTNYLQ

SEQ ID NO: 142 - protein, 461 aa, synthetic construct
MATAQTRKISVLMFPWLAHGHLSPFLELSKKLANRNFHVYFCSTPVNLDSIKPKLSPEYHFSIQFVELHLPSSPELPSHYHTTNGLPPHLMKTLKKAFDMASSSFFNILKTLNPDLLIYDFLQPWAPALASSLNIPAVNFLCSSMAMSCFGLNLNKNKEIKFLFPEIYPRDYMEMKLFRVFESSSNQIKDGERAGRCIDQSFHVILAKTFRELEGKYIDYVSVKCNKKIVPVGPLVEDTIHEDDEKTMDHHHHHHDEVIKWLEKKERSTTVFVSFGSEYFLSKEEMEEIAHGLELSKVNFIWVVRFPKGEKINLEESLPEGYLERIQERGKIVEGWAPQRKILGHSSIGGFVSHCGWSSIMESMKLGVPIIAMPMNLDQPINSRIVEAAGVGIEVSRNQSGELEREEMAKTIRKVVVEREGVYVRRKAREMSDVLRKKGEEEIDGVVDELVQLCDMKTNYL

SEQ ID NO: 143 - protein, 452 aa, synthetic construct
MDLKRRSIRVLMLPWLAHGHISPFLELAKKLTNRNFLIYFCSTPINLNSIKPKLSSKYSFSIQLVELHLPSLPELPPHYHTTNGLPLHLMNTLKTAFDMASPSFLNILKTLKPDLLICDHLQPWAPSLASSLNIPAIIFPTNSAIMMAFSLHHAKNPGEEFPFPSININDDMVKSINFLHSASNGLTDMDRVLQCLERSSNTMLLKTFRQLEAKYVDYSSALLKKKIVLAGPLVQVPDNEDEKIEIIKWLDSRGQSSTVFVSFGSEYFLSKEEREDIAHGLELSKVNFIWVVRFPVGEKVKLEEALPNGFAERIGERGLVVEGWAPQAMILSHSSIGGFVSHCGWSSMMESMKFGVPIIAMPMHIDQPLNARLVEDVGVGLEIKRNKDGRFEREELARVIKEVLVYKNGDAVRSKAREMSEHIKKNGDQEIDGVADALVKLCEMKTNSLNQD

SEQ ID NO: 144 - protein, 460 aa, synthetic construct
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 145 - protein, 458 aa, synthetic construct
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 146 - protein, 481 aa, synthetic construct
MDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 147 - protein, 485 aa, synthetic construct
MYNVTYHQNSKAMATSDSIVDDRKQLHVATFPWLAFGHILPFLQLSKLIAEKGHKVSFLSTTRNIQRLSSHISPLINVVQLTLPRVQELPEDAEATTDVHPEDIQYLKKAVDGLQPEVTRFLEQHSPDWIIYDFTHYWLPSIAASLGISRAYFCVITPWTIAYLAPSSDAMINDSDGRTTVEDLTTPPKWFPFPTKVCWRKHDLARMEPYEAPGISDGYRMGMVFKGSDCLLFKCYHEFGTQWLPLLETLHQVPVVPVGLLPPEIPGDEKDETWVSIKKWLDGKQKGSVVYVALGSEALVSQTEVVELALGLELSGLPFVWAYRKPKGPAKSDSVELPDGFVERTRDRGLVWTSWAPQLRILSHESVCGFLTHCGSGSIVEGLMFGHPLIMLPIFCDQPLNARLLEDKQVGIEIPRNEEDGCLTKESVARSLRSVVVENEGEIYKANARALSKIYNDTKVEKEYVSQFVDYLEKNARAVAIDHES

SEQ ID NO: 148 - protein, 473 aa, synthetic construct
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEVPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNTRAVAIDHES

SEQ ID NO: 149 - protein, 473 aa, synthetic construct
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 150 - protein, 462 aa, synthetic construct
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 151 - protein, 490 aa, synthetic construct
MGSISEMVFETCPSPNPIHVMLVSFQGQGHVNPLLRLGKLIASKGLLVTFVTTELWGKKMRQANKIVDGELKPVGSGSIRFEFFDEEWAEDDDRRADFSLYIAHLESVGIREVSKLVRRYEEANEPVSCLINNPFIPWVCHVAEEFNIPCAVLWVQSCACFSAYYHYQDGSVSFPTETEPELDVKLPCVPVLKNDEIPSFLHPSSRFTGFRQAILGQFKNLSKSFCVLIDSFDSLEREVIDYMSSLCPVKTVGPLFKVARTVTSDVSGDICKSTDKCLEWLDSRPKSSVVYISFGTVAYLKQEQIEEIAHGVLKSGLSFLWVIRPPPHDLKVETHVLPQELKESSAKGKGMIVDWCPQEQVLSHPSVACFVTHCGWNSTMESLSSGVPVVCCPQWGDQVTDAVYLIDVFKTGVRLGRGATEERVVPREEVAEKLLEATVGEKAEELRKNALKWKAEAEAAVAPGGSSDKNFREFVEKLGAGVTKTKDNGY

SEQ ID NO: 152 - protein, 481 aa, synthetic construct
MGSHVAQKQHVVCVPYPAQGHINPMMKVAKLLYAKGFHITFVNTVYNHNRLLRSRGPNAVDGLPSFRFESIPDGLPETDVDVTQDIPTLCESTMKHCLAPFKELLRQINARDDVPPVSCIVSDGCMSFTLDAAEELGVPEVLFWTTSACGFLAYLYYYRFIEKGLSPIKDESYLTKEHLDTKIDWIPSMKNLRLKDIPSFIRTTNPDDIMLNFIIREADRAKRASAIILNTFDDLEHDVIQSMKSIVPPVYSIGPLHLLEKQESGEYSEIGRTGSNLWREETECLDWLNTKARNSVVYVNFGSITVLSAKQLVEFAWGLAATGKEFLWVIRPDLVAGDEAMVPPEFLTATADRRMLASWCPQEKVLSHPAIGGFLTHCGWNSTLESLCGGVPMVCWPFFAEQQTNCKFSRDEWEVGIEIGGDVKREEVEAVVRELMDEEKGKNMREKAEEWRRLANEATEHKHGSSKLNFEMLVNKVLLGE

SEQ ID NO: 153 - protein, 438 aa, synthetic construct
MIHCGKKHICAFVTCILISASILMYSWKDPQLQNNITRKIFQATSALPASQLCRGKPAQNVITALEDNRTFIISPYFDDRESKVTRVIGIVHHEDVKQLYCWFCCQPDGKIYVARAKIDVHSDRFGFPYGAADIVCLEPENCNPTHVSIHQSPHANIDQLPSFKIKNRKSETFSVDFTVCISAMFGNYNNVLQFIQSVEMYKILGVQKVVIYKNNCSQLMEKVLKFYMEEGTVEIIPWPINSHLKVSTKWHFSMDAKDIGYYGQITALNDCIYRNMQRSKFVVLNDADEIILPLKHLDWKAMMSSLQEQNPGAGIFLFENHIFPKTVSTPVFNISSWNRVPGVNILQHVHREPDRKEVFNPKKMIIDPRQVVQTSVHSVLRAYGNSVNVPADVALVYHCRVPLQEELPRESLIRDTALWRYNSSLITNVNKVLHQTVL

SEQ ID NO: 154 - protein, 252 aa, synthetic construct
MPTLTVAMIVKNEAQDLAECLKTVDGWVDEIVIVDSGSTDDTLKIATQFNAKVYVNSDWQGFGPQRQFAQQYVTSDYVLWLDADERVTPELKASILQAVQHNQKNTVYKVSRLSEIFGKEIRYSGWYPDYVVRLYPTYLAKYGDELVHEKVHYPADSRVEKLQGDLLHFTYKNIHHYLVKSASYAKAWAMQRAKAGKKASLLDGVTHAIACFLKMYLFKAGFLDGKQGFLLAVLSAHSTFVKYADLWDRTRS

SEQ ID NO: 155 - protein, 252 aa, synthetic construct
MKKVSVLIVAKNEANHIRECIESCRFDKEVIVIDDHSADNTAEIAEGLGAKVFRRHLNGDFGAQKTFAIEQAGGEWVFLIDADERCTPELSDEISKIVRTGDYAAYFVERRNLFPNHPATHGAMRPDSVCRLMPKKGGSVQGKVHETVQTPYPERRLKHFMYHYTYDNWEQYFNKFNKYTSISAEKYREQGKPVSFVRDIILRPIWGFFKIYILNKGFLDGKMGWIMSVNHSYYTMIKYVKLYYLYKSGGKF

SEQ ID NO: 156 - protein, 309 aa, synthetic construct
MPNETLHIDIGVCTYRRPELAETLRSLAAMNVPERARLRVIVADNDAEPSARALVEGLRPEMPFDILYVHCPHSNISIARNCCLDNSTGDFLAFLDDDETVSGDWLTRLLETARTTGAAAVLGPVRAHYGPTAPRWMRSGDFHSTLPVWAKGEIRTGYTCNALLRRDAASLLGRRFKLSLGKSGGEDTDFFTGMHCAGGTIAFSPEAWVHEPVPENRASLAWLAKRRFRSGQTHGRLLAEKAHGLRQAWNIALAGAKSGFCATAAVLCFPSAARRNRFALRAVLHAGVISGLLGLKEIEQYGAREVTSA

SEQ ID NO: 157 - protein, 861 aa, synthetic construct
MCRCGRAVRSRPVCRPGQLVVRRSPRPRSRNHSRCRPLRLSVFPRPHRRVRHHCQRDLRWEPGRWIAVRWKAARSHRRFRRCPFPRQLVWPVRERHRDAGDRRNQRERRRRDAYHEISEPKFRTRKRTESFWMNKAITVIVWLLVSLCVLAIITMPVSLQTHLVATAISLILLATIKSFNGQGAWRLVALGFGTAIVLRYVYWRTTSTLPPVNQLENFIPGFLLYLAEMYSVVMLGLSLVIVSMPLPSRKTRPGSPDYRPTVDVFVPSYNEDAELLANTLAAAKNMDYPADRFTVWLLDDGGSVQKRNAANIVEAQAAQRRHEELKKLCEDLDVRYLTRERNVHAKAGNLNNGLAHSTGELVTVFDADHAPARDFLLETVGYFDEDPRLFLVQTPHFFVNPDPIERNLRTFETMPSENEMFYGIIQRGLDKWNGAFFCGSAAVLRREALQDSDGFSGVSITEDCETALALHSRGWNSVYVDKPLIAGLQPATFASFIGQRSRWAQGMMQILIFRQPLFKRGLSFTQRLCYMSSTLFWLFPFPRTIFLFAPLFYLFFDLQIFVASGGEFLAYTAAYMLVNLMMQNYLYGSFRWPWISELYEYVQTVHLLPAVVSVIFNPGKPTFKVTAKDESIAEARLSEISRPFFVIFALLLVAMAFAVWRIYSEPYKADVTLVVGGWNLLNLIFAGCALGVVSERGDKSASRRITVKRRCEVQLGGSDTWVPASIDNVSVHGLLINIFDSATNIEKGATAIVKVKPHSEGVPETMPLNVVRTVRGEGFVSIGCTFSPQRAVDHRLIADLIFANSEQWSEFQRVRRKKPGLIRGTAIFLAIALFQTQRGLYYLVRARRPAPKSAKPVGAVK

SEQ ID NO: 158 - protein, 315 aa, synthetic construct
MIKKIEKDLISVIVPIYNVEDYLVECIESLIVQTYRNIEILLINDGSTDNCATIAKEFSERDCRVIYIEKSNGGLSEARNYGIYHSKGKYLTFVDSDDKVSSDYIANLYNAIQKHDSSIAIGGYLEFYERHNSIRNYEYLDKVIPVEEALLNMYDIKTYGSIFITAWGKLFHKSIFNDLEFALNKYHEDEFFNYKAYLKANSITYIDKPLYHYRIRVGSIMNNSDNVIIARKKLDVLSALDERIKLITSLRKYSVFLQKTEIFYVNQYFRTKKFLKQQSVMFKEDNYIDAYRMYGRLLRKVKLVDKLKLIKNRFF

SEQ ID NO: 159 - protein, 416 aa, synthetic construct
MYTFILMLLDFFQNHDFHFFMLFFVFILIRWAVIYFHAVRYKSYSCSVSDEKLFSSVIIPVVDEPLNLFESVLNRISRHKPSEIIVVINGPKNERLVKLCHDFNEKLENNMTPIQCYYTPVPGKRNAIRVGLEHVDSQSDITVLVDSDTVWTPRTLSELLKPFVCDKKIGGVTTRQKILDPERNLVTMFANLLEEIRAEGTMKAMSVTGKVGCLPGRTIAFRNIVERVYTKFIEETFMGFHKEVSDDRSLTNLTLKKGYKTVMQDTSVVYTDAPTSWKKFIRQQLRWAEGSQYNNLKMTPWMIRNAPLMFFIYFTDMILPMLLISFGVNIFLLKILNITTIVYTASWWEIILYVLLGMIFSFGGRNFKAMSRMKWYYVFLIPVFIIVLSIIMCPIRLLGLMRCSDDLGWGTRNLTE

SEQ ID NO: 160 - protein, 482 aa, synthetic construct
MADAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEATQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 161 - protein, 463 aa, synthetic construct
MANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKAGSGEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKA

SEQ ID NO: 162 - protein, 458 aa, synthetic construct; identical to SEQ ID NO: 145

SEQ ID NO: 163 - protein, 463 aa, synthetic construct
MATKGSSGMSLAERFWLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKDDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAEMIASIADERLEHAETESPAAAGQGRPAAAPTFEVARMKLIR

SEQ ID NO: 164 - protein, 452 aa, synthetic construct
MAENHATFNVLMLPWLAHGHVSPYLELAKKLTARNFNVYLCSSPATLSSVRSKLTEKFSQSIHLVELHLPKLPELPAEYHTTNGLPPHLMPTLKDAFDMAKPNFCNVLKSLKPDLLIYDLLQPWAPEAASAFNIPAVVFISSSATMTSFGLHFFKNPGTKYPYGNAIFYRDYESVFVENLTRRDRDTYRVINCMERSSKIILIKGFNEIEGKYFDYFSCLTGKKVVPVGPLVQDPVLDDEDCRIMQWLNKKEKGSTVFVSFGSEYFLSKKDMEEIAHGLEVSNVDFIWVVRFPKGENIVIEETLPKGFFERVGERGLVVNGWAPQAKILTHPNVGGFVSHCGWNSVMESMKFGLPIIAMPMHLDQPINARLIEEVGAGVEVLRDSKGKLHRERMAETINKVMKEASGESVRKKARELQEKLELKGDEEIDDVVKELVQLCATKNKRNGLHYY

SEQ ID NO: 165 - protein, 484 aa, synthetic construct
MADQMAKIDEKKPHVVFIPFPAQSHIKCMLKLARILHQKGLYITFINTDTNHERLVASGGTQWLENAPGFWFKTVPDGFGSAKDDGVKPTDALRELMDYLKTNFFDLFLDLVLKLEVPATCIICDGCMTFANTIRAAEKLNIPVILFWTMAACGFMAFYQAKVLKEKEIVPVKDETYLTNGYLDMEIDWIPGMKRIRLRDLPEFILATKQNYFAFEFLFETAQLADKVSHMIIHTFEELEASLVSEIKSIFPNVYTIGPLQLLLNKITQKETNNDSYSLWKEEPECVEWLNSKEPNSVVYVNFGSLAVMSLQDLVEFGWGLVNSNHYFLWIIRANLIDGKPAVMPQELKEAMNEKGFVGSWCSQEEVLNHPAVGGFLTHCGWGSIIESLSAGVPMLGWPSIGDQRANCRQMCKEWEVGMEIGKNVKRDEVEKLVRMLMEGLEGERMRKKALEWKKSATLATCCNGSSSLDVEKLANEIKKLSRN

SEQ ID NO: 166 - protein, 384 aa, synthetic construct
MLNNSLFSRLEEIPALLKLKLGSKDYYKNNNSETLTCDNLRYCFDTLNKVSRSFATVIKQLPNELGNNVCVFYLILRALDSIEDDMNLPKELKIKLLREFHKKNYESGWNISGVGDKKEHVELLENYDKVIQSFLAIDQKNQLIITDICRKVGAGMANFVKAEIESVEDYNLYCHHVAGLVGIGLSRMFISSGLENDDFLNQDEISNSMGLFLQKTNIVRDYREDLDEGRMFWPKDIWHVYGSKINDFAINPTHDQSVLCLNHMLNNALTHATDCLAYLKHLRNENIFKFCAIPQVMAMATLCKIYSNPDVFIKNVKIRKGLAAKLILNTTSMDEVIKVYKDMLLVIESKISSDNNPVSAETIQLLKQIREYFNDETLIVRKIA

SEQ ID NO: 167 - protein, 383 aa, synthetic construct
MLNSSLFSRLEEIPALLKLKLGSINNYKNNNSENLTSKNLRYCFDTLNKVSRSFASVIKQLPNELMVNVCLFYLILRALDSIEDDMNLPKDFKINLLREFLDKNYEPGWKISGVGDKKEYVELLENYDKVIQVFLDIDPKNQLIITDICRKMGAGMAHFVEAEINSVKDYNLYCYHVAGLVGIGLSKMFLASGLENCDYLNQEEISSSMGLFLQKTNIVRDYKEDMEENRIFWPKEIWRTYASKFSDFSINPQHETSISCLNHMVNDALGHVIDCLEYLRHLRNENIFKFCAIPQVMAMATLCKVYNNPDVFIKTVKIRKGLAAKLILNTTSMDEVIKVYKGLLLDIENKIPLHNPTSDETLRLIKNIRSYCNNETMVVSKTA

SEQ ID NO: 168 - protein, 458 aa, synthetic construct
MHTTSEHNDLFDICIVGAGMAGATIATYLAPRGIKIALIDRDYAEKRRIVGELLQPGAVQTLKKMGLEHLLEGFDAQPIYGYALFNKDCEFSIEYNQDKSTNYRGVGLHNGRFLQKIREDALKQPSITQIHGTVSELIEDENHVVTGVKYKEKYTRELKTVNAKLTITSDGFFSSFRKDLTNNVKTVTSFFVGIILKDCELPYPHHGHVFLSAPTPFICYPISSTESRLLIDFPGDQAPKKEAVKHHIENNVIPFLPKEFRLCLDQALRENDYKIMPNHYMPAKPVLKKGVVLLGDALNMRHPITGGGLTAVFNDVYLLSTHLLAMPDFNDTKLIHEKVNLYYNDRYHANTNVNIMANALYGVMSNDLLKQSVFEYLRKGGDNSGGPISLLAGLNRNPTILIKHFFSVALLCLRNLFKAHKMSLTNAFYVIKDAFCIIVPLAINELRPSSFLKKNIHN

SEQ ID NO: 169 - protein, 458 aa, synthetic construct
MNTSPEHNDLFDICIVGVGMAGATIAAYLAPRGLKIALIDREYTEKRRIVGELLQPGAVQTLKKMGLEHLLEGFDAQPIYGYALFNNDKEFSISYNSDDSTEYHGVGLHNGRFLQKIREDVFKNETVTQIHGTVSELIEDKKGVVKGVTYREKHTREYKTVKAKLTVTSDGFFSNFRKDLSNNVKTVTSFFIGLVLNDCNLPFPNHGHVFLSAPTPFICYPISSTETRLLIDYPGDKAPKKDEIREHILNKVAPFLPEEFKECFANAMEDDDFKVMPNHYMPAKPVLKEGAVLLGDALNMRHPLTGGGLTAVFNDVYLLSTHLLAMPDFNDPKLLHEKLELYYQDRYHANTNVNIMANALYGVMSNDLLKQGVFEYLRKGGDNSGGPITLLAGLNRNPTLLIKHFFSVAFLCICNLSGNNKMNFTNVFRVMKDAFCIIKPLAVNELRPSSFYKKNIQL

SEQ ID NO: 170 - protein, 448 aa, synthetic construct
MESNFDICIIGAGMAGATIAAYLAPKGINIALIDHCYKEKKRIVGELLQPGAVLSLEQLGLGHLLDGIDAQPVEGYALLQGNEQTTIPYPSPNHGMGLHNGRFLQQIRASALQNSSVTQIQGKALSLLENEQNEIIGVNYRDSVSNEIKSIYAPLTITSDGFFSNFRELLSNNEKTVTSYFIGLILKDCEIPVPKHGHVFLSGPTPFICYPISSNEVRLLIDFPGGQFPRKAFLQAHLETNVTPYIPEGMQTSYRHALQEDRLKVMPNHYMAAKPKIRKGAVMLGDALNMRHPLTGGGLTAVFSDIEILSGHLLAMPDFNNNDLIYQKIEAYYRDRQYANANLNILANALYGVMSNELLKNSVFKYLQRGGVNAKESIAILAGLNKNHYSLMKQFFFVALFGAYTLVRENITNLPKATKILSDALTIIKPLAKNELSLVGIFSDYFKR

SEQ ID NO: 171 - protein, 474 aa, synthetic construct
MWAIVVGLATLAVAYYIHWINKWKDSKFNGVLPPGTMGLPLVGETLQLARPSDSLDVHPFIKKKVKRYGPIFKTCLAGRPVVVSTDAEFNNYIMLQEGRAVEMWYLDTLSKFFGLDTEWLKALGFIHKYIRSITLNHFGAESLRERFLPRIEESAKETLRYWATQPSVEVKDSAAVMVFRTSMVKMVSEDSSKLLTGGLTKKFTGLLGGFLTLPINVPGTTYNKCMKDMKEIQKKLREILEGRLASGGGSDEDFLGQAIKDKGSQQFISDDFIIQLLFSISFASFESISTTLTLVLNYLADHPDVVKELEAEHEAIRNARADPDGPITWEEYKSMTFTLHVIFETLRLGSVTPALLRKTTKELQINGYTIPEGWTVMLVTASRHRDPAVYKDPHTFNPWRWKELDSITIQKNFMPFGGGLRHCAGAEYSKVYLCTFLHILFTKYRWTKLKGGKVARAHILSFEDGLHVKFTPKE

SEQ ID NO: 172 - protein, 512 aa, synthetic construct
MATLMTIDLQNCFIFIILSLLCYYLLFKKQKGSRAGCVLPPSPPSLPIIGHLHLLLSNLTHKSLQNISTKFGSFLYLRVVNLPIVLVSSPSVAYEIYKTHDVNVSSRVATSLGDSLFLGSSGFITAPYGDYWKFMKKMVATKLLRPQAIEQSRGGRAEELQMFYENLLDKAMKKESIEVSKEAMKLTNNIICRMSMGRSCSDENGEAERVRELLVKSTALTKKIFFANMFPRIPLFKKEIMGVSSEFDDLLERLLVEHEERVEEHENKDMMDLLLEAYRDENAEYKISRKQIKSLFVEIFLGGTDTSAQTVQWILAELINKPNILERIREEIDSVVGKSRLMKETDLPNLPYLQATVKEGLRMHPPSPLLVRTFQESCEVKGFYMPEKTMLVINVYALMRDPDTWEDPNEFKPERFLLSSRSRQEDEKEQGMMKYLPFGAGRRGCPGSNLAYLFVGIAVGVMVQCFDWKIKEDKVNMEETTAGMNLAMAHPFKCTPVVRNDPLTLNLENPSS

SEQ ID NO: 173 - protein, 498 aa, synthetic construct
MIVDFQNCSIFILLCFFTFLCYSVFFFFKKTNDLGPSPPSLPIIGHLHHFLSGLPHKAFQKISTKYGPLLHLHIFSFPIVLVSSPTMAHEIFTTHDLNISSRNTPAIDESLLFGPSGFTVAPYGDYVKFIKKLLATKLLRPRAIEKSRGVRAEELKQFYLKVQDKALKKESIEIGKETMKFTNNMICRMSIGRSFSEENGEVETLRELIIKSFALSKQILFVNVLRRPLEMLGLMSLFKKDIMDVSRGFDELLERVLAEHEEKREEDQDMDMMDLLLEACRDENAEYKITRNQIKSLFVEIFLGGTDTSAHTTQWTMAELVNNPNILGRLRDEIDLVVGKERLIQETDLPNLPYLQAVVKEGLRLHPPAPLLVRMFDKKCVIKDFFKVPEKTTLVVNVYGVMRDPDSWEDPNEFKPERFLTSKQEEDKVLKYLPFAAGRRGCPATNVGYIFVGISIGMMVQCFDWSIKEKVSMEEVYAGMSLSMAHPPTCTPVSRLSL

SEQ ID NO: 174 - protein, 502 aa, synthetic construct
MDFFSAFLLLLLTVLILLQIRTRRRNLPPSPPSLPIIGHLHLLKRPIHRNFHKIAAEYGPIFSLRFGSRLAVIVSSLDIAEECFTKNDLIFANRPRLLISKHLGYNCTTMATSPYGDHWRNLRRLAAIEIFSTARLNSSLSIRKDEIQRLLLKLHSGSSGEFTKVELKTMFSELAFNALMRIVAGKRYYGDEVSDEEEAREFRGLMEEISLHGGASHWVDFMPILKWIGGGGFEKSLVRLTKRTDKFMQALIEERRNKKVLERKNSLLDRLLELQASEPEYYTDQIIKGLVLVLLRAGTDTSAVTLNWAMAQLLNNPELLAKAKAELDTKIGQDRPVDEPDLPNLSYLQAIVSETLRLHPAAPMLLSHYSSADCTVAGYDIPRGTTLLVNAWAIHRDPKLWDDPTSFRPERFLGAANELQSKKLIAFGLGRRSCPGDTMALRFVGLTLGLLIQCYQWKKCGDEKVDMGEGGGITIHKAKPLEAMCKARPAMYKLLLNALDKI

SEQ ID NO: 175 - protein, 514 aa, synthetic construct
MATMMIFDFQNCFIFIILCFVSLLCYTILFKKQESSRTGCVLPPSPPSLPIIGHLHLLLSSLTHKSLHNISSKFGPFLYLRVVNLPIVLVSSASVAYEIYKTQDVNVSSRVATSLGDSLFLGSSGFITAPYGDYWKFMKKMVATKLLRPQAIEQSRGGRAEELQGLYENLLDKAMKKESIEISKEAMKFTNNIICRMSMGRSCSDENGEAEIVRELLVKSTALTKKIFFANMFPRIPLFKKEIMGVSNQFDELLERLLVEHEERVEEHENKDMMDLLLEAFRDEHAEYKISRKQIKSLFVEIFLGGTDTSAQTVQWIMAELINKPSIIEKIREEIDSVVGKTRLIKETDLPKLPYLQVVVKEGLRMHPPSPLVVRTFQESCEVKGFYMPEKTMLVINVYALMRDPESWEDPNEFKPERFLPSSKSRQDEEKEQGLKYLPFGAGRRGCPGSNLAYLFVGLAVGVMVQCFDWKIKEDKVNMEETTAGMNLAMAHPFKCTPVVRIDPLTFNLKSPSP

SEQ ID NO: 176 - protein, 499 aa, synthetic construct
MAPMTIDFQTCFIFILLSFFSFFCYFFFFKKTNDLGPSPPSLPIIGHLHHFLSVLPHKAFQQISTKYGPLLHLRIFSFPIVLVSSATMAYEIFTTHDLNISSRNAPAIDESLVFGSSGFIVSPYGDYVKFIKKLLATKLLRPRAIEKSRGVRAEELKQFYLKLHDKALKKESIEIGNETMKFTNNMICGMSMGRSCSEENGETETVRGLINKSFALSRKILFVNVLRRPLEKLGLLSLFKKDILDVSNRFDELLERILLEHEEKPEEEQDMDMMDLLLEASRDENAEYKITRNQIKALFVEIFMGGTDTSAHTTQWTMAELVNNPNSLEKLRDEIDMVVGKSRLIQETDLPNLPYLQAVVKEGLRLHPPAPLLVRMFEKKCVIKDFFNVPEKTTLVVNLYGVMRDPDSWEDPNEFKPERFLTSKQEEEKTLKYLPFAAGRRGCPATNVAYIFVGISIGMMVQCFDWSIKDKVSMEEVYAGMSLSMAHPPKFTPVSRLSL

SEQ ID NO: 177 - protein, 522 aa, synthetic construct
MVDPYAVGWIICSLTTIVALYNFVFYRQNRSDKTTPTTTENITTATGDCRSLNPNGDVDIVIVGAGVAGSALAYTLGKDGRRVLVIERDLNEPDRIVGELLQPGGYLKLIELGLEDCVEKIDAQQVFGYALFKDGKHTRLSYPLEKFHSDIAGRSFHNGRFIQRMREKAASLPNVQLVQGTVTSLLEENGTIKGVQYKTKDAQELSACAPLTIVCDGCFSNLRRNLCNPKVEVPSCFVGLVLENCELPCANHGHVILGDPSPVLFYPISSTEIRCLVDVPGQKVPSISNGEMAKYLKEVVAPQVPPELHDAFIAAVDKGNIRTMPNRSMPAAPYPTPGALLMGDAFNMRHPLTGGGMTVALSDIVVLRNLLKPLRDLNDAPSLCKYLESFYTLRKPVASTINTLAGALYKVFCASPDPARKEMRQACFDYLSLGGLFSEGPVSLLSGLNPRPLSLVLHFFAVAIYGVGRLLLPFPSPKRIWIGVRLIASASGIILPIIKAEGIRQMFFPATVPAYYRTPPAA

SEQ ID NO: 178 - protein, 525 aa, synthetic construct
MDLYLLGWILSSVLSLFALYCLVFDGNRSRANAEKQIQRGYSVTTDAGDVKSEKLNGDADVIIVGAGIAGAALAHTLGKDGRRVRVIERDLSEPDRIVGELLQPGGYLKLVELGLADCVDNIDAQKVFGYALFKDGKHTRLSYPLEKFHADVSGRSFHNGRFIQRMREKAASLLNVNLEQGTVTSLLEEKGTIKGVQYKNKDGQELTAYAPLTIVCDGCFSNLRRSLCNPKVDNPSCFVGLVLENCELPCANHGHVILGDPSPILFYPISSTEIRCLVDVPGQKVPSISNGDMTKYLKLTVAPQVPPELYDAFIAAVDKGNIRTMPNKSMPADPCPTPGAVLMGDAFNMRHPLTGGGMTVALSDIVVLRNLLRPLRDLNDAPALCKYLESFYTLRKPVASTINTLAGALYKVFSSSPDQARREMRQACFDYLSLGGLFSEGPISLLSGLNPRPLSLVLHFFAVAVFGVGRLLLPFPSPKRVWIGARLLSAASGIILPIIKAEGIRQMFFPVTVPAYYRAPPTSQE

SEQ ID NO: 179 - protein, 519 aa, synthetic construct
MIDPYGFGWITCTLITLAALYNFLFSRKNHSDSTTTENITTATGECRSFNPNGDVDIIIVGAGVAGSALAYTLGKDGRRVLIIERDLNEPDRIVGELLQPGGYLKLIELGLDDCVEKIDAQKVFGYALFKDGKHTRLSYPLEKFHSDIAGRSFHNGRFILRMREKAASLPNVRLEQGTVTSLLEENGTIKGVQYKTKDAQEFSACAPLTIVCDGCFSNLRRSLCNPKVEVPSCFVGLVLENCELPCADHGHVILGDPSPVLFYPISSTEIRCLVDVPGQKVPSISNGEMAKYLKTVVAPQVPPELHAAFIAAVDKGHIRTMPNRSMPADPYPTPGALLMGDAFNMRHPLTGGGMTVALSDIVVLRNLLKPLRDLNDASSLCKYLESFYTLRKPVASTINTLAGALYKVFCASPDPARKEMRQACFDYLSLGGLFSEGPVSLLSGLNPCPLSLVLHFFAVAIYGVGRLLLPFPSPKRLWIGIRLIASASGIILPIIKAEGIRQMFFPATVPAYYRAPPDA

SEQ ID NO: 180 - protein, 525 aa, synthetic construct
MDLYNIGWILSSVLSLFALYNLIFAGKKNYDVNEKVNQREDSVTSTDAGEIKSDKLNGDADVIIVGAGIAGAALAHTLGKDGRRVHIIERDLSEPDRIVGELLQPGGYLKLVELGLQDCVDNIDAQRVFGYALFKDGKHTRLSYPLEKFHSDVSGRSFHGRFIQRMREKAASLPNVNMEQGTVISLLEEKGTIKGVQYKNKDGQALTAYAPLTIVCDGCFSNLRRSLCNPKVDNPSCFVGLILENCELPCANHGHVILGDPSPILFYPISSTEIRCLVDVPGTKVPSISNGDMTKYLKTTVAPQVPPELYDAFIAAVDKGNIRTMPNRSMPADPRPTPGAVLMGDAFNMRHPLTGGGMTVALSDIVVLRNLLKPMRDLNDAPTLCKYLESFYTLRKPVASTINTLAGALYKVFSASPDEARKEMRQACFDYLSLGGLFSEGPISLLSGLNPRPLSLVLHFFAVAVFGVGRLLLPFPSPKRVWIGARLLSGASGIILPIIKAEGIRQMFFPATVPAYYRAPPVNAF

SEQ ID NO: 181 - protein, 461 aa, synthetic construct
MSKSRSNYDVIIVGAGIAGCALAHGLSTLSRATPLRIAIVERSLAEPDRIVGELLQPGGVMALQRLGMEGCLEGIDAVKVHGYCVVENGTSVHIPYPGVHEGRSFHHGRFIMKLREAARAARGVELVEATVTELIPREGGKGIAGVRVARKGKDGEEDTTEALGAALVVVADGCFSNFRAAVMGGAAVKPETKSHFVGAILKDARLPIPNHGTVALVKGFGPVLLYQISEHDTRMLVDVKAPLPADLKVCAHILSNIVPQLPAALHLPIQRALDAERLRRMPNSFLPPVEQGATRGAVLVGDAWNMRHPLTGGGMTVALNDVVVLRDLLGSVGDLGDWRQVASTVNILSVALYDLFGADGELQVLRTGCFKYFERGGDCIDGPVSLLSGIAPSPMLLAYHFFSVAFYSIYVIAVGAQNGSAKQVLAVPGALQYPALCVKGLRVFYTACVVFGPLLWTELRW

SEQ ID NO: 182 - protein, 498 aa, synthetic construct
MHPTHYDVVIVGAGVAGSSLAHALATLPREKPLQIALIERSFEEPDRIVGELLQPGGVDALKTLKMTSSVEGIDAITVTGYILVESGDMVRIPYPKGKEGRSFHHGRFIMGLRRVALENPNVHPIEATAADLIECPCTGQVIGVRATSKTAPAPSSIDAQQTPPAPFSVYGDLVIVADGCFSNFRNVVMGKAACKATTKSYFVGTILKDAVLPVAGHGTVILPQGSGPVLLYQISEHDTRMLIDIQHPLPSDLRAHILTNILPQLPASIQGVVSDAFTKDRIRRMPNSFLPSVQQGSPLSKKGVILLGDSWNMRHPLTGGGMTVALNDVVYLRSIFASIQNLDDWDEIRYALRHWHWGRKPLSSTINILSGTLYGLFEKDDDDYRALRKGCFKYFQLGGKCIDDPVSLLSGLSPSPLLLSSHFFAVILYAIWVVFTHPRVGSSMSANPADVKRVYDIPSADEYPQLTLKGIRMFSQACGVFLPVLWSEIRWWAPCESS

SEQ ID NO: 183 - protein, 524 aa, synthetic construct
MSKSRSNYDVIIVGAGIAGCALAHGLSTLSRATPLRIAIVERSLAEPDRIVGELLQPGGVMALQRLGMEGCLEGIDAVKVHGYCVVENGTSVHIPYPGVHEGRSFHHGRFIMKLREAARAARGVELVEATVTELIPREGGKGIAGVRVARKGKDGEEDTTEALGAALVVVADGCFSNFRAAVMGGAAVKPETKSHFVGAILKDARLPIPNHGTVALVKGFGPVLLYQISEHDTRMLVDVKAPLPADLKAHILSNIVPQLPAALHLPIQRALDAERLRRMPNSFLPPVEQGATRGAVLVGDAWNMRHPLTGGGMTVALNDVVVLRDLLGSVGDLGDWRQVRRALHRWHWDRKPLASTVNILSVALYDLFGADGEELQVLRTGCFKYFERGGDCIDGPVSLLSGIAPSPMLLAYHFFSVAFYSIYVMFAHPQPVAQSKAVGAQNGSAKQVLAVPGALQYPALCVKGLRVFYTACVVFGPLLWTELRWWTAAEASRGRLLVMSLVPLLLLLGAANYGIPGMGLLGVL

SEQ ID NO: 184 - protein, 317 aa, synthetic construct
MAEKIEHTTVTTNGIKMHVASIGSGPIILFLHGFPELWYTWRHQLLSLSSLGYRCVAPDLRGYGDSDAPPSPESYTVFHIVGDLVGLLDALGVDKVFLVGHDWGAMIAWNFCLFRPDRIKALVNLSIPYHPRNPKVKTVDGYRALFGDDFYICRFQVPGEAEAHFAQMDTAKVMKKFLTTRDPNPPCIPRETGLKALPDPPALPSWLSEDEINYFATKFSQKGFTGGLNYYRAMNLNWELMAPWTGLQIQVPVKFIVGDLDITYHIPGVKEYLQNGGFKKNVPFLQELVVMEGVAHFINQEKPQEISMHIYDFIKKF

SEQ ID NO: 185 - protein, 317 aa, synthetic construct
MAEKIEHTTVTTNGIKMHVASIGSGPIILFLHGFPELWYTWRRQLLSLSSLGYRCVAPDLRGYGDSDAPPSPESYTVFHVVGDLVGLLDALGVDKVFLVGHDWGAMIAWNFCLFRPDRIKALVNLSVPYHPRNPKVKTVDGYRALFGDDFYICRFQVPGEAEAHFAQMDTAKVLKKFLTTRDPNPPCIPKETGLKALPDPPALPSWLSEDEINYFATKFNQKGFTGGLNYYRAMNLNWELMAPWTGLQIQVPVKFIVGDLDITYHIPGVKEYLQNGGFKKNVPFLQELVVMEGVAHFINQEKPQEISMHIYDFVKKF

SEQ ID NO: 186 - protein, 317 aa, synthetic construct
MAENIEHTTVQTNGIKMHVAAIGTGPPVLLLHGFPELWYSWRHQLLYLSSAGYRAIAPDLRGYGDTDAPPSPSSYTALHIVGDLVGLLDVLGIEKVFLIGHDWGAIIAWYFCLFRPDRIKALVNLSVQFFPRNPTTPFVKGFRAVLGDQFYMVRFQEPGKAEEEFASVDIREFFKNVLSNRDPQAPYLPNEVKFEGVPPPALAPWLTPEDIDVYADKFAETGFTGGLNYYRAFDRTWELTAPWTGARIGVPVKFIVGDLDLTYHFPGAQKYIHGEGFKKAVPGLEEVVVMEDTSHFINQERPHEINSHIHDFFSKFC

SEQ ID NO: 187 - protein, 316 aa, synthetic construct
MADKIQHSTISTNGINIHFASIGSGPVVLFLHGFPELWYSWRHQLLFLASKGFRAIAPDLRGFGDSDAPPSPSSYTPHHIVGDLIGLLDHLGIDQVFLVGHDWGAMMAWYFCLFRPDRVKALVNLSVHYTPRNPAGSPLAVTRRYLGDDFYICKFQEPGVAEADFGSVDTATMMKKFLTMRDPRPAIIPNGFKTLLETPEILPSWLTEEDIEYFASKFSKTGFTGGFNYYRALDITWELTGPWSRAQIKVPTKFIVGDLDLVYNFPGAKEYIHGGGFKKDVPLLEDVVVIEGAAHFINQEKPDEISSLIYDFITKF

SEQ ID NO: 188 - protein, 325 aa, synthetic construct
MAEKIEHTTIPTNGINMHVASIGSGPAVLFLHGFPQLWYSWRHQLLFLASKGFRALAPDLRGFGDTDAPPSPSSYTFLHIIGDLIGLLDHLGLEKVFLVGHDWGAMIAWYFCLFRPDRVKALVNLSVYYIKRHPSISFVDGFRAVAGDNFYICQFQEAGVAEADFGRVDTATMMKKFMGMRDPEAPLIFTKEKGFSSMETPDPLPCWLTEEDIDFFATKFSKTGFTGGFNYYRALNLSWELTAAWNGSKIEVPVKFIVGDLDLVYHFPGAKQYIHGGEFKKDVPFLEEVVVIKDAAHFIHQEKPHQINSLIYHFINKFSTSTSPA

SEQ ID NO: 189 - protein, 316 aa, synthetic construct
MAEKIEHTTINTNGVNLHVASIGTGPAVLFLHGFPELWYSWRHQMLALSSLGYRAIAPDLRGYGDSDAPPSPESYSSLHIVGDLVGLIDQLGIDQIFLVGHDWGAVIAWQFCLFRPDRVKALVNMSVPFRPRHPTRKPIETFRALFGDDYYVCRFQAPGEVEEDFASDDTANLLKKFYGGRNPRPPCVPKEIGFKGLKAPELPSWLSEEDLNYFAEKFNQRGFTGGLNYYRALDLTWELTAAWTGVQVKVPTKFIVGDLDITYHIPGAKEYINEGGLKKDVPYLQEVVVMEGVAHFVNQEKAEEVSAHIHDFIKKF

SEQ ID NO: 190 - protein, 318 aa, synthetic construct
MAEKIEHTWVNTNGIKMHVASIGSGPAVLFLHGFPELWYSWRHQLLSLSAQGYRCIAPDLRGYGDTDAPPSPSSYSALHIVSDLVGLLDALRIDQVFLVGHDWGAAMAWYFCLFRPDRIKALVNMSVVFRPRNPKWKPLQSLRAMLGDDYYICRFQKPGEAEEEFARAGTSRIIKTFLVSRDPRPPCVPKEIGFGGSPNLQLALPSWLTEEDVNYYASKFDQKGFTGGLNYYRAIDLTWELTAPWTGVQIKVPVKFIVGDLDVTYNTPGVKEYIHGGGFKKEVPFLQELVVMEGVAHFINQERPDEISAHIHDFIKKF

SEQ ID NO: 191 - protein, 757 aa, synthetic construct
MAWKLKVAEGGTPWLRTLNNHVGRQVWEFDPHSGSPQDLDDIETARRNFHDNRFTHKHSDDLLMRLQFAKENPMNEVLPKVKVKDVEDVTEEAVATTLRRGLNFYSTIQSHDGHWPGDLGGPMFLMPGLVITLSVTGALNAVLTDEHRKEMRRYLYNHQNKDGGWGLHIEGPSTMFGSVLCYVTLRLLGEGPNDGEGDMERGRDWILEHGGATYITSWGKMWLSVLGVFEWSGNNPMPPEIWLLPYALPVHPGRMWCHCRMVYLPMSYLYGKRFVGPITPTVLSLRKELFTVPYHDIDWNQARNLCAKEDLYYPHPLVQDILWATLHKFVEPVFMNWPGKKLREKAIKTAIEHIHYEDENTRYICIGPVNKVLNMLCCWVEDPNSEAFKLHLPRIYDYLWVAEDGMKMQGYNGSQLWDTAFAAQAIISTNLIDEFGPTLKKAHAFIKNSQVSEDCPGDLSKWYRHISKGAWPFSTADHGWPISDCTAEGLKAVLLLSKIAPEIVGEPLDSKRLYDAVNVILSLQNENGGLATYELTRSYTWLEIINPAETFGDIVIDCPYVECTSAAIQALATFGKLYPGHRREEIQCCIEKAVAFIEKIQASDGSWYGSWGVCFTYGTWFGIKGLIAAGKNFSNCLSIRKACEFLLSKQLPSGGWAESYLSCQNKVYSNLEGNRSHVVNTGWAMLALIEAEQAKRDPTPLHRAAVCLINSQLENGDFPQEEIMGVFNKNCMITYAAYRCIFPIWALGEYRRVLQAC

SEQ ID NO: 192 - protein, 767 aa, synthetic construct
MAWQLKIGADTVPSDPSNAGGWLSTLNNHVGRQVWHFHPELGSPEDLQQIQQARQHFSDHRFEKKHSADLLMRMQFAKENSSFVNLPQVKVKDKEDVTEEAVTRTLRRAINFYSTIQADDGHWPGDLGGPMFLIPGLVITLSITGALNAVLSTEHQREICRYLYNHQNKDGGWGLHIEGPSTMFGSVLNYVTLRLLGEEAEDGQGAVDKARKWILDHGGAAAITSWGKMWLSVLGVYEWAGNNPLPPELWLLPYLLPCHPGRMWCHCRMVYLPMCYLYGKRFVGPITPIIRSLRKELYLVPYHEVDWNKARNQCAKEDLYYPHPLVQDILWATLHHVYEPLFMHWPAKRLREKALQSVMQHIHYEDENTRYICIGPVNKVLNMLCCWAEDPHSEAFKLHIPRIYDYLWIAEDGMKMQGYNGSQLWDTAFAVQAIISTELAEEYETTLRKAHKYIKDSQVLEDCPGDLQSWYRHISKGAWPFSTADHGWPISDCTAEGLKAVLLLSKLPSEIVGKSIDEQQLYNAVNVILSLQNTDGGFATYELTRSYRWLELMNPAETFGDIVIDYPYVECSSAAIQALAAFKKLYPGHRRDEIDNCIAEAADFIESIQATDGSWYGSWGVCFTYGGWFGIRGLVAAGRRYNNCSSLRKACDFLLSKELAAGGWGESYLSCQNKVYTNIKDDRPHIVNTGWAMLSLIDAGQSERDPTPLHRAARVLINSQMEDGDFPQEEIMGVFNKNCMISYSAYRNIFPIWALGEYRSRVLKPLK

SEQ ID NO: 193 - protein, 761 aa, synthetic construct
MAWKLKVAEGRDARLRTINGHVGRQIWEFDPDLGTDNERAEVEAVREKFRNNRFEKKHSSDLLMRLQLAKENPVSSYLTQVKLEENEDITEEAVTMTLRRALNFHSSIQSFDGHWAGDLGGPMFLMPGLVISLYITGVLNTVLSSEHQREMCRYLYNHQNEDGGWGLHIEGPSTVFGSTLTYITLRLLGENVEDGDGAMEKGRKWILDHGGATYITSWGKMWLSVLGVFDWSGNNPLPPEMWLLPYFLPVHPGRMWCHCRMVYLPMSYLYGKRFVGKITPLVLSLRNEIYTVSYNQIDWNKARNLCAKEDLYYPHPMVQDILWATLHKFVEPILMHWPGTLLREKALNTTMQHIHYEDESTRYICIGPVNKVLNMLCCWVDDPDSEAFKLHLPRISDYLWIAEDGMKCQGYNGSQLWDTAFAVQAYIATNLSDEFGPVLTKAHEYIKNSQVPDDCSGDLSFWYRHISKGAWPFSTGDHGWPISDCTAEGLKASLLLSRISPEVVGKPLNAKRFYDAVNVILSLMNSDGSFATYELTRSYTWLEMINPAETFGDIVIDYPYVECTSAAIQSLVAFTKLYPGHRREEIDECITKAAKFIESIQKKDGSWYGSWAVCFTYGLWFGIKGLIAAGKTYKNSSAIRKACEFLLSKQLASGGWGESYLSCQDKVYTNLEGNRAHAVNTGWAMLSLIDAGQAERDPSPLHRAARVLINSQMGNGDFPQEEIMGVFNRNCMISYSAYRNIFPIWALGEYRCKVLASKGHE

SEQ ID NO: 194 - protein, 475 aa, synthetic construct
MAWTILLGLATLAIAYYIHWVNKWKDSKFNGVLPPGTMGLPLIGETIQLSRPSDSLDVHPFIQRKVKRYGPIFKTCLAGRPVVVSTDAEFNHYIMLQEGRAVEMWYLDTLSKFFGLDTEWLKALGLIHKYIRSITLNHFGAESLRERFLPRIEESARETLHYWSTQTSVEVKESAAAMVFRTSIVKMFSEDSSKLLTEGLTKKFTGLLGGFLTLPLNLPGTTYHKCIKDMKQIQKKLKDILEERLAKGVKIDEDFLGQAIKDKESQQFISEEFIIQLLFSISFASFESISTTLTLILNFLADHPDVVKELEAEHEAIRKARADPDGPITWEEYKSMNFTLNVICETLRLGSVTPALLRKTTKEIQIKGYTIPEGWTVMLVTASRHRDPEVYKDPDTFNPWRWKELDSITIQKNFMPFGGGLRHCAGAEYSKVYLCTFLHILFTKYRWRKLKGGKIARAHILRFEDGLYVNFTPKE

SEQ ID NO: 195 - protein, 485 aa, synthetic construct
MALLSEYGLFLAKIVTVVLAIAAIAAIIHWVNKWKDSKFNGVLPPGTMGLPLIGETIQLSRPSDSLDVHPFIQRKVKRYGPIFKTCLAGRPVVVSTDAEFNHYIMLQEGRAVEMWYLDTLSKFFGLDTEWLKALGLIHKYIRSITLNHFGAESLRERFLPRIEESARETLHYWSTQTSVEVKESAAAMVFRTSIVKMFSEDSSKLLTEGLTKKFTGLLGGFLTLPLNLPGTTYHKCIKDMKQIQKKLKDILEERLAKGVKIDEDFLGQAIKDKESQQFISEEFIIQLLFSISFASFESISTTLTLILNFLADHPDVVKELEAEHEAIRKARADPDGPITWEEYKSMNFTLNVICETLRLGSVTPALLRKTTKEIQIKGYTIPEGWTVMLVTASRHRDPEVYKDPDTFNPWRWKELDSITIQKNFMPFGGGLRHCAGAEYSKVYLCTFLHILFTKYRWRKLKGGKIARAHILRFEDGLYVNFTPKE

SEQ ID NO: 196 - protein, 482 aa, synthetic construct
MAQDLRLILIIVGAIAIIALLVHGFHWVNKWKDSKFNGVLPPGTMGLPLIGETIQLSRPSDSLDVHPFIQRKVKRYGPIFKTCLAGRPVVVSTDAEFNHYIMLQEGRAVEMWYLDTLSKFFGLDTEWLKALGLIHKYIRSITLNHFGAESLRERFLPRIEESARETLHYWSTQTSVEVKESAAAMVFRTSIVKMFSEDSSKLLTEGLTKKFTGLLGGFLTLPLNLPGTTYHKCIKDMKQIQKKLKDILEERLAKGVKIDEDFLGQAIKDKESQQFISEEFIIQLLFSISFASFESISTTLTLILNFLADHPDVVKELEAEHEAIRKARADPDGPITWEEYKSMNFTLNVICETLRLGSVTPALLRKTTKEIQIKGYTIPEGWTVMLVTASRHRDPEVYKDPDTFNPWRWKELDSITIQKNFMPFGGGLRHCAGAEYSKVYLCTFLHILFTKYRWRKLKGGKIARAHILRFEDGLYVNFTPKE

SEQ ID NO: 197 - protein, 475 aa, synthetic construct
MAWTILLGLATLAIAYYIHWVNKWKDSKFNGVLPPGTMGLPLIGETIQFSRPSDSLDVHPFIQRKVKRYGPIFKTCIAGRPVVVSTDAEFNHYIMLQEGRAVEMWYLDTFSKFLGLDTEWLKALGLIHKYIRSITLNHFGAESLRERFLPRIEESARETLHYWSTQTSVEVKESAAAMVFRTSIVKMFSEDSSKLLTEGLTKKFTGLLGGFLTLPLNLPGTTYHKCIKDMKQIQKKLKDILEERLAKGVKIDEDFLGQAIKDKESQQFISEEFIIQLLFSISFASFASISTTLTLILNFLADHPDVVKELEAEHEAIRKARADPDGPITWEEYKSMNFTLNVICETLRLGSVTPALLRKTTKEIQIKGYTIPEGWTVMLVTASRHRDPEVYKDPDTFNPWRWKELDSITIQKNFMPFGGGLRHCAGAEYSKVYLCTFLHILFTKYRWRKLKGGKIARALILRFEDGLYVNFTPKE

SEQ ID NO: 198 - protein, 485 aa, synthetic construct
MALLSEYGLFLAKIVTVVLAIAAIAAIIHWVNKWKDSKFNGVLPPGTMGLPLIGETIQFSRPSDSLDVHPFIQRKVKRYGPIFKTCIAGRPVVVSTDAEFNHYIMLQEGRAVEMWYLDTFSKFLGLDTEWLKALGLIHKYIRSITLNHFGAESLRERFLPRIEESARETLHYWSTQTSVEVKESAAAMVFRTSIVKMFSEDSSKLLTEGLTKKFTGLLGGFLTLPLNLPGTTYHKCIKDMKQIQKKLKDILEERLAKGVKIDEDFLGQAIKDKESQQFISEEFIIQLLFSISFASFASISTTLTLILNFLADHPDVVKELEAEHEAIRKARADPDGPITWEEYKSMNFTLNVICETLRLGSVTPALLRKTTKEIQIKGYTIPEGWTVMLVTASRHRDPEVYKDPDTFNPWRWKELDSITIQKNFMPFGGGLRHCAGAEYSKVYLCTFLHILFTKYRWRKLKGGKIARALILRFEDGLYVNFTPKE

SEQ ID NO: 199 - protein, 485 aa, synthetic construct
MALLSEYGLFLAKIVTVVLAIAAIAAIIHWINKWKDSKFNGVLPPGTMGLPLVGETLQLARPSDSLDVHPFIKKKVKRYGPIFKTCLAGRPVVVSTDAEFNNYIMLQEGRAVEMWYLDTLSKFFGLDTEWLKALGFIHKYIRSITLNHFGAESLRERFLPRIEESAKETLRYWATQPSVEVKDSAAVMVFRTSMVKMVSEDSSKLLTGGLTKKFTGLLGGFLTLPINVPGTTYNKCMKDMKEIQKKLREILEGRLASGGGSDEDFLGQAIKDKGSQQFISDDFIIQLLFSISFASFESISTTLTLVLNYLADHPDVVKELEAEHEAIRNARADPDGPITWEEYKSMTFTLHVIFETLRLGSVTPALLRKTTKELQINGYTIPEGWTVMLVTASRHRDPAVYKDPHTFNPWRWKELDSITIQKNFMPFGGGLRHCAGAEYSKVYLCTFLHILFTKYRWTKLKGGKVARAHILSFEDGLHVKFTPKE

SEQ ID NO: 200 - protein, 465 aa, synthetic construct
MALLLAVFHWINKWKDSKFNGVLPPGTMGLPLVGETLQLARPSDSLDVHPFIKKKVKRYGPIFKTCLAGRPVVVSTDAEFNNYIMLQEGRAVEMWYLDTLSKFFGLDTEWLKALGFIHKYIRSITLNHFGAESLRERFLPRIEESAKETLRYWATQPSVEVKDSAAVMVFRTSMVKMVSEDSSKLLTGGLTKKFTGLLGGFLTLPINVPGTTYNKCMKDMKEIQKKLREILEGRLASGGGSDEDFLGQAIKDKGSQQFISDDFIIQLLFSISFASFESISTTLTLVLNYLADHPDVVKELEAEHEAIRNARADPDGPITWEEYKSMTFTLHVIFETLRLGSVTPALLRKTTKELQINGYTIPEGWTVMLVTASRHRDPAVYKDPHTFNPWRWKELDSITIQKNFMPFGGGLRHCAGAEYSKVYLCTFLHILFTKYRWTKLKGGKVARAHILSFEDGLHVKFTPKE

SEQ ID NO: 201 - protein, 709 aa, synthetic construct
MAQSSSVKVSTFDLMSAILRGRSMDQTNVSFESGESPALAMLIENRELVMILTTSVAVLIGCFVVLLWRRSSGKSGKVTEPPKPLMVKTEPEPEVDDGKKKVSIFYGTQTGTAEGFAKALAEEAKVRYEKASFKVIDLDDYAADDEEYEEKLKKETLTFFFLATYGDGEPTDNAARFYKWFMEGKERGDWLKNLHYGVFGLGNRQYEHFNRIAKVVDDTIAEQGGKRLIPVGLGDDDQCIEDDFAAWRELLWPELDQLLQDEDGTTVATPYTAAVLEYRVVFHDSPDASLLDKSFSKSNGHAVHDAQHPCRANVAVRRELHTPASDRSCTHLEFDISGTGLVYETGDHVGVYCENLIEVVEEAEMLLGLSPDTFFSIHTDKEDGTPLSGSSLPPPFPPCTLRRALTQYADLLSSPKKSSLLALAAHCSDPSEADRLRHLASPSGKDEYAQWVVASQRSLLEVMAEFPSAKPPIGAFFAGVAPRLQPRYYSISSSPRMAPSRIHVTCALVFEKTPVGRIHKGVCSTWMKNAVPLDESRDCSWAPIFVRQSNFKLPADTKVPVLMIGPGTGLAPFRGFLQERLALKEAGAELGPAILFFGCRNRQMDYIYEDELNNFVETGALSELIVAFSREGPKKEYVQHKMMEKASDIWNMISQEGYIYVCGDAKGMARDVHRTLHTIVQEQGSLDSSKTESMVKNLQMNGRYLRDVW

SEQ ID NO: 202 - protein, 496 aa, synthetic construct
MATEKTHQFHPSLHFVLFPFMAQGHMIPMIDIARLLAQRGVTITIVTTPHNAARFKNVLNRAIESGLAINILHVKFPYQEFGLPEGKENIDSLDSTELMVPFFKAVNLLEDPVMKLMEEMKPRPSCLISDWCLPYTSIIAKNFNIPKIVFHGMGCFNLLCMHVLRRNLEILENVKSDEEYFLVPSFPDRVEFTKLQLPVKANASGDWKEIMDEMVKAEYTSYGVIVNTFQELEPPYVKDYKEAMDGKVWSIGPVSLCNKAGADKAERGSKAAIDQDECLQWLDSKEEGSVLYVCLGSICNLPLSQLKELGLGLEESRRSFIWVIRGSEKYKELFEWMLESGFEERIKERGLLIKGWAPQVLILSHPSVGGFLTHCGWNSTLEGITSGIPLITWPLFGDQFCNQKLVVQVLKAGVSAGVEEVMKWGEEDKIGVLVDKEGVKKAVEELMGDSDDAKERRRRVKELGELAHKAVEKGGSSHSNITLLLQDIMQLAQFKN

SEQ ID NO: 203 - protein, 438 aa, synthetic construct
MAKEEFDICIIGAGMAGATISAYLAPKGIKIALIDRCYKEKKRIVGELLQPGAVLSLEQMGLSHLLDGFEAQTVKGYALLQGNEKTTIPYPSQHEGIGLHNGRFLQQIRASALENSSVTQIHGKALQLLENERNEIIGVSYRESITSQIKSIYAPLTITSDGFASNFRAHLSNNQKTVTSYFIGLILKDCEMPFPKHGHVFLSGPTPFICYPISDNEVRLLIDFPGEQLPRKNLLQEHLDTNVTPYIPECMRSSYAQAIQEGGFKVMPNHYMAAKPIVRKGAVLLGDALNMRHPLTGGGLTAVFSDIQILSAHLLAMPDFKNTDLIHEKIEAYYRDRKRANANLNILANALYAVMSNDLLKTAVFKYLQCGGANAQESIALLAGLNRKHFSLIKQYCFLAVFGACNLLQQSISNIPKALKLLKDAFVIIKPLIKNELS

SEQ ID NO: 204 - protein, 466 aa, synthetic construct
MAQAESERMRVVMFPWLAHGHINPYLELAKRLIASASGDHHLDVVVHLVSTPANLAPLAHHQTDRLRLVELHLPSLPDLPPALHTTKGLPARLMPVLKRACDLAAPRFGALLDELCPDILVYDFIQPWAPLEAEARGVPAFHFATCGAAATAFFIHCLKTDRPPSAFPFESISLGGVDEDAKYTALVTVREDSTALVAERDRLPLSLERSSGFVAVKSSADIERKYMEYLSQLLGKEIIPTGPLLVDSGGSEEQRDGGRIMRWLDGEEPGSVVFVSFGSEYFMSEHQMAQMARGLELSGVPFLWVVRFPNAEDDARGAARSMPPGFEPELGLVVEGWAPQRRILSHPSCGAFLTHCGWSSVLESMAAGVPMVALPLHIDQPLNANLAVELGAAAARVKQERFGEFTAEEVARAVRAAVKGKEGEAARRRARELQEVVARNNGNDGQIATLLQRMARLCGKDQAVPN

SEQ ID NO: 205 - protein, 476 aa, synthetic construct
MAEANDGGKMHVVMLPWLAFGHVLPFTEFAKRVARQGHRVTLLSAPRNTRRLIDIPPGLAGLIRVVHVPLPRVDGLPEHAEATIDLPSDHLRPCLRRAFDAAFERELSRLLQEEAKPDWVLVDYASYWAPTAAARHGVPCAFLSLFGAAALSFFGTPETLLGIGRHAKTEPAHLTVVPEYVPFPTTVAYRGYEARELFEPGMVPDDSGVSEGYRFAKTIEGCQLVGIRSSSEFEPEWLRLLGELYRKPVIPVGLFPPAPQDDVAGHEATLRWLDGQAPSSVVYAAFGSEVKLTGAQLQRIALGLEASGLPFIWAFRAPTSTETGAASGGLPEGFEERLAGRGVVCRGWVPQVKFLAHASVGGFLTHAGWNSIAEGLAHGVRLVLLPLVFEQGLNARNIVDKNIGVEVARDEQDGSFAAGDIAAALRRVMVEDEGEGFGAKVKELAKVFGDDEVNDQCVREFLMHLSDHSKKNQGQD

SEQ ID NO: 206 - protein, 463 aa, synthetic construct; identical to SEQ ID NO: 163

SEQ ID NO: 207 - protein, 453 aa, synthetic construct
MAENHATFNVLMLPWLAHGHVSPYLELAMKLTARNFNVYLCSSPATLSSVRSKLTEKFSQSIHLVELHLPKLPELPAEYHTTNGLPPHLMPTLKDAFDMAKPNFCNVLKSLKPDLLIYDLLQPWAPEAASAFNIPAVVFISSSATMTSFGLHFFKNPGTKYPYGNTIFYRDYESVFVENLKKRDRDTYRVVNCMERSSKIILIKGFKEIEGKYFDYFSCLTGKKVVPVGPLVQDPVLDDEDCRIMQWLNKKEKGSTVFVSFGSEYFLSKEDMEEIAHGLELSNVDFIWVVRFPKGENIVIEETLPKGFFERVGERGLVVNGWAPQAKILTHPNVGGFVSHCGWNSVMESMKFGLPIVAMPMHLDQPINARLIEEVGAGVEVLRDSKGKLHRERMAETINKVTKEASGEPARKKARELQEKLELKGDEEIDDVVKELVQLCATKNKRNGLHCYN

SEQ ID NO: 208 - protein, 452 aa, synthetic construct
MAENHATFNVLMLPWLAHGHVSPYLELAKKLTARNFNVYLCSSPATLSSVRSKLTEKFSQSIHLVELHLPKLPELPAEYHTTNGLPPHLMPTLKDAFDMAEPNFCNVLKSLKPDLLIYDLLQPWAPEAASAFNIPAVVFISSSATMTSFGLHFFKNPGTKYPYGNTIFYRDYESVFVENLKRRDRDTYRVVNCMERSSKIILIKGFKEIEGKYFDYFSCLTGKKVVPVGPLVQDPVLDDEDCRIMQWLNKKEKGSTVFVSFGSEYFLSKEDMEEIAHGLELSNVDFIWVVRFPKGENIVIEETLPKGFFERVGERGLVVNGWAPQAKILTHPNVGGFVSHCGWNSVMESMKFGLPIIAMPMHLDQPINARLIEEVGAGVEVLRDSKGKLHRERMAETINKVTKEASGESVRKKARELQEKLELKGDEEIDDVVKELVQLCATKNKRNGLHYN

SEQ ID NO: 209 - protein, 452 aa, synthetic construct
MAENHATFNVLMLPWLAHGHVSPYLELAKKLTARNFNVYLCSSPATLSSVRSKLTEKFSQSIHLVELHLPKLPELPAEYHTTNGLPPHLMPTLKDAFDMAKPNFCNVLKSLKPDLLIYDLLQPWAPEAASAFNIPAVVFISSSATMTSFGLHFFKNPGTKYPYGNAIFYRDYESVFVENLTRRDRDTYRVINCMERSSKIILIKGFNEIEGKYFDYFSCLTGKKVVPVGPLVQDPVLDDEDCEIMQWLNKKEKVSTVFVSFGSEYFLSKKDMEEIAHGLELSNVDFIWVVRFPKGENIVIEETLPKGFFERVGERGLVVNGWAPQAKILTHPNVGGFVSHCGWNSVMESMKFGLPIIAMPMHLDQPINARLIEEVGAGVEVLRDSKGKLHRERMAETINKVMKEASGESVRKKARELQEKMDLKGDEEIDDVVKELVQLCATKNKRNGLHYY

SEQ ID NO: 210 - protein, 453 aa, synthetic construct
MADAAQQGDTTTILMLPWLGYGHLSAFLELAKSLSRRNFHIYFCSTSVNLDAIKPKLPSSFSDSIQFVELHLPSSPEFPPHLHTTNGLPPTLMPALHQAFSMAAQHFESILQTLAPHLLIYDSLQPWAPRVASSLKIPAINFNTTGVFVISQGLHPIHYPHSKFPFSEFVLHNHWKAMYSTADGASTERTRKRGEAFLYCLHASCSVILINSFRELEGKYMDYLSVLLNKKVVPVGPLVYEPNQDGEDEGYSSIKNWLDKKEPSSTVFVSFGSEYFPSKEEMEEIAHGLEASEVNFIWVVRFPQGDNTSGIEDALPKGFLERAGERGMVVKGWAPQAKILKHWSTGGFVSHCGWNSVMESMMFGVPIIGVPMHVDQPFNAGLVEEAGVGVEAKRDPDGKIQRDEVAKLIKEVVVEKTREDVRKKAREMSEILRSKGEEKFDEMVAEISLLLKI

SEQ ID NO: 211 - protein, 474 aa, synthetic construct
MAQAERERLRVLMFPWLAHGHINPYLELATRLTTTSSSQIDVVVHLVSTPVNLAAVAHRRTDRISLVELHLPELPGLPPALHTTKHLPPRLMPALKRACDLAAPAFGALLDELSPDVVLYDFIQPWAPLEAAARGVPAVHFSTCSAAATAFFLHFLDGGGGGGGRGAFPFEAISLGGAEEDARYTMLTCRDDGTALLPKGERLPLSFARSSEFVAVKTCVEIESKYMDYLSKLVGKEIIPCGPLLVDSGDVSAGSEADGVMRWLDGQEPGSVVLVSFGSEYFMTEKQLAEMARGLELSGAAFVWVVRFPQQSPDGDEDDHGAAAARAMPPGFAPARGLVVEGWAPQRRVLSHRSCGAFLTHCGWSSVMESMSAGVPMVALPLHIDQPVGANLAAELGVAARVRQERFGEFEAEEVARAVRAVMRGGEALRRRATELREVVARRDAECDEQIGALLHRMARLCGKGTGRAAQLGH

SEQ ID NO: 212 - protein, 357 aa, synthetic construct
MASQVHRILNCRGTRIHAVADSPPDQQGPLVVLLHGFPESWYSWRHQIPALAGAGYRVVAIDQRGYGRSSKYRVQKAYRIKELVGDVVGVLDSYGAEQAFVVGHDWGAPVAWTFAWLHPDRCAGVVGISVPFAGRGVIGLPGSPFGERRPSDYHLELAGPGRVWYQDYFAVQDGIITEIEEDLRGWLLGLTYTVSGEGMMAATKAAVDAGVDLESMDPIDVIRAGPLCMAEGARLKDAFVYPETMPAWFTEADLDFYTGEFERSGFGGPLSFYHNIDNDWHDLADQQGKPLTPPALFIGGQYDVGTIWGAQAIERAHEVMPNYRGTHMIADVGHWIQQEAPEETNRLLLDFLGGLRP

SEQ ID NO: 213 - protein, 443 aa, synthetic construct
MADNQNGRISIALLPFLAHGHISPFFELAKQLAKRNCNVFLCSTPINLSSIKDKDSSASIKLVELHLPSSPDLPPHYHTTNGLPSHLMLPLRNAFETAGPTFSEILKTLNPDLLIYDFNPSWAPEIASSHNIPAVYFLTTAAASSSIGLHAFKNPGEKYPFPDFYDNSNITPEPPSADNMKLLHDFIACFERSCDIILIKSFRELEGKYIDLLSTLSDKTLVPVGPLVQDPMGHNEDPKTEQIINWLDKRAESTVVFVCFGSEYFLSNEELEEVAIGLEISTVNFIWAVRLIEGEKKGILPEGFVQRVGDRGLVVEGWAPQARILGHSSTGGFVSHCGWSSIAESMKFGVPVIAMARHLDQPLNGKLAAEVGVGMEVVRDENGKYKREGIAEVIRKVVVEKSGEVIRRKARELSEKMKEKGEQEIDRALEELVQICKKKKDEQ

SEQ ID NO: 214 - protein, 499 aa, synthetic construct
MAHHHHHHVGTGSNDDDDKSPDPNWASTSELVFIPSPGAGHLPPTVELAKLLLHRDQRLSVTIIVMNLWLGPKHNTEARPCVPSLRFVDIPCDESTMALISPNTFISAFVEHHKPRVRDIVRGIIESDSVRLAGFVLDMFCMPMSDVANEFGVPSYNYFTSGAATLGLMFHLQWKRDHEGYDATELKNSDTELSVPSYVNPVPAKVLPEVVLDKEGGSKMFLDLAERIRESKGIIVNSCQAIERHALEYLSSNNNGIPPVFPVGPILNLENKKDDAKTDEIMRWLNEQPESSVVFLCFGSMGSFNEKQVKEIAVAIERSGHRFLWSLRRPTPKEKIEFPKEYENLEEVLPEGFLKRTSSIGKVIGWAPQMAVLSHPSVGGFVSHCGWNSTLESMWCGVPMAAWPLYAEQTLNAFLLVVELGLAAEIRMDYRTDTKAGYDGGMEVTVEEIEDGIRKLMSDGEIRNKVKDVKEKSRAAVVEGGSSYASIGKFIEHVSNVTI

SEQ ID NO: 215 - protein, 463 aa, synthetic construct
MADSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 216 - protein, 494 aa, synthetic construct
MASEKTLQVHPPLHFVLFPFMAQGHMIPMVDIARLLAQRGATVTIVTTRYNAGRFENVLSRAVESGLPINIVHVKFPYEEVGLPKGKENIDSLDSMELMVPFFKAVNMLQDPVVKLMEEMESRPSCIISDLLLPYTSKIAKKFNIPKIVFHGISCFCLLCVHVLRRNLEILTNLKSDKEYFLVPSFPDRVEFTKPQVTVETNASGDWKEFLDEMVEAEDTSYGVIINTFEELEPAYVKDYKDARAGNVWSIGPVSLCNKAGVDKAERGNKATIDQDECLKWLDSKEEGSVLYVCLGSICNLPLVQLKELGLGLEESQRPFIWVIRGWEKYNELSEWMVESGFEERIRERGLLIRGWAPQVLILSHPSVGGFLTHCGWNSTVEGITSGVPLITWPLFGDQFCNQTLVVQVLKAGVSVGVEEVMKWGEEEKIGVLVDKEGVKKAVEDLMGESDDAKERTKRVKELGGLAHKAVEEGGSSHSNITLFLQDIRQVQSV

SEQ ID NO: 217 - protein, 478 aa, synthetic construct
MADVAEEQPLKIYFIPYLAAGHMIPLCDIATLFASRGHHVTIITTPSNAQTLRESHHFRVQTIQFPSQEVGLPAGVQNLTAVTNLDDSYKIYHATMLLRKHIEDFVERDPPDCIVADFLFPWVDDVATKLHIPRLVFNGFTLFTICAMESHKAHPLPVDAASGSFVIPDFPHHVTINSTPPKRTKEFVDPLLTEAFKSHGFLINSFVELDGEECVEHYERITGGHKAWHLGPAFLVHRTAQDRGEKSVVSTQECLSWLDSKRDNSVLYICFGTICYFPDKQLYEIASAIEASGHEFIWVVPEKRGNADESEEEKEKWLPKGFEERNNGKKGMIIRGWAPQVAILGHPAVGGFLTHCGWNSTVEAVSAGVPMITWPVHSDQYFNEKLITQVRGIGVEVGAEEWIVTAFRETEKLVGRDRIERAVRRVMDGGDEAVQIRRRARELGEMARQAVQEGGSSHTNLTALINDLKRWRDSKQLN

SEQ ID NO: 218 - protein, 491 aa, synthetic construct
MAVFQANQPHFVLFPLMAQGHIIPMIDIARLLAQRGAIVTIFTTPKNASRFTSVLSRAVSSGLQIRLVHLHFPSKEAGLPEGCENLDMVASHDMICNIFQAIRMLQKQAEELFETLTPKPSCIISDFCIPWTTQVAEKHHIPRISFHGFSCFCLHCMLKIHTSKVLEGITSESEYFTVPGIPDQIQVTKQQVPGPMIDEMKEFGEQMRDAEIRSYGVIINTFEELEKAYVNDYKKERNGKVWCIGPVSLCNKDGLDKAQRGNKASISEHHCLEWLDLQQPNSVIYVCLGSLCNLTPPQLMELALGLEATKRPFIWVIREGNKFEELEKWISEEGFEERIKGRGLIIRGWAPQVLILSHPSIGGFLTHCGWNSTLEGVTAGVPMVTWPLFADQFLNEKLVTQVLRIGVSLGVDVPLKWGEEEKVGVQVKKEGIEKAICMVMDEGEESKERRERAKELSEMAKRAVEKDGSSHLNMTMLIQDIMQQSSSKVET

SEQ ID NO: 219 - protein, 759 aa, synthetic construct
MAWKLKIAEGGDPWLRTTNDHIGRQIWEFDPTLGSVEELAEIEKLRKTFRDNRFEKKHSADLLMRSQFAKENSVSVFPPKVNIKDVEDITEDKVTNVLRRAIGFHSTLQADDGHWPGDLGGPMFLLPGLVITLSITGALNAVLSKEHKREMCRYLYNHQNIDGGWGLHIEGHSTMFGSALNYVTLRLLGEGANDGEGAMEKGRKWILDHGGATAITSWGKFWLSVLGVFEWPGNNPLPPEMWLLPYFLPVHPGRMWCHCRMVYLPMSYLYGKRFVGPITSTVLALRKELFTVPYHDIDWNEARNLCAKEDLYYPHPLIQDVLWATLDKFVEPVLMSWPGKKLREKALRTAMEHIHYEDENTRYICIGPVNKVLNMLCCWVEDPNSEAFKLHLPRIQDYLWIAEDGMKMQGYNGSQLWDAAFTVQAIMSTNLIEEFGPTLKKGHIFIKKSQVLDNCYGDLDYWYRHISKGAWPFSTADHGWPISDCTAEGLKAALLLSKLPSEIVDEPLDAKRFYDAVNVILSLMNADGSFATYELTRSYSWLELINPAETFGDIVIDYPYVECTSAAIQALVAFKRLYPGHRRDEVQGCIDKAAAFLEKIQEADGSWYGSWAVCFTYGTWFGVKGLVAAGKNYSNCSSIRKACNFLLSKQLASGGWGESYLSCVDKVYTNLEGNRSHVVNTGWAMLALIDAEQAKRDPTPLHRAARVLINSQMENGEFPQQEIMGVFNRNCMITYAAYRNIFPIWALGEYRCRVLKVET

SEQ ID NO: 220 - protein, 765 aa, synthetic construct
MAWRLKVGAESVGEKEEKWLKSISNHLGRQVWEFCAHQPTASPNHLQQIDNARNHFRNNRFHRKQSSDLFLAIQNEKEIANVTKGGGIKVKEEEDVRKETVKNTVERALSFYSAIQTNDGNWASDLGGPMFLLPGLVIALYVTGVLNSVLSKHHRQEMCRYLYNHQNEDGGWGLHIEGTSTMFGSALNYVALRLLGEDADGGEGGAMTKARSWILDRGGATAITSWGKLWLSVLGVYEWSGNNPLPPEFWLLPYCLPFHPGRMWCHCRMVYLPMSYLYGKRFVGPITPIVLSLRKELYTIPYHEIDWNRSRNTCAKEDLYYPHPKMQDILWGSIYHLYEPLFTRWPGKRLREKALQMAMKHIHYEDENSRYICLGPVNKVLNMLCCWVEDPYSDAFKFHLQRVPDYLWVAEDGMRMQGYNGSQLWDTAFSVQAIISTKLIDSFGTTLKKAHDFVKDSQIQQDCPGDPNVWFRHIHKGAWPFSTRDHGWLISDCTAEGLKASLMLSKLPSKIVGEPLEKSRLCDAVNVLLSLQNENGGFASYELTRSYPWLELINPAETFGDIVIDYPYVECTSATMEALTLFKKLHPGHRTKEIDIAVARAANFLENMQRTDGSWYGCWGVCFTYAGWFGIKGLVAAGRTYNSCVAIRKACDFLLSKELPGGGWGESYLSCQNKVYTNLEGNRPHLVNTAWVLMALIEAGQAERDPAPLHRAARLLINSQLENGDFPQEEIMGVFNKNCMITYAAYRNIFPIWALGEYFHRVLTE

SEQ ID NO: 221 - protein, 520 aa, synthetic construct
MEMSSSVAATISIWMVVVCIVGVGWRVVNWVWLRPKKLEKRLREQGLAGNSYRLLFGDLKERAAMEEQANSKPINFSHDIGPRVFPSMYKTIQNYGKNSYMWLGPYPRVHIMDPQQLKTVFTLVYDIQKPNLNPLIKFLLDGIVTHEGEKWAKHRKIINPAFHLEKLKDMIPAFFHSCNEIVNEWERLISKEGSCELDVMPYLQNLAADAISRTAFGSSYEEGKMIFQLLKELTDLVVKVAFGVYIPGWRFLPTKSNNKMKEINRKIKSLLLGIINKRQKAMEEGEAGQSDLLGILMESNSNEIQGEGNNKEDGMSIEDVIEECKVFYIGGQETTARLLIWTMILLSSHTEWQERARTEVLKVFGNKKPDFDGLSRLKVVTMILNEVLRLYPPASMLTRIIQKETRVGKLTLPAGVILIMPIILIHRDHDLWGEDANEFKPERFSKGVSKAAKVQPAFFPFGWGPRICMGQNFAMIEAKMALSLILQRFSFELSSSYVHAPTVVFTTQPQHGAHIVLRKL